SEQUENCE LISTING (718 sequences)

SEQ ID NO: 1-17 - skipped (17 entries)

SEQ ID NO: 18 - DNA, 86 bp, synthetic construct
  source 1..86: mol_type = other DNA
gtttaagagctatgctggaaacagcatagcaagtttaaataaggctagtccgttatcaacttgaaaaagtggcaccgagtcggtgc

SEQ ID NO: 19 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 20 - protein, 7 aa, synthetic construct
PKKKRKV

SEQ ID NO: 21 - protein, 5 aa, synthetic construct
GGGGS

SEQ ID NO: 22 - protein, 5 aa, synthetic construct
GGGGG

SEQ ID NO: 23 - protein, 5 aa, synthetic construct
GGAGG

SEQ ID NO: 24 - protein, 7 aa, synthetic construct
GGGGSSS

SEQ ID NO: 25 - protein, 7 aa, synthetic construct
GGGGAAA

SEQ ID NO: 26 - protein, 1368 aa, Streptococcus pyogenes [sequence omitted]

SEQ ID NO: 27 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 28 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - DNA, 3159 bp, synthetic construct
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 31 - DNA, 3159 bp, synthetic construct
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 32 - DNA, 4107 bp, synthetic construct
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 33 - DNA, 3158 bp, synthetic construct
  source 1..3158: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 34 - DNA, 3159 bp, synthetic construct
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 35 - DNA, 3159 bp, synthetic construct
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 36 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 37 - DNA, 3242 bp, synthetic construct
  source 1..3242: mol_type = other DNA
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 38 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 39 - DNA, 3156 bp, synthetic construct
  source 1..3156: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 40 - DNA, 7009 bp, synthetic construct
  source 1..7009: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 41 - protein, 2414 aa, synthetic construct
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 42 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 43 - protein, 1497 aa, synthetic construct
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 44 - DNA, 4491 bp, synthetic construct
  source 1..4491: mol_type = other DNA
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 45 - protein, 65 aa, synthetic construct
RTLVTFKDVFVDFTREEWKLLDTAQQILYRNVMLENYKNLVSLGYQLTKPDVILRLEKGEEPWLV

SEQ ID NO: 46 - DNA, 195 bp, synthetic construct
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 47 - protein, 1519 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 48 - DNA, 4560 bp, synthetic construct
  source 1..4560: mol_type = other DNA
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 49 - protein, 1190 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 50 - DNA, 3570 bp, synthetic construct
  source 1..3570: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 51 - protein, 718 aa, synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 52 - DNA, 2154 bp, synthetic construct
  source 1..2154: mol_type = other DNA
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 53 - protein, 378 aa, synthetic construct
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 54 - DNA, 1134 bp, synthetic construct
  source 1..1134: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 55 - protein, 522 aa, synthetic construct
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - DNA, 1566 bp, synthetic construct
  source 1..1566: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 57 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 58 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 59 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 60 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 61 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 62 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 63 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTD

SEQ ID NO: 64 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 65 - DNA, 498 bp, synthetic construct
  source 1..498: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgac

SEQ ID NO: 66 - DNA, 498 bp, synthetic construct
  source 1..498: mol_type = other DNA
tccgaagtcgagttttcccatgagtactggatgagacacgcattgactctcgcaaagagggctctcgatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtaacgcgctgttgtgttacttttttcgcatgcccaggcaggtctttaacgcccagaaaaaagcacaatcctctactgac

SEQ ID NO: 67 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgac

SEQ ID NO: 68 - DNA, 498 bp, synthetic construct
  source 1..498: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 69 - DNA, 594 bp, synthetic construct
  source 1..594: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttct

SEQ ID NO: 70 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgac

SEQ ID NO: 71 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcgtgtctttaacgcccagaaaaaagcacaatcctctactgac

SEQ ID NO: 72 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgac

SEQ ID NO: 73 - DNA, 1981 bp, synthetic construct
  source 1..1981: mol_type = other DNA
ctcttctgtcaaccccacgcgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagggactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttggaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactgtctatccgtgttcctgaaccagttatgtgtgttgcatgagaaaacgccagtaagtgacagagtcaccaaatgctgcacagaatccttggtgaacgggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaa

SEQ ID NO: 74 - DNA, 1257 bp, synthetic construct
  source 1..1257: mol_type = other DNA
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 75 - DNA, 1277 bp, synthetic construct
  source 1..1277: mol_type = other DNA
agggccctcctatggaccctgcccgctcccctcccattgtccacggctgtccgcccacccccattctccaagcttcagccccctccttagttcggcatctgcacagcactgaagaacctgggaatcagaccctgagaccctgagcaatcccaggtccagcgccagccctatcatgaccaaggagtatcaagaccttcagcatctggacaatgaggagagtgaccaccatcagctcagaaaagggccacctcctccccagcccctcctgcagcgtctctgctccggacctcgcctcctcctgctctccctgggcctcagcctcctgctgcttgtggttgtctgtgtgatcggatcccaaaactcccagctgcaggaggagctgcggggcctgagagagacgttcagcaacttcacagcgagcacggaggcccaggtcaagggcttgagcacccagggaggcaatgtgggaagaaagatgaagtcgctagagtcccagctggagaaacagcagaaggacctgagtgaagatcactccagcctgctgctccacgtgaagcagttcgtgtctgacctgcggagcctgagctgtcagatggcggcgctccagggcaatggctcagaaaggacctgctgcccggtcaactgggtggagcacgagcgcagctgctactggttctctcgctccgggaaggcctgggctgacgccgacaactactgccggctggaggacgcgcacctggtggtggtcacgtcctgggaggagcagaaatttgtccagcaccacataggccctgtgaacacctggatgggcctccacgaccaaaacgggccctggaagtgggtggacgggacggactacgagacgggcttcaagaactggaggccggagcagccggacgactggtacggccacgggctcggaggaggcgaggactgtgcccacttcaccgacgacggccgctggaacgacgacgtctgccagaggccctaccgctgggtctgcgagacagagctggacaaggccagccaggagccacctctcctttaatttatttcttcaatgcctcgacctgccgcaggggtccgggattgggaatccgcccatctggggcctcttctgctttctcgggaattttcatctaggattttaagggaaggggaaggatagggtgatgttccgaaggtgaggagcttgaaacccgtggcgctttctgcagttcacaatgataacctgcaaactgcagaaagcgccacgggtttcaagctcctcaccttcg

SEQ ID NO: 76 - DNA, 1944 bp, synthetic construct
  source 1..1944: mol_type = other DNA
gcggccgcgtcgacgctagaggggctgcttgcgccaggcgccggccgccccactgcgggtccctggcggccggtgtctgaggagtcggagagccgaggcggccagaccgtgcgccccgcgcttctcccgaggccgttccgggtctgaactgtaacagggaggggcctcgcaggagcagcagcgggcgagttaaagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtnttactt

SEQ ID NO: 77 - DNA, 2289 bp, synthetic construct
  source 1..2289: mol_type = other DNA
gcagagagggcactgggaggaggcagtgggagggcggagggcgggggccttcggggtgggcgcccagggtagggcaggtggccgcggcgtggaggcagggagaatgcgactctccaaaaccctcgtcgacatggacatggccgactacagtgctgcactggacccagcctacaccaccctggaatttgagaatgtgcaggtgttgacgatgggcaatgatttgttgccgctgcgtctcgccagattgaggcatcccctccgacatcactggagcatatctggaggggtggacagttctccacagggagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacgtggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctagcaagccgctggggcttgggggctccactggctccccccagccccctaagagagcacctggtgatcacgtggtcacggcaaaggaagacgtgatgccaggaccagtcccagagcaggaatgggaaggatgaagggcccgagaacatggcctaaggcacatcccactgcaccctgacgccctgctctgataacaagactttgacttggggagaccctctactgccttggacaactttctcatgttgaagccactgccttcaccttcaccttcatccatgtccaacccccgacttcatcccaaaggacagccgcctggagatgacttgagccttacttaaacccagctcccttcttccctagcctggtgcttctcctctcctagccccggtcatggtgtccagacagagccctgtgaggctgggtccaattgtggcacttggggcaccttgctcctccttctgctgctgcccccacctctgctgcctccctctgctgtcaccttgctcagccatcccgtcttctccaacaccacctctacagaggccaaggaggccttggaaacgattcccccagtcattctgggaacatgttgtaagcactgactgggaccaggcaccaggcagggtctagaaggctgtggtgagggaagacgcctttctcctccaacccaac

SEQ ID NO: 78 - protein, 609 aa, synthetic construct [sequence omitted]

SEQ ID NO: 79 - protein, 418 aa, synthetic construct
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 80 - protein, 291 aa, synthetic construct
MTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 81 - protein, 457 aa, synthetic construct
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 82 - protein, 474 aa, synthetic construct
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 83 - DNA, 405 bp, synthetic construct
  source 1..405: mol_type = other DNA
atgatgctgagtccggaccaagcagcagattcggaccatcccagctcggcgcactcggatccggagtccctgggcggcacggacaccaaggtgctcggcagcgtgtcggacctggagccggtggaggaggccgagggcgacggcaagggcggcagccgagccgcgctctacccgcacccgcagcagctgagccgcgaggagaagcgccgccgccggcgcgccacggccaagtaccgctcggcccacgccacccgcgagcgcatccgcgtggaagccttcaacttggccttcgccgagctccgcaaattgctgcccacgctgcccccggacaagaagctctccaagatcgagatcctgcgcctggccatctgctacatctcctatctcaaccacgtcctggacgtg

SEQ ID NO: 84 - DNA, 906 bp, synthetic construct
  source 1..906: mol_type = other DNA
atggacgtgttggctagttatagtatattccaggagctacaacttgtccacgacaccggctacttctcagctttaccatccctggaggagacctggcagcagacatgccttgaattggaacgctacctacagacggagccccggaggatctcagagacctttggtgaggacttggactgtttcctccacgcttcccctcccccgtgcattgaggaaagcttccgtcgcttagaccccctgctgctccccgtggaagcggccatctgtgagaagagctcggcagtggacatcttgctctctcgggacaagttgctatctgagacctgcctcagcctccagccggccagctcttctctagacagctacacagccgtcaaccaggcccagctcaacgcagtgacctcattaacgcccccatcgtcccctgagctcagccgccatctggtcaaaacctcacaaactctctctgccgtggatggcacggtgacgttgaaactggtggccaagaaggctgctctcagctccgtaaaggtgggaggggtcgcaacagctgcagcagccgtgacggctgcgggggccgttaagagtggacagagcgacagtgaccaaggagggctgggggctgaagcatgtcccgaaaacaagaagagggttcaccgctgtcagtttaacgggtgccggaaagtttatacaaaaagctcccacttaaaggcccaccagaggactcacacaggtgagaagccttataagtgctcatgggagggatgtgagtggcgttttgcacgaagcgatgagctcacgaggcactacaggaaacacacaggtgcaaagcccttcaaatgcaaccactgcgacaggtgtttttccaggtctgaccatcttgccctccacatgaagagacatatc

SEQ ID NO: 85 - DNA, 615 bp, synthetic construct
  source 1..615: mol_type = other DNA
atggcggcagcggctgggggcgcggcgagccgccggggtgccgggcggccctgccccttctccatcgagcacatcctctccagcctgcccgagcggagcctcccggcccgggccgcctgcccaccgcagcccgccggtcgccagagccccgcgaagccagaggagcccggggcgcccgaggctgcgccctgcgcctgctgctgctgctgcggcccccgcgcggcgccctgcgggcccccagaggcggccgccgggctgggcgctcgtctggcgtggccgctgaggctgggaccggcggtgcccttgtctctgggtgcgccagccggaggttccggggcgctcccgggcgcggtcggcccgggttcgcagcggcgcacgaggcgccaccgcaccatcttcagcgaagagcagctgcaggcgctcgaggcgcttttcgtgcagaaccagtatcctgacgtgagtacgcgcgagcgcctggccggccgcatccgccttcgcgaggagcgcgtggaggtctggttcaagaaccgccgggccaaatggcgacaccagaagcgcgcgtcggcttccgcgaggctcctgcccggcgtcaagaagtccccgaaggggagctgc

SEQ ID NO: 86 - DNA, 1515 bp, synthetic construct
  source 1..1515: mol_type = other DNA
atgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacatgtgcagctactgcaggtgatcaagaagacggagacagacatgagtcttcacccgctcctgcaggagatctacaaggacttgtac

SEQ ID NO: 87 - DNA, 1848 bp, synthetic construct
  source 1..1848: mol_type = other DNA
atgtcgaggcgcaagcaggcgaaaccccagcacatcaactcggaggaggaccagggcgagcagcagccgcagcagcagaccccggagtttgcagatgcggccccagcggcgcccgcggcgggggagctgggtgctccagtgaaccacccagggaatgacgaggtggcgagtgaggatgaagccacagtaaagcggcttcgtcgggaggagacgcacgtctgtgagaaatgctgtgcggagttcttcagcatctctgagttcctggaacataagaaaaattgcactaaaaatccacctgtcctcatcatgaatgacagcgaggggcctgtgccttcagaagacttctccggagctgtactgagccaccagcccaccagtcccggcagtaaggactgtcacagggagaatggcggcagctcagaggacatgaaggagaagccggatgcggagtctgtggtgtacctaaagacagagacagccctgccacccaccccccaggacataagctatttagccaaaggcaaagtggccaacactaatgtgaccttgcaggcactacggggcaccaaggtggcggtgaatcagcggagcgcggatgcactccctgcccccgtgcctggtgccaacagcatcccgtgggtcctcgagcagatcttgtgtctgcagcagcagcagctacagcagatccagctcaccgagcagatccgcatccaggtgaacatgtgggcctcccacgccctccactcaagcggggcaggggccgacactctgaagaccttgggcagccacatgtctcagcaggtttctgcagctgtggctttgctcagccagaaagctggaagccaaggtctgtctctggatgccttgaaacaagccaagctacctcacgccaacatcccttctgccaccagctccctgtccccagggctggcacccttcactctgaagccggatgggacccgggtgctcccgaacgtcatgtcccgcctcccgagcgctttgcttcctcaggccccgggctcggtgctcttccagagccctttctccactgtggcgctagacacatccaagaaagggaaggggaagccaccgaacatctccgcggtggatgtcaaacccaaagacgaggcggccctctacaagcacaagtgtcggagcagtctcccttccacgtttatccgagccccgccgacctatgtcaaggttgaagttcctggcacatttgtgggaccctcgacattgtccccagggatgacccctttgttagcagcccagccacgccgacaggccaagcaacatggctgcacacggtgtgggaagaacttctcgtctgctagcgctcttcagatccacgagcggactcacactggagagaagccttttgtgtgcaacatttgtgggcgagcttttaccaccaaaggcaacttaaaggttcactacatgacacacggggcgaacaataactcagcccgccgtggaaggaagttggccatcgagaacaccatggctctgttaggtacggacggaaaaagagtctcagaaatctttcccaaggaaatcctggccccttcagtgaatgtggaccctgttgtgtggaaccagtacaccagcatgctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacaccagtttcctcacttcctggaagaaaacaagattgcggtcagc

SEQ ID NO: 88 - DNA, 1326 bp, synthetic construct
  source 1..1326: mol_type = other DNA
atgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccattgctctggtctgatcctgcagataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtc

SEQ ID NO: 89 - DNA, 1305 bp, synthetic construct
  source 1..1305: mol_type = other DNA
atgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcatttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtc

SEQ ID NO: 90 - DNA, 1410 bp, synthetic construct
  source 1..1410: mol_type = other DNA
atgtggcgagaatgtgattggggtcttggagcagtcaagtctgatctggcctgtgttccttcagctaaaaggcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccattgctctggtctgatcctgcagataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtc

SEQ ID NO: 91 - DNA, 1524 bp, synthetic construct
  source 1..1524: mol_type = other DNA
atgtcctcggacgacaggcacctgggctccagctgcggctccttcatcaagactgagccgtccagcccgtcctcgggcatcgatgccctcagccaccacagccccagtggctcgtccgacgccagcggcggctttggcctggccctgggcacccacgccaacggtctggactcgccacccatgtttgcaggcgccgggctgggaggcaccccatgccgcaagagctacgaggactgtgccagcggcatcatggaggactcggccatcaagtgcgagtacatgctcaacgccatccccaagcgcctgtgcctcgtgtgcggggacattgcctctggctaccactacggcgtggcctcctgcgaggcttgcaaggccttcttcaagaggactatccaagggaacattgagtacagctgcccggccaccaacgagtgcgagatcaccaaacggaggcgcaagtcctgccaggcctgccgcttcatgaaatgcctcaaagtggggatgctgaaggaaggtgtgcgccttgatcgagtgcgtggaggccgtcagaaatacaagcgacggctggactcagagagcagcccatacctgagcttacaaatttctccacctgctaaaaagccattgaccaagattgtctcatacctactggtggctgagccggacaagctctatgccatgcctccccctggtatgcctgagggggacatcaaggccctgaccactctctgtgacctggcagaccgagagcttgtggtcatcattggctgggccaagcacatcccaggcttctcaagcctctccctgggggaccagatgagcctgctgcagagtgcctggatggaaatcctcatcctgggcatcgtgtaccgctcgctgccctatgacgacaagctggtgtacgctgaggactacatcatggatgaggagcactcccgcctcgcggggctgctggagctctaccgggccatcctgcagctggtacgcaggtacaagaagctcaaggtggagaaggaggagtttgtgacgctcaaggccctggccctcgccaactccgattccatgtacatcgaggatctagaggctgtccagaagctgcaggacctgctgcacgaggcactgcaggactacgagctgagccagcgccatgaggagccctggaggacgggcaagctgctgctgacactgccgctgctgcggcagacggccgccaaggccgtgcagcacttctatagcgtcaaactgcagggcaaagtgcccatgcacaaactcttcctggagatgctggaggccaaggttggccaagagcagcttagaggatctcccaaggatgaaagaatgtcaagccatgatggaaaatgccccttccaatcagctgccttcacaagcagggatcagagcaactccccggggatccccaatccacgcccttctagtccaacccccctcaatgagagaggcaggcagatctcacccagcactaggacaccaggaggccagggaaagcatctctggctcaccatg

SEQ ID NO: 92 - DNA, 1311 bp, synthetic construct
  source 1..1311: mol_type = other DNA
atgtcgtccatcttgccattcacgccgccagttgtgaagagactgctgggatggaagaagtcagctggtgggtctggaggagcaggcggaggagagcagaatgggcaggaagaaaagtggtgtgagaaagcagtgaaaagtctggtgaagaagctaaagaaaacaggacgattagatgagcttgagaaagccatcaccactcaaaactgtaatactaaatgtgttaccataccaaggtctcttgatggtcgtctccaggtatcccatcgaaaaggattgccacatgttatatattgccgattatggcgctggcctgatcttcacagtcatcatgaactcaaggcaattgaaaactgcgaatatgcttttaatcttaaaaaggatgaagtatgtgtaaacccttaccactatcagagagttgagacaccagttttgcctccagtattagtgccccgacacaccgagatcctaacagaacttccgcctctggatgactatactcactccattccagaaaacactaacttcccagcaggaattgagccacagagtaattatattccagaaacgccacctcctggatatatcagtgaagatggagaaacaagtgaccaacagttgaatcaaagtatggacacaggctctccagcagaactatctcctactactctttcccctgttaatcatagcttggatttacagccagttacttactcagaacctgcattttggtgttcgatagcatattatgaattaaatcagagggttggagaaaccttccatgcatcacagccctcactcactgtagatggctttacagacccatcaaattcagagaggttctgcttaggtttactctccaatgttaaccgaaatgccacggtagaaatgacaagaaggcatataggaagaggagtgcgcttatactacataggtggggaagtttttgctgagtgcctaagtgatagtgcaatctttgtgcagagccccaattgtaatcagagatatggctggcaccctgcaacagtgtgtaaaattccaccaggctgtaatctgaagatcttcaacaaccaggaatttgctgctcttctggctcagtctgttaatcagggttttgaagccgtctatcagctaactagaatgtgcaccataagaatgagttttgtgaaagggtggggagcagaataccgaaggcagacggtaacaagtactccttgctggattgaacttcatctgaatggacctctacagtggttggacaaagtattaactcagatgggatccccttcagtgcgttgctcaagcatgtca

SEQ ID NO: 93 - DNA, 849 bp, synthetic construct
  source 1..849: mol_type = other DNA
atggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctc

SEQ ID NO: 94 - DNA, 807 bp, synthetic construct
  source 1..807: mol_type = other DNA
atgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacaggaggaaaaagaaatgcattcggtacttacccggagaaggccgctgccccagccccgttccttccgatgacagtgctc

SEQ ID NO: 95 - DNA, 804 bp, synthetic construct
  source 1..804: mol_type = other DNA
atgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacagaccctggctcgcctaagaaatgccgtgctcgctttggcctcaaccagcagacggattggtgtggtccgtgcaga

SEQ ID NO: 96 - DNA, 807 bp, synthetic construct
  source 1..807: mol_type = other DNA
atgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacagagacaaactggcccagagaactcaaggatggtaatggacaagagtcactgtccatgtcttcttcctctagcccagct

SEQ ID NO: 97 - DNA, 828 bp, synthetic construct
  source 1..828: mol_type = other DNA
atgaagagactagcagctcgctgctttgctggcttgttaattttatccccactaactgtgatttctgatagccggcctgctgatagtggtaaggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgag

SEQ ID NO: 98 - DNA, 960 bp, synthetic construct
  source 1..960: mol_type = other DNA
atggagctgtgtgagttcgccttcaatatgaagaaggacgaggtctgcgtgaatccctaccactaccagagagtagagacaccagttctacctcctgtgttggtgccacgccacacagagatcccggccgagttccccccactggacgactacagccattccatccccgaaaacactaacttccccgcaggcatcgagccccagagcaatattccagagaccccaccccctggctacctgagtgaagatggagaaaccagtgaccaccagatgaaccacagcatggacgcaggttctccaaacctatccccgaatccgatgtccccagcacataataacttggacctgcagccagttacctactgcgagccggccttctggtgctccatctcctactacgagctgaaccagcgcgtcggggagacattccacgcctcgcagccatccatgactgtggatggcttcaccgacccctccaattcggagcgcttctgcctagggctgctctccaatgtcaacaggaatgcagcagtggagctgacacggagacacatcggaagaggcgtgcggctctactacatcggaggggaggtcttcgcagagtgcctcagtgacagcgctatttttgtccagtctcccaactgtaaccagcgctatggctggcacccggccaccgtctgcaagatcccaccaggatgcaacctgaagatcttcaacaaccaggagttcgctgccctcctggcccagtcggtcaaccagggctttgaggctgtctaccagttgacccgaatgtgcaccatccgcatgagcttcgtcaaaggctggggagcggagtacaggagacagactgtgaccagtaccccctgctggattgagctgcacctgaatgggcctttgcagtggcttgacaaggtcctcacccagatgggctccccaagcatccgctgttccagtgtgtct

SEQ ID NO: 99 - DNA, 954 bp, synthetic construct
  source 1..954: mol_type = other DNA
atgacccttgatcatcagatcatcaatccaactcttaaatggtcacaacctgcagtgccaagtggtgggcctcttgtgcagcatgcacacacaactctggacagtgatgctggcctcacagaaaacccactcaccaagttactagctattgggaaagaagatgacaatgcacaatggcatttatctggaagtattttggatgtgtatagcggtgaacaaggaatttcaccaattaacatggggcttacaagtgcttcttgtccaagtagtctaccaatgaaaagagaaattacagaaactgacactagagctttagcaaaagagagacaaaaaaaggacaaccacaacctcattgaaagaagaagaaggtataatattaattaccgaatcaaggagcttggcactcttattccaaagtctaatgatcctgatatgcgctggaacaaaggaaccattctaaaagcatcagtggagtacatcaagtggctacaaaaagaacaacagagagcccgagaattggaacacagacagaagaaattagagcaggctaacaggcgacttctacttcggattcaggaactagaaattcaggctcgtactcatggtctgccaaccctggcttcacttggcacggttgatttaggtgctcatgtcaccaaacagcagagccatcctgagcagaattcagtagactattgccaacaactgactgtgtctcaggggccaagccctgagctctgtgatcaagctatagccttttctgatcctttgtcatacttcacagatttatcatttagtgctgcattgaaagaggaacaaagattggatggcatgctattggatgacacaatctctccatttggaacagatcctctgctatctgccacttcccctgcagtttccaaagaaagcagtaggagaagtagctttagctcagatgatggtgatgaatta

SEQ ID NO: 100 - DNA, 1041 bp, synthetic construct
  source 1..1041: mol_type = other DNA
atgacccttgatcatcagatcatcaatccaactcttaaatggtcacaacctgcagtgccaagtggtgggcctcttgtgcagcatgcacacacaactctggacagtgatgctggcctcacagaaaacccactcaccaagttactagctattgggaaagaagatgacaatgcacaatggcatatggaggacgttattgaggatataatcggtatggaatcaagttttaaagaggaaggagcagactctcctctgctaatgcaaagaacattatctggaagtattttggatgtgtatagcggtgaacaaggaatttcaccaattaacatggggcttacaagtgcttcttgtccaagtagtctaccaatgaaaagagaaattacagaaactgacactagagctttagcaaaagagagacaaaaaaaggacaaccacaacctcattgaaagaagaagaaggtataatattaattaccgaatcaaggagcttggcactcttattccaaagtctaatgatcctgatatgcgctggaacaaaggaaccattctaaaagcatcagtggagtacatcaagtggctacaaaaagaacaacagagagcccgagaattggaacacagacagaagaaattagagcaggctaacaggcgacttctacttcggattcaggaactagaaattcaggctcgtactcatggtctgccaaccctggcttcacttggcacggttgatttaggtgctcatgtcaccaaacagcagagccatcctgagcagaattcagtagactattgccaacaactgactgtgtctcaggggccaagccctgagctctgtgatcaagctatagccttttctgatcctttgtcatacttcacagatttatcatttagtgctgcattgaaagaggaacaaagattggatggcatgctattggatgacacaatctctccatttggaacagatcctctgctatctgccacttcccctgcagtttccaaagaaagcagtaggagaagtagctttagctcagatgatggtgatgaatta

SEQ ID NO: 101 - DNA, 936 bp, synthetic construct
  source 1..936: mol_type = other DNA
atggcggacggcgacagcggcagcgagcgcggcggcggcggtgggccgtgcgggttccagcccgcgtcccgcggcggcggcgagcaagagacgcaggagctggcctcgaagcggctggacatccagaacaagcgcttctacttagatgtgaagcagaacgccaagggccgcttcctcaagatcgccgaggtgggcgcgggcggttccaagagccgcctcacgctgtccatggcggtggccgccgagttccgcgactcgctgggcgacttcatagaacactacgcgcagctgggccctagcagccccgagcagctggcggcgggcgccgaggagggcggcgggccgcggcgcgcgctcaagagcgaattcttggtgcgtgagaaccgcaagtactacctggacctcaaggagaaccagcgcggccgcttcctgcgcatccgccaaacggtcaaccgcggcggtggcggcttcggcgcgggccccgggccgggcggcttgcagagcggccagaccatcgcgctgcctgcgcagggcctcatcgagttccgcgacgcgctggcgaagctcatagacgactacggaggcgaggacgacgagctggcaggcggcccgggaggcggcgccgggggcccagggggcggcctgtatggagagctcccggagggcacctccatcaccgtggactccaagcgcttcttcttcgatgtgggctgcaacaaatacggggtgtttctgcgagtgagcgaggtgaagccgtcctaccgcaatgccatcaccgtacccttcaaagcctggggcaagttcggaggcgccttttgccggtatgcggatgagatgaaagaaatccaggaacgacagagggataagctttatgagcgacgtggtgggggcagcggcggcggcgaagagtcagagggtgaggaggtggatgaggat

SEQ ID NO: 102 - DNA, 1926 bp, synthetic construct
  source 1..1926: mol_type = other DNA
atggctgaggcggccccggccccgacatctgaatgggactccgagtgccttacatccctgcagccccttcctcttcctacacccccagcagcaaatgaggcacacctgcagacagcagctatctctctgtggacagtggtggccgccgtgcaggctatagagaggaaggtggagatccacagccggcgactcctacacctagaaggtcggacagggacagcagagaagaaactagccagctgtgaaaagacagttaccgagcttgggaaccagctggagggcaagtgggccgtgctgggaaccctgctgcaggagtacgggctgctgcagaggcggctggagaacttggagaacctgctgcgcaacaggaacttctggatcctgcggctccctccaggtattaagggagatatcccaaaggtgcctgtggcatttgatgatgtctccatctacttttccactccagagtgggaaaaattagaagaatggcaaaaggaactttacaagaatatcatgaagggcaactacgagtctctcatctccatggattatgctataaatcaacctgatgtcttatctcagattcaaccagaaggggaacataatacagaggaccaggcagggccagaggaaagtgagattcccacagaccccagtgaagagcctggtatttcaacatcagatattctgtcttggattaaacaagaagaagagcctcaggttggggccccaccggagtccaaggagagtgacgtgtacaaaagcacttatgctgatgaagagcttgtcatcaaagctgaaggccttgctagatcctcgttgtgccctgaggttccagtccctttctcttctccaccagcagcagcaaaggatgctttttcagatgtggctttcaaaagccagcagtctacatccatgacaccttttggacgtccagccactgacctgcctgaagcctctgagggacaagtgacttttactcagttgggtagctatcccctcccacctccagttggcgagcaggtgttctcatgccaccactgtggcaagaatctcagccaagacatgttgctgacccaccaatgtagccatgctactgagcaccccttaccctgtgcccagtgccctaagcactttactccacaggcggacctcagcagcacctcccaggaccatgccagcgagacaccccccacctgcccacactgtgccaggacttttactcacccatcaagacttacctaccatcttcgggtccataacagcactgagcgtcctttcccctgtcctgattgccccaagcgctttgctgaccaggctcgactcaccagccaccggagagctcatgcaagcgaaaggcccttccgctgtgcccagtgcggcaggagcttcagcttgaaaatcagcctcctgctccaccagcggggtcatgcacaagagcgccctttctcctgccctcagtgtggcattgacttcaacggccactcggccctgatccgccaccagatgatccacacaggcgagcgtccttacccctgcactgactgcagtaagagcttcatgcgcaaggagcacctgctgaaccaccggcggctgcacacaggcgagcggcccttcagttgtcctcactgtggcaagagcttcatccgcaagcaccacctaatgaaacaccagcgcatccacaccggggagcggccctacccctgctcctactgtggcaggagcttccgctacaaacagacactcaaggaccacctccgttcaggccacaatggaggctgtgggggtgatagtgacccatcaggtcagccacccaacccaccaggtcccctcataactgggcttgaaacttctggcctgggtgtcaacactgaaggtctagagaccaaccagtggtatggggaagggagtggagggggagttttg

SEQ ID NO: 103 - DNA, 579 bp, synthetic construct
  source 1..579: mol_type = other DNA
atggacggcggcacactgcccaggtccgcgccccctgcgccccccgtccctgtcggctgcgctgcccggcggagacccgcgtccccggaactgttgcgctgcagccggcggcggcgaccggccaccgcagagaccggaggcggcgcagcggccgtagcgcggcgcaatgagcgcgagcgcaaccgcgtgaagctggtgaacttgggcttccaggcgctgcggcagcacgtgccgcacggcggcgccagcaagaagctgagcaaggtggagacgctgcgctcagccgtggagtacatccgcgcgctgcagcgcctgctggccgagcacgacgccgtgcgcaacgcgctggcgggagggctgaggccgcaggccgtgcggccgtctgcgccccgcgggccgccagggaccaccccggtcgccgcctcgccctcccgcgcttcttcgtccccgggccgcgggggcagctcggagcccggctccccgcgttccgcctactcgtcggacgacagcggctgcgaaggcgcgctgagtcctgcggagcgcgagctactcgacttctccagctggttagggggctac

SEQ ID NO: 104 - DNA, 1260 bp, synthetic construct
  source 1..1260: mol_type = other DNA
atgatgtattctcccatctgtctcactcaggatgaatttcacccattcatcgaggcacttcttccacatgtccgtgcaattgcctatacttggttcaacctgcaggctcgaaaacgcaagtactttaaaaagcatgagaagcgaatgtcaaaggatgaagaaagagcagtcaaagatgagcttctcagtgaaaagcctgaaatcaaacagaagtgggcatccaggctccttgccaaactgcgcaaagatattcgccaggagtatcgagaggactttgtgctcaccgtgactggcaagaagcacccgtgctgtgtcttatccaatcccgaccagaagggtaagattaggagaatcgactgcctgcgacaggcagacaaagtctggcgtctggatctagtcatggtgatcctgttcaaaggcatccccttggaaagtaccgatggagagcggctcatgaaatccccacattgcacaaacccagcactttgtgtccagccacatcatatcacagtatcagttaaggagcttgatttgtttttggcatactacgtgcaggagcaagattctggacaatcaggaagtccaagccacaatgatcctgccaagaatcctccaggttaccttgaggatagttttgtaaaatctggagtcttcaatgtatcagaacttgtaagagtatccagaacgcccataacccagggaactggagtcaacttcccaattggagaaatcccaagccaaccatactatcatgacatgaactcgggggtcaatcttcagaggtctctgtcttctccaccaagcagcaaaagacccaaaactatatccatagatgaaaatatggaaccaagtcctacaggagacttttacccctctccaagttcaccagctgctggaagtcgaacatggcacgaaagagatcaagatatgtcttctccgactactatgaagaagcctgaaaagccattgttcagctctgcatctccacaggattcttccccaagactgagcactttcccccagcaccaccatcccggaatacctggagttgcacacagtgtcatctcaactcgaactccacctccaccttcaccgttgccatttccaacacaagctatccttcctccagccccatcgagctacttttctcatccaacaatcagatatcctccccacctgaatcctcaggatactctgaagaactatgtaccttcttatgacccatccagtccacaaaccagccagtcctggtacctgggc

SEQ ID NO: 105 - DNA, 1002 bp, synthetic construct
  source 1..1002: mol_type = other DNA
atgagatacaagacatccttggtgatgaggaaacgattacggctttaccgaaacactcttaaagagtcaagtagcagctctggacaccatggcccccagctcaccgccgcctccagcccctcggtgttcccgggcctccacgaggagcctccccaggcctcccccagccgtcctttgaatggactcctgcgtctggggctccctggagacatgtacgcgcggccggagcccttcccgccagggcctgcggcccgcagcgacgccctggcagctgccgcagccctgcatggctacgggggcatgaacctgacggtgaacctcgctgcgccccacggtcctggcgctttcttccgctacatgcgccagcccatcaaacaggagctcatctgcaagtggctggcggccgacggcaccgcgaccccgagcctctgctccaaaactttcagcaccatgcacgagctggtcacgcacgtcaccgtggagcacgtcggcggcccggaacaggccaaccacatttgcttctgggaggagtgtccgcgccagggaaagcccttcaaagccaaatacaaacttgtaaatcacatccgcgtgcacacgggcgagaagcccttcccttgtcctttcccggggtgtgggaaggtctttgctagatcagaaaatctcaaaatacacaaacgaactcacacaggcgagaagcccttcagatgcgagttcgagggctgcgagcggcgcttcgccaacagcagcgaccgtaagaagcattcgcacgtgcacactagcgacaagccatacacgtgcaaggtgcggggctgcgacaagtgctacacgcaccccagctcgctgcgtaagcacatgaaggtgcacgggcgctcgccgccgcccagctctggctacgattcggctacaccgtctgccctcgtgtcgccctcgtcggactgcggccacaagtcccaggtggcctcctcggcggcggtggcggcgcgtaccgccgacttgagcgaa

SEQ ID NO: 106 - DNA, 2583 bp, synthetic construct
  source 1..2583: mol_type = other DNA
atgaaccagccgggcggcgcggcggctccgcaggctgacggagccagtgcagccggaaggaaaagcactgcgagcagggagcgcttgaagcgcagccagaagagcaccaaggtggagggcccagagccagtgccagccgaggcctcgctgagtgccgagcaaggaacgatgacggaggtgaaggtgaagacagagctgcccgatgactacatccaggaggtgatctggcagggcgaggccaaggaggagaagaaggcggtcagcaaggatgggaccagcgacgtgcctgccgagatctgcgtggtgatcggcggcgtccgcaaccagcagacccttgggtcttacgaatgcggaatctgtggcaagaagtacaagtattacaactgcttccagacccacgtgcgggcgcaccgagacaccgaagccacctcaggggagggagcctcccaaagcaacaatttcaggtacacatgtgatatctgcgggaagaagtacaaatactacagctgtttccaagagcaccgagacctgcacgcagtggatgtgtttagtgtggaaggggcccctgagaaccgggcagaccccttcgaccaaggtgtcgtggccacggacgaggtgaaggaggagcccccggagccattccagaaaatcgggccaatgaacaatattacatcagacatttttaagaagaaagaagttaggcagtgccaaaagagagaaactggcaattacacctgtgaattctgcggcaaacagtacaagtactacactccctaccaggagcatgtggccttacacgcccccatcaaaagcgctttcagtcggagagtagaaggcaaagcacaaaaccactttgaagagacgaacagcagttcgcagaactccagcgaaccctacacctgcggcgcctgtgggatccagttccagttctacaacaacctgttggagcacatgcagtcccatgcagctgacaatgaaaacaacattgcctccaaccagtcccgatcgccacctgctgttgtagaagagaagtggaaacctcaggcccagaggaacagtgccaataacaccactaccagtggtttaacacccaacagcatgatccccgaaaaggagcggcagaacatcgcagagcggctgttgagggtcatgtgtgccgacctgggtgcactgagcgtggtcagcgggaaggagttcctgaagttggcccagaccttagtagacagtggtgcccgctatggggccttctcggtcactgaaatcctgggcaacttcaacacgctggcgctgaagcacctgccacgcatgtacaaccaggtgaaggtgaaagtgacctgtgccttgggcagcaatgcctgcctaggcatcggtgtcacctgccactcccagagtgttggccctgactcctgctacatcctcacagcctaccaggccgagggcaaccacatcaagagctatgtgcttggtgtgaagggtgcggacattcgcgacagcggtgaccttgtgcaccactgggtgcagaacgtgctgtcggagttcgtgatgtcggagatcaggacagtgtacgtgacggattgccgggtgagcacgtccgccttctccaaggccggcatgtgccttcgctgctcagcctgtgccttgaactcggtggtgcagagcgtgctgagcaagcggacactgcaggcccgcagcatgcacgaggtcatcgagctgctcaacgtgtgcgaggacctggcgggctccacgggcctggccaaggagaccttcgggtcgctggaggagacgtctccaccaccctgctggaactcggtgacggactcactgttgctggtgcatgagcgctatgagcagatctgcgagttctacagccgggccaagaagatgaacctcatccagagcctcaacaagcacctgctcagcaacctggcggccatcctgacgccggtgaagcaggcagtcatcgagctgagcaacgagagccagcccaccctgcagctggtgctgcccacctacgtcaggctggagaagctgttcacggccaaggccaacgacgcaggcactgtcagcaagctctgccacctcttcctggaggcgctcaaggagaacttcaaggtgcacccggcccacaaggtggccatgatcctggacccgcagcagaagctgcggcctgtgccaccctaccagcacgaggagatcatcggcaaggtctgtgagctcatcaacgaggtgaaggagtcctgggccgaggaggccgacttcgagcccgctgccaagaagccccgctctgctgccgtcgagaaccccgcagctcaggaagatgatcggctaggcaaaaatgaagtgtacgattacctgcaggagcccctcttccaggctacccctgatctcttccagtactggtcgtgcgttacccaaaagcacacaaaactcgccaagctcgccttctggctcctggcggttccggccgtgggcgccagaagcgggtgtgtaaatatgtgtgaacaagcgcttctaatcaaacggaggcggctgctcagtccagaagatatgaataaactcatgtttctgaaatccaacatgctt

SEQ ID NO: 107 - DNA, 1371 bp, synthetic construct
  source 1..1371: mol_type = other DNA
atgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctct

SEQ ID NO: 108 - DNA, 1389 bp, synthetic construct
  source 1..1389: mol_type = other DNA
atgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctct

SEQ ID NO: 109 - DNA, 1383 bp, synthetic construct
  source 1..1383: mol_type = other DNA
atggactattcgtacgacgaggacctggacgagctgtgccccgtgtgcggggacaaggtgtccggctaccactacggactgctcacgtgtgagagctgcaagggcttcttcaagcgcacggtgcagaacaacaagcactacacgtgcaccgagagccagagctgcaagatcgacaagacgcagcgcaagcgctgtcccttctgccgcttccagaaatgcctgacggtggggatgcgcctggaagccgtgcgcgctgaccgtatgaggggtggccggaacaagtttgggccgatgtacaagcgggaccgggccctgaaacagcagaagaaggcacagattcgggccaatggcttcaagctggagacagggcccccgatgggggtgcccccgccgccccctcccgcaccggactacgtgctgcctcccagcctgcatgggcctgagcccaagggcctggccgccggtccacctgctgggccactgggcgactttggggccccagcactgcccatggccgtgcccggtgcccacgggccactggctggctacctctaccctgcctttcctggccgtgccatcaagtctgagtacccggagccttatgccagccccccacagcctgggctgccgtacggctacccagagcccttctctggagggcccaacgtgcctgagctcatcctgcagctgctgcagctggagccggatgaggaccaggtgcgggcccgcatcttgggctgcctgcaggagcccaccaaaagccgccccgaccagccggcggccttcggcctcctgtgcagaatggccgaccagaccttcatctccatcgtggactgggcacgcaggtgcatggtcttcaaggagctggaggtggccgaccagatgacgctgctgcagaactgctggagcgagctgctggtgttcgaccacatctaccgccaggtccagcacggcaaggagggcagcatcctgctggtcaccgggcaggaggtggagctgaccacagtggccacccaggcgggctcgctgctgcacagcctggtgttgcgggcgcaggagctggtgctgcagctgcttgcgctgcagctggaccggcaggagtttgtctgcctcaagttcatcatcctcttcagcctggatttgaagttcctgaataaccacatcctggtgaaagacgctcaggagaaggccaacgccgccctgcttgactacaccctgtgccactacccgcactgcggggacaaattccagcagctgctgctgtgcctggtggaggtgcgggccctgagcatgcaggccaaggagtacctgtaccacaagcacctgggcaacgagatgccccgcaacaacctgctcatcgaaatgctgcaagccaagcagact

SEQ ID NO: 110 - DNA, 1050 bp, synthetic construct
  source 1..1050: mol_type = other DNA
atgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcc

SEQ ID NO: 111 - DNA, 1356 bp, synthetic construct
  source 1..1356: mol_type = other DNA
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatc

SEQ ID NO: 112 - DNA, 1347 bp, synthetic construct
  source 1..1347: mol_type = other DNA
atgtcggactggggccagggcttcccccaggacccaccagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatc

SEQ ID NO: 113 - DNA, 1185 bp, synthetic construct
  source 1..1185: mol_type = other DNA
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgct

SEQ ID NO: 114 - DNA, 1224 bp, synthetic construct
  source 1..1224: mol_type = other DNA
atgaataccacagacaacggtgtcaactgtctgtgtgctatctgtggggacagagcaacaggaaaacactatggggcatccagctgtgatgggtgcaagggtttcttcagacgcagcattcgtaagagtcacgtttattcttgcaggttcagtcggcaatgtgttgttgacaaggacaaaaggaatcaatgtagatattgtcgattaagaaagtgttttagagcgggaatgaaaaaagaagctgtacaaaatgaacgtgacagaataagcaccagaagaagcacatttgatggcagcaacatcccctccattaacacactggcacaagctgaagttcggtctcgccagatctcagtctcaagccctgggtcaagcactgacataaacgttaagaaaattgcaagtattggtgatgtctgtgaatctatgaaacagcagctcttagtcttggtggaatgggctaaatatattcctgccttctgtgaattaccattggatgatcaggtggcactgttgagagctcacgcaggggagcacttactgcttggagctacaaagagatccatgatgtataaagatattttgcttttgggaaacaactatgttattcaccgcaacagctgtgaagttgagattagccgtgtggccaatcgtgttctagatgagctggttagaccatttcaagaaatccagattgatgacaatgagtatgcttgtttaaaggcaattgtattttttgatccagatgcaaaagggctaagcgatccagtaaaaattaagaacatgaggttccaagtgcagatcggtttggaggactacatcaatgatcggcagtatgactcccgggggaggtttggagagttgcttctgctcctgcccacactgcagagcatcacgtggcaaatgattgagcaaatacagtttgttaaactttttgggatggttaaaattgacaatctacttcaggaaatgctattaggtggggcttccaatgatggcagtcatctccatcatccaatgcatccacatttgtctcaagacccattaactggacaaactatacttttaggtcccatgtcaacactggttcatgcagaccagatctcaactcctgaaaccccactcccttccccaccacaaggctctgggcaagaacagtacaaaatagctgcaaaccaagcatcagtcatttcacaccagcatctctccaaacaaaagcaattg

SEQ ID NO: 115 - DNA, 1224 bp, synthetic construct; identical to SEQ ID NO: 114
  source 1..1224: mol_type = other DNA

SEQ ID NO: 116 - DNA, 1542 bp, synthetic construct
  source 1..1542: mol_type = other DNA
atggtgtgggaagtgaagacaaatcagatgcctaatgcagtacagaaactcctgttggtgatggacaagagagcctcaggaatgaatgactcattggagttgctgcagtgtaatgagaatttgccatcttcacctggatataactcctgtgatgaacacatggagcttgatgaccttcctgaacttcaggcagttcaaagtgatcctacccaatctggcatgtaccagctgagttcagatgtttcacatcaagaatacccaagatcatcttggaaccaaaatacctcagacataccagaaactacttaccgtgaaaatgaggtggactggctaacagaattggcaaatatcgcgaccagtccacaaagtccactgatgcagtgctcattttacaatagatcatctcctgtacacatcatagccactagcaaaagtttacattcctatgcacgccctccaccagtgtcctcttcttcgaagagtgaaccagccttccctcatcaccattggaaggaggaaacaccagtaagacacgaaagggcaaatagtgagtcagaatctggcattttctgcatgtcctccctgtcagatgatgatgatttgggatggtgcaattcctggccttcaactgtctggcactgttttttgaaaggcacacgactgtgctttcataagggaagcaataaggaatggcaagatgttgaagattttgctagagctgaaggctgtgataatgaggaagatcttcaaatgggcattcacaagggctatggttctgatggtctaaagttgttatcacatgaagaaagtgtatcatttggcgagtctgtactgaagttgacttttgatcctggtacagtagaagatggtttacttaccgtagagtgtaagctggaccaccctttctatgttaaaaataaaggttggtcatcattttatccaagcttgactgtggtacagcatggcattccatgttgtgaagttcatattggcgatgtatgtctacctcctggacaccccgatgccattaattttgatgattcaggtgtttttgatacatttaaaagctatgacttcacacctatggattcttctgcagtttatgtgttaagtagtatggctcgccagcgtcgtgcatctttgtcttgtggaggacctggtggtcaagactttgcaagatctggattcagtaaaaactgtggctcacctggatcatcacagctctcttccaattctttgtatgctaaagctgtcaaaaaccacagctcagggactgtgagtgccacttctcctaataagtgcaaaagaccaatgaatgccttcatgctttttgccaaaaaatacagagttgaatatactcagatgtatccagggaaagataacagagccataagtgtgatccttggtgacaggtggaagaaaatgaagaatgaagagagaagaatgtacacattagaagcaaaggctttggctgaagaacagaaacgtttaaatcctgactgttggaagaggaaaagaaccaattcaggctcacaacaacat

SEQ ID NO: 117 - DNA, 399 bp, synthetic construct
  source 1..399: mol_type = other DNA
atgatgctcaactcagacaccatggagctggacctgccgcccacccactcagagactgagtcgggcttcagtgactgtgggggcggggcgggccctgatggtgccgggcctgggggtccgggagggggccaggcccgaggcccagagccgggagagcctggccggaaagacctgcagcatctgagccgcgaggagcgccggcgccggcgccgcgccacagccaagtaccgcacggcccacgccacgcgagaacgcatccgcgtggaagccttcaacctggccttcgccgagctgcgcaagctgctgcctacgctgccccccgacaagaagctctccaagattgagattctgcgcctggccatctgctatatctcctacctgaaccacgtgctggacgtc

SEQ ID NO: 118 - DNA, 1485 bp, synthetic construct
  source 1..1485: mol_type = other DNA
atgtcttctaattcagatactggggatttacaagagtctttaaagcacggacttacacctattgtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagca

SEQ ID NO: 119 - DNA, 1407 bp, synthetic construct
  source 1..1407: mol_type = other DNA
atggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagca

SEQ ID NO: 120 - DNA, 414 bp, synthetic construct
  source 1..414: mol_type = other DNA
atgggagacaagaagagccccaccaggaaacctcgacctgtctcccagttggttgcacagcaggttactcagcagtttgtgcctcctacacagtcaaagaaagagaaaaaagataaagtagaaaaagaaaaaagtgaaaaggaaacaactagcaaaaagaatagccataagaaaaccaggccaagattgaaaaatgtggatcggagtagtgctcagcatttggaagttactgttggagatctgacagtcattattacagactttaaggagaaaacaaagtcaccgcctgcatctagtgctgcttctgcagatcaacacagtcaaagcggctctagctctgataacacagagagaggaatgtccaggtcatcttcacccagaggagaagcctcatcattgaatggagaatctcat

SEQ ID NO: 121 - protein, 135 aa, synthetic construct
MMLSPDQAADSDHPSSAHSDPESLGGTDTKVLGSVSDLEPVEEAEGDGKGGSRAALYPHPQQLSREEKRRRRRATAKYRSAHATRERIRVEAFNLAFAELRKLLPTLPPDKKLSKIEILRLAICYISYLNHVLDV

SEQ ID NO: 122 - protein, 302 aa, synthetic construct
MDVLASYSIFQELQLVHDTGYFSALPSLEETWQQTCLELERYLQTEPRRISETFGEDLDCFLHASPPPCIEESFRRLDPLLLPVEAAICEKSSAVDILLSRDKLLSETCLSLQPASSSLDSYTAVNQAQLNAVTSLTPPSSPELSRHLVKTSQTLSAVDGTVTLKLVAKKAALSSVKVGGVATAAAAVTAAGAVKSGQSDSDQGGLGAEACPENKKRVHRCQFNGCRKVYTKSSHLKAHQRTHTGEKPYKCSWEGCEWRFARSDELTRHYRKHTGAKPFKCNHCDRCFSRSDHLALHMKRHI

SEQ ID NO: 123 - protein, 205 aa, synthetic construct
MAAAAGGAASRRGAGRPCPFSIEHILSSLPERSLPARAACPPQPAGRQSPAKPEEPGAPEAAPCACCCCCGPRAAPCGPPEAAAGLGARLAWPLRLGPAVPLSLGAPAGGSGALPGAVGPGSQRRTRRHRTIFSEEQLQALEALFVQNQYPDVSTRERLAGRIRLREERVEVWFKNRRAKWRHQKRASASARLLPGVKKSPKGSC

SEQ ID NO: 124 - protein, 505 aa, synthetic construct
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 125 - protein, 616 aa, synthetic construct
MSRRKQAKPQHINSEEDQGEQQPQQQTPEFADAAPAAPAAGELGAPVNHPGNDEVASEDEATVKRLRREETHVCEKCCAEFFSISEFLEHKKNCTKNPPVLIMNDSEGPVPSEDFSGAVLSHQPTSPGSKDCHRENGGSSEDMKEKPDAESVVYLKTETALPPTPQDISYLAKGKVANTNVTLQALRGTKVAVNQRSADALPAPVPGANSIPWVLEQILCLQQQQLQQIQLTEQIRIQVNMWASHALHSSGAGADTLKTLGSHMSQQVSAAVALLSQKAGSQGLSLDALKQAKLPHANIPSATSSLSPGLAPFTLKPDGTRVLPNVMSRLPSALLPQAPGSVLFQSPFSTVALDTSKKGKGKPPNISAVDVKPKDEAALYKHKCRSSLPSTFIRAPPTYVKVEVPGTFVGPSTLSPGMTPLLAAQPRRQAKQHGCTRCGKNFSSASALQIHERTHTGEKPFVCNICGRAFTTKGNLKVHYMTHGANNNSARRGRKLAIENTMALLGTDGKRVSEIFPKEILAPSVNVDPVVWNQYTSMLNGGLAVKTNEISVIQSGGVPTLPVSLGATSVVNNATVSKMDGSQSGISADVEKPSATDGVPKHQFPHFLEENKIAVS

SEQ ID NO: 126 - protein, 442 aa, synthetic construct
MSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPLLWSDPADNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 127 - protein, 435 aa, synthetic construct
MSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 128 - protein, 470 aa, synthetic construct
MWRECDWGLGAVKSDLACVPSAKRLLCRMSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPLLWSDPADNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 129 - protein, 508 aa, synthetic construct
MSSDDRHLGSSCGSFIKTEPSSPSSGIDALSHHSPSGSSDASGGFGLALGTHANGLDSPPMFAGAGLGGTPCRKSYEDCASGIMEDSAIKCEYMLNAIPKRLCLVCGDIASGYHYGVASCEACKAFFKRTIQGNIEYSCPATNECEITKRRRKSCQACRFMKCLKVGMLKEGVRLDRVRGGRQKYKRRLDSESSPYLSLQISPPAKKPLTKIVSYLLVAEPDKLYAMPPPGMPEGDIKALTTLCDLADRELVVIIGWAKHIPGFSSLSLGDQMSLLQSAWMEILILGIVYRSLPYDDKLVYAEDYIMDEEHSRLAGLLELYRAILQLVRRYKKLKVEKEEFVTLKALALANSDSMYIEDLEAVQKLQDLLHEALQDYELSQRHEEPWRTGKLLLTLPLLRQTAAKAVQHFYSVKLQGKVPMHKLFLEMLEAKVGQEQLRGSPKDERMSSHDGKCPFQSAAFTSRDQSNSPGIPNPRPSSPTPLNERGRQISPSTRTPGGQGKHLWLTM

SEQ ID NO: 130 - protein, 437 aa, synthetic construct
MSSILPFTPPVVKRLLGWKKSAGGSGGAGGGEQNGQEEKWCEKAVKSLVKKLKKTGRLDELEKAITTQNCNTKCVTIPRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 131 - protein, 283 aa, synthetic construct
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 132 - protein, 269 aa, synthetic construct
MYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTGGKRNAFGTYPEKAAAPAPFLPMTVL

SEQ ID NO: 133 - protein, 268 aa, synthetic construct
MYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTDPGSPKKCRARFGLNQQTDWCGPCR

SEQ ID NO: 134 - protein, 269 aa, synthetic construct
MYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTETNWPRELKDGNGQESLSMSSSSSPA

SEQ ID NO: 135 - protein, 276 aa, synthetic construct
MKRLAARCFAGLLILSPLTVISDSRPADSGKAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 136 - protein, 320 aa, synthetic construct
MELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 137 - protein, 318 aa, synthetic construct
MTLDHQIINPTLKWSQPAVPSGGPLVQHAHTTLDSDAGLTENPLTKLLAIGKEDDNAQWHLSGSILDVYSGEQGISPINMGLTSASCPSSLPMKREITETDTRALAKERQKKDNHNLIERRRRYNINYRIKELGTLIPKSNDPDMRWNKGTILKASVEYIKWLQKEQQRARELEHRQKKLEQANRRLLLRIQELEIQARTHGLPTLASLGTVDLGAHVTKQQSHPEQNSVDYCQQLTVSQGPSPELCDQAIAFSDPLSYFTDLSFSAALKEEQRLDGMLLDDTISPFGTDPLLSATSPAVSKESSRRSSFSSDDGDEL

SEQ ID NO: 138 - protein, 347 aa, synthetic construct
MTLDHQIINPTLKWSQPAVPSGGPLVQHAHTTLDSDAGLTENPLTKLLAIGKEDDNAQWHMEDVIEDIIGMESSFKEEGADSPLLMQRTLSGSILDVYSGEQGISPINMGLTSASCPSSLPMKREITETDTRALAKERQKKDNHNLIERRRRYNINYRIKELGTLIPKSNDPDMRWNKGTILKASVEYIKWLQKEQQRARELEHRQKKLEQANRRLLLRIQELEIQARTHGLPTLASLGTVDLGAHVTKQQSHPEQNSVDYCQQLTVSQGPSPELCDQAIAFSDPLSYFTDLSFSAALKEEQRLDGMLLDDTISPFGTDPLLSATSPAVSKESSRRSSFSSDDGDEL

SEQ ID NO: 139 - protein, 312 aa, synthetic construct
MADGDSGSERGGGGGPCGFQPASRGGGEQETQELASKRLDIQNKRFYLDVKQNAKGRFLKIAEVGAGGSKSRLTLSMAVAAEFRDSLGDFIEHYAQLGPSSPEQLAAGAEEGGGPRRALKSEFLVRENRKYYLDLKENQRGRFLRIRQTVNRGGGGFGAGPGPGGLQSGQTIALPAQGLIEFRDALAKLIDDYGGEDDELAGGPGGGAGGPGGGLYGELPEGTSITVDSKRFFFDVGCNKYGVFLRVSEVKPSYRNAITVPFKAWGKFGGAFCRYADEMKEIQERQRDKLYERRGGGSGGGEESEGEEVDED

SEQ ID NO: 140 - protein, 642 aa, synthetic construct
MAEAAPAPTSEWDSECLTSLQPLPLPTPPAANEAHLQTAAISLWTVVAAVQAIERKVEIHSRRLLHLEGRTGTAEKKLASCEKTVTELGNQLEGKWAVLGTLLQEYGLLQRRLENLENLLRNRNFWILRLPPGIKGDIPKVPVAFDDVSIYFSTPEWEKLEEWQKELYKNIMKGNYESLISMDYAINQPDVLSQIQPEGEHNTEDQAGPEESEIPTDPSEEPGISTSDILSWIKQEEEPQVGAPPESKESDVYKSTYADEELVIKAEGLARSSLCPEVPVPFSSPPAAAKDAFSDVAFKSQQSTSMTPFGRPATDLPEASEGQVTFTQLGSYPLPPPVGEQVFSCHHCGKNLSQDMLLTHQCSHATEHPLPCAQCPKHFTPQADLSSTSQDHASETPPTCPHCARTFTHPSRLTYHLRVHNSTERPFPCPDCPKRFADQARLTSHRRAHASERPFRCAQCGRSFSLKISLLLHQRGHAQERPFSCPQCGIDFNGHSALIRHQMIHTGERPYPCTDCSKSFMRKEHLLNHRRLHTGERPFSCPHCGKSFIRKHHLMKHQRIHTGERPYPCSYCGRSFRYKQTLKDHLRSGHNGGCGGDSDPSGQPPNPPGPLITGLETSGLGVNTEGLETNQWYGEGSGGGVL

SEQ ID NO: 141 - protein, 193 aa, synthetic construct
MDGGTLPRSAPPAPPVPVGCAARRRPASPELLRCSRRRRPATAETGGGAAAVARRNERERNRVKLVNLGFQALRQHVPHGGASKKLSKVETLRSAVEYIRALQRLLAEHDAVRNALAGGLRPQAVRPSAPRGPPGTTPVAASPSRASSSPGRGGSSEPGSPRSAYSSDDSGCEGALSPAERELLDFSSWLGGY

SEQ ID NO: 142 - protein, 420 aa, synthetic construct
MMYSPICLTQDEFHPFIEALLPHVRAIAYTWFNLQARKRKYFKKHEKRMSKDEERAVKDELLSEKPEIKQKWASRLLAKLRKDIRQEYREDFVLTVTGKKHPCCVLSNPDQKGKIRRIDCLRQADKVWRLDLVMVILFKGIPLESTDGERLMKSPHCTNPALCVQPHHITVSVKELDLFLAYYVQEQDSGQSGSPSHNDPAKNPPGYLEDSFVKSGVFNVSELVRVSRTPITQGTGVNFPIGEIPSQPYYHDMNSGVNLQRSLSSPPSSKRPKTISIDENMEPSPTGDFYPSPSSPAAGSRTWHERDQDMSSPTTMKKPEKPLFSSASPQDSSPRLSTFPQHHHPGIPGVAHSVISTRTPPPPSPLPFPTQAILPPAPSSYFSHPTIRYPPHLNPQDTLKNYVPSYDPSSPQTSQSWYLG

SEQ ID NO: 143 - protein, 334 aa, synthetic construct
MRYKTSLVMRKRLRLYRNTLKESSSSSGHHGPQLTAASSPSVFPGLHEEPPQASPSRPLNGLLRLGLPGDMYARPEPFPPGPAARSDALAAAAALHGYGGMNLTVNLAAPHGPGAFFRYMRQPIKQELICKWLAADGTATPSLCSKTFSTMHELVTHVTVEHVGGPEQANHICFWEECPRQGKPFKAKYKLVNHIRVHTGEKPFPCPFPGCGKVFARSENLKIHKRTHTGEKPFRCEFEGCERRFANSSDRKKHSHVHTSDKPYTCKVRGCDKCYTHPSSLRKHMKVHGRSPPPSSGYDSATPSALVSPSSDCGHKSQVASSAAVAARTADLSE

SEQ ID NO: 144 - protein, 861 aa, synthetic construct
MNQPGGAAAPQADGASAAGRKSTASRERLKRSQKSTKVEGPEPVPAEASLSAEQGTMTEVKVKTELPDDYIQEVIWQGEAKEEKKAVSKDGTSDVPAEICVVIGGVRNQQTLGSYECGICGKKYKYYNCFQTHVRAHRDTEATSGEGASQSNNFRYTCDICGKKYKYYSCFQEHRDLHAVDVFSVEGAPENRADPFDQGVVATDEVKEEPPEPFQKIGPMNNITSDIFKKKEVRQCQKRETGNYTCEFCGKQYKYYTPYQEHVALHAPIKSAFSRRVEGKAQNHFEETNSSSQNSSEPYTCGACGIQFQFYNNLLEHMQSHAADNENNIASNQSRSPPAVVEEKWKPQAQRNSANNTTTSGLTPNSMIPEKERQNIAERLLRVMCADLGALSVVSGKEFLKLAQTLVDSGARYGAFSVTEILGNFNTLALKHLPRMYNQVKVKVTCALGSNACLGIGVTCHSQSVGPDSCYILTAYQAEGNHIKSYVLGVKGADIRDSGDLVHHWVQNVLSEFVMSEIRTVYVTDCRVSTSAFSKAGMCLRCSACALNSVVQSVLSKRTLQARSMHEVIELLNVCEDLAGSTGLAKETFGSLEETSPPPCWNSVTDSLLLVHERYEQICEFYSRAKKMNLIQSLNKHLLSNLAAILTPVKQAVIELSNESQPTLQLVLPTYVRLEKLFTAKANDAGTVSKLCHLFLEALKENFKVHPAHKVAMILDPQQKLRPVPPYQHEEIIGKVCELINEVKESWAEEADFEPAAKKPRSAAVENPAAQEDDRLGKNEVYDYLQEPLFQATPDLFQYWSCVTQKHTKLAKLAFWLLAVPAVGARSGCVNMCEQALLIKRRRLLSPEDMNKLMFLKSNML

SEQ ID NO: 145 - protein, 457 aa, synthetic construct; identical to SEQ ID NO: 81

SEQ ID NO: 146 - protein, 463 aa, synthetic construct
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 147 - protein, 461 aa, synthetic construct
MDYSYDEDLDELCPVCGDKVSGYHYGLLTCESCKGFFKRTVQNNKHYTCTESQSCKIDKTQRKRCPFCRFQKCLTVGMRLEAVRADRMRGGRNKFGPMYKRDRALKQQKKAQIRANGFKLETGPPMGVPPPPPPAPDYVLPPSLHGPEPKGLAAGPPAGPLGDFGAPALPMAVPGAHGPLAGYLYPAFPGRAIKSEYPEPYASPPQPGLPYGYPEPFSGGPNVPELILQLLQLEPDEDQVRARILGCLQEPTKSRPDQPAAFGLLCRMADQTFISIVDWARRCMVFKELEVADQMTLLQNCWSELLVFDHIYRQVQHGKEGSILLVTGQEVELTTVATQAGSLLHSLVLRAQELVLQLLALQLDRQEFVCLKFIILFSLDLKFLNNHILVKDAQEKANAALLDYTLCHYPHCGDKFQQLLLCLVEVRALSMQAKEYLYHKHLGNEMPRNNLLIEMLQAKQT

SEQ ID NO: 148 - protein, 350 aa, synthetic construct
MLGSVKMEAHDLAEWSYYPEAGEVYSPVTPVPTMAPLNSYMTLNPLSSPYPPGGLPASPLPSGPLAPPAPAAPLGPTFPGLGVSGGSSSSGYGAPGPGLVHGKEMPKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKVKKGGSGAATTTRNGTGSAASTTTPAATVTSPPQPPPPAPEPEAQGGEDVGALDCGSPASSTPYFTGLELPGELKLDAPYNFNHPFSINNLMSEQTPAPPKLDVGFGGYGAEGGEPGVYYQGLYSRSLLNAS

SEQ ID NO: 149 - protein, 452 aa, synthetic construct
MVSVNAPLGAPVESSYDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 150 - protein, 449 aa, synthetic construct
MSDWGQGFPQDPPDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 151 - protein, 395 aa, synthetic construct
MVSVNAPLGAPVESSYDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGPCQAQEGRGWSGDSPGDRPHTVSSPLSSLASPLCRFGQVA

SEQ ID NO: 152 - protein, 408 aa, synthetic construct
MNTTDNGVNCLCAICGDRATGKHYGASSCDGCKGFFRRSIRKSHVYSCRFSRQCVVDKDKRNQCRYCRLRKCFRAGMKKEAVQNERDRISTRRSTFDGSNIPSINTLAQAEVRSRQISVSSPGSSTDINVKKIASIGDVCESMKQQLLVLVEWAKYIPAFCELPLDDQVALLRAHAGEHLLLGATKRSMMYKDILLLGNNYVIHRNSCEVEISRVANRVLDELVRPFQEIQIDDNEYACLKAIVFFDPDAKGLSDPVKIKNMRFQVQIGLEDYINDRQYDSRGRFGELLLLLPTLQSITWQMIEQIQFVKLFGMVKIDNLLQEMLLGGASNDGSHLHHPMHPHLSQDPLTGQTILLGPMSTLVHADQISTPETPLPSPPQGSGQEQYKIAANQASVISHQHLSKQKQL

SEQ ID NO: 153 - protein, 408 aa, synthetic construct; identical to SEQ ID NO: 152

SEQ ID NO: 154 - protein, 514 aa, synthetic construct
MVWEVKTNQMPNAVQKLLLVMDKRASGMNDSLELLQCNENLPSSPGYNSCDEHMELDDLPELQAVQSDPTQSGMYQLSSDVSHQEYPRSSWNQNTSDIPETTYRENEVDWLTELANIATSPQSPLMQCSFYNRSSPVHIIATSKSLHSYARPPPVSSSSKSEPAFPHHHWKEETPVRHERANSESESGIFCMSSLSDDDDLGWCNSWPSTVWHCFLKGTRLCFHKGSNKEWQDVEDFARAEGCDNEEDLQMGIHKGYGSDGLKLLSHEESVSFGESVLKLTFDPGTVEDGLLTVECKLDHPFYVKNKGWSSFYPSLTVVQHGIPCCEVHIGDVCLPPGHPDAINFDDSGVFDTFKSYDFTPMDSSAVYVLSSMARQRRASLSCGGPGGQDFARSGFSKNCGSPGSSQLSSNSLYAKAVKNHSSGTVSATSPNKCKRPMNAFMLFAKKYRVEYTQMYPGKDNRAISVILGDRWKKMKNEERRMYTLEAKALAEEQKRLNPDCWKRKRTNSGSQQH

SEQ ID NO: 155 - protein, 133 aa, synthetic construct
MMLNSDTMELDLPPTHSETESGFSDCGGGAGPDGAGPGGPGGGQARGPEPGEPGRKDLQHLSREERRRRRRATAKYRTAHATRERIRVEAFNLAFAELRKLLPTLPPDKKLSKIEILRLAICYISYLNHVLDV

SEQ ID NO: 156 - protein, 541 aa, synthetic construct
MSSNSDTGDLQESLKHGLTPIGAGLPDRHGSPIPARGRLVMLPKVETEALGLARSHGEQGQMPENMQVSQFKMVNYSYDEDLEELCPVCGDKVSGYHYGLLTCESCKGFFKRTVQNNKRYTCIENQNCQIDKTQRKRCPYCRFQKCLSVGMKLEAVRADRMRGGRNKFGPMYKRDRALKQQKKALIRANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 157 - protein, 469 aa, synthetic construct
MVNYSYDEDLEELCPVCGDKVSGYHYGLLTCESCKGFFKRTVQNNKRYTCIENQNCQIDKTQRKRCPYCRFQKCLSVGMKLEAVRADRMRGGRNKFGPMYKRDRALKQQKKALIRANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 158 - protein, 138 aa, synthetic construct
MGDKKSPTRKPRPVSQLVAQQVTQQFVPPTQSKKEKKDKVEKEKSEKETTSKKNSHKKTRPRLKNVDRSSAQHLEVTVGDLTVIITDFKEKTKSPPASSAASADQHSQSGSSSDNTERGMSRSSSPRGEASSLNGESH

SEQ ID NO: 159 - DNA, 20 bp, synthetic construct
  source 1..20: mol_type = other DNA
ctggacatgagtcacttaat

SEQ ID NO: 160 - DNA, 20 bp, synthetic construct
  source 1..20: mol_type = other DNA
caattaagtgactcatgtcc

SEQ ID NO: 161 - DNA, 20 bp, synthetic construct
  source 1..20: mol_type = other DNA
tttatggcacagattcagtg

SEQ ID NO: 162 - DNA, 20 bp, synthetic construct
  source 1..20: mol_type = other DNA
cttgacatctgtcacagtgt

SEQ ID NO: 163 - DNA, 20 bp, synthetic construct
  source 1..20: mol_type = other DNA
aatcctattggacagaaacc

SEQ ID NO: 164 - DNA, 20 bp, synthetic construct
  source 1..20: mol_type = other DNA
acttgacatctgtcacagtg

SEQ ID NO: 165 - DNA, 20 bp, synthetic construct
  source 1..20: mol_type = other DNA
gagataaaggcttaggaagg

SEQ ID NO: 166 - DNA, 20 bp, synthetic construct
  source 1..20: mol_type = other DNA
acggctagatactgtttagg

SEQ ID NO: 167 - DNA, 20 bp, synthetic construct
  source 1..20: mol_type = other DNA
tggacatgagtcacttaatg

SEQ ID NO: 168 - DNA, 20 bp, synthetic construct
  source 1..20: mol_type = other DNA
tatttaatgggaaatcctat

SEQ ID NO: 169 - DNA, 20 bp, synthetic construct
  source 1..20: mol_type = other DNA
tctgctccgggctccccatg

SEQ ID NO: 170 - DNA, 20 bp, synthetic construct
  source 1..20: mol_type = other DNA
tgcgcggagaaccgaacggg

SEQ ID NO: 171 - DNA, 20 bp, synthetic construct
  source 1..20: mol_type = other DNA
cgagcgcaagggacagggag

SEQ ID NO: 172 - DNA, 20 bp, synthetic construct
  source 1..20: mol_type = other DNA
gggaagactacaaactagca

SEQ ID NO: 173 - DNA, 20 bp, synthetic construct
  source 1..20: mol_type = other DNA
actacaaactagcaaggatg

SEQ ID NO: 174 - DNA, 20 bp, synthetic construct
  source 1..20: mol_type = other DNA
gactacaaactagcaaggat

SEQ ID NO: 175 - DNA, 20 bp, synthetic construct
  source 1..20: mol_type = other DNA
accatgtaaggtaaacaggg

SEQ ID NO: 176 - DNA, 20 bp, synthetic construct
  source 1..20: mol_type = other DNA
caaactagcaaggatggggt

SEQ ID NO: 177 - DNA, 20 bp, synthetic construct
  source 1..20: mol_type = other DNA
agactacaaactagcaagga

SEQ ID NO: 178 - DNA, 20 bp, synthetic construct
  source 1..20: mol_type = other DNA
acaaactagcaaggatgggg

SEQ ID NO: 179 - DNA, 20 bp, synthetic construct
  source 1..20: mol_type = other DNA
gccctgcctccgcgcgcggg

SEQ ID NO: 180 - DNA, 20 bp, synthetic construct
  source 1..20: mol_type = other DNA
aggcagggccgccgcagtcg

SEQ ID NO: 181 - DNA, 20 bp, synthetic construct
  source 1..20: mol_type = other DNA
gccctgccagcttgagcggg

SEQ ID NO: 182 - DNA, 20 bp, synthetic construct
  source 1..20: mol_type = other DNA
ggtcggggcgccctccgagg

SEQ ID NO: 183 - DNA, 20 bp, synthetic construct
  source 1..20: mol_type = other DNA
cgctgcgggattaacccgcg

SEQ ID NO: 184 - DNA, 20 bp, synthetic construct
  source 1..20: mol_type = other DNA
gcgctaatcctcgactgcgg

SEQ ID NO: 185 - DNA, 20 bp, synthetic construct
  source 1..20: mol_type = other DNA
gccgggcgtccagtccacgc

SEQ ID NO: 186 - DNA, 20 bp, synthetic construct
  source 1..20: mol_type = other DNA
ggccgggcgtccagtccacg

SEQ ID NO: 187 - DNA, 20 bp, synthetic construct
  source 1..20: mol_type = other DNA
aacgcgctaatcctcgactg

SEQ ID NO: 188 - DNA, 20 bp, synthetic construct
  source 1..20: mol_type = other DNA
ggtggggcaggaggacgcgg

SEQ ID NO: 189 - DNA, 20 bp, synthetic construct
  source 1..20: mol_type = other DNA
cctgggagcctaactgaggg

SEQ ID NO: 190 - DNA, 20 bp, synthetic construct
  source 1..20: mol_type = other DNA
aggtcccttggtggaaggtg

SEQ ID NO: 191 - DNA, 20 bp, synthetic construct
  source 1..20: mol_type = other DNA
agtcgtggtactttacgcct

SEQ ID NO: 192 - DNA, 20 bp, synthetic construct
  source 1..20: mol_type = other DNA
ttacgcctcggtgtttaggg

SEQ ID NO: 193 - DNA, 20 bp, synthetic construct
  source 1..20: mol_type = other DNA
aggctcctccctaaacaccg

SEQ ID NO: 194 - DNA, 20 bp, synthetic construct
  source 1..20: mol_type = other DNA
cctcctgggagcctaactgg

SEQ ID NO: 195 - DNA, 20 bp, synthetic construct
  source 1..20: mol_type = other DNA
ggaaggtgggtgtgtagtcg

SEQ ID NO: 196 - DNA, 20 bp, synthetic construct
  source 1..20: mol_type = other DNA
agaaatctcaggtcccttgg

SEQ ID NO: 197 - DNA, 20 bp, synthetic construct
  source 1..20: mol_type = other DNA
tacacacccaccttccacca

SEQ ID NO: 198 - DNA, 20 bp, synthetic construct
  source 1..20: mol_type = other DNA
gcctctacccccctcagttg

SEQ ID NO: 199 - DNA, 20 bp, synthetic construct
  source 1..20: mol_type = other DNA
agccctcattgatcagggag

SEQ ID NO: 200 - DNA, 20 bp, synthetic construct
  source 1..20: mol_type = other DNA
gcttatttaaatgatctctg

SEQ ID NO: 201 - DNA, 20 bp, synthetic construct
  source 1..20: mol_type = other DNA
taagccctcattgatcaggg

SEQ ID NO: 202 - DNA, 20 bp, synthetic construct
  source 1..20: mol_type = other DNA
gggacacgggtcacatctcg

SEQ ID NO: 203 - DNA, 20 bp, synthetic construct
  source 1..20: mol_type = other DNA
aagccctcattgatcaggga

SEQ ID NO: 204 - DNA, 20 bp, synthetic construct
  source 1..20: mol_type = other DNA
aaataagccctcattgatca

SEQ ID NO: 205 - DNA, 20 bp, synthetic construct
  source 1..20: mol_type = other DNA
acaaaggcgtgttcatcagg

SEQ ID NO: 206 - DNA, 20 bp, synthetic construct
  source 1..20: mol_type = other DNA
acccctccctgatcaatgag

SEQ ID NO: 207 - DNA, 20 bp, synthetic construct
  source 1..20: mol_type = other DNA
tcacccctccctgatcaatg

SEQ ID NO: 208 - DNA, 20 bp, synthetic construct
  source 1..20: mol_type = other DNA
ggcggggacgaaatcagagg

SEQ ID NO: 209 - DNA, 20 bp, synthetic construct
  source 1..20: mol_type = other DNA
ttctcctcgggtggttacgg

SEQ ID NO: 210 - DNA, 20 bp, synthetic construct
  source 1..20: mol_type = other DNA
gtcgcgggagcccagttaag

SEQ ID NO: 211 - DNA, 20 bp, synthetic construct
  source 1..20: mol_type = other DNA
cctccctcctgattgggcag

SEQ ID NO: 212 - DNA, 20 bp, synthetic construct
  source 1..20: mol_type = other DNA
atcaggagggaggcgacggg

SEQ ID NO: 213 - DNA, 20 bp, synthetic construct
  source 1..20: mol_type = other DNA
cctctgcccaatcaggaggg

SEQ ID NO: 214 - DNA, 20 bp, synthetic construct
  source 1..20: mol_type = other DNA
aggccccccaatcggctgcg

SEQ ID NO: 215 - DNA, 20 bp, synthetic construct
  source 1..20: mol_type = other DNA
cagccgcgcagccgattggg

SEQ ID NO: 216 - DNA, 20 bp, synthetic construct
  source 1..20: mol_type = other DNA
ctttacatatgcagtcccac

SEQ ID NO: 217 - DNA, 20 bp, synthetic construct
  source 1..20: mol_type = other DNA
taaccacccgaggagaaccg

SEQ ID NO: 218 - DNA, 20 bp, synthetic construct
  source 1..20: mol_type = other DNA
aggctgccaggttctcctcg

SEQ ID NO: 219 - DNA, 20 bp, synthetic construct
  source 1..20: mol_type = other DNA
aaagtgacagaatcaagctg

SEQ ID NO: 220 - DNA, 20 bp, synthetic construct
  source 1..20: mol_type = other DNA
cccatggttccagagtcggg

SEQ ID NO: 221 - DNA, 20 bp, synthetic construct
  source 1..20: mol_type = other DNA
gaggggcaacccccgactcg

SEQ ID NO: 222 - DNA, 20 bp, synthetic construct
  source 1..20: mol_type = other DNA
tgacagaatcaagctggggt

SEQ ID NO: 223 - DNA, 20 bp, synthetic construct
  source 1..20: mol_type = other DNA
gtgacagaatcaagctgggg

SEQ ID NO: 224 - DNA, 20 bp, synthetic construct
  source 1..20: mol_type = other DNA
ggccctcaaacatggagagg

SEQ ID NO: 225 - DNA, 20 bp, synthetic construct
  source 1..20: mol_type = other DNA
gacagaatcaagctggggtg

SEQ ID NO: 226 - DNA, 20 bp, synthetic construct
  source 1..20: mol_type = other DNA
ttaaagtgacagaatcaagc

SEQ ID NO: 227 - DNA, 20 bp, synthetic construct
  source 1..20: mol_type = other DNA
taaagtgacagaatcaagct

SEQ ID NO: 228 - DNA, 20 bp, synthetic construct
  source 1..20: mol_type = other DNA
acccccgactctggaaccag

SEQ ID NO: 229 - DNA, 20 bp, synthetic construct
  source 1..20: mol_type = other DNA
cagccgcccgttagggcgcg

SEQ ID NO: 230 - DNA, 20 bp, synthetic construct
  source 1..20: mol_type = other DNA
cacgccggccgaaagccgcg

SEQ ID NO: 231 - DNA, 20 bp, synthetic construct
  source 1..20: mol_type = other DNA
tgcctcgcgccctaacgggg

SEQ ID NO: 232 - DNA, 20 bp, synthetic construct
  source 1..20: mol_type = other DNA
tcttccatacccgccccgcg

SEQ ID NO: 233 - DNA, 20 bp, synthetic construct
  source 1..20: mol_type = other DNA
gtgcctcgcgccctaacggg

SEQ ID NO: 234 - DNA, 20 bp, synthetic construct
  source 1..20: mol_type = other DNA
gggcgggtatggaagacgga

SEQ ID NO: 235 - DNA, 20 bp, synthetic construct
  source 1..20: mol_type = other DNA
aagccgcgcggggcgggtag

SEQ ID NO: 236 - DNA, 20 bp, synthetic construct
  source 1..20: mol_type = other DNA
ggcgggtatggaagacggag

SEQ ID NO: 237 - DNA, 20 bp, synthetic construct
  source 1..20: mol_type = other DNA
aggccgagcaccgcgggaag

SEQ ID NO: 238 - DNA, 20 bp, synthetic construct
  source 1..20: mol_type = other DNA
cgcggggcgggtatggaaga

SEQ ID NO: 239 - DNA, 20 bp, synthetic construct
  source 1..20: mol_type = other DNA
gggcgagtcacgtggggggg

SEQ ID NO: 240 - DNA, 20 bp, synthetic construct
  source 1..20: mol_type = other DNA
gcggggcgagtcacgtgggg

SEQ ID NO: 241 - DNA, 20 bp, synthetic construct
  source 1..20: mol_type = other DNA
cgcggggcgagtcacgtggg

SEQ ID NO: 242 - DNA, 20 bp, synthetic construct
  source 1..20: mol_type = other DNA
gggcgcggggcgagtcacgt

SEQ ID NO: 243 - DNA, 20 bp, synthetic construct
  source 1..20: mol_type = other DNA
tgaccgagactctccatcag

SEQ ID NO: 244 - DNA, 20 bp, synthetic construct
  source 1..20: mol_type = other DNA
cgcaggcggcgacacacagg

SEQ ID NO: 245 - DNA, 20 bp, synthetic construct
  source 1..20: mol_type = other DNA
atcccgtggctcccggcccg

SEQ ID NO: 246 - DNA, 20 bp, synthetic construct
  source 1..20: mol_type = other DNA
acgggattgggccgcgcacg

SEQ ID NO: 247 - DNA, 20 bp, synthetic construct
  source 1..20: mol_type = other DNA
tcccgtggctcccggcccgg

SEQ ID NO: 248 - DNA, 20 bp, synthetic construct
  source 1..20: mol_type = other DNA
ccgtgcgcggcccaatcccg

SEQ ID NO: 249 - DNA, 20 bp, synthetic construct
  source 1..20: mol_type = other DNA
gaggccgggacgcgcccact

SEQ ID NO: 250 - DNA, 20 bp, synthetic construct
  source 1..20: mol_type = other DNA
ctctcccgggagaggagagg

SEQ ID NO: 251 - DNA, 20 bp, synthetic construct
  source 1..20: mol_type = other DNA
gtctctctcagggtgcgctc

SEQ ID NO: 252 - DNA, 20 bp, synthetic construct
  source 1..20: mol_type = other DNA
cggaagagcgagccctttag

SEQ ID NO: 253 - DNA, 20 bp, synthetic construct
  source 1..20: mol_type = other DNA
ctcccgggagaggagaggcg

SEQ ID NO: 254 - DNA, 20 bp, synthetic construct
  source 1..20: mol_type = other DNA
ccctttaaggctatgccgag

SEQ ID NO: 255 - DNA, 20 bp, synthetic construct
  source 1..20: mol_type = other DNA
gacccccgccctccacagcg

SEQ ID NO: 256 - DNA, 20 bp, synthetic construct
  source 1..20: mol_type = other DNA
cctttaaggctatgccgagt

SEQ ID NO: 257 - DNA, 20 bp, synthetic construct
  source 1..20: mol_type = other DNA
aggccgggacgcgcccactg

SEQ ID NO: 258 - DNA, 20 bp, synthetic construct
  source 1..20: mol_type = other DNA
ccactcggcatagccttaag

SEQ ID NO: 259 - DNA, 20 bp, synthetic construct
  source 1..20: mol_type = other DNA
gaacccgcgcccggacgggc

SEQ ID NO: 260 - DNA, 20 bp, synthetic construct
  source 1..20: mol_type = other DNA
gggagcccgagtttaggaag

SEQ ID NO: 261 - DNA, 20 bp, synthetic construct
  source 1..20: mol_type = other DNA
gcgtcttccggcgcccgcgg

SEQ ID NO: 262 - DNA, 20 bp, synthetic construct
  source 1..20: mol_type = other DNA
gccaactgcggcgcgtcttc

SEQ ID NO: 263 - DNA, 20 bp, synthetic construct
  source 1..20: mol_type = other DNA
gcgccaacccgcgcccggac

SEQ ID NO: 264 - DNA, 20 bp, synthetic construct
  source 1..20: mol_type = other DNA
gtcgcctcctccgcgggcgc

SEQ ID NO: 265 - DNA, 20 bp, synthetic construct
  source 1..20: mol_type = other DNA
gcccgagtttaggaagagga

SEQ ID NO: 266 - DNA, 20 bp, synthetic construct
  source 1..20: mol_type = other DNA
gctccctgcccgtccgggcg

SEQ ID NO: 267 - DNA, 20 bp, synthetic construct
  source 1..20: mol_type = other DNA
gccgctcctccctgcccgtc

SEQ ID NO: 268 - DNA, 20 bp, synthetic construct
  source 1..20: mol_type = other DNA
ggcgccgcggggctcctcca

SEQ ID NO: 269 - DNA, 20 bp, synthetic construct
  source 1..20: mol_type = other DNA
tctgcgcatcaaagctagcg

SEQ ID NO: 270 - DNA, 20 bp, synthetic construct
  source 1..20: mol_type = other DNA
agaggggcctcgggcgggag

SEQ ID NO: 271 - DNA, 20 bp, synthetic construct
  source 1..20: mol_type = other DNA
acccgggcggcggcagtccg

SEQ ID NO: 272 - DNA, 20 bp, synthetic construct
  source 1..20: mol_type = other DNA
ccccgccaccctcctcctcc

SEQ ID NO: 273 - DNA, 20 bp, synthetic construct
  source 1..20: mol_type = other DNA
ccgcccgggtcgcccacgtg

SEQ ID NO: 274 - DNA, 20 bp, synthetic construct
  source 1..20: mol_type = other DNA
cgggaggaggagggtggcgg

SEQ ID NO: 275 - DNA, 20 bp, synthetic construct
  source 1..20: mol_type = other DNA
ggtgaggccgcagaggcgga

SEQ ID NO: 276 - DNA, 20 bp, synthetic construct
  source 1..20: mol_type = other DNA
cgcagatccctccgcctctg

SEQ ID NO: 277 - DNA, 20 bp, synthetic construct
  source 1..20: mol_type = other DNA
gcggggcggtgaggccgcag

SEQ ID NO: 278 - DNA, 20 bp, synthetic construct
  source 1..20: mol_type = other DNA
ccgggaggaggagggtggcg

SEQ ID NO: 279 - DNA, 20 bp, synthetic construct
  source 1..20: mol_type = other DNA
aaaagagaagtggctcattg

SEQ ID NO: 280 - DNA, 20 bp, synthetic construct
  source 1..20: mol_type = other DNA
gtgctctttctcaaattagg

SEQ ID NO: 281 - DNA, 20 bp, synthetic construct
  source 1..20: mol_type = other DNA
ttagggcatttcctgaaccc

SEQ ID NO: 282 - DNA, 20 bp, synthetic construct
  source 1..20: mol_type = other DNA
ggttggaatccagttagttg

SEQ ID NO: 283 - DNA, 20 bp, synthetic construct
  source 1..20: mol_type = other DNA
gttaattatggagccacaag

SEQ ID NO: 284 - DNA, 20 bp, synthetic construct
  source 1..20: mol_type = other DNA
ggcatttcctgaaccctggt

SEQ ID NO: 285 - DNA, 20 bp, synthetic construct
  source 1..20: mol_type = other DNA
cttaactgaaattccaacca

SEQ ID NO: 286 - DNA, 20 bp, synthetic construct
  source 1..20: mol_type = other DNA
ccttaactgaaattccaacc

SEQ ID NO: 287 - DNA, 20 bp, synthetic construct
  source 1..20: mol_type = other DNA
cctggttggaatttcagtta

SEQ ID NO: 288 - DNA, 20 bp, synthetic construct
  source 1..20: mol_type = other DNA
ccaacaagagcttttactgg

SEQ ID NO: 289 - DNA, 20 bp, synthetic construct
  source 1..20: mol_type = other DNA
gcgggcggcaagctgagagt

SEQ ID NO: 290 - DNA, 20 bp, synthetic construct
  source 1..20: mol_type = other DNA
gcgcagaggggtgacgtcag

SEQ ID NO: 291 - DNA, 20 bp, synthetic construct
  source 1..20: mol_type = other DNA
gtaaggctggcaagtcccca

SEQ ID NO: 292 - DNA, 20 bp, synthetic construct
  source 1..20: mol_type = other DNA
ggggcggctgagtcacgcta

SEQ ID NO: 293 - DNA, 20 bp, synthetic construct
  source 1..20: mol_type = other DNA
gggcgcgtagacagacgcag

SEQ ID NO: 294 - DNA, 20 bp, synthetic construct
  source 1..20: mol_type = other DNA
gcgcgcgccggaagtggcta

SEQ ID NO: 295 - DNA, 20 bp, synthetic construct
  source 1..20: mol_type = other DNA
ggccccggaagcgagaagga

SEQ ID NO: 296 - DNA, 20 bp, synthetic construct
  source 1..20: mol_type = other DNA
gctcgcggccggaagcgccg

SEQ ID NO: 297 - DNA, 20 bp, synthetic construct
  source 1..20: mol_type = other DNA
ggcgggacggagtgacttca

SEQ ID NO: 298 - DNA, 20 bp, synthetic construct
  source 1..20: mol_type = other DNA
gcgcggcggcgtggcgcaga

SEQ ID NO: 299 - DNA, 20 bp, synthetic construct
  source 1..20: mol_type = other DNA
caccgaagcccaagagctgg

SEQ ID NO: 300 - DNA, 20 bp, synthetic construct
  source 1..20: mol_type = other DNA
tgtggggagggttgttattg

SEQ ID NO: 301 - DNA, 20 bp, synthetic construct
  source 1..20: mol_type = other DNA
gtgagctgaaccttctcgtg

SEQ ID NO: 302 - DNA, 20 bp, synthetic construct
  source 1..20: mol_type = other DNA
accgaagcccaagagctggg

SEQ ID NO: 303 - DNA, 20 bp, synthetic construct
  source 1..20: mol_type = other DNA
gcgccccctgccggcccctg

SEQ ID NO: 304 - DNA, 20 bp, synthetic construct
  source 1..20: mol_type = other DNA
ccgaagcccaagagctgggg

SEQ ID NO: 305 - DNA, 20 bp, synthetic construct
  source 1..20: mol_type = other DNA
ccagctcttgggcttcggtg

SEQ ID NO: 306 - DNA, 20 bp, synthetic construct
  source 1..20: mol_type = other DNA
cacaccgaagcccaagagct

SEQ ID NO: 307 - DNA, 20 bp, synthetic construct
  source 1..20: mol_type = other DNA
agtgcaccgggaaccgcgcg

SEQ ID NO: 308 - DNA, 20 bp, synthetic construct
  source 1..20: mol_type = other DNA
agctcttgggcttcggtgtg

SEQ ID NO: 309 - DNA, 20 bp, synthetic construct
  source 1..20: mol_type = other DNA
caaacgggtggcccacgatg

SEQ ID NO: 310 - DNA, 20 bp, synthetic construct
  source 1..20: mol_type = other DNA
ccagggtctcagccaatcgt

SEQ ID NO: 311 - DNA, 20 bp, synthetic construct
  source 1..20: mol_type = other DNA
ccaaacgggtggcccacgat

SEQ ID NO: 312 - DNA, 20 bp, synthetic construct
  source 1..20: mol_type = other DNA
gagccgcgccctgcgcgatg

SEQ ID NO: 313 - DNA, 20 bp, synthetic construct
  source 1..20: mol_type = other DNA
gcgtacgcgcttcctcaatg

SEQ ID NO: 314 - DNA, 20 bp, synthetic construct
  source 1..20: mol_type = other DNA
tgcgcgattggccaaacggg

SEQ ID NO: 315 - DNA, 20 bp, synthetic construct
  source 1..20: mol_type = other DNA
cgtacgcgcttcctcaatgg

SEQ ID NO: 316 - DNA, 20 bp, synthetic construct
  source 1..20: mol_type = other DNA
gcgcccagcccagtcgcggg

SEQ ID NO: 317 - DNA, 20 bp, synthetic construct
  source 1..20: mol_type = other DNA
gccagggtctcagccaatcg

SEQ ID NO: 318 - DNA, 20 bp, synthetic construct
  source 1..20: mol_type = other DNA
cccgtttggccaatcgcgca

SEQ ID NO: 319 - DNA, 20 bp, synthetic construct
  source 1..20: mol_type = other DNA
catggttctgtagcaccggt

SEQ ID NO: 320 - DNA, 20 bp, synthetic construct
  source 1..20: mol_type = other DNA
agctcgagcggggtccagag

SEQ ID NO: 321 - DNA, 20 bp, synthetic construct
  source 1..20: mol_type = other DNA
cctctccctcatttgttaag

SEQ ID NO: 322 - DNA, 20 bp, synthetic construct
  source 1..20: mol_type = other DNA
caccgaaatacctcttccag

SEQ ID NO: 323 - DNA, 20 bp, synthetic construct
  source 1..20: mol_type = other DNA
aggaggaaggtgggggggcg

SEQ ID NO: 324 - DNA, 20 bp, synthetic construct
  source 1..20: mol_type = other DNA
gaatgcgcgtttgcaggagg

SEQ ID NO: 325 - DNA, 20 bp, synthetic construct
  source 1..20: mol_type = other DNA
ttccagggcgggttttcggg

SEQ ID NO: 326 - DNA, 20 bp, synthetic construct
  source 1..20: mol_type = other DNA
tgtggagaggctggtgcaaa

SEQ ID NO: 327 - DNA, 20 bp, synthetic construct
  source 1..20: mol_type = other DNA
aaggaatgcgcgtttgcagg

SEQ ID NO: 328 - DNA, 20 bp, synthetic construct
  source 1..20: mol_type = other DNA
atttgttaaaggtatcctac

SEQ ID NO: 329 - DNA, 20 bp, synthetic construct
  source 1..20: mol_type = other DNA
gccgtgagctccccgattgg

SEQ ID NO: 330 - DNA, 20 bp, synthetic construct
  source 1..20: mol_type = other DNA
ccccgattggggctcggtcg

SEQ ID NO: 331 - DNA, 20 bp, synthetic construct
  source 1..20: mol_type = other DNA
gccccaatcggggagctcag

SEQ ID NO: 332 - DNA, 20 bp, synthetic construct
  source 1..20: mol_type = other DNA
tgagctccccgattggggct

SEQ ID NO: 333 - DNA, 20 bp, synthetic construct
  source 1..20: mol_type = other DNA
ggccgtgagctccccgattg

SEQ ID NO: 334 - DNA, 20 bp, synthetic construct
  source 1..20: mol_type = other DNA
ggccggaccgagccccaatg

SEQ ID NO: 335 - DNA, 20 bp, synthetic construct
  source 1..20: mol_type = other DNA
agccccaatcggggagctca

SEQ ID NO: 336 - DNA, 20 bp, synthetic construct
  source 1..20: mol_type = other DNA
aattcgctgacgtgtaggag

SEQ ID NO: 337 - DNA, 20 bp, synthetic construct
  source 1..20: mol_type = other DNA
tacacgtcagcgaattcagc

SEQ ID NO: 338 - DNA, 20 bp, synthetic construct
  source 1..20: mol_type = other DNA
gccggaccgagccccaatcg

SEQ ID NO: 339 - DNA, 20 bp, synthetic construct
  source 1..20: mol_type = other DNA
ctcggcggcagcggctgccg

SEQ ID NO: 340 - DNA, 20 bp, synthetic construct
  source 1..20: mol_type = other DNA
cccccacccccggcccgcac

SEQ ID NO: 341 - DNA, 20 bp, synthetic construct
  source 1..20: mol_type = other DNA
gaggggcggctcggcggcag

SEQ ID NO: 342 - DNA, 20 bp, synthetic construct
  source 1..20: mol_type = other DNA
ggggcgcgcggggggggcgc

SEQ ID NO: 343 - DNA, 20 bp, synthetic construct
  source 1..20: mol_type = other DNA
tgccgcggcgcggccggtgc

SEQ ID NO: 344 - DNA, 20 bp, synthetic construct
  source 1..20: mol_type = other DNA
cggcagcggctgccgcggcg

SEQ ID NO: 345 - DNA, 20 bp, synthetic construct
  source 1..20: mol_type = other DNA
cggaacgcaagaggttaatg

SEQ ID NO: 346 - DNA, 20 bp, synthetic construct
  source 1..20: mol_type = other DNA
cccgcgaggggcggctcggg

SEQ ID NO: 347 - DNA, 20 bp, synthetic construct
  source 1..20: mol_type = other DNA
gccgccgagccgcccctcgg

SEQ ID NO: 348 - DNA, 20 bp, synthetic construct
  source 1..20: mol_type = other DNA
tcagggagccggctctcggg

SEQ ID NO: 349 - DNA, 20 bp, synthetic construct
  source 1..20: mol_type = other DNA
gagggagcgcgagagaggga

SEQ ID NO: 350 - DNA, 20 bp, synthetic construct
  source 1..20: mol_type = other DNA
agagggagcgcgagagaggg

SEQ ID NO: 351 - DNA, 20 bp, synthetic construct
  source 1..20: mol_type = other DNA
ctctcgcgctccctctccct

SEQ ID NO: 352 - DNA, 20 bp, synthetic construct
  source 1..20: mol_type = other DNA
ggaggcggtagttggaagtg

SEQ ID NO: 353 - DNA, 20 bp, synthetic construct
  source 1..20: mol_type = other DNA
atgggctgccccgggtctgg

SEQ ID NO: 354 - DNA, 20 bp, synthetic construct
  source 1..20: mol_type = other DNA
caggccagcgccccgcggtg

SEQ ID NO: 355 - DNA, 20 bp, synthetic construct
  source 1..20: mol_type = other DNA
cggcagcccgtgagcgcggg

SEQ ID NO: 356 - DNA, 20 bp, synthetic construct
  source 1..20: mol_type = other DNA
gggaggaggggacggtgctt

SEQ ID NO: 357 - DNA, 20 bp, synthetic construct
  source 1..20: mol_type = other DNA
cggaggcggtagttggaagt

SEQ ID NO: 358 - DNA, 20 bp, synthetic construct
  source 1..20: mol_type = other DNA
tgattgctggtcgtttgttg

SEQ ID NO: 359 - DNA, 20 bp, synthetic construct
  source 1..20: mol_type = other DNA
aagagaaacaccaacaaaga

SEQ ID NO: 360 - DNA, 20 bp, synthetic construct
  source 1..20: mol_type = other DNA
aggcctgcagagtcacgtgg

SEQ ID NO: 361 - DNA, 20 bp, synthetic construct
  source 1..20: mol_type = other DNA
acaccaacaaagaaggcgag

SEQ ID NO: 362 - DNA, 20 bp, synthetic construct
  source 1..20: mol_type = other DNA
agaggcctgcagagtcacgt

SEQ ID NO: 363 - DNA, 20 bp, synthetic construct
  source 1..20: mol_type = other DNA
tgcccccacgtgactctgcg

SEQ ID NO: 364 - DNA, 20 bp, synthetic construct
  source 1..20: mol_type = other DNA
ggcctgcagagtcacgtggg

SEQ ID NO: 365 - DNA, 20 bp, synthetic construct
  source 1..20: mol_type = other DNA
aatacccctatctatctggg

SEQ ID NO: 366 - DNA, 20 bp, synthetic construct
  source 1..20: mol_type = other DNA
agagaaacaccaacaaagag

SEQ ID NO: 367 - DNA, 20 bp, synthetic construct
  source 1..20: mol_type = other DNA
gagaggcctgcagagtcacg

SEQ ID NO: 368 - DNA, 20 bp, synthetic construct
  source 1..20: mol_type = other DNA
gaggcctgcagagtcacgtg

SEQ ID NO: 369 - DNA, 20 bp, synthetic construct
  source 1..20: mol_type = other DNA
gggcggcgcgctcggactgg

SEQ ID NO: 370 - DNA, 20 bp, synthetic construct
  source 1..20: mol_type = other DNA
atcgccaggagacagacctc

SEQ ID NO: 371 - DNA, 20 bp, synthetic construct
  source 1..20: mol_type = other DNA
agccccaggacgcgacagcg

SEQ ID NO: 372 - DNA, 20 bp, synthetic construct
  source 1..20: mol_type = other DNA
agacagacctcaggcgccgg

SEQ ID NO: 373 - DNA, 20 bp, synthetic construct
  source 1..20: mol_type = other DNA
ccctcccgcctccccgaggg

SEQ ID NO: 374 - DNA, 20 bp, synthetic construct
  source 1..20: mol_type = other DNA
gctcccctcccgcctccccg

SEQ ID NO: 375 - DNA, 20 bp, synthetic construct
  source 1..20: mol_type = other DNA
agacagacctcaggcgccgg

SEQ ID NO: 376 - DNA, 20 bp, synthetic construct
  source 1..20: mol_type = other DNA
gtctcctggcgatcccgcag

SEQ ID NO: 377 - DNA, 20 bp, synthetic construct
  source 1..20: mol_type = other DNA
gagacagacctcaggcgccg

SEQ ID NO: 378 - DNA, 20 bp, synthetic construct
  source 1..20: mol_type = other DNA
gggggtgattcaggagtgag

SEQ ID NO: 379 - DNA, 20 bp, synthetic construct
  source 1..20: mol_type = other DNA
gcctgacttggggtgacaag

SEQ ID NO: 380 - DNA, 20 bp, synthetic construct
  source 1..20: mol_type = other DNA
gtgctaattacaactgctgg

SEQ ID NO: 381 - DNA, 20 bp, synthetic construct
  source 1..20: mol_type = other DNA
gggaaccgggaaactgcggg

SEQ ID NO: 382 - DNA, 20 bp, synthetic construct
  source 1..20: mol_type = other DNA
gttaatcggtcccccgccgg

SEQ ID NO: 383 - DNA, 20 bp, synthetic construct
  source 1..20: mol_type = other DNA
ccagcctatccaccggcggg

SEQ ID NO: 384 - DNA, 20 bp, synthetic construct
  source 1..20: mol_type = other DNA
ccgccggtggataggctggg

SEQ ID NO: 385 - DNA, 20 bp, synthetic construct
  source 1..20: mol_type = other DNA
ccgcccagcctatccaccgg

SEQ ID NO: 386 - DNA, 20 bp, synthetic construct
  source 1..20: mol_type = other DNA
gccccgcccagcctatccac

SEQ ID NO: 387 - DNA, 20 bp, synthetic construct
  source 1..20: mol_type = other DNA
atggttaatcggtcccccgc

SEQ ID NO: 388 - DNA, 20 bp, synthetic construct
  source 1..20: mol_type = other DNA
gcccagcctatccaccggcg

SEQ ID NO: 389 - DNA, 20 bp, synthetic construct
  source 1..20: mol_type = other DNA
acagggaaatgcgcgcgccg

SEQ ID NO: 390 - DNA, 20 bp, synthetic construct
  source 1..20: mol_type = other DNA
gggaaatgcgcgcgccgagg

SEQ ID NO: 391 - DNA, 20 bp, synthetic construct
  source 1..20: mol_type = other DNA
gccccgcgctggaccgctcg

SEQ ID NO: 392 - DNA, 20 bp, synthetic construct
  source 1..20: mol_type = other DNA
tccctgttcccaaggccccg

SEQ ID NO: 393 - DNA, 20 bp, synthetic construct
  source 1..20: mol_type = other DNA
tccctgttcccaaggcccca

SEQ ID NO: 394 - DNA, 20 bp, synthetic construct
  source 1..20: mol_type = other DNA
aatgaaaactgaaagcagcg

SEQ ID NO: 395 - DNA, 20 bp, synthetic construct
  source 1..20: mol_type = other DNA
cccgaccgccagtcgccctg

SEQ ID NO: 396 - DNA, 20 bp, synthetic construct
  source 1..20: mol_type = other DNA
gggctccccagggtgtaggg

SEQ ID NO: 397 - DNA, 20 bp, synthetic construct
  source 1..20: mol_type = other DNA
caaggccccagggcgactgg

SEQ ID NO: 398 - DNA, 20 bp, synthetic construct
  source 1..20: mol_type = other DNA
cggcctcccgagaccgcagg

SEQ ID NO: 399 - DNA, 20 bp, synthetic construct
  source 1..20: mol_type = other DNA
ggaccacggtaccacccaat

SEQ ID NO: 400 - DNA, 20 bp, synthetic construct
  source 1..20: mol_type = other DNA
ggtaccacccaatgggaaag

SEQ ID NO: 401 - DNA, 20 bp, synthetic construct
  source 1..20: mol_type = other DNA
gaccacggtaccacccaatg

SEQ ID NO: 402 - DNA, 20 bp, synthetic construct
  source 1..20: mol_type = other DNA
gtaccacccaatgggaaagg

SEQ ID NO: 403 - DNA, 20 bp, synthetic construct
  source 1..20: mol_type = other DNA
tcttcccctttcccattggg

SEQ ID NO: 404 - DNA, 20 bp, synthetic construct
  source 1..20: mol_type = other DNA
cggaccacggtaccacccaa

SEQ ID NO: 405 - DNA, 20 bp, synthetic construct
  source 1..20: mol_type = other DNA
ggaaaggggaagaaccgaag

SEQ ID NO: 406 - DNA, 20 bp, synthetic construct
  source 1..20: mol_type = other DNA
tgtccccgaaatggattgtg

SEQ ID NO: 407 - DNA, 20 bp, synthetic construct
  source 1..20: mol_type = other DNA
acggtaccacccaatgggaa

SEQ ID NO: 408 - DNA, 20 bp, synthetic construct
  source 1..20: mol_type = other DNA
tttcccattgggtggtaccg

SEQ ID NO: 409 - DNA, 20 bp, synthetic construct
  source 1..20: mol_type = other DNA
ccccgcccccagctcccgag

SEQ ID NO: 410 - DNA, 20 bp, synthetic construct
  source 1..20: mol_type = other DNA
tcacgctgggactccgaatg

SEQ ID NO: 411 - DNA, 20 bp, synthetic construct
  source 1..20: mol_type = other DNA
ctgtttaatattgatgaggg

SEQ ID NO: 412 - DNA, 20 bp, synthetic construct
  source 1..20: mol_type = other DNA
agggatctgggaacccctcg

SEQ ID NO: 413 - DNA, 20 bp, synthetic construct
  source 1..20: mol_type = other DNA
ggctgtttaatattgatgag

SEQ ID NO: 414 - DNA, 20 bp, synthetic construct
  source 1..20: mol_type = other DNA
cacacccccagctctccatg

SEQ ID NO: 415 - DNA, 20 bp, synthetic construct
  source 1..20: mol_type = other DNA
gctgtttaatattgatgagg

SEQ ID NO: 416 - DNA, 20 bp, synthetic construct
  source 1..20: mol_type = other DNA
ccagccaatggagagctggg

SEQ ID NO: 417 - DNA, 20 bp, synthetic construct
  source 1..20: mol_type = other DNA
ctcatcaatattaaacagcc

SEQ ID NO: 418 - DNA, 20 bp, synthetic construct
  source 1..20: mol_type = other DNA
gccaggcagccattggcaga

SEQ ID NO: 419 - DNA, 20 bp, synthetic construct
  source 1..20: mol_type = other DNA
actctatgcaggttgataag

SEQ ID NO: 420 - DNA, 20 bp, synthetic construct
  source 1..20: mol_type = other DNA
aaagaggtgatgacatcaag

SEQ ID NO: 421 - DNA, 20 bp, synthetic construct
  source 1..20: mol_type = other DNA
ctcatcacatgactctatgc

SEQ ID NO: 422 - DNA, 20 bp, synthetic construct
  source 1..20: mol_type = other DNA
tgattaactcagaagtggag

SEQ ID NO: 423 - DNA, 20 bp, synthetic construct
  source 1..20: mol_type = other DNA
tcatcacatgactctatgcg

SEQ ID NO: 424 - DNA, 20 bp, synthetic construct
  source 1..20: mol_type = other DNA
tgctatgcagcacagagagg

SEQ ID NO: 425 - DNA, 20 bp, synthetic construct
  source 1..20: mol_type = other DNA
ccatgtatccagtttcatga

SEQ ID NO: 426 - DNA, 20 bp, synthetic construct
  source 1..20: mol_type = other DNA
atggtttctaatcagaagaa

SEQ ID NO: 427 - DNA, 20 bp, synthetic construct
  source 1..20: mol_type = other DNA
tggaaagttgtaaaactgag

SEQ ID NO: 428 - DNA, 20 bp, synthetic construct
  source 1..20: mol_type = other DNA
agaaatgattaactcagaag

SEQ ID NO: 429 - DNA, 20 bp, synthetic construct
  source 1..20: mol_type = other DNA
gtggccgcgtttgaagtctc

SEQ ID NO: 430 - DNA, 20 bp, synthetic construct
  source 1..20: mol_type = other DNA
ggcagccgggggtaccaagg

SEQ ID NO: 431 - DNA, 20 bp, synthetic construct
  source 1..20: mol_type = other DNA
ggctgctggttggccggcga

SEQ ID NO: 432 - DNA, 20 bp, synthetic construct
  source 1..20: mol_type = other DNA
gcccgccggctaactcgccg

SEQ ID NO: 433 - DNA, 20 bp, synthetic construct
  source 1..20: mol_type = other DNA
gcccgcggcgagttagccgg

SEQ ID NO: 434 - DNA, 20 bp, synthetic construct
  source 1..20: mol_type = other DNA
ggcgctccagagcccctctg

SEQ ID NO: 435 - DNA, 20 bp, synthetic construct
  source 1..20: mol_type = other DNA
ggcgccggagacttcaaacg

SEQ ID NO: 436 - DNA, 20 bp, synthetic construct
  source 1..20: mol_type = other DNA
gggtaccaagggggcaacgg

SEQ ID NO: 437 - DNA, 20 bp, synthetic construct
  source 1..20: mol_type = other DNA
ggggcgcacgtgaccctcgc

SEQ ID NO: 438 - DNA, 20 bp, synthetic construct
  source 1..20: mol_type = other DNA
ggggtagctaaggacagcgg

SEQ ID NO: 439 - RNA, 20 nt, synthetic construct
  source 1..20: mol_type = other RNA
ctggacatgagtcacttaat

SEQ ID NO: 440 - RNA, 20 nt, synthetic construct
  source 1..20: mol_type = other RNA
caattaagtgactcatgtcc

SEQ ID NO: 441 - RNA, 20 nt, synthetic construct
  source 1..20: mol_type = other RNA
tttatggcacagattcagtg

SEQ ID NO: 442 - RNA, 20 nt, synthetic construct
  source 1..20: mol_type = other RNA
cttgacatctgtcacagtgt

SEQ ID NO: 443 - RNA, 20 nt, synthetic construct
  source 1..20: mol_type = other RNA
aatcctattggacagaaacc

SEQ ID NO: 444 - RNA, 20 nt, synthetic construct
  source 1..20: mol_type = other RNA
acttgacatctgtcacagtg

SEQ ID NO: 445 - RNA, 20 nt, synthetic construct
  source 1..20: mol_type = other RNA
gagataaaggcttaggaagg

SEQ ID NO: 446 - RNA, 20 nt, synthetic construct
  source 1..20: mol_type = other RNA
acggctagatactgtttagg

SEQ ID NO: 447 - RNA, 20 nt, synthetic construct
  source 1..20: mol_type = other RNA
tggacatgagtcacttaatg

SEQ ID NO: 448 - RNA, 20 nt, synthetic construct
  source 1..20: mol_type = other RNA
tatttaatgggaaatcctat

SEQ ID NO: 449 - RNA, 20 nt, synthetic construct
  source 1..20: mol_type = other RNA
tctgctccgggctccccatg

SEQ ID NO: 450 - RNA, 20 nt, synthetic construct
  source 1..20: mol_type = other RNA
tgcgcggagaaccgaacggg

SEQ ID NO: 451 - RNA, 20 nt, synthetic construct
  source 1..20: mol_type = other RNA
cgagcgcaagggacagggag

SEQ ID NO: 452 - RNA, 20 nt, synthetic construct
  source 1..20: mol_type = other RNA
gggaagactacaaactagca

SEQ ID NO: 453 - RNA, 20 nt, synthetic construct
  source 1..20: mol_type = other RNA
actacaaactagcaaggatg

SEQ ID NO: 454 - RNA, 20 nt, synthetic construct
  source 1..20: mol_type = other RNA
gactacaaactagcaaggat

SEQ ID NO: 455 - RNA, 20 nt, synthetic construct
  source 1..20: mol_type = other RNA
accatgtaaggtaaacaggg

SEQ ID NO: 456 - RNA, 20 nt, synthetic construct
  source 1..20: mol_type = other RNA
caaactagcaaggatggggt

SEQ ID NO: 457 - RNA, 20 nt, synthetic construct
  source 1..20: mol_type = other RNA
agactacaaactagcaagga

SEQ ID NO: 458 - RNA, 20 nt, synthetic construct
  source 1..20: mol_type = other RNA
acaaactagcaaggatgggg

SEQ ID NO: 459 - RNA, 20 nt, synthetic construct
  source 1..20: mol_type = other RNA
gccctgcctccgcgcgcggg

SEQ ID NO: 460 - RNA, 20 nt, synthetic construct
  source 1..20: mol_type = other RNA
aggcagggccgccgcagtcg

SEQ ID NO: 461 - RNA, 20 nt, synthetic construct
  source 1..20: mol_type = other RNA
gccctgccagcttgagcggg

SEQ ID NO: 462 - RNA, 20 nt, synthetic construct
  source 1..20: mol_type = other RNA
ggtcggggcgccctccgagg

SEQ ID NO: 463 - RNA, 20 nt, synthetic construct
  source 1..20: mol_type = other RNA
cgctgcgggattaacccgcg

SEQ ID NO: 464 - RNA, 20 nt, synthetic construct
  source 1..20: mol_type = other RNA
gcgctaatcctcgactgcgg

SEQ ID NO: 465 - RNA, 20 nt, synthetic construct
  source 1..20: mol_type = other RNA
gccgggcgtccagtccacgc

SEQ ID NO: 466 - RNA, 20 nt, synthetic construct
  source 1..20: mol_type = other RNA
ggccgggcgtccagtccacg

SEQ ID NO: 467 - RNA, 20 nt, synthetic construct
  source 1..20: mol_type = other RNA
aacgcgctaatcctcgactg

SEQ ID NO: 468 - RNA, 20 nt, synthetic construct
  source 1..20: mol_type = other RNA
ggtggggcaggaggacgcgg

SEQ ID NO: 469 - RNA, 20 nt, synthetic construct
  source 1..20: mol_type = other RNA
cctgggagcctaactgaggg

SEQ ID NO: 470 - RNA, 20 nt, synthetic construct
  source 1..20: mol_type = other RNA
aggtcccttggtggaaggtg

SEQ ID NO: 471 - RNA, 20 nt, synthetic construct
  source 1..20: mol_type = other RNA
agtcgtggtactttacgcct

SEQ ID NO: 472 - RNA, 20 nt, synthetic construct
  source 1..20: mol_type = other RNA
ttacgcctcggtgtttaggg

SEQ ID NO: 473 - RNA, 20 nt, synthetic construct
  source 1..20: mol_type = other RNA
aggctcctccctaaacaccg

SEQ ID NO: 474 - RNA, 20 nt, synthetic construct
  source 1..20: mol_type = other RNA
cctcctgggagcctaactgg

SEQ ID NO: 475 - RNA, 20 nt, synthetic construct
  source 1..20: mol_type = other RNA
ggaaggtgggtgtgtagtcg

SEQ ID NO: 476 - RNA, 20 nt, synthetic construct
  source 1..20: mol_type = other RNA
agaaatctcaggtcccttgg

SEQ ID NO: 477 - RNA, 20 nt, synthetic construct
  source 1..20: mol_type = other RNA
tacacacccaccttccacca

SEQ ID NO: 478 - RNA, 20 nt, synthetic construct
  source 1..20: mol_type = other RNA
gcctctacccccctcagttg

SEQ ID NO: 479 - RNA, 20 nt, synthetic construct
  source 1..20: mol_type = other RNA
agccctcattgatcagggag

SEQ ID NO: 480 - RNA, 20 nt, synthetic construct
  source 1..20: mol_type = other RNA
gcttatttaaatgatctctg

SEQ ID NO: 481 - RNA, 20 nt, synthetic construct
  source 1..20: mol_type = other RNA
taagccctcattgatcaggg

SEQ ID NO: 482 - RNA, 20 nt, synthetic construct
  source 1..20: mol_type = other RNA
gggacacgggtcacatctcg

SEQ ID NO: 483 - RNA, 20 nt, synthetic construct
  source 1..20: mol_type = other RNA
aagccctcattgatcaggga

SEQ ID NO: 484 - RNA, 20 nt, synthetic construct
  source 1..20: mol_type = other RNA
aaataagccctcattgatca

SEQ ID NO: 485 - RNA, 20 nt, synthetic construct
  source 1..20: mol_type = other RNA
acaaaggcgtgttcatcagg

SEQ ID NO: 486 - RNA, 20 nt, synthetic construct
  source 1..20: mol_type = other RNA
acccctccctgatcaatgag

SEQ ID NO: 487 - RNA, 20 nt, synthetic construct
  source 1..20: mol_type = other RNA
tcacccctccctgatcaatg

SEQ ID NO: 488 - RNA, 20 nt, synthetic construct
  source 1..20: mol_type = other RNA
ggcggggacgaaatcagagg

SEQ ID NO: 489 - RNA, 20 nt, synthetic construct
  source 1..20: mol_type = other RNA
ttctcctcgggtggttacgg

SEQ ID NO: 490 - RNA, 20 nt, synthetic construct
  source 1..20: mol_type = other RNA
gtcgcgggagcccagttaag

SEQ ID NO: 491 - RNA, 20 nt, synthetic construct
  source 1..20: mol_type = other RNA
cctccctcctgattgggcag

SEQ ID NO: 492 - RNA, 20 nt, synthetic construct
  source 1..20: mol_type = other RNA
atcaggagggaggcgacggg

SEQ ID NO: 493 - RNA, 20 nt, synthetic construct
  source 1..20: mol_type = other RNA
cctctgcccaatcaggaggg

SEQ ID NO: 494 - RNA, 20 nt, synthetic construct
  source 1..20: mol_type = other RNA
aggccccccaatcggctgcg

SEQ ID NO: 495 - RNA, 20 nt, synthetic construct
  source 1..20: mol_type = other RNA
cagccgcgcagccgattggg

SEQ ID NO: 496 - RNA, 20 nt, synthetic construct
  source 1..20: mol_type = other RNA
ctttacatatgcagtcccac

SEQ ID NO: 497 - RNA, 20 nt, synthetic construct
  source 1..20: mol_type = other RNA
taaccacccgaggagaaccg

SEQ ID NO: 498 - RNA, 20 nt, synthetic construct
  source 1..20: mol_type = other RNA
aggctgccaggttctcctcg

SEQ ID NO: 499 - RNA, 20 nt, synthetic construct
  source 1..20: mol_type = other RNA
aaagtgacagaatcaagctg

SEQ ID NO: 500 - RNA, 20 nt, synthetic construct
  source 1..20: mol_type = other RNA
cccatggttccagagtcggg

SEQ ID NO: 501 - RNA, 20 nt, synthetic construct
  source 1..20: mol_type = other RNA
gaggggcaacccccgactcg

SEQ ID NO: 502 - RNA, 20 nt, synthetic construct
  source 1..20: mol_type = other RNA
tgacagaatcaagctggggt

SEQ ID NO: 503 - RNA, 20 nt, synthetic construct
  source 1..20: mol_type = other RNA
gtgacagaatcaagctgggg

SEQ ID NO: 504 - RNA, 20 nt, synthetic construct
  source 1..20: mol_type = other RNA
ggccctcaaacatggagagg

SEQ ID NO: 505 - RNA, 20 nt, synthetic construct
  source 1..20: mol_type = other RNA
gacagaatcaagctggggtg

SEQ ID NO: 506 - RNA, 20 nt, synthetic construct
  source 1..20: mol_type = other RNA
ttaaagtgacagaatcaagc

SEQ ID NO: 507 - RNA, 20 nt, synthetic construct
  source 1..20: mol_type = other RNA
taaagtgacagaatcaagct

SEQ ID NO: 508 - RNA, 20 nt, synthetic construct
  source 1..20: mol_type = other RNA
acccccgactctggaaccag

SEQ ID NO: 509 - RNA, 20 nt, synthetic construct
  source 1..20: mol_type = other RNA
cagccgcccgttagggcgcg

SEQ ID NO: 510 - RNA, 20 nt, synthetic construct
  source 1..20: mol_type = other RNA
cacgccggccgaaagccgcg

SEQ ID NO: 511 - RNA, 20 nt, synthetic construct
  source 1..20: mol_type = other RNA
tgcctcgcgccctaacgggg

SEQ ID NO: 512 - RNA, 20 nt, synthetic construct
  source 1..20: mol_type = other RNA
tcttccatacccgccccgcg

SEQ ID NO: 513 - RNA, 20 nt, synthetic construct
  source 1..20: mol_type = other RNA
gtgcctcgcgccctaacggg

SEQ ID NO: 514 - RNA, 20 nt, synthetic construct
  source 1..20: mol_type = other RNA
gggcgggtatggaagacgga

SEQ ID NO: 515 - RNA, 20 nt, synthetic construct
  source 1..20: mol_type = other RNA
aagccgcgcggggcgggtag

SEQ ID NO: 516 - RNA, 20 nt, synthetic construct
  source 1..20: mol_type = other RNA
ggcgggtatggaagacggag

SEQ ID NO: 517 - RNA, 20 nt, synthetic construct
  source 1..20: mol_type = other RNA
aggccgagcaccgcgggaag

SEQ ID NO: 518 - RNA, 20 nt, synthetic construct
  source 1..20: mol_type = other RNA
cgcggggcgggtatggaaga

SEQ ID NO: 519 - RNA, 20 nt, synthetic construct
  source 1..20: mol_type = other RNA
gggcgagtcacgtggggggg

SEQ ID NO: 520 - RNA, 20 nt, synthetic construct
  source 1..20: mol_type = other RNA
gcggggcgagtcacgtgggg

SEQ ID NO: 521 - RNA, 20 nt, synthetic construct
  source 1..20: mol_type = other RNA
cgcggggcgagtcacgtggg

SEQ ID NO: 522 - RNA, 20 nt, synthetic construct
  source 1..20: mol_type = other RNA
gggcgcggggcgagtcacgt

SEQ ID NO: 523 - RNA, 20 nt, synthetic construct
  source 1..20: mol_type = other RNA
tgaccgagactctccatcag

SEQ ID NO: 524 - RNA, 20 nt, synthetic construct
  source 1..20: mol_type = other RNA
cgcaggcggcgacacacagg

SEQ ID NO: 525 - RNA, 20 nt, synthetic construct
  source 1..20: mol_type = other RNA
atcccgtggctcccggcccg

SEQ ID NO: 526 - RNA, 20 nt, synthetic construct
  source 1..20: mol_type = other RNA
acgggattgggccgcgcacg

SEQ ID NO: 527 - RNA, 20 nt, synthetic construct
  source 1..20: mol_type = other RNA
tcccgtggctcccggcccgg

SEQ ID NO: 528 - RNA, 20 nt, synthetic construct
  source 1..20: mol_type = other RNA
ccgtgcgcggcccaatcccg

SEQ ID NO: 529 - RNA, 20 nt, synthetic construct
  source 1..20: mol_type = other RNA
gaggccgggacgcgcccact

SEQ ID NO: 530 - RNA, 20 nt, synthetic construct
  source 1..20: mol_type = other RNA
ctctcccgggagaggagagg

SEQ ID NO: 531 - RNA, 20 nt, synthetic construct
  source 1..20: mol_type = other RNA
gtctctctcagggtgcgctc

SEQ ID NO: 532 - RNA, 20 nt, synthetic construct
  source 1..20: mol_type = other RNA
cggaagagcgagccctttag

SEQ ID NO: 533 - RNA, 20 nt, synthetic construct
  source 1..20: mol_type = other RNA
ctcccgggagaggagaggcg

SEQ ID NO: 534 - RNA, 20 nt, synthetic construct
  source 1..20: mol_type = other RNA
ccctttaaggctatgccgag

SEQ ID NO: 535 - RNA, 20 nt, synthetic construct
  source 1..20: mol_type = other RNA
gacccccgccctccacagcg

SEQ ID NO: 536 - RNA, 20 nt, synthetic construct
  source 1..20: mol_type = other RNA
cctttaaggctatgccgagt

SEQ ID NO: 537 - RNA, 20 nt, synthetic construct
  source 1..20: mol_type = other RNA
aggccgggacgcgcccactg

SEQ ID NO: 538 - RNA, 20 nt, synthetic construct
  source 1..20: mol_type = other RNA
ccactcggcatagccttaag

SEQ ID NO: 539 - RNA, 20 nt, synthetic construct
  source 1..20: mol_type = other RNA
gaacccgcgcccggacgggc

SEQ ID NO: 540 - RNA, 20 nt, synthetic construct
  source 1..20: mol_type = other RNA
gggagcccgagtttaggaag

SEQ ID NO: 541 - RNA, 20 nt, synthetic construct
  source 1..20: mol_type = other RNA
gcgtcttccggcgcccgcgg

SEQ ID NO: 542 - RNA, 20 nt, synthetic construct
  source 1..20: mol_type = other RNA
gccaactgcggcgcgtcttc

SEQ ID NO: 543 - RNA, 20 nt, synthetic construct
  source 1..20: mol_type = other RNA
gcgccaacccgcgcccggac

SEQ ID NO: 544 - RNA, 20 nt, synthetic construct
  source 1..20: mol_type = other RNA
gtcgcctcctccgcgggcgc

SEQ ID NO: 545 - RNA, 20 nt, synthetic construct
  source 1..20: mol_type = other RNA
gcccgagtttaggaagagga

SEQ ID NO: 546 - RNA, 20 nt, synthetic construct
  source 1..20: mol_type = other RNA
gctccctgcccgtccgggcg

SEQ ID NO: 547 - RNA, 20 nt, synthetic construct
  source 1..20: mol_type = other RNA
gccgctcctccctgcccgtc

SEQ ID NO: 548 - RNA, 20 nt, synthetic construct
  source 1..20: mol_type = other RNA
ggcgccgcggggctcctcca

SEQ ID NO: 549 - RNA, 20 nt, synthetic construct
  source 1..20: mol_type = other RNA
tctgcgcatcaaagctagcg

SEQ ID NO: 550 - RNA, 20 nt, synthetic construct
  source 1..20: mol_type = other RNA
agaggggcctcgggcgggag

SEQ ID NO: 551 - RNA, 20 nt, synthetic construct
  source 1..20: mol_type = other RNA
acccgggcggcggcagtccg

SEQ ID NO: 552 - RNA, 20 nt, synthetic construct
  source 1..20: mol_type = other RNA
ccccgccaccctcctcctcc

SEQ ID NO: 553 - RNA, 20 nt, synthetic construct
  source 1..20: mol_type = other RNA
ccgcccgggtcgcccacgtg

SEQ ID NO: 554 - RNA, 20 nt, synthetic construct
  source 1..20: mol_type = other RNA
cgggaggaggagggtggcgg

SEQ ID NO: 555 - RNA, 20 nt, synthetic construct
  source 1..20: mol_type = other RNA
ggtgaggccgcagaggcgga

SEQ ID NO: 556 - RNA, 20 nt, synthetic construct
  source 1..20: mol_type = other RNA
cgcagatccctccgcctctg

SEQ ID NO: 557 - RNA, 20 nt, synthetic construct
  source 1..20: mol_type = other RNA
gcggggcggtgaggccgcag

SEQ ID NO: 558 - RNA, 20 nt, synthetic construct
  source 1..20: mol_type = other RNA
ccgggaggaggagggtggcg

SEQ ID NO: 559 - RNA, 20 nt, synthetic construct
  source 1..20: mol_type = other RNA
aaaagagaagtggctcattg

SEQ ID NO: 560 - RNA, 20 nt, synthetic construct
  source 1..20: mol_type = other RNA
gtgctctttctcaaattagg

SEQ ID NO: 561 - RNA, 20 nt, synthetic construct
  source 1..20: mol_type = other RNA
ttagggcatttcctgaaccc

SEQ ID NO: 562 - RNA, 20 nt, synthetic construct
  source 1..20: mol_type = other RNA
ggttggaatccagttagttg

SEQ ID NO: 563 - RNA, 20 nt, synthetic construct
  source 1..20: mol_type = other RNA
gttaattatggagccacaag

SEQ ID NO: 564 - RNA, 20 nt, synthetic construct
  source 1..20: mol_type = other RNA
ggcatttcctgaaccctggt

SEQ ID NO: 565 - RNA, 20 nt, synthetic construct
  source 1..20: mol_type = other RNA
cttaactgaaattccaacca

SEQ ID NO: 566 - RNA, 20 nt, synthetic construct
  source 1..20: mol_type = other RNA
ccttaactgaaattccaacc

SEQ ID NO: 567 - RNA, 20 nt, synthetic construct
  source 1..20: mol_type = other RNA
cctggttggaatttcagtta

SEQ ID NO: 568 - RNA, 20 nt, synthetic construct
  source 1..20: mol_type = other RNA
ccaacaagagcttttactgg

SEQ ID NO: 569 - RNA, 20 nt, synthetic construct
  source 1..20: mol_type = other RNA
gcgggcggcaagctgagagt

SEQ ID NO: 570 - RNA, 20 nt, synthetic construct
  source 1..20: mol_type = other RNA
gcgcagaggggtgacgtcag

SEQ ID NO: 571 - RNA, 20 nt, synthetic construct
  source 1..20: mol_type = other RNA
gtaaggctggcaagtcccca

SEQ ID NO: 572 - RNA, 20 nt, synthetic construct
  source 1..20: mol_type = other RNA
ggggcggctgagtcacgcta

SEQ ID NO: 573 - RNA, 20 nt, synthetic construct
  source 1..20: mol_type = other RNA
gggcgcgtagacagacgcag

SEQ ID NO: 574 - RNA, 20 nt, synthetic construct
  source 1..20: mol_type = other RNA
gcgcgcgccggaagtggcta

SEQ ID NO: 575 - RNA, 20 nt, synthetic construct
  source 1..20: mol_type = other RNA
ggccccggaagcgagaagga

SEQ ID NO: 576 - RNA, 20 nt, synthetic construct
  source 1..20: mol_type = other RNA
gctcgcggccggaagcgccg

SEQ ID NO: 577 - RNA, 20 nt, synthetic construct
  source 1..20: mol_type = other RNA
ggcgggacggagtgacttca

SEQ ID NO: 578 - RNA, 20 nt, synthetic construct
  source 1..20: mol_type = other RNA
gcgcggcggcgtggcgcaga

SEQ ID NO: 579 - RNA, 20 nt, synthetic construct
  source 1..20: mol_type = other RNA
caccgaagcccaagagctgg

SEQ ID NO: 580 - RNA, 20 nt, synthetic construct
  source 1..20: mol_type = other RNA
tgtggggagggttgttattg

SEQ ID NO: 581 - RNA, 20 nt, synthetic construct
  source 1..20: mol_type = other RNA
gtgagctgaaccttctcgtg

SEQ ID NO: 582 - RNA, 20 nt, synthetic construct
  source 1..20: mol_type = other RNA
accgaagcccaagagctggg

SEQ ID NO: 583 - RNA, 20 nt, synthetic construct
  source 1..20: mol_type = other RNA
gcgccccctgccggcccctg

SEQ ID NO: 584 - RNA, 20 nt, synthetic construct
  source 1..20: mol_type = other RNA
ccgaagcccaagagctgggg

SEQ ID NO: 585 - RNA, 20 nt, synthetic construct
  source 1..20: mol_type = other RNA
ccagctcttgggcttcggtg

SEQ ID NO: 586 - RNA, 20 nt, synthetic construct
  source 1..20: mol_type = other RNA
cacaccgaagcccaagagct

SEQ ID NO: 587 - RNA, 20 nt, synthetic construct
  source 1..20: mol_type = other RNA
agtgcaccgggaaccgcgcg

SEQ ID NO: 588 - RNA, 20 nt, synthetic construct
  source 1..20: mol_type = other RNA
agctcttgggcttcggtgtg

SEQ ID NO: 589 - RNA, 20 nt, synthetic construct
  source 1..20: mol_type = other RNA
caaacgggtggcccacgatg

SEQ ID NO: 590 - RNA, 20 nt, synthetic construct
  source 1..20: mol_type = other RNA
ccagggtctcagccaatcgt

SEQ ID NO: 591 - RNA, 20 nt, synthetic construct
  source 1..20: mol_type = other RNA
ccaaacgggtggcccacgat

SEQ ID NO: 592 - RNA, 20 nt, synthetic construct
  source 1..20: mol_type = other RNA
gagccgcgccctgcgcgatg

SEQ ID NO: 593 - RNA, 20 nt, synthetic construct
  source 1..20: mol_type = other RNA
gcgtacgcgcttcctcaatg

SEQ ID NO: 594 - RNA, 20 nt, synthetic construct
  source 1..20: mol_type = other RNA
tgcgcgattggccaaacggg

SEQ ID NO: 595 - RNA, 20 nt, synthetic construct
  source 1..20: mol_type = other RNA
cgtacgcgcttcctcaatgg

SEQ ID NO: 596 - RNA, 20 nt, synthetic construct
  source 1..20: mol_type = other RNA
gcgcccagcccagtcgcggg

SEQ ID NO: 597 - RNA, 20 nt, synthetic construct
  source 1..20: mol_type = other RNA
gccagggtctcagccaatcg

SEQ ID NO: 598 - RNA, 20 nt, synthetic construct
  source 1..20: mol_type = other RNA
cccgtttggccaatcgcgca

SEQ ID NO: 599 - RNA, 20 nt, synthetic construct
  source 1..20: mol_type = other RNA
catggttctgtagcaccggt

SEQ ID NO: 600 - RNA, 20 nt, synthetic construct
  source 1..20: mol_type = other RNA
agctcgagcggggtccagag

SEQ ID NO: 601 - RNA, 20 nt, synthetic construct
  source 1..20: mol_type = other RNA
cctctccctcatttgttaag

SEQ ID NO: 602 - RNA, 20 nt, synthetic construct
  source 1..20: mol_type = other RNA
caccgaaatacctcttccag

SEQ ID NO: 603 - RNA, 20 nt, synthetic construct
  source 1..20: mol_type = other RNA
aggaggaaggtgggggggcg

SEQ ID NO: 604 - RNA, 20 nt, synthetic construct
  source 1..20: mol_type = other RNA
gaatgcgcgtttgcaggagg

SEQ ID NO: 605 - RNA, 20 nt, synthetic construct
  source 1..20: mol_type = other RNA
ttccagggcgggttttcggg

SEQ ID NO: 606 - RNA, 20 nt, synthetic construct
  source 1..20: mol_type = other RNA
tgtggagaggctggtgcaaa

SEQ ID NO: 607 - RNA, 20 nt, synthetic construct
  source 1..20: mol_type = other RNA
aaggaatgcgcgtttgcagg

SEQ ID NO: 608 - RNA, 20 nt, synthetic construct
  source 1..20: mol_type = other RNA
atttgttaaaggtatcctac

SEQ ID NO: 609 - RNA, 20 nt, synthetic construct
  source 1..20: mol_type = other RNA
gccgtgagctccccgattgg

SEQ ID NO: 610 - RNA, 20 nt, synthetic construct
  source 1..20: mol_type = other RNA
ccccgattggggctcggtcg

SEQ ID NO: 611 - RNA, 20 nt, synthetic construct
  source 1..20: mol_type = other RNA
gccccaatcggggagctcag

SEQ ID NO: 612 - RNA, 20 nt, synthetic construct
  source 1..20: mol_type = other RNA
tgagctccccgattggggct

SEQ ID NO: 613 - RNA, 20 nt, synthetic construct
  source 1..20: mol_type = other RNA
ggccgtgagctccccgattg

SEQ ID NO: 614 - RNA, 20 nt, synthetic construct
  source 1..20: mol_type = other RNA
ggccggaccgagccccaatg

SEQ ID NO: 615 - RNA, 20 nt, synthetic construct
  source 1..20: mol_type = other RNA
agccccaatcggggagctca

SEQ ID NO: 616 - RNA, 20 nt, synthetic construct
  source 1..20: mol_type = other RNA
aattcgctgacgtgtaggag

SEQ ID NO: 617 - RNA, 20 nt, synthetic construct
  source 1..20: mol_type = other RNA
tacacgtcagcgaattcagc

SEQ ID NO: 618 - RNA, 20 nt, synthetic construct
  source 1..20: mol_type = other RNA
gccggaccgagccccaatcg

SEQ ID NO: 619 - RNA, 20 nt, synthetic construct
  source 1..20: mol_type = other RNA
ctcggcggcagcggctgccg

SEQ ID NO: 620 - RNA, 20 nt, synthetic construct
  source 1..20: mol_type = other RNA
cccccacccccggcccgcac

SEQ ID NO: 621 - RNA, 20 nt, synthetic construct
  source 1..20: mol_type = other RNA
gaggggcggctcggcggcag

SEQ ID NO: 622 - RNA, 20 nt, synthetic construct
  source 1..20: mol_type = other RNA
ggggcgcgcggggggggcgc

SEQ ID NO: 623 - RNA, 20 nt, synthetic construct
  source 1..20: mol_type = other RNA
tgccgcggcgcggccggtgc

SEQ ID NO: 624 - RNA, 20 nt, synthetic construct
  source 1..20: mol_type = other RNA
cggcagcggctgccgcggcg

SEQ ID NO: 625 - RNA, 20 nt, synthetic construct
  source 1..20: mol_type = other RNA
cggaacgcaagaggttaatg

SEQ ID NO: 626 - RNA, 20 nt, synthetic construct
  source 1..20: mol_type = other RNA
cccgcgaggggcggctcggg

SEQ ID NO: 627 - RNA, 20 nt, synthetic construct
  source 1..20: mol_type = other RNA
gccgccgagccgcccctcgg

SEQ ID NO: 628 - RNA, 20 nt, synthetic construct
  source 1..20: mol_type = other RNA
tcagggagccggctctcggg

SEQ ID NO: 629 - RNA, 20 nt, synthetic construct
  source 1..20: mol_type = other RNA
gagggagcgcgagagaggga

SEQ ID NO: 630 - RNA, 20 nt, synthetic construct
  source 1..20: mol_type = other RNA
agagggagcgcgagagaggg

SEQ ID NO: 631 - RNA, 20 nt, synthetic construct
  source 1..20: mol_type = other RNA
ctctcgcgctccctctccct

SEQ ID NO: 632 - RNA, 20 nt, synthetic construct
  source 1..20: mol_type = other RNA
ggaggcggtagttggaagtg

SEQ ID NO: 633 - RNA, 20 nt, synthetic construct
  source 1..20: mol_type = other RNA
atgggctgccccgggtctgg

SEQ ID NO: 634 - RNA, 20 nt, synthetic construct
  source 1..20: mol_type = other RNA
caggccagcgccccgcggtg

SEQ ID NO: 635 - RNA, 20 nt, synthetic construct
  source 1..20: mol_type = other RNA
cggcagcccgtgagcgcggg

SEQ ID NO: 636 - RNA, 20 nt, synthetic construct
  source 1..20: mol_type = other RNA
gggaggaggggacggtgctt

SEQ ID NO: 637 - RNA, 20 nt, synthetic construct
  source 1..20: mol_type = other RNA
cggaggcggtagttggaagt

SEQ ID NO: 638 - RNA, 20 nt, synthetic construct
  source 1..20: mol_type = other RNA
tgattgctggtcgtttgttg

SEQ ID NO: 639 - RNA, 20 nt, synthetic construct
  source 1..20: mol_type = other RNA
aagagaaacaccaacaaaga

SEQ ID NO: 640 - RNA, 20 nt, synthetic construct
  source 1..20: mol_type = other RNA
aggcctgcagagtcacgtgg

SEQ ID NO: 641 - RNA, 20 nt, synthetic construct
  source 1..20: mol_type = other RNA
acaccaacaaagaaggcgag

SEQ ID NO: 642 - RNA, 20 nt, synthetic construct
  source 1..20: mol_type = other RNA
agaggcctgcagagtcacgt

SEQ ID NO: 643 - RNA, 20 nt, synthetic construct
  source 1..20: mol_type = other RNA
tgcccccacgtgactctgcg

SEQ ID NO: 644 - RNA, 20 nt, synthetic construct
  source 1..20: mol_type = other RNA
ggcctgcagagtcacgtggg

SEQ ID NO: 645 - RNA, 20 nt, synthetic construct
  source 1..20: mol_type = other RNA
aatacccctatctatctggg

SEQ ID NO: 646 - RNA, 20 nt, synthetic construct
  source 1..20: mol_type = other RNA
agagaaacaccaacaaagag

SEQ ID NO: 647 - RNA, 20 nt, synthetic construct
  source 1..20: mol_type = other RNA
gagaggcctgcagagtcacg

SEQ ID NO: 648 - RNA, 20 nt, synthetic construct
  source 1..20: mol_type = other RNA
gaggcctgcagagtcacgtg

SEQ ID NO: 649 - RNA, 20 nt, synthetic construct
  source 1..20: mol_type = other RNA
gggcggcgcgctcggactgg

SEQ ID NO: 650 - RNA, 20 nt, synthetic construct
  source 1..20: mol_type = other RNA
atcgccaggagacagacctc

SEQ ID NO: 651 - RNA, 20 nt, synthetic construct
  source 1..20: mol_type = other RNA
agccccaggacgcgacagcg

SEQ ID NO: 652 - RNA, 20 nt, synthetic construct
  source 1..20: mol_type = other RNA
agacagacctcaggcgccgg

SEQ ID NO: 653 - RNA, 20 nt, synthetic construct
  source 1..20: mol_type = other RNA
ccctcccgcctccccgaggg

SEQ ID NO: 654 - RNA, 20 nt, synthetic construct
  source 1..20: mol_type = other RNA
gctcccctcccgcctccccg

SEQ ID NO: 655 - RNA, 20 nt, synthetic construct
  source 1..20: mol_type = other RNA
agacagacctcaggcgccgg

SEQ ID NO: 656 - RNA, 20 nt, synthetic construct
  source 1..20: mol_type = other RNA
gtctcctggcgatcccgcag

SEQ ID NO: 657 - RNA, 20 nt, synthetic construct
  source 1..20: mol_type = other RNA
gagacagacctcaggcgccg

SEQ ID NO: 658 - RNA, 20 nt, synthetic construct
  source 1..20: mol_type = other RNA
gggggtgattcaggagtgag

SEQ ID NO: 659 - RNA, 20 nt, synthetic construct
  source 1..20: mol_type = other RNA
gcctgacttggggtgacaag

SEQ ID NO: 660 - RNA, 20 nt, synthetic construct
  source 1..20: mol_type = other RNA
gtgctaattacaactgctgg

SEQ ID NO: 661 - RNA, 20 nt, synthetic construct
  source 1..20: mol_type = other RNA
gggaaccgggaaactgcggg

SEQ ID NO: 662 - RNA, 20 nt, synthetic construct
  source 1..20: mol_type = other RNA
gttaatcggtcccccgccgg

SEQ ID NO: 663 - RNA, 20 nt, synthetic construct
  source 1..20: mol_type = other RNA
ccagcctatccaccggcggg

SEQ ID NO: 664 - RNA, 20 nt, synthetic construct
  source 1..20: mol_type = other RNA
ccgccggtggataggctggg

SEQ ID NO: 665 - RNA, 20 nt, synthetic construct
  source 1..20: mol_type = other RNA
ccgcccagcctatccaccgg

SEQ ID NO: 666 - RNA, 20 nt, synthetic construct
  source 1..20: mol_type = other RNA
gccccgcccagcctatccac

SEQ ID NO: 667 - RNA, 20 nt, synthetic construct
  source 1..20: mol_type = other RNA
atggttaatcggtcccccgc

SEQ ID NO: 668 - RNA, 20 nt, synthetic construct
  source 1..20: mol_type = other RNA
gcccagcctatccaccggcg

SEQ ID NO: 669 - RNA, 20 nt, synthetic construct
  source 1..20: mol_type = other RNA
acagggaaatgcgcgcgccg

SEQ ID NO: 670 - RNA, 20 nt, synthetic construct
  source 1..20: mol_type = other RNA
gggaaatgcgcgcgccgagg

SEQ ID NO: 671 - RNA, 20 nt, synthetic construct
  source 1..20: mol_type = other RNA
gccccgcgctggaccgctcg

SEQ ID NO: 672 - RNA, 20 nt, synthetic construct
  source 1..20: mol_type = other RNA
tccctgttcccaaggccccg

SEQ ID NO: 673 - RNA, 20 nt, synthetic construct
  source 1..20: mol_type = other RNA
tccctgttcccaaggcccca

SEQ ID NO: 674 - RNA, 20 nt, synthetic construct
  source 1..20: mol_type = other RNA
aatgaaaactgaaagcagcg

SEQ ID NO: 675 - RNA, 20 nt, synthetic construct
  source 1..20: mol_type = other RNA
cccgaccgccagtcgccctg

SEQ ID NO: 676 - RNA, 20 nt, synthetic construct
  source 1..20: mol_type = other RNA
gggctccccagggtgtaggg

SEQ ID NO: 677 - RNA, 20 nt, synthetic construct
  source 1..20: mol_type = other RNA
caaggccccagggcgactgg

SEQ ID NO: 678 - RNA, 20 nt, synthetic construct
  source 1..20: mol_type = other RNA
cggcctcccgagaccgcagg

SEQ ID NO: 679 - RNA, 20 nt, synthetic construct
  source 1..20: mol_type = other RNA
ggaccacggtaccacccaat

SEQ ID NO: 680 - RNA, 20 nt, synthetic construct
  source 1..20: mol_type = other RNA
ggtaccacccaatgggaaag

SEQ ID NO: 681 - RNA, 20 nt, synthetic construct
  source 1..20: mol_type = other RNA
gaccacggtaccacccaatg

SEQ ID NO: 682 - RNA, 20 nt, synthetic construct
  source 1..20: mol_type = other RNA
gtaccacccaatgggaaagg

SEQ ID NO: 683 - RNA, 20 nt, synthetic construct
  source 1..20: mol_type = other RNA
tcttcccctttcccattggg

SEQ ID NO: 684 - RNA, 20 nt, synthetic construct
  source 1..20: mol_type = other RNA
cggaccacggtaccacccaa

SEQ ID NO: 685 - RNA, 20 nt, synthetic construct
  source 1..20: mol_type = other RNA
ggaaaggggaagaaccgaag

SEQ ID NO: 686 - RNA, 20 nt, synthetic construct
  source 1..20: mol_type = other RNA
tgtccccgaaatggattgtg

SEQ ID NO: 687 - RNA, 20 nt, synthetic construct
  source 1..20: mol_type = other RNA
acggtaccacccaatgggaa

SEQ ID NO: 688 - RNA, 20 nt, synthetic construct
  source 1..20: mol_type = other RNA
tttcccattgggtggtaccg

SEQ ID NO: 689 - RNA, 20 nt, synthetic construct
  source 1..20: mol_type = other RNA
ccccgcccccagctcccgag

SEQ ID NO: 690 - RNA, 20 nt, synthetic construct
  source 1..20: mol_type = other RNA
tcacgctgggactccgaatg

SEQ ID NO: 691 - RNA, 20 nt, synthetic construct
  source 1..20: mol_type = other RNA
ctgtttaatattgatgaggg

SEQ ID NO: 692 - RNA, 20 nt, synthetic construct
  source 1..20: mol_type = other RNA
agggatctgggaacccctcg

SEQ ID NO: 693 - RNA, 20 nt, synthetic construct
  source 1..20: mol_type = other RNA
ggctgtttaatattgatgag

SEQ ID NO: 694 - RNA, 20 nt, synthetic construct
  source 1..20: mol_type = other RNA
cacacccccagctctccatg

SEQ ID NO: 695 - RNA, 20 nt, synthetic construct
  source 1..20: mol_type = other RNA
gctgtttaatattgatgagg

SEQ ID NO: 696 - RNA, 20 nt, synthetic construct
  source 1..20: mol_type = other RNA
ccagccaatggagagctggg

SEQ ID NO: 697 - RNA, 20 nt, synthetic construct
  source 1..20: mol_type = other RNA
ctcatcaatattaaacagcc

SEQ ID NO: 698 - RNA, 20 nt, synthetic construct
  source 1..20: mol_type = other RNA
gccaggcagccattggcaga

SEQ ID NO: 699 - RNA, 20 nt, synthetic construct
  source 1..20: mol_type = other RNA
actctatgcaggttgataag

SEQ ID NO: 700 - RNA, 20 nt, synthetic construct
  source 1..20: mol_type = other RNA
aaagaggtgatgacatcaag

SEQ ID NO: 701 - RNA, 20 nt, synthetic construct
  source 1..20: mol_type = other RNA
ctcatcacatgactctatgc

SEQ ID NO: 702 - RNA, 20 nt, synthetic construct
  source 1..20: mol_type = other RNA
tgattaactcagaagtggag

SEQ ID NO: 703 - RNA, 20 nt, synthetic construct
  source 1..20: mol_type = other RNA
tcatcacatgactctatgcg

SEQ ID NO: 704 - RNA, 20 nt, synthetic construct
  source 1..20: mol_type = other RNA
tgctatgcagcacagagagg

SEQ ID NO: 705 - RNA, 20 nt, synthetic construct
  source 1..20: mol_type = other RNA
ccatgtatccagtttcatga

SEQ ID NO: 706 - RNA, 20 nt, synthetic construct
  source 1..20: mol_type = other RNA
atggtttctaatcagaagaa

SEQ ID NO: 707 - RNA, 20 nt, synthetic construct
  source 1..20: mol_type = other RNA
tggaaagttgtaaaactgag

SEQ ID NO: 708 - RNA, 20 nt, synthetic construct
  source 1..20: mol_type = other RNA
agaaatgattaactcagaag

SEQ ID NO: 709 - RNA, 20 nt, synthetic construct
  source 1..20: mol_type = other RNA
gtggccgcgtttgaagtctc

SEQ ID NO: 710 - RNA, 20 nt, synthetic construct
  source 1..20: mol_type = other RNA
ggcagccgggggtaccaagg

SEQ ID NO: 711 - RNA, 20 nt, synthetic construct
  source 1..20: mol_type = other RNA
ggctgctggttggccggcga

SEQ ID NO: 712 - RNA, 20 nt, synthetic construct
  source 1..20: mol_type = other RNA
gcccgccggctaactcgccg

SEQ ID NO: 713 - RNA, 20 nt, synthetic construct
  source 1..20: mol_type = other RNA
gcccgcggcgagttagccgg

SEQ ID NO: 714 - RNA, 20 nt, synthetic construct
  source 1..20: mol_type = other RNA
ggcgctccagagcccctctg

SEQ ID NO: 715 - RNA, 20 nt, synthetic construct
  source 1..20: mol_type = other RNA
ggcgccggagacttcaaacg

SEQ ID NO: 716 - RNA, 20 nt, synthetic construct
  source 1..20: mol_type = other RNA
gggtaccaagggggcaacgg

SEQ ID NO: 717 - RNA, 20 nt, synthetic construct
  source 1..20: mol_type = other RNA
ggggcgcacgtgaccctcgc

SEQ ID NO: 718 - RNA, 20 nt, synthetic construct
  source 1..20: mol_type = other RNA
ggggtagctaaggacagcgg

(19) United States
(12) Patent Application Publication
(10) Pub. No.: US 2026/0317724 A1
(43) Pub. Date: Oct. 15, 2026

(54) COMPOSITIONS AND METHODS FOR ENGINEERING MATURE IPSC-DERIVED AND ESC-DERIVED HEPATOCYTES

(71) Applicant: Duke University, Durham, NC (US)

(72) Inventors: Charles A. Gersbach, Chapel Hill, NC (US); Helen Streff, Durham, NC (US)

(21) Appl. No.: 19/480,763

(22) PCT Filed: May 2, 2024
(86) PCT No.: PCT/US2024/027546

§ 371 Date: Oct. 31, 2025

Related U.S. Application Data
(60) Provisional application No. 63/499,646, filed on May 2, 2023.
(60) Provisional application No. 63/506,009, filed on Jun. 2, 2023.
(60) Provisional application No. 63/622,392, filed on Jan. 18, 2024.

(51) Int. Cl. C12N 5/071; A61K 31/7105; A61K 38/46; C12N 9/22; C12N 15/11

(52) U.S. Cl. CPC C12N 5/067; A61K 31/7105; A61K 38/465; C12N 9/226; C12N 15/11; C12N 2310/20; C12N 2506/45

(57) ABSTRACT

Disclosed herein are compositions and methods for inducing differentiation of an induced pluripotent stem cell (iPSC) or an embryonic stem cell (ESC) into a hepatocyte. The compositions and methods may also be used to promote maturation of a hepatocyte. The compositions and methods may include modulators of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof, or a gene product thereof. The compositions and methods may be used for liver transplantations.

### CROSS-REFERENCE TO RELATED APPLICATIONS

This application claims priority to U.S. Provisional Patent Application No. 63/499,646, filed May 2, 2023, U.S. Provisional Patent Application No. 63/506,009, filed Jun. 2, 2023, and U.S. Provisional Patent Application No. 63/622,392, filed Jan. 18, 2024, the entire contents of each of which are hereby incorporated by reference.

### STATEMENT REGARDING FEDERALLY SPONSORED RESEARCH

This invention was made with government support under grant HG012053 awarded by the National Institutes of Health. This invention was made with government support under grant UM1HG013053 awarded by the National Institutes of Health. This invention was made with government support under grant DGF 2139754 awarded by the National Science Foundation. The government has certain rights in the invention.

### FIELD

This disclosure relates to genes to target for repression or activation to induce the differentiation of cells into hepatocytes as well as the maturation of hepatocytes.

### Introduction

The liver is unique in its ability to regenerate after external damage from various toxins or genetic defects. However, after prolonged chronic damage, the liver loses the ability to regenerate and becomes irreversibly cirrhotic. Due to the shortage of liver donors for transplantations and the scarcity of primary human hepatocytes for in vitro toxicology studies, there is a growing interest in the in vitro reprogramming of induced pluripotent stem cells (IPCSs) to mature hepatocytes, termed iPSC derived hepatocytes (iHeps). However, current methods for producing iHeps yield immature cells with significant differences in gene expression and epigenetic signatures compared to primary human hepatocytes (PHHs) (Si-Tayeb, K. et al. Hepatology 2010, 51, 297-305, incorporated herein by reference). See FIG. 1A. There is a need for a reliable means to generate iHeps.

### SUMMARY

In an aspect, the disclosure relates to a method for inducing differentiation of an induced pluripotent stem cell (IPSC) or an embryonic stem cell (ESC) into a hepatocyte and/or for promoting maturation of a hepatocyte. The method may include administering to a cell or a subject an activator of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof.

In an aspect, the disclosure relates to a composition comprising a modulator of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof. In some embodiments, the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof. In some embodiments, the gene is selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, NR5A1, HBP1, NHLH1, or YAF2, or a combination thereof, and the composition further comprises a modulator of a gene selected from FOXA2, HNF4A, NR5A2, FOXA3, or HNF4G, or a combination thereof, or the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof, and the composition further comprises a modulator of a gene selected from FOXA2, HNF4A, NR5A2, FOXA3, or HNF4G, or a combination thereof. In some embodiments, the modulator induces differentiation of an induced pluripotent stem cell (iPSC) or an embryonic stem cell (ESC) into a hepatocyte, or promotes maturation of an IPSC-derived hepatocyte, or promotes maturation of an ESC-derived hepatocyte, or a combination thereof. In some embodiments, the composition modulates gene expression within the IPSC or the ESC or the hepatocyte. In some embodiments, the composition increases expression of albumin, alpha-1 antitrypsin, ASGR1, SERPINA1, Foxa2, a p450 enzyme, or Hnf4a, or a combination thereof, in the IPSC or in the ESC or in the iPSC-derived hepatocyte or in the ESC-derived hepatocyte. In some embodiments, the modulator is an activator. In some embodiments, the activator comprises a polypeptide, or a polynucleotide, or a combination thereof. In some embodiments, the activator comprises a polypeptide sequence selected from SEQ ID NOs: 121-158 or a fragment thereof, or polynucleotide sequence selected from SEQ ID NOs: 83-120 or a fragment thereof. In some embodiments, the modulator comprises a DNA targeting composition. The DNA targeting composition may include (a) a Cas9 protein and at least one guide RNA (gRNA) that targets the Cas9 protein to the gene or a regulatory element thereof; or (b) a fusion protein, wherein the fusion protein comprises two heterologous polypeptide domains, wherein the first polypeptide domain comprises a zinc finger protein or a TALE or a Cas protein selected from a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity, wherein when the first polypeptide domain comprises a Cas9 protein the DNA targeting composition further comprises at least one guide RNA (gRNA) that targets the Cas9 protein to the gene or a regulatory element thereof. In a further aspect, the disclosure relates to a DNA targeting composition. The DNA targeting composition may include a Cas9 protein or a fusion protein, wherein the fusion protein comprises two heterologous polypeptide domains, wherein the first polypeptide domain comprises a zinc finger protein or a TALE or a Cas protein selected from a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity thereof; and at least one guide RNA (gRNA) that targets the Cas protein to a target gene or a regulatory element thereof when the DNA targeting composition comprises a Cas protein, wherein the gene is selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof. In some embodiments, the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4. In some embodiments, the composition comprises a first gRNA targeting a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, NR5A1, HBP1, NHLH1, or YAF2 or a combination thereof, and a second gRNA targeting a gene selected from FOXA2, HNF4A, NR5A2, FOXA3, or HNF4G, or a combination thereof, or the composition comprises a first gRNA targeting a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof, and a second gRNA targeting a gene selected from FOXA2, HNF4A, NR5A2, FOXA3, or HNF4G, or a combination thereof. In some embodiments, the gRNA is encoded by a polynucleotide comprising a sequence selected from SEQ ID NOs: 159-438, or comprises a sequence selected from SEQ ID NOs: 439-718. In some embodiments, the Cas protein comprises a Streptococcus pyogenes Cas9 protein, or a Staphylococcus aureus Cas9 protein, or any fragment thereof. In some embodiments, the Cas9 protein comprises the amino acid sequence of one of SEQ ID NOs: 26-29, or any fragment thereof, and/or the Cas9 protein is encoded by a polynucleotide comprising a sequence selected from SEQ ID NOs: 30-39, and/or the Cas9 protein comprises an amino acid sequence having at least 90% or greater identity to a sequence selected from SEQ ID NOs: 26-29, or any fragment thereof, and/or the Cas9 protein is encoded by a polynucleotide comprising a sequence having at least 90% or greater identity to a sequence selected from SEQ ID NOs: 30-39, or any fragment thereof, and/or the Cas9 protein comprises an amino acid sequence having one, two, three, four, five or more changes selected from amino acid substitutions, insertions, or deletions, relative to a sequence selected from SEQ ID NOs: 26-29, or any fragment thereof, and/or the Cas9 protein is encoded by a polynucleotide comprising a sequence having one, two, three, four, five or more changes selected from nucleotide substitutions, insertions, or deletions, relative to a sequence selected from SEQ ID NOS: 30-39, or any fragment thereof. In some embodiments, the fusion protein comprises more than one second polypeptide domain. In some embodiments, the second polypeptide domain comprises a VP16 protein, or VP64, or p65 domain of NF kappa B transcription activator activity, or Tet1, or VPH, or VPR, or Rta, or a p300 protein, or a fragment thereof. In some embodiments, the second polypeptide domain comprises the amino acid sequence of SEQ ID NO: 41, 42, 53, or 55, or any fragment thereof, and/or the second polypeptide domain is encoded by a polynucleotide comprising the sequence of SEQ ID NO: 54 or 56, and/or the second polypeptide domain comprises an amino acid sequence having at least 90% or greater identity to SEQ ID NO: 41, 42, 53, or 55, or any fragment thereof, and/or the second polypeptide domain is encoded by a polynucleotide comprising a sequence having at least 90% or greater identity to SEQ ID NO: 54 or 56, or any fragment thereof, and/or the second polypeptide domain comprises an amino acid sequence having one, two, three, four, five or more changes selected from amino acid substitutions, insertions, or deletions, relative to SEQ ID NO: 41, 42, 53, or 55, or any fragment thereof, and/or the second polypeptide domain is encoded by a polynucleotide comprising a sequence having one, two, three, four, five or more changes selected from nucleotide substitutions, insertions, or deletions, relative to SEQ ID NO: 54 or 56, or any fragment thereof. In some embodiments, the fusion protein comprises the amino acid sequence of SEQ ID NO: 43, or any fragment thereof, and/or the fusion protein is encoded by a polynucleotide comprising the sequence of SEQ ID NO: 44, and/or the fusion protein comprises an amino acid sequence having at least 90% or greater identity to SEQ ID NO: 43, or any fragment thereof, and/or the fusion protein is encoded by a polynucleotide comprising a sequence having at least 90% or greater identity to SEQ ID NO: 44, or any fragment thereof, and/or the fusion protein comprises an amino acid sequence having one, two, three, four, five or more changes selected from amino acid substitutions, insertions, or deletions, relative to SEQ ID NO: 43, or any fragment thereof, and/or the fusion protein is encoded by a polynucleotide comprising a sequence having one, two, three, four, five or more changes selected from nucleotide substitutions, insertions, or deletions, relative to SEQ ID NO: 44.

Another aspect of the disclosure provides an isolated polynucleotide sequence encoding a composition as detailed herein.

Another aspect of the disclosure provides a vector comprising an isolated polynucleotide sequence as detailed herein.

Another aspect of the disclosure provides an isolated cell comprising a composition as detailed herein, or an isolated polynucleotide as detailed herein, or a vector as detailed herein, or a combination thereof.

Another aspect of the disclosure provides a pharmaceutical composition comprising a composition as detailed herein, or an isolated polynucleotide as detailed herein, or a vector as detailed herein, or an isolated cell as detailed herein, or a combination thereof.

Another aspect of the disclosure provides a therapy for inducing differentiation of an induced pluripotent stem cell (IPSC) or an embryonic stem cell (ESC) into a hepatocyte and/or for promoting maturation of a hepatocyte. The therapy may include a composition as detailed herein, or an isolated polynucleotide as detailed herein, or a vector as detailed herein, or an isolated cell as detailed herein, or a pharmaceutical composition as detailed herein, or a combination thereof.

Another aspect of the disclosure provides a method of inducing differentiation of an induced pluripotent stem cell (iPSC) or an embryonic stem cell (ESC) into a hepatocyte. The method may include administering to a cell or a subject a composition as detailed herein, or an isolated polynucleotide sequence as detailed herein, or a vector as detailed herein, or a pharmaceutical composition as detailed herein, or a combination thereof. In some embodiments, the composition or isolated polynucleotide sequence or vector or pharmaceutical composition is administered to an IPSC or an ESC or a hepatocyte, and the expression of albumin, alpha-1 antitrypsin, ASGR1, SERPINA1, Foxa2, a p450 enzyme, or Hnf4a, or a combination thereof, is thereby increased in the iPSC or the ESC or the iPSC-derived hepatocyte or the ESC-derived hepatocyte. In some embodiments, the cell is an induced pluripotent stem cell (IPSC). In some embodiments, the cell is an embryonic stem cell (ESC).

Another aspect of the disclosure provides a method of promoting maturation of a hepatocyte. The method may include administering to a cell or a subject a composition as detailed herein, or an isolated polynucleotide sequence as detailed herein, or a vector as detailed herein, or a pharmaceutical composition as detailed herein, or a combination thereof. In some embodiments, the composition or isolated polynucleotide sequence or vector or pharmaceutical composition is administered to an iPSC or an ESC or a hepatocyte, and the expression of albumin, alpha-1 antitrypsin, ASGR1, SERPINA1, Foxa2, a p450 enzyme, or Hnf4a, or a combination thereof, is thereby increased in the IPSC or the ESC or the IPSC-derived hepatocyte or the ESC-derived hepatocyte.

The disclosure provides for other aspects and embodiments that will be apparent in light of the following detailed description and accompanying figures.

### BRIEF DESCRIPTION OF THE DRAWINGS

FIGS. 1A-1B show schematics of protocols for generation of specific cell types. FIG. 1A is a schematic showing a protocol for generating iPSC-derived hepatocytes (iHeps). The starting cells (IPSCs) and each cell type that they are differentiated to become iHeps are shown above the timeline. The days of incubation and the components that the cells are incubated with for the designated time period are shown below the timeline. FIG. 1B is a schematic showing iHep characterization.

FIGS. 2A-2E show comparative results for iPSCs, iHeps, and primary human hepatocytes (PHHs). FIG. 2A is a graph showing qPCR results comparing iPSCs to iHeps to PHHs relative to IPSCs for various markers. Asterisks represent significance. FIG. 2B is a volcano plot showing DESeq2 results of PHH versus iHep RNAseq transcript fold changes with positive and negative fold changes, respectively. FIG. 2C is a UMAP representation of iHeps assessed through multiomic RNA and ATACseq clustered by RNAseq with AFP gene expression highlighted in dark gray. FIG. 2D is a UMAP representation of iHeps assessed through multiomic RNA and ATACseq clustered by RNAseq with Hnf4a gene expression highlighted in dark gray. FIG. 2E is a UMAP representation of iHeps assessed through multiomic RNA and ATACseq clustered by RNAseq with Foxa2 gene expression highlighted in dark gray.

FIGS. 3A-3F show a process for screening and the results of screening. FIG. 3A is a schematic showing a process by which TF open reading frame (ORF) screening can be conducted. FIG. 3B is a volcano plot showing DESeq2 results comparing the ORF distributions in the high and low bins from a FACS screen. 39 hits came out as being significantly overrepresented in the high bin while 71 hits came out as being significantly overrepresented in the low bin. FIG. 3C is a violin plot showing DESeq2 results from the top and bottom 10% bin gRNA abundance of the CRISPRa albumin screen with gRNAs with adjusted p-values <0.05 highlighted in gray and non-target in black. Positive fold change represents overrepresentation in the high 10% albumin bin. FIG. 3D is a violin plot showing results from an ORF albumin screen. FIG. 3E is a volcano plot showing DESeq2 results in MGH2069 IPSCs comparing the ORF distributions in the high and low bins from the FACS screen. 27 hits came out as being significantly overrepresented in the high bin while 178 hits came out as being significantly overrepresented in the low bin. FIG. 3F is a Venn diagram showing a comparison of the significant TFs from the albumin screens.

FIGS. 4A-4C show analyses of hits. FIG. 4A is a graph showing albumin qPCR fold change results for high bin hits relative to mCherry/GFP negative controls. Significance from ordinary one-way ANOVAs with multiple comparisons is indicated by the colors in the legend with asterisks corresponding to significance. FIG. 4B is a graph showing SERPINA1 qPCR fold change results for high bin hits relative to mCherry/GFP negative controls. Significance from ordinary one-way ANOVAs with multiple comparisons is indicated by the colors in the legend with asterisks corresponding to significance. FIG. 4C is a heatmap of select hits analyzed by RNAseq relative to GFP with DESeq2. Fold change for genes of interest is indicated by color.

FIGS. 5A-5B show qPCR results of hit gRNAs. FIG. 5A is a graph showing albumin qPCR results for hit gRNAs with significance shown with asterisks compared to the negative control gRNA. FIG. 5B is a graph showing AFP qPCR results with fold change shown relative to negative control.

FIGS. 6A-6E show hits and analysis thereof using ELISA. FIG. 6A is a heatmap showing select hits analyzed by RNAseq relative to GFP with DESeq2. Fold change for functional genes of interest is indicated by color. FIG. 6B is a graph showing results from an ELISA using supernatant albumin. FIG. 6C is a graph showing results from a supernatant urea assay normalized by cell number relative to Thy1.1. FIG. 6D is a graph showing results from an ELISA using supernatant SERPINA1 normalized by cell number relative to Thy1.1.

FIG. 6E is a graph showing results from an ELISA using supernatant fibrinogen for hit ORFs normalized by cell number relative to Thy1.1.

FIG. 7 is a schematic showing future studies to be conducted using scRNAseq screens, in vitro functional characterization, and engraftment validation.

FIG. 8 is a UMAP representation of a population of cells, with arrows indicating the progression to hepatocyte cellular profiles.

FIGS. 9A-9C show validation of ORF hits in MGH iPSC lines. FIG. 9A is a correlation plot showing WTC11 vs MGH2069 IPSC screen with coloring indicating whether a given ORF was a hit in both, one, or neither screen (“ns”). FIG. 9B is a graph showing validation of hit ORFs in MGH2069 albumin IPSC line with fold change relative to mCherry/GFP negative control. Dark gray indicates that the ORF came out of both screens whereas light gray indicates it was a hit in only the WTC11 screen. FIG. 9C is a graph showing validation of hit ORFs in MGH2069 Serpina1 IPSC line with fold change relative to mCherry/GFP negative control. Dark gray indicates that the ORF came out of both screens whereas light gray indicates it was a hit in only the WTC11 screen.

FIGS. 10A-10B show validation of ORF hits in embryonic stem cell (ESC) lines. FIG. 10A is a graph showing validation of hit ORFs in H9 albumin ESC line with fold change relative to mCherry/GFP negative control. FIG. 10B is a graph showing validation of hit ORFs in H9 Serpina1 ESC line with fold change relative to mCherry/GFP negative control.

FIGS. 11A-11B show that hit ORFs display a polygonal homogenous albumin stain indicative of a hepatocyte. FIG. 11A is images showing 20× immunofluorescence of hit ORFs FOXA2 and NR5A2 as well as negative control Thy1.1 stained for DAPI in blue and albumin in red. FIG. 11B is images showing 20× immunofluorescence of hit ORFs KLF7 and NHLH2 as well as negative control Thy1.1 stained for DAPI in blue and albumin in red.

### DETAILED DESCRIPTION

Provided herein are compositions and methods for inducing differentiation of a cell such as an induced pluripotent stem cell (IPSC) or an embryonic stem cell (ESC) to a mature hepatocyte, termed iPSC-derived hepatocytes (iHep) or ESC-derived hepatocytes, as well as compositions and methods for promoting maturation of a hepatocyte. The compositions and methods may include increasing or decreasing gene expression or a gene product such as transcription factors. The gene may be selected from, for example, NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof.

Transcriptions factors (TFs) are central mediators of cellular reprogramming and differentiation. As described herein, a Staphylococcus aureus Cas9 (SaCas9)-based epigenome editors were used for targeted gene activation in induced pluripotent stem cells (IPSCs) or embryonic stem cells (ESCs). These tools were leveraged to profile the effects of a panel of genes with CRISPR activation (CRISPRa) screens on hepatocyte differentiation and maturation. The single-cell multiomic RNA- and ATAC-seq profile of iHeps detailed herein indicated substantial transcriptomic and epigenetic heterogeneity within the final cell population. These epigenetic differences likely contribute to the reported deficiencies in using iHeps with conventional methods. Epigenetic editing using CRISPR-dCas9 allows the precise dissection and programming of intricate cellular pathways. To address the limitations of current protocols for iHep generation, CRISPR activation screens and ORF screens of transcription factor and epigenetic modifier genes were used. Fluorescence-activated cell sorting (FACS) for cells that upregulate albumin or alpha-fetoprotein generated a list of guide RNAs (gRNAs) and corresponding gene targets implicated in mature and immature liver phenotypes, respectively. Several independent gRNA hits from the screens targeted the same gene promoters, strongly implicating these transcription factors in iHep differentiation and/or maturity. Validation experiments confirmed the ability of CRISPR-based activation of screen hits to upregulate the expression of mature liver genes. Phenotypic validation of the iHeps in vitro and in vivo in mouse models may test the function of the engineered iHeps. The results detailed herein illuminated several novel targets for promoting maturation of a hepatocyte. Collectively, the gene targets identified and described herein may be used for liver transplantations.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Allogeneic” refers to any material derived from another subject of the same species. Allogeneic cells are genetically distinct and immunologically incompatible yet belong to the same species. Typically, “allogeneic” is used to define cells, such as stem cells, that are transplanted from a donor to a recipient of the same species.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Autologous” refers to any material derived from a subject and re-introduced to the same subject.

“Binding region” as used herein refers to the region within a target region that is recognized and bound by the CRISPR/Cas-based gene editing system.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, bladder, cervix, uterus, skin, epithelium, muscle, kidney, liver, lymph, bone, blood, ovary, prostate, lung, brain, head and neck, and/or breast. Cancer may include medullablastoma, non-small cell lung cancer, and/or mesothelioma. In some embodiments, the cancer includes liver cancer. In some embodiments, the cancer includes leukemia. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the leukemia is chronic myeloid leukemia (CML). In some embodiments, the leukemia is acute myeloid leukemia (AML).

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.) The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

The term “shRNA” stands for short hairpin RNA or small hairpin RNA. A shRNA is an artificial RNA molecule with a tight hairpin turn that can be used to silence target gene expression via RNA interference (RNAI). Expression of shRNA in cells may be facilitated by delivery of plasmids or viral or bacterial vectors. The shRNA is processed by Dicer into SiRNA.

The term “siRNA” stands for small interfering RNA siRNA, sometimes also known as short interfering RNA or silencing RNA. A SIRNA is a class of double-stranded RNA molecule. The siRNA may be natural or artificial. The siRNA forms a complex with the RNA-induced silencing complex (RISC). The antisense (guide) strand of siRNA directs RISC to mRNA that has a complementary sequence, and then the mRNA is cleaved by RISC or its translation is repressed.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment. The subject may have a disease or condition. In some embodiments, the subject has cancer. In some embodiments, the subject is human.

“Substantially identical” can mean that a first and second amino acid or polynucleotide sequence are at least 60%, 65%, 70%, 75%, 80%, 85%, 90%, 95%, 96%, 97%, 98%, or 99%, or less than 100% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is for a transcription factor that promotes differentiation of a cell into a hepatocyte or maturation of a hepatocyte.

“Target region” as used herein refers to the region of the target gene to which the CRISPR/Cas9-based gene editing or targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto. A variant can be a polynucleotide sequence that is substantially identical over the full length of the full polynucleotide sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the polynucleotide sequence or a fragment thereof.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of #2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within 2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the amino acid sequence or a fragment thereof.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. MODULATORS OF GENES

Provided herein are agents that modulate a gene or the gene product thereof. The agent may be referred to as a modulator. The modulator may be an activator or an inhibitor. In some embodiments, the modulator is an activator. The modulator may activate or inhibit a gene or gene product, and thereby promote or induce differentiation or maturation of a cell. The cell may be an induced pluripotent stem cell (IPSC), or an embryonic stem cell (ESC), or a hepatocyte such as a mature hepatocyte. In some embodiments, a mature hepatocyte may be an IPSC-derived hepatocyte (IHep). In some embodiments, a mature hepatocyte may be an embryonic stem cell (ESC)-derived hepatocyte. For example, modulation of the gene or gene product may induce or promote differentiation of an induced pluripotent stem cell (IPSC) or an embryonic stem cell (ESC) into a hepatocyte. In some embodiments, modulation of the gene induces or promotes maturation of an iPSC-derived hepatocyte or an ESC-derived hepatocyte. Modifying or modulating may include increasing or decreasing gene expression, for example. In some embodiments, the compositions and methods modify the expression of a target gene within a cell. The compositions and methods detailed herein may engineer or modify the gene expression programs within cells by engineering the cells directly. Markers of the promoted or increased differentiation of a cell into a hepatocyte or promoted or increased maturation of a hepatocyte may include, for example, albumin, alpha-1 antitrypsin, ASGR1, SERPINA1, Foxa2, or Hnf4a, or a combination thereof. In some embodiments, the compositions and methods comprise an agent that increases expression of albumin, alpha-1 antitrypsin, ASGR1, SERPINA1, Foxa2, Hnf4a, or a p450 enzyme, or a combination thereof, in the cell. Expression of a marker such as albumin, alpha-1 antitrypsin, ASGR1, SERPINA1, Foxa2, or Hnf4a, or a combination thereof, may be determined, measured, and/or detected by any suitable means in the art, including, for example, ELISA, immunohistochemistry, flow cytometry, FACS, DNA or RNA sequencing, and hybridization of reporters or probes to RNA transcripts. Sequences for exemplary markers are shown in TABLE 1.

TABLE 1
Sequences for markers of iPSC or ESC differentiation into hepatocytes
and maturation of iPSC-derived or ESC-derived hepatocytes.

| Marker | DNA Sequence | Protein Sequence |
|---|---|---|
| Albumin | CTCTTCTGTCAACCCCACGCGCCTTTGGCACAATGAA | MKWVTFISLLFLFSSA |
| | GTGGGTAACCTTTATTTCCCTTCTTTTTCTCTTTAGC | YSRGVFRRDAHKSEVA |
| | TCGGCTTATTCCAGGGGTGTGTTTCGTCGAGATGCAC | HRFKDLGEENFKALVL |
| | ACAAGAGTGAGGTTGCTCATCGGTTTAAAGATTTGGG | IAFAQYLQQCPFEDHV |
| | AGAAGAAAATTTCAAAGCCTTGGTGTTGATTGCCTTT | KLVNEVTEFAKTCVAD |
| | GCTCAGTATCTTCAGCAGTGTCCATTTGAAGATCATG | ESAENCDKSLHTLFGD |
| | TAAAATTAGTGAATGAAGTAACTGAATTTGCAAAAAC | KLCTVATLRETYGEMA |
| | ATGTGTTGCTGATGAGTCAGCTGAAAATTGTGACAAA | DCCAKQEPERNECFLQ |
| | TCACTTCATACCCTTTTTGGAGACAAATTATGCACAG | HKDDNPNLPRLVRPEV |
| | TTGCAACTCTTCGTGAAACCTATGGTGAAATGGCTGA | DVMCTAFHDNEETFLK |
| | CTGCTGTGCAAAACAAGAACCTGAGAGAAATGAATGC | KYLYEIARRHPYFYAP |
| | TTCTTGCAACACAAAGATGACAACCCAAACCTCCCCC | ELLFFAKRYKAAFTEC |
| | GATTGGTGAGACCAGAGGTTGATGTGATGTGCACTGC | CQAADKAACLLPKLDE |
| | TTTTCATGACAATGAAGAGACATTTTTGAAAAAATAC | LRDEGKASSAKQGLKC |
| | TTATATGAAATTGCCAGAAGACATCCTTACTTTTATG | ASLQKFGERAFKAWAV |
| | CCCCGGAACTCCTTTTCTTTGCTAAAAGGTATAAAGC | ARLSQRFPKAEFAEVS |
| | TGCTTTTACAGAATGTTGCCAAGCTGCTGATAAAGCT | KLVTDLTKVHTECCHG |
| | GCCTGCCTGTTGCCAAAGCTCGATGAACTTCGGGATG | DLLECADDRADLAKYI |
| | AAGGGAAGGCTTCGTCTGCCAAACAGGGACTCAAGTG | CENQDSISSKLKECCE |
| | TGCCAGTCTCCAAAAATTTGGAGAAAGAGCTTTCAAA | KPLLEKSHCIAEVEND |
| | GCATGGGCAGTAGCTCGCCTGAGCCAGAGATTTCCCA | EMPADLPSLAADFVGS |
| | AAGCTGAGTTTGCAGAAGTTTCCAAGTTAGTGACAGA | KDVCKNYAEAKDVFLG |
| | TCTTACCAAAGTCCACACGGAATGCTGCCATGGAGAT | MFLYEYARRHPDYSVV |
| | CTGCTTGAATGTGCTGATGACAGGGCGGACCTTGCCA | LLERLAKTYETTLEKC |
| | AGTATATCTGTGAAAATCAAGATTCGATCTCCAGTAA | CAAADPHECYAKVFDE |
| | ACTGAAGGAATGCTGTGAAAAACCTCTGTTGGAAAAA | FKPLVEEPQNLIKQNC |
| | TCCCACTGCATTGCCGAAGTGGAAAATGATGAGATGC | ELFEQLGEYKFQNALL |
| | CTGCTGACTTGCCTTCATTAGCTGCTGATTTTGTTGG | VRYTKKVPQVSTPTLV |
| | AAGTAAGGATGTTTGCAAAAACTATGCTGAGGCAAAG | EVSRNLGKVGSKCCKH |
| | GATGTCTTCCTGGGCATGTTTTTGTATGAATATGCAA | PEAKRMPCAEDCLSVF |
| | GAAGGCATCCTGATTACTCTGTCGTGCTGCTGCTGAG | LNQLCVLHEKTPVSDR |
| | ACTTGCCAAGACATATGAAACCACTCTAGAGAAGTGC | VTKCCTESLVNGRPCF |
| | TGTGCCGCTGCAGATCCTCATGAATGCTATGCCAAAG | SALEVDETYVPKEFNA |
| | TGTTCGATGAATTTAAACCTCTTGTGGAAGAGCCTCA | ETFTFHADICTLSEKE |
| | GAATTTAATCAAACAAAATTGTGAGCTTTTTGAGCAG | RQIKKQTALVELVKHK |
| | CTTGGAGAGTACAAATTCCAGAATGCGCTATTAGTTC | PKATKEQLKAVMDDFA |
| | GTTACACCAAGAAAGTACCCCAAGTGTCAACTCCAAC | AFVEKCCKADDKETCF |
| | TCTTGTAGAGGTCTCAAGAAACCTAGGAAAAGTGGGC | AEEGKKLVAASQAALG |
| | AGCAAATGTTGTAAACATCCTGAAGCAAAAAGAATGC | L |
| | CCTGTGCAGAAGACTGTCTATCCGTGTTCCTGAACCA | (SEQ ID NO: 78) |
| | GTTATGTGTGTTGCATGAGAAAACGCCAGTAAGTGAC | |
| | AGAGTCACCAAATGCTGCACAGAATCCTTGGTGAACG | |
| | GGCGACCATGCTTTTCAGCTCTGGAAGTCGATGAAAC | |
| | ATACGTTCCCAAAGAGTTTAATGCTGAAACATTCACC | |
| | TTCCATGCAGATATATGCACACTTTCTGAGAAGGAGA | |
| | GACAAATCAAGAAACAAACTGCACTTGTTGAGCTTGT | |
| | GAAACACAAGCCCAAGGCAACAAAAGAGCAACTGAAA | |
| | GCTGTTATGGATGATTTCGCAGCTTTTGTAGAGAAGT | |
| | GCTGCAAGGCTGACGATAAGGAGACCTGCTTTGCCGA | |
| | GGAGGGTAAAAAACTTGTTGCTGCAAGTCAAGCTGCC | |
| | TTAGGCTTATAACATCACATTTAAAAGCATCTCAGCC | |
| | TACCATGAGAATAAGAGAAAGAAAATGAAGATCAAAA | |
| | GCTTATTCATCTGTTTTTCTTTTTCGTTGGTGTAAAG | |
| | CCAACACCCTGTCTAAAAAA | |
| | (SEQ ID NO: 73) | |
| Alpha-1 | ATGCCGTCTTCTGTCTCGTGGGGCATCCTCCTGCTGG | MPSSVSWGILLLAGLC |
| Antitrypsin/ | CAGGCCTGTGCTGCCTGGTCCCTGTCTCCCTGGCTGA | CLVPVSLAEDPQGDAA |
| SERPINA1 | GGATCCCCAGGGAGATGCTGCCCAGAAGACAGATACA | QKTDTSHHDQDHPTEN |
| | TCCCACCATGATCAGGATCACCCAACCTTCAACAAGA | KITPNLAEFAFSLYRQ |
| | TCACCCCCAACCTGGCTGAGTTCGCCTTCAGCCTATA | LAHQSNSTNIFFSPVS |
| | CCGCCAGCTGGCACACCAGTCCAACAGCACCAATATC | IATAFAMLSLGTKADT |
| | TTCTTCTCCCCAGTGAGCATCGCTACAGCCTTTGCAA | HDEILEGLNFNLTEIP |
| | TGCTCTCCCTGGGGACCAAGGCTGACACTCACGATGA | EAQIHEGFQELLRTLN |
| | AATCCTGGAGGGCCTGAATTTCAACCTCACGGAGATT | QPDSQLQLTTGNGLFL |
| | CCGGAGGCTCAGATCCATGAAGGCTTCCAGGAACTCC | SEGLKLVDKFLEDVKK |
| | TCCGTACCCTCAACCAGCCAGACAGCCAGCTCCAGCT | LYHSEAFTVNFGDTEE |
| | GACCACCGGCAATGGCCTGTTCCTCAGCGAGGGCCTG | AKKQINDYVEKGTQGK |
| | AAGCTAGTGGATAAGTTTTTGGAGGATGTTAAAAAGT | IVDLVKELDRDTVFAL |
| | TGTACCACTCAGAAGCCTTCACTGTCAACTTCGGGGA | VNYIFFKGKWERPFEV |
| | CACCGAAGAGGCCAAGAAACAGATCAACGATTACGTG | KDTEEEDFHVDQVTTV |
| | GAGAAGGGTACTCAAGGGAAAATTGTGGATTTGGTCA | KVPMMKRLGMFNIQHC |
| | AGGAGCTTGACAGAGACACAGTTTTTGCTCTGGTGAA | KKLSSWVLLMKYLGNA |
| | TTACATCTTCTTTAAAGGCAAATGGGAGAGACCCTTT | TAIFFLPDEGKLQHLE |
| | GAAGTCAAGGACACCGAGGAAGAGGACTTCCACGTGG | NELTHDIITKFLENED |
| | ACCAGGTGACCACCGTGAAGGTGCCTATGATGAAGCG | RRSASLHLPKLSITGT |
| | TTTAGGCATGTTTAACATCCAGCACTGTAAGAAGCTG | YDLKSVLGQLGITKVF |
| | TCCAGCTGGGTGCTGCTGATGAAATACCTGGGCAATG | SNGADLSGVTEEAPLK |
| | CCACCGCCATCTTCTTCCTGCCTGATGAGGGGAAACT | LSKAVHKAVLTIDEKG |
| | ACAGCACCTGGAAAATGAACTCACCCACGATATCATC | TEAAGAMFLEAIPMSI |
| | ACCAAGTTCCTGGAAAATGAAGACAGAAGGTCTGCCA | PPEVKFNKPFVFLMIE |
| | GCTTACATTTACCCAAACTGTCCATTACTGGAACCTA | QNTKSPLFMGKVVNPT |
| | TGATCTGAAGAGCGTCCTGGGTCAACTGGGCATCACT | QK |
| | AAGGTCTTCAGCAATGGGGCTGACCTCTCCGGGGTCA | (SEQ ID NO: 79) |
| | CAGAGGAGGCACCCCTGAAGCTCTCCAAGGCCGTGCA | |
| | TAAGGCTGTGCTGACCATCGACGAGAAAGGGACTGAA | |
| | GCTGCTGGGGCCATGTTTTTAGAGGCCATACCCATGT | |
| | CTATCCCCCCCGAGGTCAAGTTCAACAAACCCTTTGT | |
| | CTTCTTAATGATTGAACAAAATACCAAGTCTCCCCTC | |
| | TTCATGGGAAAAGTGGTGAATCCCACCCAAAAATAA | |
| | (SEQ ID NO: 74) | |
| ASGR1 | AGGGCCCTCCTATGGACCCTGCCCGCTCCCCTCCCAT | MTKEYQDLQHLDNEES |
| | TGTCCACGGCTGTCCGCCCACCCCCATTCTCCAAGCT | DHHQLRKGPPPPQPLL |
| | TCAGCCCCCTCCTTAGTTCGGCATCTGCACAGCACTG | QRLCSGPRLLLLSIGL |
| | AAGAACCTGGGAATCAGACCCTGAGACCCTGAGCAAT | SLLLLVVVCVIGSQNS |
| | CCCAGGTCCAGCGCCAGCCCTATCATGACCAAGGAGT | QLQEELRGLRETFSNF |
| | ATCAAGACCTTCAGCATCTGGACAATGAGGAGAGTGA | TASTEAQVKGLSTQGG |
| | CCACCATCAGCTCAGAAAAGGGCCACCTCCTCCCCAG | NVGRKMKSLESQLEKQ |
| | CCCCTCCTGCAGCGTCTCTGCTCCGGACCTCGCCTCC | QKDLSEDHSSLLLHVK |
| | TCCTGCTCTCCCTGGGCCTCAGCCTCCTGCTGCTTGT | QFVSDLRSLSCQMAAL |
| | GGTTGTCTGTGTGATCGGATCCCAAAACTCCCAGCTG | QGNGSERTCCPVNWVE |
| | CAGGAGGAGCTGCGGGGCCTGAGAGAGACGTTCAGCA | HERSCYWFSRSGKAWA |
| | ACTTCACAGCGAGCACGGAGGCCCAGGTCAAGGGCTT | DADNYCRLEDAHLVVV |
| | GAGCACCCAGGGAGGCAATGTGGGAAGAAAGATGAAG | TSWEEQKFVQHHIGPV |
| | TCGCTAGAGTCCCAGCTGGAGAAACAGCAGAAGGACC | NTWMGLHDQNGPWKWV |
| | TGAGTGAAGATCACTCCAGCCTGCTGCTCCACGTGAA | DGTDYETGFKNWRPEQ |
| | GCAGTTCGTGTCTGACCTGCGGAGCCTGAGCTGTCAG | PDDWYGHGLGGGEDCA |
| | ATGGCGGCGCTCCAGGGCAATGGCTCAGAAAGGACCT | HFTDDGRWNDDVCQRP |
| | GCTGCCCGGTCAACTGGGTGGAGCACGAGCGCAGCTG | YRWVCETELDKASQEP |
| | CTACTGGTTCTCTCGCTCCGGGAAGGCCTGGGCTGAC | PLL |
| | GCCGACAACTACTGCCGGCTGGAGGACGCGCACCTGG | (SEQ ID NO: 80) |
| | TGGTGGTCACGTCCTGGGAGGAGCAGAAATTTGTCCA | |
| | GCACCACATAGGCCCTGTGAACACCTGGATGGGCCTC | |
| | CACGACCAAAACGGGCCCTGGAAGTGGGTGGACGGGA | |
| | CGGACTACGAGACGGGCTTCAAGAACTGGAGGCCGGA | |
| | GCAGCCGGACGACTGGTACGGCCACGGGCTCGGAGGA | |
| | GGCGAGGACTGTGCCCACTTCACCGACGACGGCCGCT | |
| | GGAACGACGACGTCTGCCAGAGGCCCTACCGCTGGGT | |
| | CTGCGAGACAGAGCTGGACAAGGCCAGCCAGGAGCCA | |
| | CCTCTCCTTTAATTTATTTCTTCAATGCCTCGACCTG | |
| | CCGCAGGGGTCCGGGATTGGGAATCCGCCCATCTGGG | |
| | GCCTCTTCTGCTTTCTCGGGAATTTTCATCTAGGATT | |
| | TTAAGGGAAGGGGAAGGATAGGGTGATGTTCCGAAGG | |
| | TGAGGAGCTTGAAACCCGTGGCGCTTTCTGCAGTTCA | |
| | CAATGATAACCTGCAAACTGCAGAAAGCGCCACGGGT | |
| | TTCAAGCTCCTCACCTTCG | |
| | (SEQ ID NO: 75) | |
| FOXA2 | GCGGCCGCGTCGACGCTAGAGGGGCTGCTTGCGCCAG | MLGAVKMEGHEPSDWS |
| | GCGCCGGCCGCCCCACTGCGGGTCCCTGGCGGCCGGT | SYYAEPEGYSSVSNMN |
| | GTCTGAGGAGTCGGAGAGCCGAGGCGGCCAGACCGTG | AGLGMNGMNTYMSMSA |
| | CGCCCCGCGCTTCTCCCGAGGCCGTTCCGGGTCTGAA | AAMGSGSGNMSAGSMN |
| | CTGTAACAGGGAGGGGCCTCGCAGGAGCAGCAGCGGG | MSSYVGAGMSPSLAGM |
| | CGAGTTAAAGTATGCTGGGAGCGGTGAAGATGGAAGG | SPGAGAMAGMGGSAGA |
| | GCACGAGCCGTCCGACTGGAGCAGCTACTATGCAGAG | AGVAGMGPHLSPSLSP |
| | CCCGAGGGCTACTCCTCCGTGAGCAACATGAACGCCG | LGGQAAGAMGGLAPYA |
| | GCCTGGGGATGAACGGCATGAACACGTACATGAGCAT | NMNSMSPMYGQAGLSR |
| | GTCGGCGGCCGCCATGGGCAGCGGCTCGGGCAACATG | ARDPKTYRRSYTHAKP |
| | AGCGCGGGCTCCATGAACATGTCGTCGTACGTGGGCG | PYSYISLITMAIQQSP |
| | CTGGCATGAGCCCGTCCCTGGGGGGATGTCCCCCGG | NKMLTLSEIYQWIMDL |
| | CGCGGGCGCCATGGCGGGCATGGGCGGCTCGGCCGGG | FPFYRQNQQRWQNSIR |
| | GCGGCCGGCGTGGGGGGCATGGGGCCGCACTTGAGTC | HSLSFNDCFLKVPRSP |
| | CCAGCCTGAGCCCGCTCGGGGGGCAGGCGGCCGGGGC | DKPGKGSFWTLHPDSG |
| | CATGGGCGGCCTGGCCCCCTACGCCAACATGAACTCC | NMFENGCYLRRQKRFK |
| | ATGAGCCCCATGTACGGGCAGGCGGGCCTGAGCCGCG | CEKQLALKEAAGAAGS |
| | CCCGCGACCCCAAGACCTACAGGCGCAGCTACACGCA | GKKAAAGAQASQAQLG |
| | CGCAAAGCCGCCCTACTCGTACATCTCGCTCATCACC | EAAGPASETPAGTESP |
| | ATGGCCATCCAGCAGAGCCCCAACAAGATGCTGACGC | HSSASPCQEHKRGGLG |
| | TGAGCGAGATCTACCAGTGGATCATGGACCTCTTCCC | ELKGTPAAALSPPEPA |
| | CTTCTACCGGCAGAACCAGCAGCGCTGGCAGAACTCC | PSPGQQQQAAAHLLGP |
| | ATCCGCCACTCGCTCTCCTTCAACGACTGTTTCCTGA | PHHPGLPPEAHLKPEH |
| | AGGTGCCCCGCTCGCCCGACAAGCCCGGCAAGGGCTC | HYAFNHPFSINNLMSS |
| | CTTCTGGACCCTGCACCCTGACTCGGGCAACATGTTC | EQQHHHSHHHHQPHKM |
| | GAGAACGGCTGCTACCTGCGCCGCCAGAAGCGCTTCA | DLKAYEQVMHYPGYGS |
| | AGTGCGAGAAGCAGCTGGCGCTGAAGGAGGCCGCAGG | PMPGSLAMGPVTNKTG |
| | CGCCGCCGGCAGCGGCAAGAAGGCGGCCGCCGGAGCC | LDASPLAADTSYYQGV |
| | CAGGCCTCACAGGCTCAACTCGGGGAGGCCGCCGGGC | YSRPIMNSS |
| | CGGCCTCCGAGACTCCGGGGGGCACCGAGTCGCCTCA | (SEQ ID NO: 81) |
| | CTCGAGCGCCTCCCCGTGCCAGGAGCACAAGCGAGGG | |
| | GGCCTGGGAGAGCTGAAGGGGACGCCGGCTGCGGCGC | |
| | TGAGCCCCCCAGAGCCGGCGCCCTCTCCCGGGCAGCA | |
| | GCAGCAGGCCGCGGCCCACCTGCTGGGCCCGCCCCAC | |
| | CACCCGGGCCTGCCGCCTGAGGCCCACCTGAAGCCGG | |
| | AACACCACTACGCCTTCAACCACCCGTTCTCCATCAA | |
| | CAACCTCATGTCCTCGGAGCAGCAGCACCACCACAGC | |
| | CACCACCACCACCAACCCCACAAAATGGACCTCAAGG | |
| | CCTACGAACAGGTGATGCACTACCCCGGCTACGGTTC | |
| | CCCCATGCCTGGCAGCTTGGCCATGGGCCCGGTCACG | |
| | AACAAAACGGGCCTGGACGCCTCGCCCCTGGCCGCAG | |
| | ATACCTCCTACTACCAGGGGGTGTACTCCCGGCCCAT | |
| | TATGAACTCCTCTTAAGAAGACGACGGCTTCAGGCCC | |
| | GGCTAACTCTGGCACCCCGGATCGAGGACAAGTGAGA | |
| | GAGCAAGTGGGGGTCGAGACTTTGGGGAGACGGTGTT | |
| | GCAGAGACGCAAGGGAGAAGAAATCCATAACACCCCC | |
| | ACCCCAACACCCCCAAGACAGCAGTCTTCTTCACCCG | |
| | CTGCAGCCGTTCCGTCCCAAACAGAGGGCCACACAGA | |
| | TACCCCACGTTCTATATAAGGAGGAAAACGGGAAAGA | |
| | ATATAAAGTTAAAAAAAAGCCTCCGGTTTCCACTACT | |
| | GTGTAGACTCCTGCTTCTTCAAGCACCTGCAGATTCT | |
| | GATTTTTTTGTTGTTGTTGTTCTCCTCCATTGCTGTT | |
| | GTTGCAGGGAAGTNTTACTT | |
| | (SEQ ID NO: 76) | |
| HNF4A | GCAGAGAGGGCACTGGGAGGAGGCAGTGGGAGGGCGG | MRLSKTLVDMDMADYS |
| | AGGGCGGGGGCCTTCGGGGTGGGCGCCCAGGGTAGGG | AALDPAYTTLEFENVQ |
| | CAGGTGGCCGCGGCGTGGAGGCAGGGAGAATGCGACT | VLTMGNDTSPSEGTNL |
| | CTCCAAAACCCTCGTCGACATGGACATGGCCGACTAC | NAPNSLGVSALCAICG |
| | AGTGCTGCACTGGACCCAGCCTACACCACCCTGGAAT | DRATGKHYGASSCDGC |
| | TTGAGAATGTGCAGGTGTTGACGATGGGCAATGATTT | KGFFRRSVRKNHMYSC |
| | GTTGCCGCTGCGTCTCGCCAGATTGAGGCATCCCCTC | RFSRQCVVDKDKRNQC |
| | CGACATCACTGGAGCATATCTGGAGGGGTGGACAGTT | RYCRLKKCFRAGMKKE |
| | CTCCACAGGGAGACACGTCCCCATCAGAAGGCACCAA | AVQNERDRISTRRSSY |
| | CCTCAACGCGCCCAACAGCCTGGGTGTCAGCGCCCTG | EDSSLPSINALLQAEV |
| | TGTGCCATCTGCGGGGACCGGGCCACGGGCAAACACT | LSRQITSPVSGINGDI |
| | ACGGTGCCTCGAGCTGTGACGGCTGCAAGGGCTTCTT | RAKKIASIADVCESMK |
| | CCGGAGGAGCGTGCGGAAGAACCACATGTACTCCTGC | EQLLVLVEWAKYIPAF |
| | AGATTTAGCCGGCAGTGCGTGGTGGACAAAGACAAGA | CELPLDDQVALLRAHA |
| | GGAACCAGTGCCGCTACTGCAGGCTCAAGAAATGCTT | GEHLLLGATKRSMVFK |
| | CCGGGCTGGCATGAAGAAGGAAGCCGTCCAGAATGAG | DVLLLGNDYIVPRHCP |
| | CGGGACCGGATCAGCACTCGAAGGTCAAGCTATGAGG | ELAEMSRVSIRILDEL |
| | ACAGCAGCCTGCCCTCCATCAATGCGCTCCTGCAGGC | VLPFQELQIDDNEYAY |
| | GGAGGTCCTGTCCCGACAGATCACCTCCCCCGTCTCC | LKAIIFFDPDAKGLSD |
| | GGGATCAACGGCGACATTCGGGCGAAGAAGATTGCCA | PGKIKRLRSQVQVSLE |
| | GCATCGCAGATGTGTGTGAGTCCATGAAGGAGCAGCT | DYINDRQYDSRGRFGE |
| | GCTGGTTCTCGTTGAGTGGGCCAAGTACATCCCAGCT | LLLLLPTLQSITWQMI |
| | TTCTGCGAGCTCCCCCTGGACGACCAGGTGGCCCTGC | EQIQFIKLFGMAKIDN |
| | TCAGAGCCCATGCTGGCGAGCACCTGCTGCTCGGAGC | LLQEMLLGGSPSDAPH |
| | CACCAAGAGATCCATGGTGTTCAAGGACGTGCTGCTC | AHHPLHPHLMQEHMGT |
| | CTAGGCAATGACTACATTGTCCCTCGGCACTGCCCGG | NVIVANTMPTHLSNGQ |
| | AGCTGGCGGAGATGAGCCGGGTGTCCATACGCATCCT | MCEWPRPRGQAATPET |
| | TGACGAGCTGGTGCTGCCCTTCCAGGAGCTGCAGATC | PQPSPPGGSGSEPYKL |
| | GATGACAATGAGTATGCCTACCTCAAAGCCATCATCT | LPGAVATIVKPLSAIP |
| | TCTTTGACCCAGATGCCAAGGGGCTGAGCGATCCAGG | QPTITKQEVI |
| | GAAGATCAAGCGGCTGCGTTCCCAGGTGCAGGTGAGC | (SEQ ID NO: 82) |
| | TTGGAGGACTACATCAACGACCGCCAGTATGACTCGC | |
| | GTGGCCGCTTTGGAGAGCTGCTGCTGCTGCTGCCCAC | |
| | CTTGCAGAGCATCACGTGGCAGATGATCGAGCAGATC | |
| | CAGTTCATCAAGCTCTTCGGCATGGCCAAGATTGACA | |
| | ACCTGTTGCAGGAGATGCTGCTGGGAGGGTCCCCCAG | |
| | CGATGCACCCCATGCCCACCACCCCCTGCACCCTCAC | |
| | CTGATGCAGGAACATATGGGAACCAACGTCATCGTTG | |
| | CCAACACAATGCCCACTCACCTCAGCAACGGACAGAT | |
| | GTGTGAGTGGCCCCGACCCAGGGGACAGGCAGCCACC | |
| | CCTGAGACCCCACAGCCCTCACCGCCAGGTGGCTCAG | |
| | GGTCTGAGCCCTATAAGCTCCTGCCGGGAGCCGTCGC | |
| | CACAATCGTCAAGCCCCTCTCTGCCATCCCCCAGCCG | |
| | ACCATCACCAAGCAGGAAGTTATCTAGCAAGCCGCTG | |
| | GGGCTTGGGGGCTCCACTGGCTCCCCCCAGCCCCCTA | |
| | AGAGAGCACCTGGTGATCACGTGGTCACGGCAAAGGA | |
| | AGACGTGATGCCAGGACCAGTCCCAGAGCAGGAATGG | |
| | GAAGGATGAAGGGCCCGAGAACATGGCCTAAGGCACA | |
| | TCCCACTGCACCCTGACGCCCTGCTCTGATAACAAGA | |
| | CTTTGACTTGGGGAGACCCTCTACTGCCTTGGACAAC | |
| | TTTCTCATGTTGAAGCCACTGCCTTCACCTTCACCTT | |
| | CATCCATGTCCAACCCCCGACTTCATCCCAAAGGACA | |
| | GCCGCCTGGAGATGACTTGAGCCTTACTTAAACCCAG | |
| | CTCCCTTCTTCCCTAGCCTGGTGCTTCTCCTCTCCTA | |
| | GCCCCGGTCATGGTGTCCAGACAGAGCCCTGTGAGGC | |
| | TGGGTCCAATTGTGGCACTTGGGGCACCTTGCTCCTC | |
| | CTTCTGCTGCTGCCCCCACCTCTGCTGCCTCCCTCTG | |
| | CTGTCACCTTGCTCAGCCATCCCGTCTTCTCCAACAC | |
| | CACCTCTACAGAGGCCAAGGAGGCCTTGGAAACGATT | |
| | CCCCCAGTCATTCTGGGAACATGTTGTAAGCACTGAC | |
| | TGGGACCAGGCACCAGGCAGGGTCTAGAAGGCTGTGG | |
| | TGAGGGAAGACGCCTTTCTCCTCCAACCCAAC | |
| | (SEQ ID NO: 77) | |

The agent, or the composition or the method comprising the agent, may target a gene or a regulatory element thereof. Regulatory elements include, for example, promoters and enhancers. Regulatory elements may be within 1000 base pairs of the transcription start site. Regulatory elements may be within 600 base pairs of the transcription start site. The agent, or the composition or the method comprising the agent, may modify the expression of a gene. For example, the agent, or the composition or the method comprising the agent, may reduce, inhibit, decrease, activate, increase, or enhance the expression or activity of a gene. The agent, or the composition or the method comprising the agent, may directly or indirectly modulate the activity of the gene's protein product. For example, the agent, or the composition or the method comprising the agent, may increase or decrease the activity or binding or enzymatic activity of the gene's protein product, or inhibit the binding of the gene's protein product to another molecule or ligand, or increase the binding of the gene's protein product to another molecule or ligand, or increase or decrease the level or amount of the gene's protein product, or increase or decrease the level or amount of the expressed protein, or increase or decrease the degradation of the gene's protein product, or a combination thereof.

The targeted gene may be a transcription factor. The targeted gene may be selected from, for example, NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a regulatory element thereof, or a region thereof, or a combination thereof. In some embodiments, the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a regulatory element thereof, or a region thereof, or a combination thereof.

In some embodiments, the targeted gene is selected from ALX1, ASCL2, ATF3, ATF3, BARX2, BCL6, CARHSP1, CDIP1, CDK2, CREB3, CREB3L3, CREBZF, CREM, CSDC2, CTBP2, DACH2, DBX1, DLX1, DLX3, DLX5, DMRTC2, DPRX, EN1, EN2, ESRRA, ESRRB, ESRRG, ETV3L, ETV4, EWSR1, FOS, FOSB, FOSL1, FOXA2, FOXA3, FOXB1, FOXD4L5, FOXP2, FOXP3, FOXP4, GATA2, GATA3, GCM1, GCM2, GRHL1, GRHL3, GSC2, HBP1, HNF1B, HNF4A, HNF4G, HOXA1, HOXA3, HOXA5, HOXA6, HOXA7, HOXB1, HOXB3, HOXB5, HOXB6, HOXB7, HOXB8, HOXC4, HOXC5, HOXC6, HOXC8, HOXD3, HOXD4, HOXD8, IKZF5, IRF4, IRF5, IRF6, IRX3, ISL1, KLF1, KLF4, KLF5, KLF6, KLF7, LBX2, LCOR, LHX3, LHX4, LHX6, LHX8, LMX1A, LMX1B, MAFG, MAFK, MBD3, MEIS2, MITF, MIXL1, MSC, MSGN1, MYCL, NANOG, NFIB, NHLH1, NHLH2, NKX1-2, NR1H2, NR1H3, NR1H4, NR112, NR113, NR3C1, NR3C2, NR5A1, NR5A2, OSR1, OTP, OVOL1, OVOL3, PAX2, PAX3, PAX5, PAX7, PAX8, PITX2, PKNOX1, PKNOX2, POU1F1, POU2F1, POU2F2, POU2F3, POU6F1, PPARD, PPARG, PURB, RARA, RELA, RUNX1, RUNX1T1, SALL4, SMAD2, SMAD3, SMAD9, SMARCA2, SOHLH1, SOX13, SOX14, SP7, SPDEF, TBX15, TBX18, TBX20, TBX22, TCF21, TCF7, TFAP2A, TFAP2B, TFAP2C, TFEB, TFEC, TLE1, TPRX1, YAF2, ZBTB32, ZBTB9, ZGLP1, ZIC4, ZMIZ2, ZNF195, ZNF275, ZNF398, ZNF500, ZNF581, ZNF618, ZNF683, and ZNF747, or a regulatory element thereof, or a region thereof, or a combination thereof.

In some embodiments, a first modulator and a second modulator are included. In some embodiments, a first modulator targets a gene that is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, NR5A1, HBP1, NHLH1, or YAF2, or a regulatory element thereof, or a region thereof, or a combination thereof, and a second modulator targeting a gene selected from FOXA2, HNF4A, NR5A2, FOXA3, or HNF4G, or a regulatory element thereof, or a region thereof, or a combination thereof, is further included.

In some embodiments, a first modulator targets a gene that is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a regulatory element thereof, or a region thereof, or a combination thereof, and a second modulator targeting a gene selected from FOXA2, HNF4A, NR5A2, FOXA3, or HNF4G, or a regulatory element thereof, or a region thereof, or a combination thereof, is further included.

The first and second modulators may be administered at the same time or sequentially. The second modulator may be administered before or after or concomitantly with the first modulator. The second modulator may be administered at least about 1 minute, at least about 2 minutes, at least about 5 minutes, at least about 10 minutes, at least about 30 minutes, at least about 1 hour, at least about 2 hours, at least about 6 hours, at least about 12 hours, at least about 24 hours, at least about 36 hours, or at least about 48 hours before or after the first modulator is administered.

The agent may comprise, for example, a polynucleotide, a polypeptide, a small molecule, a lipid, a carbohydrate, or a combination thereof. In some embodiments, the agent comprises a protein. In some embodiments, the agent comprises an antibody. The antibody may bind to a protein encoded by a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, and YAF2, or a combination thereof. In some embodiments, the agent comprises a polynucleotide. The agent may comprise a polynucleotide encoding the gene or a fragment thereof or a polynucleotide comprising a cDNA of the gene or a fragment thereof selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, and YAF2, or a combination thereof. For example, the polynucleotide may comprise a sequence selected from SEQ ID NOs: 83-120, or a fragment thereof or a complement thereof. The agent may comprise a polypeptide comprising a protein product of the gene, or a fragment thereof, selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, YAF2, or a combination thereof. For example, the polypeptide may comprise a sequence selected from SEQ ID NOs: 121-158, or a fragment thereof. In some embodiments, the agent comprises a DNA targeting composition as detailed herein or at least one component thereof. Examples of genes for modulating cells are shown in TABLE 2.

TABLE 2
Exemplary genes and sequences for producing mature hepatocytes.

| Gene | ORF DNA Sequence | Protein Sequence |
|---|---|---|
| NHLH2 | ATGATGCTGAGTCCGGACCAAGCAGCAGATTCGGACCATCC | MMLSPDQAADSDHPS |
| | CAGCTCGGCGCACTCGGATCCGGAGTCCCTGGGCGGCACGG | SAHSDPESLGGTDTK |
| | ACACCAAGGTGCTCGGCAGCGTGTCGGACCTGGAGCCGGTG | VLGSVSDLEPVEEAE |
| | GAGGAGGCCGAGGGCGACGGCAAGGGCGGCAGCCGAGCCGC | GDGKGGSRAALYPHP |
| | GCTCTACCCGCACCCGCAGCAGCTGAGCCGCGAGGAGAAGC | QQLSREEKRRRRRAT |
| | GCCGCCGCCGGCGCGCCACGGCCAAGTACCGCTCGGCCCAC | AKYRSAHATRERIRV |
| | GCCACCCGCGAGCGCATCCGCGTGGAAGCCTTCAACTTGGC | EAFNLAFAELRKLLP |
| | CTTCGCCGAGCTCCGCAAATTGCTGCCCACGCTGCCCCCGG | TLPPDKKLSKIEILR |
| | ACAAGAAGCTCTCCAAGATCGAGATCCTGCGCCTGGCCATC | LAICYISYLNHVLDV |
| | TGCTACATCTCCTATCTCAACCACGTCCTGGACGTG | (SEQ ID NO: 121) |
| | (SEQ ID NO: 83) | |
| KLF7 | ATGGACGTGTTGGCTAGTTATAGTATATTCCAGGAGCTACA | MDVLASYSIFQELQL |
| | ACTTGTCCACGACACCGGCTACTTCTCAGCTTTACCATCCC | VHDTGYFSALPSLEE |
| | TGGAGGAGACCTGGCAGCAGACATGCCTTGAATTGGAACGC | TWQQTCLELERYLQT |
| | TACCTACAGACGGAGCCCCGGAGGATCTCAGAGACCTTTGG | EPRRISETFGEDLDC |
| | TGAGGACTTGGACTGTTTCCTCCACGCTTCCCCTCCCCCGT | FLHASPPPCIEESFR |
| | GCATTGAGGAAAGCTTCCGTCGCTTAGACCCCCTGCTGCTC | RLDPLLLPVEAAICE |
| | CCCGTGGAAGCGGCCATCTGTGAGAAGAGCTCGGCAGTGGA | KSSAVDILLSRDKLL |
| | CATCTTGCTCTCTCGGGACAAGTTGCTATCTGAGACCTGCC | SETCLSLQPASSSLD |
| | TCAGCCTCCAGCCGGCCAGCTCTTCTCTAGACAGCTACACA | SYTAVNQAQLNAVTS |
| | GCCGTCAACCAGGCCCAGCTCAACGCAGTGACCTCATTAAC | LTPPSSPELSRHLVK |
| | GCCCCCATCGTCCCCTGAGCTCAGCCGCCATCTGGTCAAAA | TSQTLSAVDGTVTLK |
| | CCTCACAAACTCTCTCTGCCGTGGATGGCACGGTGACGTTG | LVAKKAALSSVKVGG |
| | AAACTGGTGGCCAAGAAGGCTGCTCTCAGCTCCGTAAAGGT | VATAAAAVTAAGAVK |
| | GGGAGGGGTCGCAACAGCTGCAGCAGCCGTGACGGCTGCGG | SGQSDSDQGGLGAEA |
| | GGGCCGTTAAGAGTGGACAGAGCGACAGTGACCAAGGAGGG | CPENKKRVHRCQFNG |
| | CTGGGGGCTGAAGCATGTCCCGAAAACAAGAAGAGGGTTCA | CRKVYTKSSHLKAHQ |
| | CCGCTGTCAGTTTAACGGGTGCCGGAAAGTTTATACAAAAA | RTHTGEKPYKCSWEG |
| | GCTCCCACTTAAAGGCCCACCAGAGGACTCACACAGGTGAG | CEWRFARSDELTRHY |
| | AAGCCTTATAAGTGCTCATGGGAGGGATGTGAGTGGCGTTT | RKHTGAKPFKCNHCD |
| | TGCACGAAGCGATGAGCTCACGAGGCACTACAGGAAACACA | RCFSRSDHLALHMKR |
| | CAGGTGCAAAGCCCTTCAAATGCAACCACTGCGACAGGTGT | HI |
| | TTTTCCAGGTCTGACCATCTTGCCCTCCACATGAAGAGACA | (SEQ ID NO: 122) |
| | TATC | |
| | (SEQ ID NO: 84) | |
| GSC2 | ATGGCGGCAGCGGCTGGGGGCGCGGCGAGCCGCCGGGGTGC | MAAAAGGAASRRGAG |
| | CGGGCGGCCCTGCCCCTTCTCCATCGAGCACATCCTCTCCA | RPCPFSIEHILSSLP |
| | GCCTGCCCGAGCGGAGCCTCCCGGCCCGGGCCGCCTGCCCA | ERSLPARAACPPQPA |
| | CCGCAGCCCGCCGGTCGCCAGAGCCCCGCGAAGCCAGAGGA | GRQSPAKPEEPGAPE |
| | GCCCGGGGCGCCCGAGGCTGCGCCCTGCGCCTGCTGCTGCT | AAPCACCCCCGPRAA |
| | GCTGCGGCCCCCGCGCGGCGCCCTGCGGGCCCCCAGAGGCG | PCGPPEAAAGLGARL |
| | GCCGCCGGGCTGGGCGCTCGTCTGGCGTGGCCGCTGAGGCT | AWPLRLGPAVPLSLG |
| | GGGACCGGCGGTGCCCTTGTCTCTGGGTGCGCCAGCCGGAG | APAGGSGALPGAVGP |
| | GTTCCGGGGCGCTCCCGGGCGCGGTCGGCCCGGGTTCGCAG | GSQRRTRRHRTIFSE |
| | CGGCGCACGAGGCGCCACCGCACCATCTTCAGCGAAGAGCA | EQLQALEALFVQNQY |
| | GCTGCAGGCGCTCGAGGCGCTTTTCGTGCAGAACCAGTATC | PDVSTRERLAGRIRL |
| | CTGACGTGAGTACGCGCGAGCGCCTGGCCGGCCGCATCCGC | REERVEVWFKNRRAK |
| | CTTCGCGAGGAGCGCGTGGAGGTCTGGTTCAAGAACCGCCG | WRHQKRASASARLLP |
| | GGCCAAATGGCGACACCAGAAGCGCGCGTCGGCTTCCGCGA | GVKKSPKGSC |
| | GGCTCCTGCCCGGCGTCAAGAAGTCCCCGAAGGGGAGCTGC | (SEQ ID NO: 123) |
| | (SEQ ID NO: 85) | |
| PPARG | ATGGGTGAAACTCTGGGAGATTCTCCTATTGACCCAGAAAG | MGETLGDSPIDPESD |
| | CGATTCCTTCACTGATACACTGTCTGCAAACATATCACAAG | SFTDTLSANISQEMT |
| | AAATGACCATGGTTGACACAGAGATGCCATTCTGGCCCACC | MVDTEMPFWPTNFGI |
| | AACTTTGGGATCAGCTCCGTGGATCTCTCCGTAATGGAAGA | SSVDLSVMEDHSHSF |
| | CCACTCCCACTCCTTTGATATCAAGCCCTTCACTACTGTTG | DIKPFTTVDFSSIST |
| | ACTTCTCCAGCATTTCTACTCCACATTACGAAGACATTCCA | PHYEDIPFTRTDPVV |
| | TTCACAAGAACAGATCCAGTGGTTGCAGATTACAAGTATGA | ADYKYDLKLQEYQSA |
| | CCTGAAACTTCAAGAGTACCAAAGTGCAATCAAAGTGGAGC | IKVEPASPPYYSEKT |
| | CTGCATCTCCACCTTATTATTCTGAGAAGACTCAGCTCTAC | QLYNKPHEEPSNSLM |
| | AATAAGCCTCATGAAGAGCCTTCCAACTCCCTCATGGCAAT | AIECRVCGDKASGFH |
| | TGAATGTCGTGTCTGTGGAGATAAAGCTTCTGGATTTCACT | YGVHACEGCKGFFRR |
| | ATGGAGTTCATGCTTGTGAAGGATGCAAGGGTTTCTTCCGG | TIRLKLIYDRCDLNC |
| | AGAACAATCAGATTGAAGCTTATCTATGACAGATGTGATCT | RIHKKSRNKCQYCRF |
| | TAACTGTCGGATCCACAAAAAAAGTAGAAATAAATGTCAGT | QKCLAVGMSHNAIRF |
| | ACTGTCGGTTTCAGAAATGCCTTGCAGTGGGGATGTCTCAT | GRMPQAEKEKLLAEI |
| | AATGCCATCAGGTTTGGGCGGATGCCACAGGCCGAGAAGGA | SSDIDQLNPESADLR |
| | GAAGCTGTTGGCGGAGATCTCCAGTGATATCGACCAGCTGA | ALAKHLYDSYIKSFP |
| | ATCCAGAGTCCGCTGACCTCCGGGCCCTGGCAAAACATTTG | LTKAKARAILTGKTT |
| | TATGACTCATACATAAAGTCCTTCCCGCTGACCAAAGCAAA | DKSPFVIYDMNSLMM |
| | GGCGAGGGCGATCTTGACAGGAAAGACAACAGACAAATCAC | GEDKIKFKHITPLQE |
| | CATTCGTTATCTATGACATGAATTCCTTAATGATGGGAGAA | QSKEVAIRIFQGCQF |
| | GATAAAATCAAGTTCAAACACATCACCCCCCTGCAGGAGCA | RSVEAVQEITEYAKS |
| | GAGCAAAGAGGTGGCCATCCGCATCTTTCAGGGCTGCCAGT | IPGFVNLDLNDQVTL |
| | TTCGCTCCGTGGAGGCTGTGCAGGAGATCACAGAGTATGCC | LKYGVHEIIYTMLAS |
| | AAAAGCATTCCTGGTTTTGTAAATCTTGACTTGAACGACCA | LMNKDGVLISEGQGF |
| | AGTAACTCTCCTCAAATATGGAGTCCACGAGATCATTTACA | MTREFLKSLRKPFGD |
| | CAATGCTGGCCTCCTTGATGAATAAAGATGGGGTTCTCATA | FMEPKFEFAVKFNAL |
| | TCCGAGGGCCAAGGCTTCATGACAAGGGAGTTTCTAAAGAG | ELDDSDLAIFIAVII |
| | CCTGCGAAAGCCTTTTGGTGACTTTATGGAGCCCAAGTTTG | LSGDRPGLLNVKPIE |
| | AGTTTGCTGTGAAGTTCAATGCACTGGAATTAGATGACAGC | DIQDNLLQALELQLK |
| | GACTTGGCAATATTTATTGCTGTCATTATTCTCAGTGGAGA | LNHPESSQLFAKLLQ |
| | CCGCCCAGGTTTGCTGAATGTGAAGCCCATTGAAGACATTC | KMTDLRQIVTEHVQL |
| | AAGACAACCTGCTACAAGCCCTGGAGCTCCAGCTGAAGCTG | LQVIKKTETDMSLHP |
| | AACCACCCTGAGTCCTCACAGCTGTTTGCCAAGCTGCTCCA | LLQEIYKDLY |
| | GAAAATGACAGACCTCAGACAGATTGTCACGGAACATGTGC | (SEQ ID NO: 124) |
| | AGCTACTGCAGGTGATCAAGAAGACGGAGACAGACATGAGT | |
| | CTTCACCCGCTCCTGCAGGAGATCTACAAGGACTTGTAC | |
| | (SEQ ID NO: 86) | |
| SALL4 | ATGTCGAGGCGCAAGCAGGCGAAACCCCAGCACATCAACTC | MSRRKQAKPQHINSE |
| | GGAGGAGGACCAGGGCGAGCAGCAGCCGCAGCAGCAGACCC | EDQGEQQPQQQTPEF |
| | CGGAGTTTGCAGATGCGGCCCCAGCGGCGCCCGCGGcGGGG | ADAAPAAPAAGELGA |
| | GAGCTGGGTGCTCCAGTGAACCACCCAGGGAATGACGAGGT | PVNHPGNDEVASEDE |
| | GGCGAGTGAGGATGAAGCCACAGTAAAGCGGCTTCGTCGGG | ATVKRLRREETHVCE |
| | AGGAGACGCACGTCTGTGAGAAATGCTGTGCGGAGTTCTTC | KCCAEFFSISEFLEH |
| | AGCATCTCTGAGTTCCTGGAACATAAGAAAAATTGCACTAA | KKNCTKNPPVLIMND |
| | AAATCCACCTGTCCTCATCATGAATGACAGCGAGGGGCCTG | SEGPVPSEDFSGAVL |
| | TGCCTTCAGAAGACTTCTCCGGAGCTGTACTGAGCCACCAG | SHQPTSPGSKDCHRE |
| | CCCACCAGTCCCGGCAGTAAGGACTGTCACAGGGAGAATGG | NGGSSEDMKEKPDAE |
| | CGGCAGCTCAGAGGACATGAAGGAGAAGCCGGATGCGGAGT | SVVYLKTETALPPTP |
| | CTGTGGTGTACCTAAAGACAGAGACAGCCCTGCCACCCACC | QDISYLAKGKVANTN |
| | CCCCAGGACATAAGCTATTTAGCCAAAGGCAAAGTGGCCAA | VTLQALRGTKVAVNQ |
| | CACTAATGTGACCTTGCAGGCACTACGGGGCACCAAGGTGG | RSADALPAPVPGANS |
| | CGGTGAATCAGCGGAGCGCGGATGCACTCCCTGCCCCCGTG | IPWVLEQILCLQQQQ |
| | CCTGGTGCCAACAGCATCCCGTGGGTCCTCGAGCAGATCTT | LQQIQLTEQIRIQVN |
| | GTGTCTGCAGCAGCAGCAGCTACAGCAGATCCAGCTCACCG | MWASHALHSSGAGAD |
| | AGCAGATCCGCATCCAGGTGAACATGTGGGCCTCCCACGCC | TLKTLGSHMSQQVSA |
| | CTCCACTCAAGCGGGGCAGGGGCCGACACTCTGAAGACCTT | AVALLSQKAGSQGLS |
| | GGGCAGCCACATGTCTCAGCAGGTTTCTGCAGCTGTGGCTT | LDALKQAKLPHANIP |
| | TGCTCAGCCAGAAAGCTGGAAGCCAAGGTCTGTCTCTGGAT | SATSSLSPGLAPFTL |
| | GCCTTGAAACAAGCCAAGCTACCTCACGCCAACATCCCTTC | KPDGTRVLPNVMSRL |
| | TGCCACCAGCTCCCTGTCCCCAGGGCTGGCACCCTTCACTC | PSALLPQAPGSVLFQ |
| | TGAAGCCGGATGGGACCCGGGTGCTCCCGAACGTCATGTCC | SPFSTVALDTSKKGK |
| | CGCCTCCCGAGCGCTTTGCTTCCTCAGGCCCCGGGCTCGGT | GKPPNISAVDVKPKD |
| | GCTCTTCCAGAGCCCTTTCTCCACTGTGGCGCTAGACACAT | EAALYKHKCRSSLPS |
| | CCAAGAAAGGGAAGGGGAAGCCACCGAACATCTCCGCGGTG | TFIRAPPTYVKVEVP |
| | GATGTCAAACCCAAAGACGAGGCGGCCCTCTACAAGCACAA | GTFVGPSTLSPGMTP |
| | GTGTCGGAGCAGTCTCCCTTCCACGTTTATCCGAGCCCCGC | LLAAQPRRQAKQHGC |
| | CGACCTATGTCAAGGTTGAAGTTCCTGGCACATTTGTGGGA | TRCGKNFSSASALQI |
| | CCCTCGACATTGTCCCCAGGGATGACCCCTTTGTTAGCAGC | HERTHTGEKPFVCNI |
| | CCAGCCACGCCGACAGGCCAAGCAACATGGCTGCACACGGT | CGRAFTTKGNLKVHY |
| | GTGGGAAGAACTTCTCGTCTGCTAGCGCTCTTCAGATCCAC | MTHGANNNSARRGRK |
| | GAGCGGACTCACACTGGAGAGAAGCCTTTTGTGTGCAACAT | LAIENTMALLGTDGK |
| | TTGTGGGCGAGCTTTTACCACCAAAGGCAACTTAAAGGTTC | RVSEIFPKEILAPSV |
| | ACTACATGACACACGGGGCGAACAATAACTCAGCCCGCCGT | NVDPVVWNQYTSMLN |
| | GGAAGGAAGTTGGCCATCGAGAACACCATGGCTCTGTTAGG | GGLAVKTNEISVIQS |
| | TACGGACGGAAAAAGAGTCTCAGAAATCTTTCCCAAGGAAA | GGVPTLPVSLGATSV |
| | TCCTGGCCCCTTCAGTGAATGTGGACCCTGTTGTGTGGAAC | VNNATVSKMDGSQSG |
| | CAGTACACCAGCATGCTCAATGGCGGTCTGGCCGTGAAGAC | ISADVEKPSATDGVP |
| | CAATGAGATCTCTGTGATCCAGAGTGGGGGGGTTCCTACCC | KHQFPHFLEENKIAV |
| | TCCCGGTTTCCTTGGGGGCCACCTCCGTTGTGAATAACGCC | S |
| | ACTGTCTCCAAGATGGATGGCTCCCAGTCGGGTATCAGTGC | (SEQ ID NO: 125) |
| | AGATGTGGAAAAACCAAGTGCTACTGACGGCGTTCCCAAAC | |
| | ACCAGTTTCCTCACTTCCTGGAAGAAAACAAGATTGCGGTC | |
| | AGC | |
| | (SEQ ID NO: 87) | |
| ESRRG_1 | ATGTCAAACAAAGATCGACACATTGATTCCAGCTGTTCGTC | MSNKDRHIDSSCSSF |
| | CTTCATCAAGACGGAACCTTCCAGCCCAGCCTCCCTGACGG | IKTEPSSPASLTDSV |
| | ACAGCGTCAACCACCACAGCCCTGGTGGCTCTTCAGACGCC | NHHSPGGSSDASGSY |
| | AGTGGGAGCTACAGTTCAACCATGAATGGCCATCAGAACGG | SSTMNGHQNGLDSPP |
| | ACTTGACTCGCCACCTCTCTACCCTTCTGCTCCTATCCTGG | LYPSAPILGGSGPVR |
| | GAGGTAGTGGGCCTGTCAGGAAACTGTATGATGACTGCTCC | KLYDDCSSTIVEDPQ |
| | AGCACCATTGTTGAAGATCCCCAGACCAAGTGTGAATACAT | TKCEYMLNSMPKRLC |
| | GCTCAACTCGATGCCCAAGAGACTGTGTTTAGTGTGTGGTG | LVCGDIASGYHYGVA |
| | ACATCGCTTCTGGGTACCACTATGGGGTAGCATCATGTGAA | SCEACKAFFKRTIQG |
| | GCCTGCAAGGCATTCTTCAAGAGGACAATTCAAGGCAATAT | NIEYSCPATNECEIT |
| | AGAATACAGCTGCCCTGCCACGAATGAATGTGAAATCACAA | KRRRKSCQACRFMKC |
| | AGCGCAGACGTAAATCCTGCCAGGCTTGCCGCTTCATGAAG | LKVGMLKEGVRLDRV |
| | TGTTTAAAAGTGGGCATGCTGAAAGAAGGGGTGCGTCTTGA | RGGRQKYKRRIDAEN |
| | CAGAGTACGTGGAGGTCGGCAGAAGTACAAGCGCAGGATAG | SPYLNPQLVQPAKKP |
| | ATGCGGAGAACAGCCCATACCTGAACCCTCAGCTGGTTCAG | LLWSDPADNKIVSHL |
| | CCAGCCAAAAAGCCATTGCTCTGGTCTGATCCTGCAGATAA | LVAEPEKIYAMPDPT |
| | CAAGATTGTCTCACATTTGTTGGTGGCTGAACCGGAGAAGA | VPDSDIKALTTLCDL |
| | TCTATGCCATGCCTGACCCTACTGTCCCCGACAGTGACATC | ADRELVVIIGWAKHI |
| | AAAGCCCTCACTACACTGTGTGACTTGGCCGACCGAGAGTT | PGFSTLSLADQMSLL |
| | GGTGGTTATCATTGGATGGGCGAAGCATATTCCAGGCTTCT | QSAWMEILILGVVYR |
| | CCACGCTGTCCCTGGCGGACCAGATGAGCCTTCTGCAGAGT | SLSFEDELVYADDYI |
| | GCTTGGATGGAAATTTTGATCCTTGGTGTCGTATACCGGTC | MDEDQSKLAGLLDLN |
| | TCTTTCGTTTGAGGATGAACTTGTCTATGCAGACGATTATA | NAILQLVKKYKSMKL |
| | TAATGGACGAAGACCAGTCCAAATTAGCAGGCCTTCTTGAT | EKEEFVTLKAIALAN |
| | CTAAATAATGCTATCCTGCAGCTGGTAAAGAAATACAAGAG | SDSMHIEDVEAVQKL |
| | CATGAAGCTGGAAAAAGAAGAATTTGTCACCCTCAAAGCTA | QDVLHEALQDYEAGQ |
| | TAGCTCTTGCTAATTCAGACTCCATGCACATAGAAGATGTT | HMEDPRRAGKMLMTL |
| | GAAGCCGTTCAGAAGCTTCAGGATGTCTTACATGAAGCGCT | PLLRQTSTKAVQHFY |
| | GCAGGATTATGAAGCTGGCCAGCACATGGAAGACCCTCGTC | NIKLEGKVPMHKLFL |
| | GAGCTGGCAAGATGCTGATGACACTGCCACTCCTGAGGCAG | EMLEAKV |
| | ACCTCTACCAAGGCCGTGCAGCATTTCTACAACATCAAACT | (SEQ ID NO: 126) |
| | AGAAGGCAAAGTCCCAATGCACAAACTTTTTTTGGAAATGT | |
| | TGGAGGCCAAGGTC | |
| | (SEQ ID NO: 88) | |
| ESRRG_2 | ATGTCAAACAAAGATCGACACATTGATTCCAGCTGTTCGTC | MSNKDRHIDSSCSSF |
| | CTTCATCAAGACGGAACCTTCCAGCCCAGCCTCCCTGACGG | IKTEPSSPASLTDSV |
| | ACAGCGTCAACCACCACAGCCCTGGTGGCTCTTCAGACGCC | NHHSPGGSSDASGSY |
| | AGTGGGAGCTACAGTTCAACCATGAATGGCCATCAGAACGG | SSTMNGHQNGLDSPP |
| | ACTTGACTCGCCACCTCTCTACCCTTCTGCTCCTATCCTGG | LYPSAPILGGSGPVR |
| | GAGGTAGTGGGCCTGTCAGGAAACTGTATGATGACTGCTCC | KLYDDCSSTIVEDPQ |
| | AGCACCATTGTTGAAGATCCCCAGACCAAGTGTGAATACAT | TKCEYMLNSMPKRLC |
| | GCTCAACTCGATGCCCAAGAGACTGTGTTTAGTGTGTGGTG | LVCGDIASGYHYGVA |
| | ACATCGCTTCTGGGTACCACTATGGGGTAGCATCATGTGAA | SCEACKAFFKRTIQG |
| | GCCTGCAAGGCATTCTTCAAGAGGACAATTCAAGGCAATAT | NIEYSCPATNECEIT |
| | AGAATACAGCTGCCCTGCCACGAATGAATGTGAAATCACAA | KRRRKSCQACRFMKC |
| | AGCGCAGACGTAAATCCTGCCAGGCTTGCCGCTTCATGAAG | LKVGMLKEGVRLDRV |
| | TGTTTAAAAGTGGGCATGCTGAAAGAAGGGGTGCGTCTTGA | RGGRQKYKRRIDAEN |
| | CAGAGTACGTGGAGGTCGGCAGAAGTACAAGCGCAGGATAG | SPYLNPQLVQPAKKP |
| | ATGCGGAGAACAGCCCATACCTGAACCCTCAGCTGGTTCAG | YNKIVSHLLVAEPEK |
| | CCAGCCAAAAAGCCATATAACAAGATTGTCTCACATTTGTT | IYAMPDPTVPDSDIK |
| | GGTGGCTGAACCGGAGAAGATCTATGCCATGCCTGACCCTA | ALTTLCDLADRELVV |
| | CTGTCCCCGACAGTGACATCAAAGCCCTCACTACACTGTGT | IIGWAKHIPGFSTLS |
| | GACTTGGCCGACCGAGAGTTGGTGGTTATCATTGGATGGGC | LADQMSLLQSAWMEI |
| | GAAGCATATTCCAGGCTTCTCCACGCTGTCCCTGGCGGACC | LILGVVYRSLSFEDE |
| | AGATGAGCCTTCTGCAGAGTGCTTGGATGGAAATTTTGATC | LVYADDYIMDEDQSK |
| | CTTGGTGTCGTATACCGGTCTCTTTCATTTGAGGATGAACT | LAGLLDLNNAILQLV |
| | TGTCTATGCAGACGATTATATAATGGACGAAGACCAGTCCA | KKYKSMKLEKEEFVT |
| | AATTAGCAGGCCTTCTTGATCTAAATAATGCTATCCTGCAG | LKAIALANSDSMHIE |
| | CTGGTAAAGAAATACAAGAGCATGAAGCTGGAAAAAGAAGA | DVEAVQKLQDVLHEA |
| | ATTTGTCACCCTCAAAGCTATAGCTCTTGCTAATTCAGACT | LQDYEAGQHMEDPRR |
| | CCATGCACATAGAAGATGTTGAAGCCGTTCAGAAGCTTCAG | AGKMLMTLPLLRQTS |
| | GATGTCTTACATGAAGCGCTGCAGGATTATGAAGCTGGCCA | TKAVQHFYNIKLEGK |
| | GCACATGGAAGACCCTCGTCGAGCTGGCAAGATGCTGATGA | VPMHKLFLEMLEAKV |
| | CACTGCCACTCCTGAGGCAGACCTCTACCAAGGCCGTGCAG | (SEQ ID NO: 127) |
| | CATTTCTACAACATCAAACTAGAAGGCAAAGTCCCAATGCA | |
| | CAAACTTTTTTTGGAAATGTTGGAGGCCAAGGTC | |
| | (SEQ ID NO: 89) | |
| ESRRG_3 | ATGTGGCGAGAATGTGATTGGGGTCTTGGAGCAGTCAAGTC | MWRECDWGLGAVKSD |
| | TGATCTGGCCTGTGTTCCTTCAGCTAAAAGGCTTCTCTGCA | LACVPSAKRLLCRMS |
| | GAATGTCAAACAAAGATCGACACATTGATTCCAGCTGTTCG | NKDRHIDSSCSSFIK |
| | TCCTTCATCAAGACGGAACCTTCCAGCCCAGCCTCCCTGAC | TEPSSPASLTDSVNH |
| | GGACAGCGTCAACCACCACAGCCCTGGTGGCTCTTCAGACG | HSPGGSSDASGSYSS |
| | CCAGTGGGAGCTACAGTTCAACCATGAATGGCCATCAGAAC | TMNGHQNGLDSPPLY |
| | GGACTTGACTCGCCACCTCTCTACCCTTCTGCTCCTATCCT | PSAPILGGSGPVRKL |
| | GGGAGGTAGTGGGCCTGTCAGGAAACTGTATGATGACTGCT | YDDCSSTIVEDPQTK |
| | CCAGCACCATTGTTGAAGATCCCCAGACCAAGTGTGAATAC | CEYMLNSMPKRLCLV |
| | ATGCTCAACTCGATGCCCAAGAGACTGTGTTTAGTGTGTGG | CGDIASGYHYGVASC |
| | TGACATCGCTTCTGGGTACCACTATGGGGTAGCATCATGTG | EACKAFFKRTIQGNI |
| | AAGCCTGCAAGGCATTCTTCAAGAGGACAATTCAAGGCAAT | EYSCPATNECEITKR |
| | ATAGAATACAGCTGCCCTGCCACGAATGAATGTGAAATCAC | RRKSCQACRFMKCLK |
| | AAAGCGCAGACGTAAATCCTGCCAGGCTTGCCGCTTCATGA | VGMLKEGVRLDRVRG |
| | AGTGTTTAAAAGTGGGCATGCTGAAAGAAGGGGTGCGTCTT | GRQKYKRRIDAENSP |
| | GACAGAGTACGTGGAGGTCGGCAGAAGTACAAGCGCAGGAT | YLNPQLVQPAKKPLL |
| | AGATGCGGAGAACAGCCCATACCTGAACCCTCAGCTGGTTC | WSDPADNKIVSHLLV |
| | AGCCAGCCAAAAAGCCATTGCTCTGGTCTGATCCTGCAGAT | AEPEKIYAMPDPTVP |
| | AACAAGATTGTCTCACATTTGTTGGTGGCTGAACCGGAGAA | DSDIKALTTLCDLAD |
| | GATCTATGCCATGCCTGACCCTACTGTCCCCGACAGTGACA | RELVVIIGWAKHIPG |
| | TCAAAGCCCTCACTACACTGTGTGACTTGGCCGACCGAGAG | FSTLSLADQMSLLQS |
| | TTGGTGGTTATCATTGGATGGGCGAAGCATATTCCAGGCTT | AWMEILILGVVYRSL |
| | CTCCACGCTGTCCCTGGCGGACCAGATGAGCCTTCTGCAGA | SFEDELVYADDYIMD |
| | GTGCTTGGATGGAAATTTTGATCCTTGGTGTCGTATACCGG | EDQSKLAGLLDLNNA |
| | TCTCTTTCGTTTGAGGATGAACTTGTCTATGCAGACGATTA | ILQLVKKYKSMKLEK |
| | TATAATGGACGAAGACCAGTCCAAATTAGCAGGCCTTCTTG | EEFVTLKAIALANSD |
| | ATCTAAATAATGCTATCCTGCAGCTGGTAAAGAAATACAAG | SMHIEDVEAVQKLQD |
| | AGCATGAAGCTGGAAAAAGAAGAATTTGTCACCCTCAAAGC | VLHEALQDYEAGQHM |
| | TATAGCTCTTGCTAATTCAGACTCCATGCACATAGAAGATG | EDPRRAGKMLMTLPL |
| | TTGAAGCCGTTCAGAAGCTTCAGGATGTCTTACATGAAGCG | LRQTSTKAVQHFYNI |
| | CTGCAGGATTATGAAGCTGGCCAGCACATGGAAGACCCTCG | KLEGKVPMHKLFLEM |
| | TCGAGCTGGCAAGATGCTGATGACACTGCCACTCCTGAGGC | LEAKV |
| | AGACCTCTACCAAGGCCGTGCAGCATTTCTACAACATCAAA | (SEQ ID NO: 128) |
| | CTAGAAGGCAAAGTCCCAATGCACAAACTTTTTTTGGAAAT | |
| | GTTGGAGGCCAAGGTC | |
| | (SEQ ID NO: 90) | |
| ESRRB | ATGTCCTCGGACGACAGGCACCTGGGCTCCAGCTGCGGCTC | MSSDDRHLGSSCGSF |
| | CTTCATCAAGACTGAGCCGTCCAGCCCGTCCTCGGGCATCG | IKTEPSSPSSGIDAL |
| | ATGCCCTCAGCCACCACAGCCCCAGTGGCTCGTCCGACGCC | SHHSPSGSSDASGGF |
| | AGCGGCGGCTTTGGCCTGGCCCTGGGCACCCACGCCAACGG | GLALGTHANGLDSPP |
| | TCTGGACTCGCCACCCATGTTTGCAGGCGCCGGGCTGGGAG | MFAGAGLGGTPCRKS |
| | GCACCCCATGCCGCAAGAGCTACGAGGACTGTGCCAGCGGC | YEDCASGIMEDSAIK |
| | ATCATGGAGGACTCGGCCATCAAGTGCGAGTACATGCTCAA | CEYMLNAIPKRLCLV |
| | CGCCATCCCCAAGCGCCTGTGCCTCGTGTGCGGGGACATTG | CGDIASGYHYGVASC |
| | CCTCTGGCTACCACTACGGCGTGGCCTCCTGCGAGGCTTGC | EACKAFFKRTIQGNI |
| | AAGGCCTTCTTCAAGAGGACTATCCAAGGGAACATTGAGTA | EYSCPATNECEITKR |
| | CAGCTGCCCGGCCACCAACGAGTGCGAGATCACCAAACGGA | RRKSCQACRFMKCLK |
| | GGCGCAAGTCCTGCCAGGCCTGCCGCTTCATGAAATGCCTC | VGMLKEGVRLDRVRG |
| | AAAGTGGGGATGCTGAAGGAAGGTGTGCGCCTTGATCGAGT | GRQKYKRRLDSESSP |
| | GCGTGGAGGCCGTCAGAAATACAAGCGACGGCTGGACTCAG | YLSLQISPPAKKPLT |
| | AGAGCAGCCCATACCTGAGCTTACAAATTTCTCCACCTGCT | KIVSYLLVAEPDKLY |
| | AAAAAGCCATTGACCAAGATTGTCTCATACCTACTGGTGGC | AMPPPGMPEGDIKAL |
| | TGAGCCGGACAAGCTCTATGCCATGCCTCCCCCTGGTATGC | TTLCDLADRELVVII |
| | CTGAGGGGGACATCAAGGCCCTGACCACTCTCTGTGACCTG | GWAKHIPGFSSLSLG |
| | GCAGACCGAGAGCTTGTGGTCATCATTGGCTGGGCCAAGCA | DQMSLLQSAWMEILI |
| | CATCCCAGGCTTCTCAAGCCTCTCCCTGGGGGACCAGATGA | LGIVYRSLPYDDKLV |
| | GCCTGCTGCAGAGTGCCTGGATGGAAATCCTCATCCTGGGC | YAEDYIMDEEHSRLA |
| | ATCGTGTACCGCTCGCTGCCCTATGACGACAAGCTGGTGTA | GLLELYRAILQLVRR |
| | CGCTGAGGACTACATCATGGATGAGGAGCACTCCCGCCTCG | YKKLKVEKEEFVTLK |
| | CGGGGCTGCTGGAGCTCTACCGGGCCATCCTGCAGCTGGTA | ALALANSDSMYIEDL |
| | CGCAGGTACAAGAAGCTCAAGGTGGAGAAGGAGGAGTTTGT | EAVQKLQDLLHEALQ |
| | GACGCTCAAGGCCCTGGCCCTCGCCAACTCCGATTCCATGT | DYELSQRHEEPWRTG |
| | ACATCGAGGATCTAGAGGCTGTCCAGAAGCTGCAGGACCTG | KLLLTLPLLRQTAAK |
| | CTGCACGAGGCACTGCAGGACTACGAGCTGAGCCAGCGCCA | AVQHFYSVKLQGKVP |
| | TGAGGAGCCCTGGAGGACGGGCAAGCTGCTGCTGACACTGC | MHKLFLEMLEAKVGQ |
| | CGCTGCTGCGGCAGACGGCCGCCAAGGCCGTGCAGCACTTC | EQLRGSPKDERMSSH |
| | TATAGCGTCAAACTGCAGGGCAAAGTGCCCATGCACAAACT | DGKCPFQSAAFTSRD |
| | CTTCCTGGAGATGCTGGAGGCCAAGGTTGGCCAAGAGCAGC | QSNSPGIPNPRPSSP |
| | TTAGAGGATCTCCCAAGGATGAAAGAATGTCAAGCCATGAT | TPLNERGRQISPSTR |
| | GGAAAATGCCCCTTCCAATCAGCTGCCTTCACAAGCAGGGA | TPGGQGKHLWLTM |
| | TCAGAGCAACTCCCCGGGGATCCCCAATCCACGCCCTTCTA | (SEQ ID NO: 129) |
| | GTCCAACCCCCCTCAATGAGAGAGGCAGGCAGATCTCACCC | |
| | AGCACTAGGACACCAGGAGGCCAGGGAAAGCATCTCTGGCT | |
| | CACCATG | |
| | (SEQ ID NO: 91) | |
| SMAD2 | ATGTCGTCCATCTTGCCATTCACGCCGCCAGTTGTGAAGAG | MSSILPFTPPVVKRL |
| | ACTGCTGGGATGGAAGAAGTCAGCTGGTGGGTCTGGAGGAG | LGWKKSAGGSGGAGG |
| | CAGGCGGAGGAGAGCAGAATGGGCAGGAAGAAAAGTGGTGT | GEQNGQEEKWCEKAV |
| | GAGAAAGCAGTGAAAAGTCTGGTGAAGAAGCTAAAGAAAAC | KSLVKKLKKTGRLDE |
| | AGGACGATTAGATGAGCTTGAGAAAGCCATCACCACTCAAA | LEKAITTQNCNTKCV |
| | ACTGTAATACTAAATGTGTTACCATACCAAGGTCTCTTGAT | TIPRSLDGRLQVSHR |
| | GGTCGTCTCCAGGTATCCCATCGAAAAGGATTGCCACATGT | KGLPHVIYCRLWRWP |
| | TATATATTGCCGATTATGGCGCTGGCCTGATCTTCACAGTC | DLHSHHELKAIENCE |
| | ATCATGAACTCAAGGCAATTGAAAACTGCGAATATGCTTTT | YAFNLKKDEVCVNPY |
| | AATCTTAAAAAGGATGAAGTATGTGTAAACCCTTACCACTA | HYQRVETPVLPPVLV |
| | TCAGAGAGTTGAGACACCAGTTTTGCCTCCAGTATTAGTGC | PRHTEILTELPPLDD |
| | CCCGACACACCGAGATCCTAACAGAACTTCCGCCTCTGGAT | YTHSIPENTNFPAGI |
| | GACTATACTCACTCCATTCCAGAAAACACTAACTTCCCAGC | EPQSNYIPETPPPGY |
| | AGGAATTGAGCCACAGAGTAATTATATTCCAGAAACGCCAC | ISEDGETSDQQLNQS |
| | CTCCTGGATATATCAGTGAAGATGGAGAAACAAGTGACCAA | MDTGSPAELSPTTLS |
| | CAGTTGAATCAAAGTATGGACACAGGCTCTCCAGCAGAACT | PVNHSLDLQPVTYSE |
| | ATCTCCTACTACTCTTTCCCCTGTTAATCATAGCTTGGATT | PAFWCSIAYYELNQR |
| | TACAGCCAGTTACTTACTCAGAACCTGCATTTTGGTGTTCG | VGETFHASQPSLTVD |
| | ATAGCATATTATGAATTAAATCAGAGGGTTGGAGAAACCTT | GFTDPSNSERFCLGL |
| | CCATGCATCACAGCCCTCACTCACTGTAGATGGCTTTACAG | LSNVNRNATVEMTRR |
| | ACCCATCAAATTCAGAGAGGTTCTGCTTAGGTTTACTCTCC | HIGRGVRLYYIGGEV |
| | AATGTTAACCGAAATGCCACGGTAGAAATGACAAGAAGGCA | FAECLSDSAIFVQSP |
| | TATAGGAAGAGGAGTGCGCTTATACTACATAGGTGGGGAAG | NCNQRYGWHPATVCK |
| | TTTTTGCTGAGTGCCTAAGTGATAGTGCAATCTTTGTGCAG | IPPGCNLKIFNNQEF |
| | AGCCCCAATTGTAATCAGAGATATGGCTGGCACCCTGCAAC | AALLAQSVNQGFEAV |
| | AGTGTGTAAAATTCCACCAGGCTGTAATCTGAAGATCTTCA | YQLTRMCTIRMSFVK |
| | ACAACCAGGAATTTGCTGCTCTTCTGGCTCAGTCTGTTAAT | GWGAEYRRQTVTSTP |
| | CAGGGTTTTGAAGCCGTCTATCAGCTAACTAGAATGTGCAC | CWIELHLNGPLQWLD |
| | CATAAGAATGAGTTTTGTGAAAGGGTGGGGAGCAGAATACC | KVLTQMGSPSVRCSS |
| | GAAGGCAGACGGTAACAAGTACTCCTTGCTGGATTGAACTT | MS |
| | CATCTGAATGGACCTCTACAGTGGTTGGACAAAGTATTAAC | (SEQ ID NO: 130) |
| | TCAGATGGGATCCCCTTCAGTGCGTTGCTCAAGCATGTCA | |
| | (SEQ ID NO: 92) | |
| KLF6 | ATGGACGTGCTCCCCATGTGCAGCATCTTCCAGGAGCTCCA | MDVLPMCSIFQELQI |
| | GATCGTGCACGAGACCGGCTACTTCTCGGCGCTGCCGTCTC | VHETGYFSALPSLEE |
| | TGGAGGAGTACTGGCAACAGACCTGCCTAGAGCTGGAACGT | YWQQTCLELERYLQS |
| | TACCTCCAGAGCGAGCCCTGCTATGTTTCAGCCTCAGAAAT | EPCYVSASEIKFDSQ |
| | CAAATTTGACAGCCAGGAAGATCTGTGGACCAAAATCATTC | EDLWTKIILAREKKE |
| | TGGCTCGGGAGAAAAAGGAGGAATCCGAACTGAAGATATCT | ESELKISSSPPEDTL |
| | TCCAGTCCTCCAGAGGACACTCTCATCAGCCCGAGCTTTTG | ISPSFCYNLETNSLN |
| | TTACAACTTAGAGACCAACAGCCTGAACTCAGATGTCAGCA | SDVSSESSDSSEELS |
| | GCGAATCCTCTGACAGCTCCGAGGAACTTTCTCCCACGGCC | PTAKFTSDPIGEVLV |
| | AAGTTTACCTCCGACCCCATTGGCGAAGTTTTGGTCAGCTC | SSGKLSSSVTSTPPS |
| | GGGAAAATTGAGCTCCTCTGTCACCTCCACGCCTCCATCTT | SPELSREPSQLWGCV |
| | CTCCGGAACTGAGCAGGGAACCTTCTCAACTGTGGGGTTGC | PGELPSPGKVRSGTS |
| | GTGCCCGGGGAGCTGCCCTCGCCAGGGAAGGTGCGCAGCGG | GKPGDKGNGDASPDG |
| | GACTTCGGGGAAGCCAGGTGACAAGGGAAATGGCGATGCCT | RRRVHRCHFNGCRKV |
| | CCCCCGACGGCAGGAGGAGGGTGCACCGGTGCCACTTTAAC | YTKSSHLKAHQRTHT |
| | GGCTGCAGGAAAGTTTACACCAAAAGCTCCCACTTGAAAGC | GEKPYRCSWEGCEWR |
| | ACACCAGCGGACGCACACAGGAGAAAAGCCTTACAGATGCT | FARSDELTRHFRKHT |
| | CATCCCAACCCTCTCACTCCCCTTTTCCAACAACTCATCAC | GAKPFKCSHCDRCFS |
| | TTAACCAGGCACTTCCGAAAGCACACCGGGGCCAAGCCTTT | RSDHLALHMKRHL |
| | TAAATGCTCCCACTGTGACAGGTGTTTTTCCAGGTCTGACC | (SEQ ID NO: 131) |
| | ACCTGGCCCTGCACATGAAGAGGCACCTC | |
| | (SEQ ID NO: 93) | |
| TCF7_1 | ATGTACAAAGAGACCGTCTACTCCGCCTTCAATCTGCTCAT | MYKETVYSAFNLLMH |
| | GCATTACCCACCCCCCTCGGGAGCAGGGCAGCACCCCCAGC | YPPPSGAGQHPQPQP |
| | CGCAGCCCCCGCTGCACAAGGCCAATCAGCCCCCCCACGGT | PLHKANQPPHGVPQL |
| | GTCCCCCAACTCTCTCTCTACGAACATTTCAACAGCCCACA | SLYEHFNSPHPTPAP |
| | TCCCACCCCTGCACCTGCGGACATCAGCCAGAAGCAAGTTC | ADISQKQVHRPLQTP |
| | ACAGGCCTCTGCAGACCCCTGACCTCTCTGGCTTCTACTCC | DLSGFYSLTSGSMGQ |
| | CTGACCTCAGGCAGCATGGGGCAGCTCCCCCACACTGTGAG | LPHTVSWFTHPSLML |
| | CTGGTTCACCCACCCATCCTTGATGCTAGGTTCTGGTGTAC | GSGVPGHPAAIPHPA |
| | CTGGTCACCCAGCAGCCATCCCCCACCCGGCCATTGTGCCC | IVPPSGKQELQPFDR |
| | CCCTCAGGGAAGCAGGAGCTGCAGCCCTTCGACCGCAACCT | NIKTQAESKAEKEAK |
| | GAAGACACAAGCAGAGTCCAAGGCAGAGAAGGAGGCCAAGA | KPTIKKPLNAFMLYM |
| | AGCCAACCATCAAGAAGCCCCTCAATGCCTTCATGCTGTAC | KEMRAKVIAECTLKE |
| | ATGAAGGAGATGAGAGCCAAGGTCATTGCAGAGTGCACACT | SAAINQILGRRWHAL |
| | TAAGGAGAGCGCTGCCATCAACCAGATCCTGGGCCGCAGGT | SREEQAKYYELARKE |
| | GGCACGCGCTGTCGCGAGAAGAGCAGGCCAAGTACTATGAG | RQLHMQLYPGWSARD |
| | CTGGCCCGCAAGGAGAGGCAGCTGCACATGCAGCTATACCC | NYGKKKRRSREKHQE |
| | AGGCTGGTCAGCGCGGGACAACTACGGGAAGAAGAAGAGGC | STTGGKRNAFGTYPE |
| | GGTCGAGGGAAAAGCACCAAGAATCCACCACAGGAGGAAAA | KAAAPAPFLPMTVL |
| | AGAAATGCATTCGGTACTTACCCGGAGAAGGCCGCTGCCCC | (SEQ ID NO: 132) |
| | AGCCCCGTTCCTTCCGATGACAGTGCTC | |
| | (SEQ ID NO: 94) | |
| TCF7_2 | ATGTACAAAGAGACCGTCTACTCCGCCTTCAATCTGCTCAT | MYKETVYSAFNLLMH |
| | GCATTACCCACCCCCCTCGGGAGCAGGGCAGCACCCCCAGC | YPPPSGAGQHPQPQP |
| | CGCAGCCCCCGCTGCACAAGGCCAATCAGCCCCCCCACGGT | PLHKANQPPHGVPQL |
| | GTCCCCCAACTCTCTCTCTACGAACATTTCAACAGCCCACA | SLYEHFNSPHPTPAP |
| | TCCCACCCCTGCACCTGCGGACATCAGCCAGAAGCAAGTTC | ADISQKQVHRPLQTP |
| | ACAGGCCTCTGCAGACCCCTGACCTCTCTGGCTTCTACTCC | DLSGFYSLTSGSMGQ |
| | CTGACCTCAGGCAGCATGGGGCAGCTCCCCCACACTGTGAG | LPHTVSWFTHPSLML |
| | CTGGTTCACCCACCCATCCTTGATGCTAGGTTCTGGTGTAC | GSGVPGHPAAIPHPA |
| | CTGGTCACCCAGCAGCCATCCCCCACCCGGCCATTGTGCCC | IVPPSGKQELQPFDR |
| | CCCTCAGGGAAGCAGGAGCTGCAGCCCTTCGACCGCAACCT | NLKTQAESKAEKEAK |
| | GAAGACACAAGCAGAGTCCAAGGCAGAGAAGGAGGCCAAGA | KPTIKKPLNAFMLYM |
| | AGCCAACCATCAAGAAGCCCCTCAATGCCTTCATGCTGTAC | KEMRAKVIAECTLKE |
| | ATGAAGGAGATGAGAGCCAAGGTCATTGCAGAGTGCACACT | SAAINQILGRRWHAL |
| | TAAGGAGAGCGCTGCCATCAACCAGATCCTGGGCCGCAGGT | SREEQAKYYELARKE |
| | GGCACGCGCTGTCGCGAGAAGAGCAGGCCAAGTACTATGAG | RQLHMQLYPGWSARD |
| | CTGGCCCGCAAGGAGAGGCAGCTGCACATGCAGCTATACCC | NYGKKKRRSREKHQE |
| | AGGCTGGTCAGCGCGGGACAACTACGGGAAGAAGAAGAGGC | STTDPGSPKKCRARF |
| | GGTCGAGGGAAAAGCACCAAGAATCCACCACAGACCCTGGC | GLNQQTDWCGPCR |
| | TCGCCTAAGAAATGCCGTGCTCGCTTTGGCCTCAACCAGCA | (SEQ ID NO: 133) |
| | GACGGATTGGTGTGGTCCGTGCAGA | |
| | (SEQ ID NO: 95) | |
| TCF7_3 | ATGTACAAAGAGACCGTCTACTCCGCCTTCAATCTGCTCAT | MYKETVYSAFNLLMH |
| | GCATTACCCACCCCCCTCGGGAGCAGGGCAGCACCCCCAGC | YPPPSGAGQHPQPQP |
| | CGCAGCCCCCGCTGCACAAGGCCAATCAGCCCCCCCACGGT | PLHKANQPPHGVPQL |
| | GTCCCCCAACTCTCTCTCTACGAACATTTCAACAGCCCACA | SLYEHFNSPHPTPAP |
| | TCCCACCCCTGCACCTGCGGACATCAGCCAGAAGCAAGTTC | ADISQKQVHRPLQTP |
| | ACAGGCCTCTGCAGACCCCTGACCTCTCTGGCTTCTACTCC | DLSGFYSLTSGSMGQ |
| | CTGACCTCAGGCAGCATGGGGCAGCTCCCCCACACTGTGAG | LPHTVSWFTHPSLML |
| | CTGGTTCACCCACCCATCCTTGATGCTAGGTTCTGGTGTAC | GSGVPGHPAAIPHPA |
| | CTGGTCACCCAGCAGCCATCCCCCACCCGGCCATTGTGCCC | IVPPSGKQELQPFDR |
| | CCCTCAGGGAAGCAGGAGCTGCAGCCCTTCGACCGCAACCT | NLKTQAESKAEKEAK |
| | GAAGACACAAGCAGAGTCCAAGGCAGAGAAGGAGGCCAAGA | KPTIKKPLNAFMLYM |
| | AGCCAACCATCAAGAAGCCCCTCAATGCCTTCATGCTGTAC | KEMRAKVIAECTLKE |
| | ATGAAGGAGATGAGAGCCAAGGTCATTGCAGAGTGCACACT | SAAINQILGRRWHAL |
| | TAAGGAGAGCGCTGCCATCAACCAGATCCTGGGCCGCAGGT | SREEQAKYYELARKE |
| | GGCACGCGCTGTCGCGAGAAGAGCAGGCCAAGTACTATGAG | RQLHMQLYPGWSARD |
| | CTGGCCCGCAAGGAGAGGCAGCTGCACATGCAGCTATACCC | NYGKKKRRSREKHQE |
| | AGGCTGGTCAGCGCGGGACAACTACGGGAAGAAGAAGAGGC | STTETNWPRELKDGN |
| | GGTCGAGGGAAAAGCACCAAGAATCCACCACAGAGACAAAC | GQESLSMSSSSSPA |
| | TGGCCCAGAGAACTCAAGGATGGTAATGGACAAGAGTCACT | (SEQ ID NO: 134) |
| | GTCCATGTCTTCTTCCTCTAGCCCAGCT | |
| | (SEQ ID NO: 96) | |
| SMARCA2 | ATGAAGAGACTAGCAGCTCGCTGCTTTGCTGGCTTGTTAAT | MKRLAARCFAGLLIL |
| | TTTATCCCCACTAACTGTGATTTCTGATAGCCGGCCTGCTG | SPLTVISDSRPADSG |
| | ATAGTGGTAAGGCCATCGAAGACGGCAATTTGGAGGAAATG | KAIEDGNLEEMEEEV |
| | GAAGAGGAAGTACGGCTTAAGAAGCGAAAAAGACGAAGAAA | RLKKRKRRRNVDKDP |
| | TGTGGATAAAGATCCTGCAAAAGAAGATGTGGAAAAAGCTA | AKEDVEKAKKRRGRP |
| | AGAAGAGAAGAGGCCGCCCTCCCGCTGAGAAACTGTCACCA | PAEKLSPNPPKLTKQ |
| | AATCCCCCCAAACTGACAAAGCAGATGAACGCTATCATCGA | MNAIIDTVINYKDSS |
| | TACTGTGATAAACTACAAAGATAGTTCAGGGCGACAGCTCA | GRQLSEVFIQLPSRK |
| | GTGAAGTCTTCATTCAGTTACCTTCAAGGAAAGAATTACCA | ELPEYYELIRKPVDF |
| | GAATACTATGAATTAATTAGGAAGCCAGTGGATTTCAAAAA | KKIKERIRNHKYRSL |
| | AATAAAGGAAAGGATTCGTAATCATAAGTACCGGAGCCTAG | GDLEKDVMLLCHNAQ |
| | GCGACCTGGAGAAGGATGTCATGCTTCTCTGTCACAACGCT | TFNLEGSQIYEDSIV |
| | CAGACGTTCAACCTGGAGGGATCCCAGATCTATGAAGACTC | LQSVFKSARQKIAKE |
| | CATCGTCTTACAGTCAGTGTTTAAGAGTGCCCGGCAGAAAA | EESEDESNEEEEEED |
| | TTGCCAAAGAGGAAGAGAGTGAGGATGAAAGCAATGAAGAG | EEESESEAKSVKVKI |
| | GAGGAAGAGGAAGATGAAGAAGAGTCAGAGTCCGAGGCAAA | KLNKKDDKGRDKGKG |
| | ATCAGTCAAGGTGAAAATTAAGCTCAATAAAAAAGATGACA | KKRPNRGKAKPVVSD |
| | AAGGCCGGGACAAAGGGAAAGGCAAGAAAAGGCCAAATCGA | FDSDEEQDEREQSEG |
| | GGAAAAGCCAAACCTGTAGTGAGCGATTTTGACAGCGATGA | SGTDDE |
| | GGAGCAGGATGAACGTGAACAGTCAGAAGGAAGTGGGACGG | (SEQ ID NO: 135) |
| | ATGATGAG | |
| | (SEQ ID NO: 97) | |
| SMAD3 | ATGGAGCTGTGTGAGTTCGCCTTCAATATGAAGAAGGACGA | MELCEFAFNMKKDEV |
| | GGTCTGCGTGAATCCCTACCACTACCAGAGAGTAGAGACAC | CVNPYHYQRVETPVL |
| | CAGTTCTACCTCCTGTGTTGGTGCCACGCCACACAGAGATC | PPVLVPRHTEIPAEF |
| | CCGGCCGAGTTCCCCCCACTGGACGACTACAGCCATTCCAT | PPLDDYSHSIPENTN |
| | CCCCGAAAACACTAACTTCCCCGCAGGCATCGAGCCCCAGA | FPAGIEPQSNIPETP |
| | GCAATATTCCAGAGACCCCACCCCCTGGCTACCTGAGTGAA | PPGYLSEDGETSDHQ |
| | GATGGAGAAACCAGTGACCACCAGATGAACCACAGCATGGA | MNHSMDAGSPNLSPN |
| | CGCAGGTTCTCCAAACCTATCCCCGAATCCGATGTCCCCAG | PMSPAHNNLDLQPVT |
| | CACATAATAACTTGGACCTGCAGCCAGTTACCTACTGCGAG | YCEPAFWCSISYYEL |
| | CCGGCCTTCTGGTGCTCCATCTCCTACTACGAGCTGAACCA | NQRVGETFHASQPSM |
| | GCGCGTCGGGGAGACATTCCACGCCTCGCAGCCATCCATGA | TVDGFTDPSNSERFC |
| | CTGTGGATGGCTTCACCGACCCCTCCAATTCGGAGCGCTTC | LGLLSNVNRNAAVEL |
| | TGCCTAGGGCTGCTCTCCAATGTCAACAGGAATGCAGCAGT | TRRHIGRGVRLYYIG |
| | GGAGCTGACACGGAGACACATCGGAAGAGGCGTGCGGCTCT | GEVFAECLSDSAIFV |
| | ACTACATCGGAGGGGAGGTCTTCGCAGAGTGCCTCAGTGAC | QSPNCNQRYGWHPAT |
| | AGCGCTATTTTTGTCCAGTCTCCCAACTGTAACCAGCGCTA | VCKIPPGCNLKIFNN |
| | TGGCTGGCACCCGGCCACCGTCTGCAAGATCCCACCAGGAT | QEFAALLAQSVNQGF |
| | GCAACCTGAAGATCTTCAACAACCAGGAGTTCGCTGCCCTC | EAVYQLTRMCTIRMS |
| | CTGGCCCAGTCGGTCAACCAGGGCTTTGAGGCTGTCTACCA | FVKGWGAEYRRQTVT |
| | GTTGACCCGAATGTGCACCATCCGCATGAGCTTCGTCAAAG | STPCWIELHLNGPLQ |
| | GCTGGGGAGCGGAGTACAGGAGACAGACTGTGACCAGTACC | WLDKVLTQMGSPSIR |
| | CCCTGCTGGATTGAGCTGCACCTGAATGGGCCTTTGCAGTG | CSSVS |
| | GCTTGACAAGGTCCTCACCCAGATGGGCTCCCCAAGCATCC | (SEQ ID NO: 136) |
| | GCTGTTCCAGTGTGTCT | |
| | (SEQ ID NO: 98) | |
| TFEC_1 | ATGACCCTTGATCATCAGATCATCAATCCAACTCTTAAATG | MTLDHQIINPTLKWS |
| | GTCACAACCTGCAGTGCCAAGTGGTGGGCCTCTTGTGCAGC | QPAVPSGGPLVQHAH |
| | ATGCACACACAACTCTGGACAGTGATGCTGGCCTCACAGAA | TTLDSDAGLTENPLT |
| | AACCCACTCACCAAGTTACTAGCTATTGGGAAAGAAGATGA | KLLAIGKEDDNAQWH |
| | CAATGCACAATGGCATTTATCTGGAAGTATTTTGGATGTGT | LSGSILDVYSGEQGI |
| | ATAGCGGTGAACAAGGAATTTCACCAATTAACATGGGGCTT | SPINMGLTSASCPSS |
| | ACAAGTGCTTCTTGTCCAAGTAGTCTACCAATGAAAAGAGA | LPMKREITETDTRAL |
| | AATTACAGAAACTGACACTAGAGCTTTAGCAAAAGAGAGAC | AKERQKKDNHNLIER |
| | AAAAAAAGGACAACCACAACCTCATTGAAAGAAGAAGAAGG | RRRYNINYRIKELGT |
| | TATAATATTAATTACCGAATCAAGGAGCTTGGCACTCTTAT | LIPKSNDPDMRWNKG |
| | TCCAAAGTCTAATGATCCTGATATGCGCTGGAACAAAGGAA | TILKASVEYIKWLQK |
| | CCATTCTAAAAGCATCAGTGGAGTACATCAAGTGGCTACAA | EQQRARELEHRQKKL |
| | AAAGAACAACAGAGAGCCCGAGAATTGGAACACAGACAGAA | EQANRRLLLRIQELE |
| | GAAATTAGAGCAGGCTAACAGGCGACTTCTACTTCGGATTC | IQARTHGLPTLASLG |
| | AGGAACTAGAAATTCAGGCTCGTACTCATGGTCTGCCAACC | TVDLGAHVTKQQSHP |
| | CTGGCTTCACTTGGCACGGTTGATTTAGGTGCTCATGTCAC | EQNSVDYCQQLTVSQ |
| | CAAACAGCAGAGCCATCCTGAGCAGAATTCAGTAGACTATT | GPSPELCDQAIAFSD |
| | GCCAACAACTGACTGTGTCTCAGGGGCCAAGCCCTGAGCTC | PLSYFTDLSFSAALK |
| | TGTGATCAAGCTATAGCCTTTTCTGATCCTTTGTCATACTT | EEQRLDGMLLDDTIS |
| | CACAGATTTATCATTTAGTGCTGCATTGAAAGAGGAACAAA | PFGTDPLLSATSPAV |
| | GATTGGATGGCATGCTATTGGATGACACAATCTCTCCATTT | SKESSRRSSFSSDDG |
| | GGAACAGATCCTCTGCTATCTGCCACTTCCCCTGCAGTTTC | DEL |
| | CAAAGAAAGCAGTAGGAGAAGTAGCTTTAGCTCAGATGATG | (SEQ ID NO: 137) |
| | GTGATGAATTA | |
| | (SEQ ID NO: 99) | |
| TFEC_2 | ATGACCCTTGATCATCAGATCATCAATCCAACTCTTAAATGG | MTLDHQIINPTLKWS |
| | TCACAACCTGCAGTGCCAAGTGGTGGGCCTCTTGTGCAGCA | QPAVPSGGPLVQHAH |
| | TGCACACACAACTCTGGACAGTGATGCTGGCCTCACAGAAA | TTLDSDAGLTENPLT |
| | ACCCACTCACCAAGTTACTAGCTATTGGGAAAGAAGATGAC | KLLAIGKEDDNAQWH |
| | AATGCACAATGGCATATGGAGGACGTTATTGAGGATATAAT | MEDVIEDIIGMESSF |
| | CGGTATGGAATCAAGTTTTAAAGAGGAAGGAGCAGACTCTC | KEEGADSPLLMQRTL |
| | CTCTGCTAATGCAAAGAACATTATCTGGAAGTATTTTGGAT | SGSILDVYSGEQGIS |
| | GTGTATAGCGGTGAACAAGGAATTTCACCAATTAACATGGG | PINMGLTSASCPSSL |
| | GCTTACAAGTGCTTCTTGTCCAAGTAGTCTACCAATGAAAA | PMKREITETDTRALA |
| | GAGAAATTACAGAAACTGACACTAGAGCTTTAGCAAAAGAG | KERQKKDNHNLIERR |
| | AGACAAAAAAAGGACAACCACAACCTCATTGAAAGAAGAAG | RRYNINYRIKELGTL |
| | AAGGTATAATATTAATTACCGAATCAAGGAGCTTGGCACTC | IPKSNDPDMRWNKGT |
| | TTATTCCAAAGTCTAATGATCCTGATATGCGCTGGAACAAA | ILKASVEYIKWLQKE |
| | GGAACCATTCTAAAAGCATCAGTGGAGTACATCAAGTGGCT | QQRARELEHRQKKLE |
| | ACAAAAAGAACAACAGAGAGCCCGAGAATTGGAACACAGAC | QANRRLLLRIQELEI |
| | AGAAGAAATTAGAGCAGGCTAACAGGCGACTTCTACTTCGG | QARTHGLPTLASLGT |
| | ATTCAGGAACTAGAAATTCAGGCTCGTACTCATGGTCTGCC | VDLGAHVTKQQSHPE |
| | AACCCTGGCTTCACTTGGCACGGTTGATTTAGGTGCTCATG | QNSVDYCQQLTVSQG |
| | TCACCAAACAGCAGAGCCATCCTGAGCAGAATTCAGTAGAC | PSPELCDQAIAFSDP |
| | TATTGCCAACAACTGACTGTGTCTCAGGGGCCAAGCCCTGA | LSYFTDLSFSAALKE |
| | GCTCTGTGATCAAGCTATAGCCTTTTCTGATCCTTTGTCAT | EQRLDGMLLDDTISP |
| | ACTTCACAGATTTATCATTTAGTGCTGCATTGAAAGAGGAA | FGTDPLLSATSPAVS |
| | CAAAGATTGGATGGCATGCTATTGGATGACACAATCTCTCC | KESSRRSSFSSDDGD |
| | ATTTGGAACAGATCCTCTGCTATCTGCCACTTCCCCTGCAG | EL |
| | TTTCCAAAGAAAGCAGTAGGAGAAGTAGCTTTAGCTCAGAT | (SEQ ID NO: 138) |
| | GATGGTGATGAATTA | |
| | (SEQ ID NO: 100) | |
| PURB | ATGGCGGACGGCGACAGCGGCAGCGAGCGCGGCGGCGGCGG | MADGDSGSERGGGGG |
| | TGGGCCGTGCGGGTTCCAGCCCGCGTCCCGCGGCGGCGGCG | PCGFQPASRGGGEQE |
| | AGCAAGAGACGCAGGAGCTGGCCTCGAAGCGGCTGGACATC | TQELASKRLDIQNKR |
| | CAGAACAAGCGCTTCTACTTAGATGTGAAGCAGAACGCCAA | FYLDVKQNAKGRFLK |
| | GGGCCGCTTCCTCAAGATCGCCGAGGTGGGCGCGGGCGGTT | IAEVGAGGSKSRLTL |
| | CCAAGAGCCGCCTCACGCTGTCCATGGCGGTGGCCGCCGAG | SMAVAAEFRDSLGDF |
| | TTCCGCGACTCGCTGGGCGACTTCATAGAACACTACGCGCA | IEHYAQLGPSSPEQL |
| | GCTGGGCCCTAGCAGCCCCGAGCAGCTGGCGGCGGGCGCCG | AAGAEEGGGPRRALK |
| | AGGAGGGCGGCGGGCCGCGGCGCGCGCTCAAGAGCGAATTC | SEFLVRENRKYYLDL |
| | TTGGTGCGTGAGAACCGCAAGTACTACCTGGACCTCAAGGA | KENQRGRFLRIRQTV |
| | GAACCAGCGCGGCCGCTTCCTGCGCATCCGCCAAACGGTCA | NRGGGGFGAGPGPGG |
| | ACCGCGGCGGTGGCGGCTTCGGCGCGGGCCCCGGGCCGGGC | LQSGQTIALPAQGLI |
| | GGCTTGCAGAGCGGCCAGACCATCGCGCTGCCTGCGCAGGG | EFRDALAKLIDDYGG |
| | CCTCATCGAGTTCCGCGACGCGCTGGCGAAGCTCATAGACG | EDDELAGGPGGGAGG |
| | ACTACGGAGGCGAGGACGACGAGCTGGCAGGCGGCCCGGGA | PGGGLYGELPEGTSI |
| | GGCGGCGCCGGGGGCCCAGGGGGCGGCCTGTATGGAGAGCT | TVDSKRFFFDVGCNK |
| | CCCGGAGGGCACCTCCATCACCGTGGACTCCAAGCGCTTCT | YGVFLRVSEVKPSYR |
| | TCTTCGATGTGGGCTGCAACAAATACGGGGTGTTTCTGCGA | NAITVPFKAWGKFGG |
| | GTGAGCGAGGTGAAGCCGTCCTACCGCAATGCCATCACCGT | AFCRYADEMKEIQER |
| | ACCCTTCAAAGCCTGGGGCAAGTTCGGAGGCGCCTTTTGCC | QRDKLYERRGGGSGG |
| | GGTATGCGGATGAGATGAAAGAAATCCAGGAACGACAGAGG | GEESEGEEVDED |
| | GATAAGCTTTATGAGCGACGTGGTGGGGGCAGCGGCGGCGG | (SEQ ID NO: 139) |
| | CGAAGAGTCAGAGGGTGAGGAGGTGGATGAGGAT | |
| | (SEQ ID NO: 101) | |
| ZNF398 | ATGGCTGAGGCGGCCCCGGCCCCGACATCTGAATGGGACTC | MAEAAPAPTSEWDSE |
| | CGAGTGCCTTACATCCCTGCAGCCCCTTCCTCTTCCTACAC | CLTSLQPLPLPTPPA |
| | CCCCAGCAGCAAATGAGGCACACCTGCAGACAGCAGCTATC | ANEAHLQTAAISLWT |
| | TCTCTGTGGACAGTGGTGGCCGCCGTGCAGGCTATAGAGAG | VVAAVQAIERKVEIH |
| | GAAGGTGGAGATCCACAGCCGGCGACTCCTACACCTAGAAG | SRRLLHLEGRTGTAE |
| | GTCGGACAGGGACAGCAGAGAAGAAACTAGCCAGCTGTGAA | KKLASCEKTVTELGN |
| | AAGACAGTTACCGAGCTTGGGAACCAGCTGGAGGGCAAGTG | QLEGKWAVLGTLLQE |
| | GGCCGTGCTGGGAACCCTGCTGCAGGAGTACGGGCTGCTGC | YGLLQRRLENLENLL |
| | AGAGGCGGCTGGAGAACTTGGAGAACCTGCTGCGCAACAGG | RNRNFWILRLPPGIK |
| | AACTTCTGGATCCTGCGGCTCCCTCCAGGTATTAAGGGAGA | GDIPKVPVAFDDVSI |
| | TATCCCAAAGGTGCCTGTGGCATTTGATGATGTCTCCATCT | YFSTPEWEKLEEWQK |
| | ACTTTTCCACTCCAGAGTGGGAAAAATTAGAAGAATGGCAA | ELYKNIMKGNYESLI |
| | AAGGAACTTTACAAGAATATCATGAAGGGCAACTACGAGTC | SMDYAINQPDVLSQI |
| | TCTCATCTCCATGGATTATGCTATAAATCAACCTGATGTCT | QPEGEHNTEDQAGPE |
| | TATCTCAGATTCAACCAGAAGGGGAACATAATACAGAGGAC | ESEIPTDPSEEPGIS |
| | CAGGCAGGGCCAGAGGAAAGTGAGATTCCCACAGACCCCAG | TSDILSWIKQEEEPQ |
| | TGAAGAGCCTGGTATTTCAACATCAGATATTCTGTCTTGGA | VGAPPESKESDVYKS |
| | TTAAACAAGAAGAAGAGCCTCAGGTTGGGGCCCCACCGGAG | TYADEELVIKAEGLA |
| | TCCAAGGAGAGTGACGTGTACAAAAGCACTTATGCTGATGA | RSSLCPEVPVPFSSP |
| | AGAGCTTGTCATCAAAGCTGAAGGCCTTGCTAGATCCTCGT | PAAAKDAFSDVAFKS |
| | TGTGCCCTGAGGTTCCAGTCCCTTTCTCTTCTCCACCAGCA | QQSTSMTPFGRPATD |
| | GCAGCAAAGGATGCTTTTTCAGATGTGGCTTTCAAAAGCCA | LPEASEGQVTFTQLG |
| | GCAGTCTACATCCATGACACCTTTTGGACGTCCAGCCACTG | SYPLPPPVGEQVFSC |
| | ACCTGCCTGAAGCCTCTGAGGGACAAGTGACTTTTACTCAG | HHCGKNLSQDMLLTH |
| | TTGGGTAGCTATCCCCTCCCACCTCCAGTTGGCGAGCAGGT | QCSHATEHPLPCAQC |
| | GTTCTCATGCCACCACTGTGGCAAGAATCTCAGCCAAGACA | PKHFTPQADLSSTSQ |
| | TGTTGCTGACCCACCAATGTAGCCATGCTACTGAGCACCCC | DHASETPPTCPHCAR |
| | TTACCCTGTGCCCAGTGCCCTAAGCACTTTACTCCACAGGC | TFTHPSRLTYHLRVH |
| | GGACCTCAGCAGCACCTCCCAGGACCATGCCAGCGAGACAC | NSTERPFPCPDCPKR |
| | CCCCCACCTGCCCACACTGTGCCAGGACTTTTACTCACCCA | FADQARLTSHRRAHA |
| | TCAAGACTTACCTACCATCTTCGGGTCCATAACAGCACTGA | SERPFRCAQCGRSFS |
| | GCGTCCTTTCCCCTGTCCTGATTGCCCCAAGCGCTTTGCTG | LKISLLLHQRGHAQE |
| | ACCAGGCTCGACTCACCAGCCACCGGAGAGCTCATGCAAGC | RPFSCPQCGIDFNGH |
| | GAAAGGCCCTTCCGCTGTGCCCAGTGCGGCAGGAGCTTCAG | SALIRHQMIHTGERP |
| | CTTGAAAATCAGCCTCCTGCTCCACCAGCGGGGTCATGCAC | YPCTDCSKSFMRKEH |
| | AAGAGCGCCCTTTCTCCTGCCCTCAGTGTGGCATTGACTTC | LLNHRRLHTGERPFS |
| | AACGGCCACTCGGCCCTGATCCGCCACCAGATGATCCACAC | CPHCGKSFIRKHHLM |
| | AGGCGAGCGTCCTTACCCCTGCACTGACTGCAGTAAGAGCT | KHQRIHTGERPYPCS |
| | TCATGCGCAAGGAGCACCTGCTGAACCACCGGCGGCTGCAC | YCGRSFRYKQTLKDH |
| | ACAGGCGAGCGGCCCTTCAGTTGTCCTCACTGTGGCAAGAG | LRSGHNGGCGGDSDP |
| | CTTCATCCGCAAGCACCACCTAATGAAACACCAGCGCATCC | SGQPPNPPGPLITGL |
| | ACACCGGGGAGCGGCCCTACCCCTGCTCCTACTGTGGCAGG | ETSGLGVNTEGLETN |
| | AGCTTCCGCTACAAACAGACACTCAAGGACCACCTCCGTTC | QWYGEGSGGGVL |
| | AGGCCACAATGGAGGCTGTGGGGGTGATAGTGACCCATCAG | (SEQ ID NO: 140) |
| | GTCAGCCACCCAACCCACCAGGTCCCCTCATAACTGGGCTT | |
| | GAAACTTCTGGCCTGGGTGTCAACACTGAAGGTCTAGAGAC | |
| | CAACCAGTGGTATGGGGAAGGGAGTGGAGGGGGAGTTTTG | |
| | (SEQ ID NO: 102) | |
| ASCL2 | ATGGACGGCGGCACACTGCCCAGGTCCGCGCCCCCTGCGCC | MDGGTLPRSAPPAPP |
| | CCCCGTCCCTGTCGGCTGCGCTGCCCGGCGGAGACCCGCGT | VPVGCAARRRPASPE |
| | CCCCGGAACTGTTGCGCTGCAGCCGGCGGCGGCGACCGGCC | LLRCSRRRRPATAET |
| | ACCGCAGAGACCGGAGGCGGCGCAGCGGCCGTAGCGCGGCG | GGGAAAVARRNERER |
| | CAATGAGCGCGAGCGCAACCGCGTGAAGCTGGTGAACTTGG | NRVKLVNLGFQALRQ |
| | GCTTCCAGGCGCTGCGGCAGCACGTGCCGCACGGCGGCGCC | HVPHGGASKKLSKVE |
| | AGCAAGAAGCTGAGCAAGGTGGAGACGCTGCGCTCAGCCGT | TLRSAVEYIRALQRL |
| | GGAGTACATCCGCGCGCTGCAGCGCCTGCTGGCCGAGCACG | LAEHDAVRNALAGGL |
| | ACGCCGTGCGCAACGCGCTGGCGGGAGGGCTGAGGCCGCAG | RPQAVRPSAPRGPPG |
| | GCCGTGCGGCCGTCTGCGCCCCGCGGGCCGCCAGGGACCAC | TTPVAASPSRASSSP |
| | CCCGGTCGCCGCCTCGCCCTCCCGCGCTTCTTCGTCCCCGG | GRGGSSEPGSPRSAY |
| | GCCGCGGGGGCAGCTCGGAGCCCGGCTCCCCGCGTTCCGCC | SSDDSGCEGALSPAE |
| | TACTCGTCGGACGACAGCGGCTGCGAAGGCGCGCTGAGTCC | RELLDFSSWLGGY |
| | TGCGGAGCGCGAGCTACTCGACTTCTCCAGCTGGTTAGGGG | (SEQ ID NO: 141) |
| | GCTAC | |
| | (SEQ ID NO: 103) | |
| NFIB | ATGATGTATTCTCCCATCTGTCTCACTCAGGATGAATTTCA | MMYSPICLTQDEFHP |
| | CCCATTCATCGAGGCACTTCTTCCACATGTCCGTGCAATTG | FIEALLPHVRAIAYT |
| | CCTATACTTGGTTCAACCTGCAGGCTCGAAAACGCAAGTAC | WFNLQARKRKYFKKH |
| | TTTAAAAAGCATGAGAAGCGAATGTCAAAGGATGAAGAAAG | EKRMSKDEERAVKDE |
| | AGCAGTCAAAGATGAGCTTCTCAGTGAAAAGCCTGAAATCA | LLSEKPEIKQKWASR |
| | AACAGAAGTGGGCATCCAGGCTCCTTGCCAAACTGCGCAAA | LLAKLRKDIRQEYRE |
| | GATATTCGCCAGGAGTATCGAGAGGACTTTGTGCTCACCGT | DFVLTVTGKKHPCCV |
| | GACTGGCAAGAAGCACCCGTGCTGTGTCTTATCCAATCCCG | LSNPDQKGKIRRIDC |
| | ACCAGAAGGGTAAGATTAGGAGAATCGACTGCCTGCGACAG | LRQADKVWRLDLVMV |
| | GCAGACAAAGTCTGGCGTCTGGATCTAGTCATGGTGATCCT | ILFKGIPLESTDGER |
| | GTTCAAAGGCATCCCCTTGGAAAGTACCGATGGAGAGCGGC | LMKSPHCTNPALCVQ |
| | TCATGAAATCCCCACATTGCACAAACCCAGCACTTTGTGTC | PHHITVSVKELDLFL |
| | CAGCCACATCATATCACAGTATCAGTTAAGGAGCTTGATTT | AYYVQEQDSGQSGSP |
| | GTTTTTGGCATACTACGTGCAGGAGCAAGATTCTGGACAAT | SHNDPAKNPPGYLED |
| | CAGGAAGTCCAAGCCACAATGATCCTGCCAAGAATCCTCCA | SFVKSGVFNVSELVR |
| | GGTTACCTTGAGGATAGTTTTGTAAAATCTGGAGTCTTCAA | VSRTPITQGTGVNFP |
| | TGTATCAGAACTTGTAAGAGTATCCAGAACGCCCATAACCC | IGEIPSQPYYHDMNS |
| | AGGGAACTGGAGTCAACTTCCCAATTGGAGAAATCCCAAGC | GVNLQRSLSSPPSSK |
| | CAACCATACTATCATGACATGAACTCGGGGGTCAATCTTCA | RPKTISIDENMEPSP |
| | GAGGTCTCTGTCTTCTCCACCAAGCAGCAAAAGACCCAAAA | TGDFYPSPSSPAAGS |
| | CTATATCCATAGATGAAAATATGGAACCAAGTCCTACAGGA | RTWHERDQDMSSPTT |
| | GACTTTTACCCCTCTCCAAGTTCACCAGCTGCTGGAAGTCG | MKKPEKPLFSSASPQ |
| | AACATGGCACGAAAGAGATCAAGATATGTCTTCTCCGACTA | DSSPRLSTFPQHHHP |
| | CTATGAAGAAGCCTGAAAAGCCATTGTTCAGCTCTGCATCT | GIPGVAHSVISTRTP |
| | CCACAGGATTCTTCCCCAAGACTGAGCACTTTCCCCCAGCA | PPPSPLPFPTQAILP |
| | CCACCATCCCGGAATACCTGGAGTTGCACACAGTGTCATCT | PAPSSYFSHPTIRYP |
| | CAACTCGAACTCCACCTCCACCTTCACCGTTGCCATTTCCA | PHLNPQDTLKNYVPS |
| | ACACAAGCTATCCTTCCTCCAGCCCCATCGAGCTACTTTTC | YDPSSPQTSQSWYLG |
| | TCATCCAACAATCAGATATCCTCCCCACCTGAATCCTCAGG | (SEQ ID NO: 142) |
| | ATACTCTGAAGAACTATGTACCTTCTTATGACCCATCCAGT | |
| | CCACAAACCAGCCAGTCCTGGTACCTGGGC | |
| | (SEQ ID NO: 104) | |
| ZIC4 | ATGAGATACAAGACATCCTTGGTGATGAGGAAACGATTACG | MRYKTSLVMRKRLRL |
| | GCTTTACCGAAACACTCTTAAAGAGTCAAGTAGCAGCTCTG | YRNTLKESSSSSGHH |
| | GACACCATGGCCCCCAGCTCACCGCCGCCTCCAGCCCCTCG | GPQLTAASSPSVFPG |
| | GTGTTCCCGGGCCTCCACGAGGAGCCTCCCCAGGCCTCCCC | LHEEPPQASPSRPLN |
| | CAGCCGTCCTTTGAATGGACTCCTGCGTCTGGGGCTCCCTG | GLLRLGLPGDMYARP |
| | GAGACATGTACGCGCGGCCGGAGCCCTTCCCGCCAGGGCCT | EPFPPGPAARSDALA |
| | GCGGCCCGCAGCGACGCCCTGGCAGCTGCCGCAGCCCTGCA | AAAALHGYGGMNLTV |
| | TGGCTACGGGGGCATGAACCTGACGGTGAACCTCGCTGCGC | NLAAPHGPGAFFRYM |
| | CCCACGGTCCTGGCGCTTTCTTCCGCTACATGCGCCAGCCC | RQPIKQELICKWLAA |
| | ATCAAACAGGAGCTCATCTGCAAGTGGCTGGCGGCCGACGG | DGTATPSLCSKTFST |
| | CACCGCGACCCCGAGCCTCTGCTCCAAAACTTTCAGCACCA | MHELVTHVTVEHVGG |
| | TGCACGAGCTGGTCACGCACGTCACCGTGGAGCACGTCGGC | PEQANHICFWEECPR |
| | GGCCCGGAACAGGCCAACCACATTTGCTTCTGGGAGGAGTG | QGKPFKAKYKLVNHI |
| | TCCGCGCCAGGGAAAGCCCTTCAAAGCCAAATACAAACTTG | RVHTGEKPFPCPFPG |
| | TAAATCACATCCGCGTGCACACGGGCGAGAAGCCCTTCCCT | CGKVFARSENLKIHK |
| | TGTCCTTTCCCGGGGTGTGGGAAGGTCTTTGCTAGATCAGA | RTHTGEKPFRCEFEG |
| | AAATCTCAAAATACACAAACGAACTCACACAGGCGAGAAGC | CERRFANSSDRKKHS |
| | CCTTCAGATGCGAGTTCGAGGGCTGCGAGCGGCGCTTCGCC | HVHTSDKPYTCKVRG |
| | AACAGCAGCGACCGTAAGAAGCATTCGCACGTGCACACTAG | CDKCYTHPSSLRKHM |
| | CGACAAGCCATACACGTGCAAGGTGCGGGGCTGCGACAAGT | KVHGRSPPPSSGYDS |
| | GCTACACGCACCCCAGCTCGCTGCGTAAGCACATGAAGGTG | ATPSALVSPSSDCGH |
| | CACGGGCGCTCGCCGCCGCCCAGCTCTGGCTACGATTCGGC | KSQVASSAAVAARTA |
| | TACACCGTCTGCCCTCGTGTCGCCCTCGTCGGACTGCGGCC | DLSE |
| | ACAAGTCCCAGGTGGCCTCCTCGGCGGCGGTGGCGGCGCGT | (SEQ ID NO: 143) |
| | ACCGCCGACTTGAGCGAA | |
| | (SEQ ID NO: 105) | |
| ZNF618 | ATGAACCAGCCGGGCGGCGCGGCGGCTCCGCAGGCTGACGG | MNQPGGAAAPQADGA |
| | AGCCAGTGCAGCCGGAAGGAAAAGCACTGCGAGCAGGGAGC | SAAGRKSTASRERLK |
| | GCTTGAAGCGCAGCCAGAAGAGCACCAAGGTGGAGGGCCCA | RSQKSTKVEGPEPVP |
| | GAGCCAGTGCCAGCCGAGGCCTCGCTGAGTGCCGAGCAAGG | AEASLSAEQGTMTEV |
| | AACGATGACGGAGGTGAAGGTGAAGACAGAGCTGCCCGATG | KVKTELPDDYIQEVI |
| | ACTACATCCAGGAGGTGATCTGGCAGGGCGAGGCCAAGGAG | WQGEAKEEKKAVSKD |
| | GAGAAGAAGGCGGTCAGCAAGGATGGGACCAGCGACGTGCC | GTSDVPAEICVVIGG |
| | TGCCGAGATCTGCGTGGTGATCGGCGGCGTCCGCAACCAGC | VRNQQTLGSYECGIC |
| | AGACCCTTGGGTCTTACGAATGCGGAATCTGTGGCAAGAAG | GKKYKYYNCFQTHVR |
| | TACAAGTATTACAACTGCTTCCAGACCCACGTGCGGGCGCA | AHRDTEATSGEGASQ |
| | CCGAGACACCGAAGCCACCTCAGGGGAGGGAGCCTCCCAAA | SNNFRYTCDICGKKY |
| | GCAACAATTTCAGGTACACATGTGATATCTGCGGGAAGAAG | KYYSCFQEHRDLHAV |
| | TACAAATACTACAGCTGTTTCCAAGAGCACCGAGACCTGCA | DVFSVEGAPENRADP |
| | CGCAGTGGATGTGTTTAGTGTGGAAGGGGCCCCTGAGAACC | FDQGVVATDEVKEEP |
| | GGGCAGACCCCTTCGACCAAGGTGTCGTGGCCACGGACGAG | PEPFQKIGPMNNITS |
| | GTGAAGGAGGAGCCCCCGGAGCCATTCCAGAAAATCGGGCC | DIFKKKEVRQCQKRE |
| | AATGAACAATATTACATCAGACATTTTTAAGAAGAAAGAAG | TGNYTCEFCGKQYKY |
| | TTAGGCAGTGCCAAAAGAGAGAAACTGGCAATTACACCTGT | YTPYQEHVALHAPIK |
| | GAATTCTGCGGCAAACAGTACAAGTACTACACTCCCTACCA | SAFSRRVEGKAQNHF |
| | GGAGCATGTGGCCTTACACGCCCCCATCAAAAGCGCTTTCA | EETNSSSQNSSEPYT |
| | GTCGGAGAGTAGAAGGCAAAGCACAAAACCACTTTGAAGAG | CGACGIQFQFYNNLL |
| | ACGAACAGCAGTTCGCAGAACTCCAGCGAACCCTACACCTG | EHMQSHAADNENNIA |
| | CGGCGCCTGTGGGATCCAGTTCCAGTTCTACAACAACCTGT | SNQSRSPPAVVEEKW |
| | TGGAGCACATGCAGTCCCATGCAGCTGACAATGAAAACAAC | KPQAQRNSANNTTTS |
| | ATTGCCTCCAACCAGTCCCGATCGCCACCTGCTGTTGTAGA | GLTPNSMIPEKERQN |
| | AGAGAAGTGGAAACCTCAGGCCCAGAGGAACAGTGCCAATA | IAERLLRVMCADLGA |
| | ACACCACTACCAGTGGTTTAACACCCAACAGCATGATCCCC | LSVVSGKEFLKLAQT |
| | GAAAAGGAGCGGCAGAACATCGCAGAGCGGCTGTTGAGGGT | LVDSGARYGAFSVTE |
| | CATGTGTGCCGACCTGGGTGCACTGAGCGTGGTCAGCGGGA | ILGNFNTLALKHLPR |
| | AGGAGTTCCTGAAGTTGGCCCAGACCTTAGTAGACAGTGGT | MYNQVKVKVTCALGS |
| | GCCCGCTATGGGGCCTTCTCGGTCACTGAAATCCTGGGCAA | NACLGIGVTCHSQSV |
| | CTTCAACACGCTGGCGCTGAAGCACCTGCCACGCATGTACA | GPDSCYILTAYQAEG |
| | ACCAGGTGAAGGTGAAAGTGACCTGTGCCTTGGGCAGCAAT | NHIKSYVLGVKGADI |
| | GCCTGCCTAGGCATCGGTGTCACCTGCCACTCCCAGAGTGT | RDSGDLVHHWVQNVL |
| | TGGCCCTGACTCCTGCTACATCCTCACAGCCTACCAGGCCG | SEFVMSEIRTVYVTD |
| | AGGGCAACCACATCAAGAGCTATGTGCTTGGTGTGAAGGGT | CRVSTSAFSKAGMCL |
| | GCGGACATTCGCGACAGCGGTGACCTTGTGCACCACTGGGT | RCSACALNSVVQSVL |
| | GCAGAACGTGCTGTCGGAGTTCGTGATGTCGGAGATCAGGA | SKRTLQARSMHEVIE |
| | CAGTGTACGTGACGGATTGCCGGGTGAGCACGTCCGCCTTC | LLNVCEDLAGSTGLA |
| | TCCAAGGCCGGCATGTGCCTTCGCTGCTCAGCCTGTGCCTT | KETFGSLEETSPPPC |
| | GAACTCGGTGGTGCAGAGCGTGCTGAGCAAGCGGACACTGC | WNSVTDSLLLVHERY |
| | AGGCCCGCAGCATGCACGAGGTCATCGAGCTGCTCAACGTG | EQICEFYSRAKKMNL |
| | TGCGAGGACCTGGCGGGCTCCACGGGCCTGGCCAAGGAGAC | IQSLNKHLLSNLAAI |
| | CTTCGGGTCGCTGGAGGAGACGTCTCCACCACCCTGCTGGA | LTPVKQAVIELSNES |
| | ACTCGGTGACGGACTCACTGTTGCTGGTGCATGAGCGCTAT | QPTLQLVLPTYVRLE |
| | GAGCAGATCTGCGAGTTCTACAGCCGGGCCAAGAAGATGAA | KLFTAKANDAGTVSK |
| | CCTCATCCAGAGCCTCAACAAGCACCTGCTCAGCAACCTGG | LCHLFLEALKENFKV |
| | CGGCCATCCTGACGCCGGTGAAGCAGGCAGTCATCGAGCTG | HPAHKVAMILDPQQK |
| | AGCAACGAGAGCCAGCCCACCCTGCAGCTGGTGCTGCCCAC | LRPVPPYQHEEIIGK |
| | CTACGTCAGGCTGGAGAAGCTGTTCACGGCCAAGGCCAACG | VCELINEVKESWAEE |
| | ACGCAGGCACTGTCAGCAAGCTCTGCCACCTCTTCCTGGAG | ADFEPAAKKPRSAAV |
| | GCGCTCAAGGAGAACTTCAAGGTGCACCCGGCCCACAAGGT | ENPAAQEDDRLGKNE |
| | GGCCATGATCCTGGACCCGCAGCAGAAGCTGCGGCCTGTGC | VYDYLQEPLFQATPD |
| | CACCCTACCAGCACGAGGAGATCATCGGCAAGGTCTGTGAG | LFQYWSCVTQKHTKL |
| | CTCATCAACGAGGTGAAGGAGTCCTGGGCCGAGGAGGCCGA | AKLAFWLLAVPAVGA |
| | CTTCGAGCCCGCTGCCAAGAAGCCCCGCTCTGCTGCCGTCG | RSGCVNMCEQALLIK |
| | AGAACCCCGCAGCTCAGGAAGATGATCGGCTAGGCAAAAAT | RRRLLSPEDMNKLMF |
| | GAAGTGTACGATTACCTGCAGGAGCCCCTCTTCCAGGCTAC | LKSNML |
| | CCCTGATCTCTTCCAGTACTGGTCGTGCGTTACCCAAAAGC | (SEQ ID NO: 144) |
| | ACACAAAACTCGCCAAGCTCGCCTTCTGGCTCCTGGCGGTT | |
| | CCGGCCGTGGGCGCCAGAAGCGGGTGTGTAAATATGTGTGA | |
| | ACAAGCGCTTCTAATCAAACGGAGGCGGCTGCTCAGTCCAG | |
| | AAGATATGAATAAACTCATGTTTCTGAAATCCAACATGCTT | |
| | (SEQ ID NO: 106) | |
| FOXA2_1 | ATGCTGGGAGCGGTGAAGATGGAAGGGCACGAGCCGTCCGA | MLGAVKMEGHEPSDW |
| | CTGGAGCAGCTACTATGCAGAGCCCGAGGGCTACTCCTCCG | SSYYAEPEGYSSVSN |
| | TGAGCAACATGAACGCCGGCCTGGGGATGAACGGCATGAAC | MNAGLGMNGMNTYMS |
| | ACGTACATGAGCATGTCGGCGGCCGCCATGGGCAGCGGCTC | MSAAAMGSGSGNMSA |
| | GGGCAACATGAGCGCGGGCTCCATGAACATGTCGTCGTACG | GSMNMSSYVGAGMSP |
| | TGGGCGCTGGCATGAGCCCGTCCCTGGCGGGGATGTCCCCC | SLAGMSPGAGAMAGM |
| | GGCGCGGGCGCCATGGCGGGCATGGGCGGCTCGGCCGGGGC | GGSAGAAGVAGMGPH |
| | GGCCGGCGTGGCGGGCATGGGGCCGCACTTGAGTCCCAGCC | LSPSLSPLGGQAAGA |
| | TGAGCCCGCTCGGGGGGCAGGCGGCCGGGGCCATGGGCGGC | MGGLAPYANMNSMSP |
| | CTGGCCCCCTACGCCAACATGAACTCCATGAGCCCCATGTA | MYGQAGLSRARDPKT |
| | CGGGCAGGCGGGCCTGAGCCGCGCCCGCGACCCCAAGACCT | YRRSYTHAKPPYSYI |
| | ACAGGCGCAGCTACACGCACGCAAAGCCGCCCTACTCGTAC | SLITMAIQQSPNKML |
| | ATCTCGCTCATCACCATGGCCATCCAGCAGAGCCCCAACAA | TLSEIYQWIMDLFPF |
| | GATGCTGACGCTGAGCGAGATCTACCAGTGGATCATGGACC | YRQNQQRWQNSIRHS |
| | TCTTCCCCTTCTACCGGCAGAACCAGCAGCGCTGGCAGAAC | LSFNDCFLKVPRSPD |
| | TCCATCCGCCACTCGCTCTCCTTCAACGACTGTTTCCTGAA | KPGKGSFWTLHPDSG |
| | GGTGCCCCGCTCGCCCGACAAGCCCGGCAAGGGCTCCTTCT | NMFENGCYLRRQKRF |
| | GGACCCTGCACCCTGACTCGGGCAACATGTTCGAGAACGGC | KCEKQLALKEAAGAA |
| | TGCTACCTGCGCCGCCAGAAGCGCTTCAAGTGCGAGAAGCA | GSGKKAAAGAQASQA |
| | GCTGGCGCTGAAGGAGGCCGCAGGCGCCGCCGGCAGCGGCA | QLGEAAGPASETPAG |
| | AGAAGGCGGCCGCCGGAGCCCAGGCCTCACAGGCTCAACTC | TESPHSSASPCQEHK |
| | GGGGAGGCCGCCGGGCCGGCCTCCGAGACTCCGGCGGGCAC | RGGLGELKGTPAAAL |
| | CGAGTCGCCTCACTCGAGCGCCTCCCCGTGCCAGGAGCACA | SPPEPAPSPGQQQQA |
| | AGCGAGGGGGCCTGGGAGAGCTGAAGGGGACGCCGGCTGCG | AAHLLGPPHHPGLPP |
| | GCGCTGAGCCCCCCAGAGCCGGCGCCCTCTCCCGGGCAGCA | EAHLKPEHHYAFNHP |
| | GCAGCAGGCCGCGGCCCACCTGCTGGGCCCGCCCCACCACC | FSINNLMSSEQQHHH |
| | CGGGCCTGCCGCCTGAGGCCCACCTGAAGCCGGAACACCAC | SHHHHQPHKMDLKAY |
| | TACGCCTTCAACCACCCGTTCTCCATCAACAACCTCATGTC | EQVMHYPGYGSPMPG |
| | CTCGGAGCAGCAGCACCACCACAGCCACCACCACCACCAAC | SLAMGPVTNKTGLDA |
| | CCCACAAAATGGACCTCAAGGCCTACGAACAGGTGATGCAC | SPLAADTSYYQGVYS |
| | TACCCCGGCTACGGTTCCCCCATGCCTGGCAGCTTGGCCAT | RPIMNSS |
| | GGGCCCGGTCACGAACAAAACGGGCCTGGACGCCTCGCCCC | (SEQ ID NO: 145) |
| | TGGCCGCAGATACCTCCTACTACCAGGGGGTGTACTCCCGG | |
| | CCCATTATGAACTCCTCT | |
| | (SEQ ID NO: 107) | |
| FOXA2_2 | ATGCACTCGGCTTCCAGTATGCTGGGAGCGGTGAAGATGGA | MHSASSMLGAVKMEG |
| | AGGGCACGAGCCGTCCGACTGGAGCAGCTACTATGCAGAGC | HEPSDWSSYYAEPEG |
| | CCGAGGGCTACTCCTCCGTGAGCAACATGAACGCCGGCCTG | YSSVSNMNAGLGMNG |
| | GGGATGAACGGCATGAACACGTACATGAGCATGTCGGCGGC | MNTYMSMSAAAMGSG |
| | CGCCATGGGCAGCGGCTCGGGCAACATGAGCGCGGGCTCCA | SGNMSAGSMNMSSYV |
| | TGAACATGTCGTCGTACGTGGGCGCTGGCATGAGCCCGTCC | GAGMSPSLAGMSPGA |
| | CTGGCGGGGATGTCCCCCGGCGCGGGCGCCATGGCGGGCAT | GAMAGMGGSAGAAGV |
| | GGGCGGCTCGGCCGGGGCGGCCGGCGTGGCGGGCATGGGGC | AGMGPHLSPSLSPLG |
| | CGCACTTGAGTCCCAGCCTGAGCCCGCTCGGGGGGCAGGCG | GQAAGAMGGLAPYAN |
| | GCCGGGGCCATGGGCGGCCTGGCCCCCTACGCCAACATGAA | MNSMSPMYGQAGLSR |
| | CTCCATGAGCCCCATGTACGGGCAGGCGGGCCTGAGCCGCG | ARDPKTYRRSYTHAK |
| | CCCGCGACCCCAAGACCTACAGGCGCAGCTACACGCACGCA | PPYSYISLITMAIQQ |
| | AAGCCGCCCTACTCGTACATCTCGCTCATCACCATGGCCAT | SPNKMLTLSEIYQWI |
| | CCAGCAGAGCCCCAACAAGATGCTGACGCTGAGCGAGATCT | MDLFPFYRQNQQRWQ |
| | ACCAGTGGATCATGGACCTCTTCCCCTTCTACCGGCAGAAC | NSIRHSLSFNDCFLK |
| | CAGCAGCGCTGGCAGAACTCCATCCGCCACTCGCTCTCCTT | VPRSPDKPGKGSFWT |
| | CAACGACTGTTTCCTGAAGGTGCCCCGCTCGCCCGACAAGC | LHPDSGNMFENGCYL |
| | CCGGCAAGGGCTCCTTCTGGACCCTGCACCCTGACTCGGGC | RRQKRFKCEKQLALK |
| | AACATGTTCGAGAACGGCTGCTACCTGCGCCGCCAGAAGCG | EAAGAAGSGKKAAAG |
| | CTTCAAGTGCGAGAAGCAGCTGGCGCTGAAGGAGGCCGCAG | AQASQAQLGEAAGPA |
| | GCGCCGCCGGCAGCGGCAAGAAGGCGGCCGCCGGAGCCCAG | SETPAGTESPHSSAS |
| | GCCTCACAGGCTCAACTCGGGGAGGCCGCCGGGCCGGCCTC | PCQEHKRGGLGELKG |
| | CGAGACTCCGGCGGGCACCGAGTCGCCTCACTCGAGCGCCT | TPAAALSPPEPAPSP |
| | CCCCGTGCCAGGAGCACAAGCGAGGGGGCCTGGGAGAGCTG | GQQQQAAAHLLGPPH |
| | AAGGGGACGCCGGCTGCGGCGCTGAGCCCCCCAGAGCCGGC | HPGLPPEAHLKPEHH |
| | GCCCTCTCCCGGGCAGCAGCAGCAGGCCGCGGCCCACCTGC | YAFNHPFSINNLMSS |
| | TGGGCCCGCCCCACCACCCGGGCCTGCCGCCTGAGGCCCAC | EQQHHHSHHHHQPHK |
| | CTGAAGCCGGAACACCACTACGCCTTCAACCACCCGTTCTC | MDLKAYEQVMHYPGY |
| | CATCAACAACCTCATGTCCTCGGAGCAGCAGCACCACCACA | GSPMPGSLAMGPVTN |
| | GCCACCACCACCACCAACCCCACAAAATGGACCTCAAGGCC | KTGLDASPLAADTSY |
| | TACGAACAGGTGATGCACTACCCCGGCTACGGTTCCCCCAT | YQGVYSRPIMNSS |
| | GCCTGGCAGCTTGGCCATGGGCCCGGTCACGAACAAAACGG | (SEQ ID NO: 146) |
| | GCCTGGACGCCTCGCCCCTGGCCGCAGATACCTCCTACTAC | |
| | CAGGGGGTGTACTCCCGGCCCATTATGAACTCCTCT | |
| | (SEQ ID NO: 108) | |
| NR5A1 | ATGGACTATTCGTACGACGAGGACCTGGACGAGCTGTGCCC | MDYSYDEDLDELCPV |
| | CGTGTGCGGGGACAAGGTGTCCGGCTACCACTACGGACTGC | CGDKVSGYHYGLLTC |
| | TCACGTGTGAGAGCTGCAAGGGCTTCTTCAAGCGCACGGTG | ESCKGFFKRTVQNNK |
| | CAGAACAACAAGCACTACACGTGCACCGAGAGCCAGAGCTG | HYTCTESQSCKIDKT |
| | CAAGATCGACAAGACGCAGCGCAAGCGCTGTCCCTTCTGCC | QRKRCPFCRFQKCLT |
| | GCTTCCAGAAATGCCTGACGGTGGGGATGCGCCTGGAAGCC | VGMRLEAVRADRMRG |
| | GTGCGCGCTGACCGTATGAGGGGTGGCCGGAACAAGTTTGG | GRNKFGPMYKRDRAL |
| | GCCGATGTACAAGCGGGACCGGGCCCTGAAACAGCAGAAGA | KQQKKAQIRANGFKL |
| | AGGCACAGATTCGGGCCAATGGCTTCAAGCTGGAGACAGGG | ETGPPMGVPPPPPPA |
| | CCCCCGATGGGGGTGCCCCCGCCGCCCCCTCCCGCACCGGA | PDYVLPPSLHGPEPK |
| | CTACGTGCTGCCTCCCAGCCTGCATGGGCCTGAGCCCAAGG | GLAAGPPAGPLGDFG |
| | GCCTGGCCGCCGGTCCACCTGCTGGGCCACTGGGCGACTTT | APALPMAVPGAHGPL |
| | GGGGCCCCAGCACTGCCCATGGCCGTGCCCGGTGCCCACGG | AGYLYPAFPGRAIKS |
| | GCCACTGGCTGGCTACCTCTACCCTGCCTTTCCTGGCCGTG | EYPEPYASPPQPGLP |
| | CCATCAAGTCTGAGTACCCGGAGCCTTATGCCAGCCCCCCA | YGYPEPFSGGPNVPE |
| | CAGCCTGGGCTGCCGTACGGCTACCCAGAGCCCTTCTCTGG | LILQLLQLEPDEDQV |
| | AGGGCCCAACGTGCCTGAGCTCATCCTGCAGCTGCTGCAGC | RARILGCLQEPTKSR |
| | TGGAGCCGGATGAGGACCAGGTGCGGGCCCGCATCTTGGGC | PDQPAAFGLLCRMAD |
| | TGCCTGCAGGAGCCCACCAAAAGCCGCCCCGACCAGCCGGC | QTFISIVDWARRCMV |
| | GGCCTTCGGCCTCCTGTGCAGAATGGCCGACCAGACCTTCA | FKELEVADQMTLLQN |
| | TCTCCATCGTGGACTGGGCACGCAGGTGCATGGTCTTCAAG | CWSELLVFDHIYRQV |
| | GAGCTGGAGGTGGCCGACCAGATGACGCTGCTGCAGAACTG | QHGKEGSILLVTGQE |
| | CTGGAGCGAGCTGCTGGTGTTCGACCACATCTACCGCCAGG | VELTTVATQAGSLLH |
| | TCCAGCACGGCAAGGAGGGCAGCATCCTGCTGGTCACCGGG | SLVLRAQELVLQLLA |
| | CAGGAGGTGGAGCTGACCACAGTGGCCACCCAGGCGGGCTC | LQLDRQEFVCLKFII |
| | GCTGCTGCACAGCCTGGTGTTGCGGGCGCAGGAGCTGGTGC | LFSLDLKFLNNHILV |
| | TGCAGCTGCTTGCGCTGCAGCTGGACCGGCAGGAGTTTGTC | KDAQEKANAALLDYT |
| | TGCCTCAAGTTCATCATCCTCTTCAGCCTGGATTTGAAGTT | LCHYPHCGDKFQQLL |
| | CCTGAATAACCACATCCTGGTGAAAGACGCTCAGGAGAAGG | LCLVEVRALSMQAKE |
| | CCAACGCCGCCCTGCTTGACTACACCCTGTGCCACTACCCG | YLYHKHLGNEMPRNN |
| | CACTGCGGGGACAAATTCCAGCAGCTGCTGCTGTGCCTGGT | LLIEMLQAKQT |
| | GGAGGTGCGGGCCCTGAGCATGCAGGCCAAGGAGTACCTGT | (SEQ ID NO: 147) |
| | ACCACAAGCACCTGGGCAACGAGATGCCCCGCAACAACCTG | |
| | CTCATCGAAATGCTGCAAGCCAAGCAGACT | |
| | (SEQ ID NO: 109) | |
| FOXA3 | ATGCTGGGCTCAGTGAAGATGGAGGCCCATGACCTGGCCGA | MLGSVKMEAHDLAEW |
| | GTGGAGCTACTACCCGGAGGCGGGCGAGGTCTACTCGCCGG | SYYPEAGEVYSPVTP |
| | TGACCCCAGTGCCCACCATGGCCCCCCTCAACTCCTACATG | VPTMAPLNSYMTLNP |
| | ACCCTGAATCCTCTAAGCTCTCCCTATCCCCCTGGGGGGCT | LSSPYPPGGLPASPL |
| | CCCTGCCTCCCCACTGCCCTCAGGACCCCTGGCACCCCCAG | PSGPLAPPAPAAPLG |
| | CACCTGCAGCCCCCCTGGGGCCCACTTTCCCAGGCCTGGGT | PTFPGLGVSGGSSSS |
| | GTCAGCGGTGGCAGCAGCAGCTCCGGGTACGGGGCCCCGGG | GYGAPGPGLVHGKEM |
| | TCCTGGGCTGGTGCACGGGAAGGAGATGCCGAAGGGGTATC | PKGYRRPLAHAKPPY |
| | GGCGGCCCCTGGCACACGCCAAGCCACCGTATTCCTATATC | SYISLITMAIQQAPG |
| | TCACTCATCACCATGGCCATCCAGCAGGCGCCGGGCAAGAT | KMLTLSEIYQWIMDL |
| | GCTGACCTTGAGTGAAATCTACCAGTGGATCATGGACCTCT | FPYYRENQQRWQNSI |
| | TCCCTTACTACCGGGAGAATCAGCAGCGCTGGCAGAACTCC | RHSLSFNDCFVKVAR |
| | ATTCGCCACTCGCTGTCTTTCAACGACTGCTTCGTCAAGGT | SPDKPGKGSYWALHP |
| | GGCGCGTTCCCCAGACAAGCCTGGCAAGGGCTCCTACTGGG | SSGNMFENGCYLRRQ |
| | CCCTACACCCCAGCTCAGGGAACATGTTTGAGAATGGCTGC | KRFKLEEKVKKGGSG |
| | TACCTGCGCCGCCAGAAACGCTTCAAGCTGGAGGAGAAGGT | AATTTRNGTGSAAST |
| | GAAAAAAGGGGGCAGCGGGGCTGCCACCACCACCAGGAACG | TTPAATVTSPPQPPP |
| | GGACAGGGTCTGCTGCCTCGACCACCACCCCCGCGGCCACA | PAPEPEAQGGEDVGA |
| | GTCACCTCCCCGCCCCAGCCCCCGCCTCCAGCCCCTGAGCC | LDCGSPASSTPYFTG |
| | TGAGGCCCAGGGCGGGGAAGATGTGGGGGCTCTGGACTGTG | LELPGELKLDAPYNF |
| | GCTCACCCGCTTCCTCCACACCCTATTTCACTGGCCTGGAG | NHPFSINNLMSEQTP |
| | CTCCCAGGGGAGCTGAAGCTGGACGCGCCCTACAACTTCAA | APPKLDVGFGGYGAE |
| | CCACCCTTTCTCCATCAACAACCTAATGTCAGAACAGACAC | GGEPGVYYQGLYSRS |
| | CAGCACCTCCCAAACTGGACGTGGGGTTTGGGGGCTACGGG | LLNAS |
| | GCTGAAGGTGGGGAGCCTGGAGTCTACTACCAGGGCCTCTA | (SEQ ID NO: 148) |
| | TTCCCGCTCTTTGCTTAATGCATCC | |
| | (SEQ ID NO: 110) | |
| HNF4A_1 | ATGGTCAGCGTGAACGCGCCCCTCGGGGCTCCAGTGGAGAGT | MVSVNAPLGAPVESS |
| | TCTTACGACACGTCCCCATCAGAAGGCACCAACCTCAACGC | YDTSPSEGTNLNAPN |
| | GCCCAACAGCCTGGGTGTCAGCGCCCTGTGTGCCATCTGCG | SLGVSALCAICGDRA |
| | GGGACCGGGCCACGGGCAAACACTACGGTGCCTCGAGCTGT | TGKHYGASSCDGCKG |
| | GACGGCTGCAAGGGCTTCTTCCGGAGGAGCGTGCGGAAGAA | FFRRSVRKNHMYSCR |
| | CCACATGTACTCCTGCAGATTTAGCCGGCAGTGCGTGGTGG | FSRQCVVDKDKRNQC |
| | ACAAAGACAAGAGGAACCAGTGCCGCTACTGCAGGCTCAAG | RYCRLKKCFRAGMKK |
| | AAATGCTTCCGGGCTGGCATGAAGAAGGAAGCCGTCCAGAA | EAVQNERDRISTRRS |
| | TGAGCGGGACCGGATCAGCACTCGAAGGTCAAGCTATGAGG | SYEDSSLPSINALLQ |
| | ACAGCAGCCTGCCCTCCATCAATGCGCTCCTGCAGGCGGAG | AEVLSRQITSPVSGI |
| | GTCCTGTCCCGACAGATCACCTCCCCCGTCTCCGGGATCAA | NGDIRAKKIASIADV |
| | CGGCGACATTCGGGCGAAGAAGATTGCCAGCATCGCAGATG | CESMKEQLLVLVEWA |
| | TGTGTGAGTCCATGAAGGAGCAGCTGCTGGTTCTCGTTGAG | KYIPAFCELPLDDQV |
| | TGGGCCAAGTACATCCCAGCTTTCTGCGAGCTCCCCCTGGA | ALLRAHAGEHLLLGA |
| | CGACCAGGTGGCCCTGCTCAGAGCCCATGCTGGCGAGCACC | TKRSMVFKDVLLLGN |
| | TGCTGCTCGGAGCCACCAAGAGATCCATGGTGTTCAAGGAC | DYIVPRHCPELAEMS |
| | GTGCTGCTCCTAGGCAATGACTACATTGTCCCTCGGCACTG | RVSIRILDELVLPFQ |
| | CCCGGAGCTGGCGGAGATGAGCCGGGTGTCCATACGCATCC | ELQIDDNEYAYLKAI |
| | TTGACGAGCTGGTGCTGCCCTTCCAGGAGCTGCAGATCGAT | IFFDPDAKGLSDPGK |
| | GACAATGAGTATGCCTACCTCAAAGCCATCATCTTCTTTGA | IKRLRSQVQVSLEDY |
| | CCCAGATGCCAAGGGGCTGAGCGATCCAGGGAAGATCAAGC | INDRQYDSRGRFGEL |
| | GGCTGCGTTCCCAGGTGCAGGTGAGCTTGGAGGACTACATC | LLLLPTLQSITWQMI |
| | AACGACCGCCAGTATGACTCGCGTGGCCGCTTTGGAGAGCT | EQIQFIKLFGMAKID |
| | GCTGCTGCTGCTGCCCACCTTGCAGAGCATCACCTGGCAGA | NLLQEMLLGGSPSDA |
| | TGATCGAGCAGATCCAGTTCATCAAGCTCTTCGGCATGGCC | PHAHHPLHPHLMQEH |
| | AAGATTGACAACCTGTTGCAGGAGATGCTGCTGGGAGGGTC | MGTNVIVANTMPTHL |
| | CCCCAGCGATGCACCCCATGCCCACCACCCCCTGCACCCTC | SNGQMCEWPRPRGQA |
| | ACCTGATGCAGGAACATATGGGAACCAACGTCATCGTTGCC | ATPETPQPSPPGGSG |
| | AACACAATGCCCACTCACCTCAGCAACGGACAGATGTGTGA | SEPYKLLPGAVATIV |
| | GTGGCCCCGACCCAGGGGACAGGCAGCCACCCCTGAGACCC | KPLSAIPQPTITKQE |
| | CACAGCCCTCACCGCCAGGTGGCTCAGGGTCTGAGCCCTAT | VI |
| | AAGCTCCTGCCGGGAGCCGTCGCCACAATCGTCAAGCCCCT | (SEQ ID NO: 149) |
| | CTCTGCCATCCCCCAGCCGACCATCACCAAGCAGGAAGTTA | |
| | TC | |
| | (SEQ ID NO: 111) | |
| HNF4A_2 | ATGTCGGACTGGGGCCAGGGCTTCCCCCAGGACCCACCAGA | MSDWGQGFPQDPPDT |
| | CACGTCCCCATCAGAAGGCACCAACCTCAACGCGCCCAACA | SPSEGTNLNAPNSLG |
| | GCCTGGGTGTCAGCGCCCTGTGTGCCATCTGCGGGGACCGG | VSALCAICGDRATGK |
| | GCCACGGGCAAACACTACGGTGCCTCGAGCTGTGACGGCTG | HYGASSCDGCKGFFR |
| | CAAGGGCTTCTTCCGGAGGAGCGTGCGGAAGAACCACATGT | RSVRKNHMYSCRFSR |
| | ACTCCTGCAGATTTAGCCGGCAGTGCGTGGTGGACAAAGAC | QCVVDKDKRNQCRYC |
| | AAGAGGAACCAGTGCCGCTACTGCAGGCTCAAGAAATGCTT | RLKKCFRAGMKKEAV |
| | CCGGGCTGGCATGAAGAAGGAAGCCGTCCAGAATGAGCGGG | QNERDRISTRRSSYE |
| | ACCGGATCAGCACTCGAAGGTCAAGCTATGAGGACAGCAGC | DSSLPSINALLQAEV |
| | CTGCCCTCCATCAATGCGCTCCTGCAGGCGGAGGTCCTGTC | LSRQITSPVSGINGD |
| | CCGACAGATCACCTCCCCCGTCTCCGGGATCAACGGCGACA | IRAKKIASIADVCES |
| | TTCGGGCGAAGAAGATTGCCAGCATCGCAGATGTGTGTGAG | MKEQLLVLVEWAKYI |
| | TCCATGAAGGAGCAGCTGCTGGTTCTCGTTGAGTGGGCCAA | PAFCELPLDDQVALL |
| | GTACATCCCAGCTTTCTGCGAGCTCCCCCTGGACGACCAGG | RAHAGEHLLLGATKR |
| | TGGCCCTGCTCAGAGCCCATGCTGGCGAGCACCTGCTGCTC | SMVFKDVLLLGNDYI |
| | GGAGCCACCAAGAGATCCATGGTGTTCAAGGACGTGCTGCT | VPRHCPELAEMSRVS |
| | CCTAGGCAATGACTACATTGTCCCTCGGCACTGCCCGGAGC | IRILDELVLPFQELQ |
| | TGGCGGAGATGAGCCGGGTGTCCATACGCATCCTTGACGAG | IDDNEYAYLKAIIFF |
| | CTGGTGCTGCCCTTCCAGGAGCTGCAGATCGATGACAATGA | DPDAKGLSDPGKIKR |
| | GTATGCCTACCTCAAAGCCATCATCTTCTTTGACCCAGATG | LRSQVQVSLEDYIND |
| | CCAAGGGGCTGAGCGATCCAGGGAAGATCAAGCGGCTGCGT | RQYDSRGRFGELLLL |
| | TCCCAGGTGCAGGTGAGCTTGGAGGACTACATCAACGACCG | LPTLQSITWQMIEQI |
| | CCAGTATGACTCGCGTGGCCGCTTTGGAGAGCTGCTGCTGC | QFIKLFGMAKIDNLL |
| | TGCTGCCCACCTTGCAGAGCATCACCTGGCAGATGATCGAG | QEMLLGGSPSDAPHA |
| | CAGATCCAGTTCATCAAGCTCTTCGGCATGGCCAAGATTGA | HHPLHPHLMQEHMGT |
| | CAACCTGTTGCAGGAGATGCTGCTGGGAGGGTCCCCCAGCG | NVIVANTMPTHLSNG |
| | ATGCACCCCATGCCCACCACCCCCTGCACCCTCACCTGATG | QMCEWPRPRGQAATP |
| | CAGGAACATATGGGAACCAACGTCATCGTTGCCAACACAAT | ETPQPSPPGGSGSEP |
| | GCCCACTCACCTCAGCAACGGACAGATGTGTGAGTGGCCCC | YKLLPGAVATIVKPL |
| | GACCCAGGGGACAGGCAGCCACCCCTGAGACCCCACAGCCC | SAIPQPTITKQEVI |
| | TCACCGCCAGGTGGCTCAGGGTCTGAGCCCTATAAGCTCCT | (SEQ ID NO: 150) |
| | GCCGGGAGCCGTCGCCACAATCGTCAAGCCCCTCTCTGCCA | |
| | TCCCCCAGCCGACCATCACCAAGCAGGAAGTTATC | |
| | (SEQ ID NO: 112) | |
| HNF4A_3 | ATGGTCAGCGTGAACGCGCCCCTCGGGGCTCCAGTGGAGAG | MVSVNAPLGAPVESS |
| | TTCTTACGACACGTCCCCATCAGAAGGCACCAACCTCAACG | YDTSPSEGTNLNAPN |
| | CGCCCAACAGCCTGGGTGTCAGCGCCCTGTGTGCCATCTGC | SLGVSALCAICGDRA |
| | GGGGACCGGGCCACGGGCAAACACTACGGTGCCTCGAGCTG | TGKHYGASSCDGCKG |
| | TGACGGCTGCAAGGGCTTCTTCCGGAGGAGCGTGCGGAAGA | FFRRSVRKNHMYSCR |
| | ACCACATGTACTCCTGCAGATTTAGCCGGCAGTGCGTGGTG | FSRQCVVDKDKRNQC |
| | GACAAAGACAAGAGGAACCAGTGCCGCTACTGCAGGCTCAA | RYCRLKKCFRAGMKK |
| | GAAATGCTTCCGGGCTGGCATGAAGAAGGAAGCCGTCCAGA | EAVQNERDRISTRRS |
| | ATGAGCGGGACCGGATCAGCACTCGAAGGTCAAGCTATGAG | SYEDSSLPSINALLQ |
| | GACAGCAGCCTGCCCTCCATCAATGCGCTCCTGCAGGCGGA | AEVLSRQITSPVSGI |
| | GGTCCTGTCCCGACAGATCACCTCCCCCGTCTCCGGGATCA | NGDIRAKKIASIADV |
| | ACGGCGACATTCGGGCGAAGAAGATTGCCAGCATCGCAGAT | CESMKEQLLVLVEWA |
| | GTGTGTGAGTCCATGAAGGAGCAGCTGCTGGTTCTCGTTGA | KYIPAFCELPLDDQV |
| | GTGGGCCAAGTACATCCCAGCTTTCTGCGAGCTCCCCCTGG | ALLRAHAGEHLLLGA |
| | ACGACCAGGTGGCCCTGCTCAGAGCCCATGCTGGCGAGCAC | TKRSMVFKDVLLLGN |
| | CTGCTGCTCGGAGCCACCAAGAGATCCATGGTGTTCAAGGA | DYIVPRHCPELAEMS |
| | CGTGCTGCTCCTAGGCAATGACTACATTGTCCCTCGGCACT | RVSIRILDELVLPFQ |
| | GCCCGGAGCTGGCGGAGATGAGCCGGGTGTCCATACGCATC | ELQIDDNEYAYLKAI |
| | CTTGACGAGCTGGTGCTGCCCTTCCAGGAGCTGCAGATCGA | IFFDPDAKGLSDPGK |
| | TGACAATGAGTATGCCTACCTCAAAGCCATCATCTTCTTTG | IKRLRSQVQVSLEDY |
| | ACCCAGATGCCAAGGGGCTGAGCGATCCAGGGAAGATCAAG | INDRQYDSRGRFGEL |
| | CGGCTGCGTTCCCAGGTGCAGGTGAGCTTGGAGGACTACAT | LLLLPTLQSITWQMI |
| | CAACGACCGCCAGTATGACTCGCGTGGCCGCTTTGGAGAGC | EQIQFIKLFGMAKID |
| | TGCTGCTGCTGCTGCCCACCTTGCAGAGCATCACCTGGCAG | NLLQEMLLGGPCQAQ |
| | ATGATCGAGCAGATCCAGTTCATCAAGCTCTTCGGCATGGC | EGRGWSGDSPGDRPH |
| | CAAGATTGACAACCTGTTGCAGGAGATGCTGCTGGGAGGTC | TVSSPLSSLASPLCR |
| | CGTGCCAAGCCCAGGAGGGGCGGGGTTGGAGTGGGGACTCC | FGQVA |
| | CCAGGAGACAGGCCTCACACAGTGAGCTCACCCCTCAGCTC | (SEQ ID NO: 151) |
| | CTTGGCTTCCCCACTGTGCCGCTTTGGGCAAGTTGCT | |
| | (SEQ ID NO: 113) | |
| HNF4G_1 | ATGAATACCACAGACAACGGTGTCAACTGTCTGTGTGCTAT | MNTTDNGVNCLCAIC |
| | CTGTGGGGACAGAGCAACAGGAAAACACTATGGGGCATCCA | GDRATGKHYGASSCD |
| | GCTGTGATGGGTGCAAGGGTTTCTTCAGACGCAGCATTCGT | GCKGFFRRSIRKSHV |
| | AAGAGTCACGTTTATTCTTGCAGGTTCAGTCGGCAATGTGT | YSCRFSRQCVVDKDK |
| | TGTTGACAAGGACAAAAGGAATCAATGTAGATATTGTCGAT | RNQCRYCRLRKCFRA |
| | TAAGAAAGTGTTTTAGAGCGGGAATGAAAAAAGAAGCTGTA | GMKKEAVQNERDRIS |
| | CAAAATGAACGTGACAGAATAAGCACCAGAAGAAGCACATT | TRRSTFDGSNIPSIN |
| | TGATGGCAGCAACATCCCCTCCATTAACACACTGGCACAAG | TLAQAEVRSRQISVS |
| | CTGAAGTTCGGTCTCGCCAGATCTCAGTCTCAAGCCCTGGG | SPGSSTDINVKKIAS |
| | TCAAGCACTGACATAAACGTTAAGAAAATTGCAAGTATTGG | IGDVCESMKQQLLVL |
| | TGATGTCTGTGAATCTATGAAACAGCAGCTCTTAGTCTTGG | VEWAKYIPAFCELPL |
| | TGGAATGGGCTAAATATATTCCTGCCTTCTGTGAATTACCA | DDQVALLRAHAGEHL |
| | TTGGATGATCAGGTGGCACTGTTGAGAGCTCACGCAGGGGA | LLGATKRSMMYKDIL |
| | GCACTTACTGCTTGGAGCTACAAAGAGATCCATGATGTATA | LLGNNYVIHRNSCEV |
| | AAGATATTTTGCTTTTGGGAAACAACTATGTTATTCACCGC | EISRVANRVLDELVR |
| | AACAGCTGTGAAGTTGAGATTAGCCGTGTGGCCAATCGTGT | PFQEIQIDDNEYACL |
| | TCTAGATGAGCTGGTTAGACCATTTCAAGAAATCCAGATTG | KAIVFFDPDAKGLSD |
| | ATGACAATGAGTATGCTTGTTTAAAGGCAATTGTATTTTTT | PVKIKNMRFQVQIGL |
| | GATCCAGATGCAAAAGGGCTAAGCGATCCAGTAAAAATTAA | EDYINDRQYDSRGRF |
| | GAACATGAGGTTCCAAGTGCAGATCGGTTTGGAGGACTACA | GELLLLLPTLQSITW |
| | TCAATGATCGGCAGTATGACTCCCGGGGGAGGTTTGGAGAG | QMIEQIQFVKLFGMV |
| | TTGCTTCTGCTCCTGCCCACACTGCAGAGCATCACGTGGCA | KIDNLLQEMLLGGAS |
| | AATGATTGAGCAAATACAGTTTGTTAAACTTTTTGGGATGG | NDGSHLHHPMHPHLS |
| | TTAAAATTGACAATCTACTTCAGGAAATGCTATTAGGTGGG | QDPLTGQTILLGPMS |
| | GCTTCCAATGATGGCAGTCATCTCCATCATCCAATGCATCC | TLVHADQISTPETPL |
| | ACATTTGTCTCAAGACCCATTAACTGGACAAACTATACTTT | PSPPQGSGQEQYKIA |
| | TAGGTCCCATGTCAACACTGGTTCATGCAGACCAGATCTCA | ANQASVISHQHLSKQ |
| | ACTCCTGAAACCCCACTCCCTTCCCCACCACAAGGCTCTGG | KQL |
| | GCAAGAACAGTACAAAATAGCTGCAAACCAAGCATCAGTCA | (SEQ ID NO: 152) |
| | TTTCACACCAGCATCTCTCCAAACAAAAGCAATTG | |
| | (SEQ ID NO: 114) | |
| HNF4G_2 | ATGAATACCACAGACAACGGTGTCAACTGTCTGTGTGCTAT | MNTTDNGVNCLCAIC |
| | CTGTGGGGACAGAGCAACAGGAAAACACTATGGGGCATCCA | GDRATGKHYGASSCD |
| | GCTGTGATGGGTGCAAGGGTTTCTTCAGACGCAGCATTCGT | GCKGFFRRSIRKSHV |
| | AAGAGTCACGTTTATTCTTGCAGGTTCAGTCGGCAATGTGT | YSCRFSRQCVVDKDK |
| | TGTTGACAAGGACAAAAGGAATCAATGTAGATATTGTCGAT | RNQCRYCRLRKCFRA |
| | TAAGAAAGTGTTTTAGAGCGGGAATGAAAAAAGAAGCTGTA | GMKKEAVQNERDRIS |
| | CAAAATGAACGTGACAGAATAAGCACCAGAAGAAGCACATT | TRRSTFDGSNIPSIN |
| | TGATGGCAGCAACATCCCCTCCATTAACACACTGGCACAAG | TLAQAEVRSRQISVS |
| | CTGAAGTTCGGTCTCGCCAGATCTCAGTCTCAAGCCCTGGG | SPGSSTDINVKKIAS |
| | TCAAGCACTGACATAAACGTTAAGAAAATTGCAAGTATTGG | IGDVCESMKQQLLVL |
| | TGATGTCTGTGAATCTATGAAACAGCAGCTCTTAGTCTTGG | VEWAKYIPAFCELPL |
| | TGGAATGGGCTAAATATATTCCTGCCTTCTGTGAATTACCA | DDQVALLRAHAGEHL |
| | TTGGATGATCAGGTGGCACTGTTGAGAGCTCACGCAGGGGA | LLGATKRSMMYKDIL |
| | GCACTTACTGCTTGGAGCTACAAAGAGATCCATGATGTATA | LLGNNYVIHRNSCEV |
| | AAGATATTTTGCTTTTGGGAAACAACTATGTTATTCACCGC | EISRVANRVLDELVR |
| | AACAGCTGTGAAGTTGAGATTAGCCGTGTGGCCAATCGTGT | PFQEIQIDDNEYACL |
| | TCTAGATGAGCTGGTTAGACCATTTCAAGAAATCCAGATTG | KAIVFFDPDAKGLSD |
| | ATGACAATGAGTATGCTTGTTTAAAGGCAATTGTATTTTTT | PVKIKNMRFQVQIGL |
| | GATCCAGATGCAAAAGGGCTAAGCGATCCAGTAAAAATTAA | EDYINDRQYDSRGRF |
| | GAACATGAGGTTCCAAGTGCAGATCGGTTTGGAGGACTACA | GELLLLLPTLQSITW |
| | TCAATGATCGGCAGTATGACTCCCGGGGGAGGTTTGGAGAG | QMIEQIQFVKLFGMV |
| | TTGCTTCTGCTCCTGCCCACACTGCAGAGCATCACGTGGCA | KIDNLLQEMLLGGAS |
| | AATGATTGAGCAAATACAGTTTGTTAAACTTTTTGGGATGG | NDGSHLHHPMHPHLS |
| | TTAAAATTGACAATCTACTTCAGGAAATGCTATTAGGTGGG | QDPLTGQTILLGPMS |
| | GCTTCCAATGATGGCAGTCATCTCCATCATCCAATGCATCC | TLVHADQISTPETPL |
| | ACATTTGTCTCAAGACCCATTAACTGGACAAACTATACTTT | PSPPQGSGQEQYKIA |
| | TAGGTCCCATGTCAACACTGGTTCATGCAGACCAGATCTCA | ANQASVISHQHLSKQ |
| | ACTCCTGAAACCCCACTCCCTTCCCCACCACAAGGCTCTGG | KQL |
| | GCAAGAACAGTACAAAATAGCTGCAAACCAAGCATCAGTCA | (SEQ ID NO: 153) |
| | TTTCACACCAGCATCTCTCCAAACAAAAGCAATTG | |
| | (SEQ ID NO: 115) | |
| HBP1 | ATGGTGTGGGAAGTGAAGACAAATCAGATGCCTAATGCAGTA | MVWEVKTNQMPNAVQ |
| | CAGAAACTCCTGTTGGTGATGGACAAGAGAGCCTCAGGAAT | KLLLVMDKRASGMND |
| | GAATGACTCATTGGAGTTGCTGCAGTGTAATGAGAATTTGC | SLELLQCNENLPSSP |
| | CATCTTCACCTGGATATAACTCCTGTGATGAACACATGGAG | GYNSCDEHMELDDLP |
| | CTTGATGACCTTCCTGAACTTCAGGCAGTTCAAAGTGATCC | ELQAVQSDPTQSGMY |
| | TACCCAATCTGGCATGTACCAGCTGAGTTCAGATGTTTCAC | QLSSDVSHQEYPRSS |
| | ATCAAGAATACCCAAGATCATCTTGGAACCAAAATACCTCA | WNQNTSDIPETTYRE |
| | GACATACCAGAAACTACTTACCGTGAAAATGAGGTGGACTG | NEVDWLTELANIATS |
| | GCTAACAGAATTGGCAAATATCGCGACCAGTCCACAAAGTC | PQSPLMQCSFYNRSS |
| | CACTGATGCAGTGCTCATTTTACAATAGATCATCTCCTGTA | PVHIIATSKSLHSYA |
| | CACATCATAGCCACTAGCAAAAGTTTACATTCCTATGCACG | RPPPVSSSSKSEPAF |
| | CCCTCCACCAGTGTCCTCTTCTTCGAAGAGTGAACCAGCCT | PHHHWKEETPVRHER |
| | TCCCTCATCACCATTGGAAGGAGGAAACACCAGTAAGACAC | ANSESESGIFCMSSL |
| | GAAAGGGCAAATAGTGAGTCAGAATCTGGCATTTTCTGCAT | SDDDDLGWCNSWPST |
| | GTCCTCCCTGTCAGATGATGATGATTTGGGATGGTGCAATT | VWHCFLKGTRLCFHK |
| | CCTGGCCTTCAACTGTCTGGCACTGTTTTTTGAAAGGCACA | GSNKEWQDVEDFARA |
| | CGACTGTGCTTTCATAAGGGAAGCAATAAGGAATGGCAAGA | EGCDNEEDLQMGIHK |
| | TGTTGAAGATTTTGCTAGAGCTGAAGGCTGTGATAATGAGG | GYGSDGLKLLSHEES |
| | AAGATCTTCAAATGGGCATTCACAAGGGCTATGGTTCTGAT | VSFGESVLKLTFDPG |
| | GGTCTAAAGTTGTTATCACATGAAGAAAGTGTATCATTTGG | TVEDGLLTVECKLDH |
| | CGAGTCTGTACTGAAGTTGACTTTTGATCCTGGTACAGTAG | PFYVKNKGWSSFYPS |
| | AAGATGGTTTACTTACCGTAGAGTGTAAGCTGGACCACCCT | LTVVQHGIPCCEVHI |
| | TTCTATGTTAAAAATAAAGGTTGGTCATCATTTTATCCAAG | GDVCLPPGHPDAINF |
| | CTTGACTGTGGTACAGCATGGCATTCCATGTTGTGAAGTTC | DDSGVFDTFKSYDFT |
| | ATATTGGCGATGTATGTCTACCTCCTGGACACCCCGATGCC | PMDSSAVYVLSSMAR |
| | ATTAATTTTGATGATTCAGGTGTTTTTGATACATTTAAAAG | QRRASLSCGGPGGQD |
| | CTATGACTTCACACCTATGGATTCTTCTGCAGTTTATGTGT | FARSGFSKNCGSPGS |
| | TAAGTAGTATGGCTCGCCAGCGTCGTGCATCTTTGTCTTGT | SQLSSNSLYAKAVKN |
| | GGAGGACCTGGTGGTCAAGACTTTGCAAGATCTGGATTCAG | HSSGTVSATSPNKCK |
| | TAAAAACTGTGGCTCACCTGGATCATCACAGCTCTCTTCCA | RPMNAFMLFAKKYRV |
| | ATTCTTTGTATGCTAAAGCTGTCAAAAACCACAGCTCAGGG | EYTQMYPGKDNRAIS |
| | ACTGTGAGTGCCACTTCTCCTAATAAGTGCAAAAGACCAAT | VILGDRWKKMKNEER |
| | GAATGCCTTCATGCTTTTTGCCAAAAAATACAGAGTTGAAT | RMYTLEAKALAEEQK |
| | ATACTCAGATGTATCCAGGGAAAGATAACAGAGCCATAAGT | RLNPDCWKRKRTNSG |
| | GTGATCCTTGGTGACAGGTGGAAGAAAATGAAGAATGAAGA | SQQH |
| | GAGAAGAATGTACACATTAGAAGCAAAGGCTTTGGCTGAAG | (SEQ ID NO: 154) |
| | AACAGAAACGTTTAAATCCTGACTGTTGGAAGAGGAAAAGA | |
| | ACCAATTCAGGCTCACAACAACAT | |
| | (SEQ ID NO: 116) | |
| NHLH1 | ATGATGCTCAACTCAGACACCATGGAGCTGGACCTGCCGCC | MMLNSDTMELDLPPT |
| | CACCCACTCAGAGACTGAGTCGGGCTTCAGTGACTGTGGGG | HSETESGFSDCGGGA |
| | GCGGGGCGGGCCCTGATGGTGCCGGGCCTGGGGGTCCGGGA | GPDGAGPGGPGGGQA |
| | GGGGGCCAGGCCCGAGGCCCAGAGCCGGGAGAGCCTGGCCG | RGPEPGEPGRKDLQH |
| | GAAAGACCTGCAGCATCTGAGCCGCGAGGAGCGCCGGCGCC | LSREERRRRRRATAK |
| | GGCGCCGCGCCACAGCCAAGTACCGCACGGCCCACGCCACG | YRTAHATRERIRVEA |
| | CGAGAACGCATCCGCGTGGAAGCCTTCAACCTGGCCTTCGC | FNLAFAELRKLLPTL |
| | CGAGCTGCGCAAGCTGCTGCCTACGCTGCCCCCCGACAAGA | PPDKKLSKIEILRLA |
| | AGCTCTCCAAGATTGAGATTCTGCGCCTGGCCATCTGCTAT | ICYISYLNHVLDV |
| | ATCTCCTACCTGAACCACGTGCTGGACGTC | (SEQ ID NO: 155) |
| | (SEQ ID NO: 117) | |
| NR5A2_1 | ATGTCTTCTAATTCAGATACTGGGGATTTACAAGAGTCTTT | MSSNSDTGDLQESLK |
| | AAAGCACGGACTTACACCTATTGTGTCTCAATTTAAAATGG | HGLTPIGAGLPDRHG |
| | TGAATTACTCCTATGATGAAGATCTGGAAGAGCTTTGTCCC | SPIPARGRLVMLPKV |
| | GTGTGTGGAGATAAAGTGTCTGGGTACCATTATGGGCTCCT | ETEALGLARSHGEQG |
| | CACCTGTGAAAGCTGCAAGGGATTTTTTAAGCGAACAGTCC | QMPENMQVSQFKMVN |
| | AAAATAATAAAAGGTACACATGTATAGAAAACCAGAACTGC | YSYDEDLEELCPVCG |
| | CAAATTGACAAAACACAGAGAAAGCGTTGTCCTTACTGTCG | DKVSGYHYGLLTCES |
| | TTTTCAAAAATGTCTAAGTGTTGGAATGAAGCTAGAAGCTG | CKGFFKRTVQNNKRY |
| | TAAGGGCCGACCGAATGCGTGGAGGAAGGAATAAGTTTGGG | TCIENQNCQIDKTQR |
| | CCAATGTACAAGAGAGACAGGGCCCTGAAGCAACAGAAAAA | KRCPYCRFQKCLSVG |
| | AGCCCTCATCCGAGCCAATGGACTTAAGCTAGAAGCCATGT | MKLEAVRADRMRGGR |
| | CTCAGGTGATCCAAGCTATGCCCTCTGACCTGACCATTTCC | NKFGPMYKRDRALKQ |
| | TCTGCAATTCAAAACATCCACTCTGCCTCCAAAGGCCTACC | QKKALIRANGLKLEA |
| | TCTGAACCATGCTGCCTTGCCTCCTACAGACTATGACAGAA | MSQVIQAMPSDLTIS |
| | GTCCCTTTGTAACATCCCCCATTAGCATGACAATGCCCCCT | SAIQNIHSASKGLPL |
| | CACGGCAGCCTGCAAGGTTACCAAACATATGGCCACTTTCC | NHAALPPTDYDRSPF |
| | TAGCCGGGCCATCAAGTCTGAGTACCCAGACCCCTATACCA | VTSPISMTMPPHGSL |
| | GCTCACCCGAGTCCATAATGGGCTATTCATATATGGATAGT | QGYQTYGHFPSRAIK |
| | TACCAGACGAGCTCTCCAGCAAGCATCCCACATCTGATACT | SEYPDPYTSSPESIM |
| | GGAACTTTTGAAGTGTGAGCCAGATGAGCCTCAAGTCCAGG | GYSYMDSYQTSSPAS |
| | CTAAAATCATGGCCTATTTGCAGCAAGAGCAGGCTAACCGA | IPHLILELLKCEPDE |
| | AGCAAGCACGAAAAGCTGAGCACCTTTGGGCTTATGTGCAA | PQVQAKIMAYLQQEQ |
| | AATGGCAGATCAAACTCTCTTCTCCATTGTCGAGTGGGCCA | ANRSKHEKLSTFGLM |
| | GGAGTAGTATCTTCTTCAGAGAACTTAAGGTTGATGACCAA | CKMADQTLFSIVEWA |
| | ATGAAGCTGCTTCAGAACTGCTGGAGTGAGCTCTTAATCCT | RSSIFFRELKVDDQM |
| | CGACCACATTTACCGACAAGTGGTACATGGAAAGGAAGGAT | KLLQNCWSELLILDH |
| | CCATCTTCCTGGTTACTGGGCAACAAGTGGACTATTCCATA | IYRQVVHGKEGSIFL |
| | ATAGCATCACAAGCCGGAGCCACCCTCAACAACCTCATGAG | VTGQQVDYSIIASQA |
| | TCATGCACAGGAGTTAGTGGCAAAACTTCGTTCTCTCCAGT | GATLNNLMSHAQELV |
| | TTGATCAACGAGAGTTCGTATGTCTGAAATTCTTGGTGCTC | AKLRSLQFDQREFVC |
| | TTTAGTTTAGATGTCAAAAACCTTGAAAACTTCCAGCTGGT | LKFLVLFSLDVKNLE |
| | AGAAGGTGTCCAGGAACAAGTCAATGCCGCCCTGCTGGACT | NFQLVEGVQEQVNAA |
| | ACACAATGTGTAACTACCCGCAGCAGACAGAGAAATTTGGA | LLDYTMCNYPQQTEK |
| | CAGCTACTTCTTCGACTACCCGAAATCCGGGCCATCAGTAT | FGQLLLRLPEIRAIS |
| | GCAGGCTGAAGAATACCTCTACTACAAGCACCTGAACGGGG | MQAEEYLYYKHLNGD |
| | ATGTGCCCTATAATAACCTTCTCATTGAAATGTTGCATGCC | VPYNNLLIEMLHAKR |
| | AAAAGAGCA | A |
| | (SEQ ID NO: 118) | (SEQ ID NO: 156) |
| NR5A2_2 | ATGGTGAATTACTCCTATGATGAAGATCTGGAAGAGCTTTG | MVNYSYDEDLEELCP |
| | TCCCGTGTGTGGAGATAAAGTGTCTGGGTACCATTATGGGC | VCGDKVSGYHYGLLT |
| | TCCTCACCTGTGAAAGCTGCAAGGGATTTTTTAAGCGAACA | CESCKGFFKRTVQNN |
| | GTCCAAAATAATAAAAGGTACACATGTATAGAAAACCAGAA | KRYTCIENQNCQIDK |
| | CTGCCAAATTGACAAAACACAGAGAAAGCGTTGTCCTTACT | TQRKRCPYCRFQKCL |
| | GTCGTTTTCAAAAATGTCTAAGTGTTGGAATGAAGCTAGAA | SVGMKLEAVRADRMR |
| | GCTGTAAGGGCCGACCGAATGCGTGGAGGAAGGAATAAGTT | GGRNKFGPMYKRDRA |
| | TGGGCCAATGTACAAGAGAGACAGGGCCCTGAAGCAACAGA | LKQQKKALIRANGLK |
| | AAAAAGCCCTCATCCGAGCCAATGGACTTAAGCTAGAAGCC | LEAMSQVIQAMPSDL |
| | ATGTCTCAGGTGATCCAAGCTATGCCCTCTGACCTGACCAT | TISSAIQNIHSASKG |
| | TTCCTCTGCAATTCAAAACATCCACTCTGCCTCCAAAGGCC | LPLNHAALPPTDYDR |
| | TACCTCTGAACCATGCTGCCTTGCCTCCTACAGACTATGAC | SPFVTSPISMTMPPH |
| | AGAAGTCCCTTTGTAACATCCCCCATTAGCATGACAATGCC | GSLQGYQTYCHFPSR |
| | CCCTCACGGCAGCCTGCAAGGTTACCAAACATATGGCCACT | AIKSEYPDPYTSSPE |
| | TTCCTAGCCGGGCCATCAAGTCTGAGTACCCAGACCCCTAT | SIMGYSYMDSYQTSS |
| | ACCAGCTCACCCGAGTCCATAATGGGCTATTCATATATGGA | PASIPHLILELLKCE |
| | TAGTTACCAGACGAGCTCTCCAGCAAGCATCCCACATCTGA | PDEPQVQAKIMAYLQ |
| | TACTGGAACTTTTGAAGTGTGAGCCAGATGAGCCTCAAGTC | QEQANRSKHEKLSTF |
| | CAGGCTAAAATCATGGCCTATTTGCAGCAAGAGCAGGCTAA | GLMCKMADQTLFSIV |
| | CCGAAGCAAGCACGAAAAGCTGAGCACCTTTGGGCTTATGT | EWARSSIFFRELKVD |
| | GCAAAATGGCAGATCAAACTCTCTTCTCCATTGTCGAGTGG | DQMKLLQNCWSELLI |
| | GCCAGGAGTAGTATCTTCTTCAGAGAACTTAAGGTTGATGA | LDHIYRQVVHGKEGS |
| | CCAAATGAAGCTGCTTCAGAACTGCTGGAGTGAGCTCTTAA | IFLVTGQQVDYSIIA |
| | TCCTCGACCACATTTACCGACAAGTGGTACATGGAAAGGAA | SQAGATLNNLMSHAQ |
| | GGATCCATCTTCCTGGTTACTGGGCAACAAGTGGACTATTC | ELVAKLRSLQFDQRE |
| | CATAATAGCATCACAAGCCGGAGCCACCCTCAACAACCTCA | FVCLKFLVLFSLDVK |
| | TGAGTCATGCACAGGAGTTAGTGGCAAAACTTCGTTCTCTC | NLENFQLVEGVQEQV |
| | CAGTTTGATCAACGAGAGTTCGTATGTCTGAAATTCTTGGT | NAALLDYTMCNYPQQ |
| | GCTCTTTAGTTTAGATGTCAAAAACCTTGAAAACTTCCAGC | TEKFGQLLLRLPEIR |
| | TGGTAGAAGGTGTCCAGGAACAAGTCAATGCCGCCCTGCTG | AISMQAEEYLYYKHL |
| | GACTACACAATGTGTAACTACCCGCAGCAGACAGAGAAATT | NGDVPYNNLLIEMLH |
| | TGGACAGCTACTTCTTCGACTACCCGAAATCCGGGCCATCA | AKRA |
| | GTATGCAGGCTGAAGAATACCTCTACTACAAGCACCTGAAC | (SEQ ID NO: 157) |
| | GGGGATGTGCCCTATAATAACCTTCTCATTGAAATGTTGCA | |
| | TGCCAAAAGAGCA | |
| | (SEQ ID NO: 119) | |
| YAF2 | ATGGGAGACAAGAAGAGCCCCACCAGGAAACCTCGACCTGT | MGDKKSPTRKPRPVS |
| | CTCCCAGTTGGTTGCACAGCAGGTTACTCAGCAGTTTGTGC | QLVAQQVTQQFVPPT |
| | CTCCTACACAGTCAAAGAAAGAGAAAAAAGATAAAGTAGAA | QSKKEKKDKVEKEKS |
| | AAAGAAAAAAGTGAAAAGGAAACAACTAGCAAAAAGAATAG | EKETTSKKNSHKKTR |
| | CCATAAGAAAACCAGGCCAAGATTGAAAAATGTGGATCGGA | PRLKNVDRSSAQHLE |
| | GTAGTGCTCAGCATTTGGAAGTTACTGTTGGAGATCTGACA | VTVGDLTVIITDFKE |
| | GTCATTATTACAGACTTTAAGGAGAAAACAAAGTCACCGCC | KTKSPPASSAASADQ |
| | TGCATCTAGTGCTGCTTCTGCAGATCAACACAGTCAAAGCG | HSQSGSSSDNTERGM |
| | GCTCTAGCTCTGATAACACAGAGAGAGGAATGTCCAGGTCA | SRSSSPRGEASSLNG |
| | TCTTCACCCAGAGGAGAAGCCTCATCATTGAATGGAGAATC | ESH |
| | TCAT | (SEQ ID NO: 158) |
| | (SEQ ID NO: 120) | |

In some embodiments, expression of the targeted gene is modulated. Gene expression may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Gene expression may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Gene expression may be modulated by about 5-95%, 10-90%, 15-85%, 20-80%, or 1.5-fold to 10-fold, relative to a control. Gene expression may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Gene expression may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Gene expression may be reduced by about 5-95%, 10-90%, 15-85%, 20-80%, or 1.5-fold to 10-fold, relative to a control. Gene expression may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Gene expression may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Gene expression may be increased by about 5-95%, 10-90%, 15-85%, 20-80%, or 1.5-fold to 10-fold, relative to a control.

In some embodiments, activity of the protein expressed from the targeted gene is modulated. Protein activity may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2~ fold, 3-fold, 4-fold, 5-fold, 6-fold, 7-fold, 8-fold, 9-fold, or 10-fold, relative to a control. Protein activity may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein activity may be modulated by about 5-95%, 10-90%, 15-85%, 20-80%, or 1.5-fold to 10-fold, relative to a control. Protein activity may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein activity may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein activity may be reduced by about 5-95%, 10-90%, 15-85%, 20-80%, or 1.5-fold to 10-fold, relative to a control. Protein activity may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein activity may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein activity may be increased by about 5-95%, 10-90%, 15-85%, 20-80%, or 1.5-fold to 10-fold, relative to a control.

In some embodiments, the level or amount of the protein expressed from the targeted gene is modulated. Protein level or amount may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein level or amount may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein level or amount may be modulated by about 5-95%, 10-90%, 15-85%, 20-80%, or 1.5-fold to 10-fold, relative to a control. Protein level or amount may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein level or amount may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein level or amount may be reduced by about 5-95%, 10-90%, 15-85%, 20-80%, or 1.5-fold to 10-fold, relative to a control. Protein level or amount may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein level or amount may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. Protein level or amount may be increased by about 5-95%, 10-90%, 15-85%, 20-80%, or 1.5-fold to 10-fold, relative to a control.

a. Polynucleotides

In some embodiments, the agent comprises a polynucleotide. Inhibitors comprising polynucleotides may be referred to as inhibitory nucleic acids or inhibitory polynucleotides. Polynucleotides may include, for example, antisense oligonucleotides (ASOs) or polynucleotides, ribozymes, short hairpin RNA (shRNA), SIRNA, single-stranded or double-stranded RNA interference (RNAI), modified bases/locked nucleic acids (LNAs), peptide nucleic acids (PNAs), and/or other oligomeric or oligonucleotides. See, for example, inhibitory nucleic acids disclosed in U.S. Patent Publication No. 2020/0216549, incorporated herein by reference. The polynucleotide may hybridize to at least a portion of a target nucleic acid, such as a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a regulatory portion thereof, or a transcribed portion thereof. Binding of the polynucleotide to the target nucleic acid may inhibit the function of the target nucleic acid.

In some embodiments, the polynucleotide is an antisense polynucleotide. Antisense polynucleotides may also be referred to as antisense oligonucleotides. Antisense polynucleotides are typically designed to block expression of a DNA or RNA target by binding to the target and halting expression at the level of transcription, translation, or splicing. Antisense polynucleotides are complementary nucleic acid sequences designed to hybridize under stringent conditions to an RNA. Polynucleotides may be chosen that are sufficiently complementary to the target in that they hybridize sufficiently well and with sufficient specificity to give the desired effect.

In some embodiments, the polynucleotide complementary to a target RNA is an interfering RNA, including but not limited to a small interfering RNA (“sIRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (for example, each strand comprises a nucleotide sequence that is complementary to a nucleotide sequence in the other strand, such as where the antisense strand and the sense strand form a duplex or double stranded structure); the antisense strand comprises a nucleotide sequence that is complementary to a nucleotide sequence in a target nucleic acid molecule or a portion thereof, and the sense strand comprises nucleotide sequence corresponding to the target nucleic acid sequence or a portion thereof. As another example, interfering RNA may be assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin, or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region has a nucleotide sequence corresponding to the target nucleic acid sequence or a portion thereof. The interfering RNA can be a circular single-stranded polynucleotide having two or more loop structures and a stem comprising self-complementary sense and antisense regions, wherein the antisense region comprises a nucleotide sequence that is complementary to a nucleotide sequence in a target nucleic acid molecule or a portion thereof, and the sense region has a nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region, and a loop region. Such an RNA molecule when expressed desirably forms a “hairpin” structure and may be referred to as an “shRNA.” The loop region may generally be between about 2 and about 10 nucleotides in length, or from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. See, for example, Brummelkamp et al. Science 2002, 296, 550-553; Lee et al. Nature Biotechnol. 2002, 20, 500-505; Miyagishi and Taira, Nature Biotechnol. 2002, 20, 497-500; Paddison et al. Genes & Dev. 2002, 16, 948-958; Paul, Nature Biotechnol, 2002, 20, 505-508; Sui, PNAS 2002, 99, 5515-5520; Yu et al. PNAS 2002, 99, 6047-6052.

The target RNA cleavage reaction guided by siRNAs may be highly sequence specific. In general, a siRNA containing a nucleotide sequence identical to a portion of the target nucleic acid may be preferred for inhibition. However, 100% sequence identity between the siRNA and the target gene may not be required. Sequence variations due to genetic mutation, strain polymorphism, or evolutionary divergence, for example, may be tolerated. For example, siRNA sequences with insertions, deletions, and single point mutations relative to the target sequence may be effective for inhibition. siRNA sequences with nucleotide analog substitutions or insertions may be effective for inhibition. siRNAs may retain specificity for their target, that is, they may not directly bind to, or directly significantly affect expression levels of, transcripts other than the intended target. In some embodiments, the agent comprises siRNA. In some embodiments, the agent comprises shRNA.

In some embodiments, the inhibitor is a ribozyme. Trans-cleaving enzymatic nucleic acid molecules such as ribozymes can be used and have shown promise as therapeutic agents for human disease (Usman & McSwiggen, Ann. Rep. Med. Chem. 1995, 30, 285-294; Christoffersen and Marr. J. Med. Chem. 1995, 38, 2023-2037). Enzymatic nucleic acid molecules can be designed to cleave specific RNA targets within the background of cellular RNA. Such a cleavage event can render the RNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA may destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, Proc. R. Soc. London, B 1979, 205, 435) have been used to evolve new nucleic acid catalysts capable of catalyzing a variety of reactions, such as cleavage and ligation of phosphodiester linkages and amide linkages (Joyce, Gene 1989, 82, 83-87; Beaudry et al. Science 1992, 257, 635-641; Joyce, Scientific American 1992, 267, 90-97; Breaker et al. TIBTECH 1994, 12, 268; Bartel et al. Science 1993, 261, 1411-1418; Szostak, TIBS 1993, 17, 89-93; Kumar et al. FASEB J. 1995, 9, 1183; Breaker, Curr. Op. Biotech. 1996, 1, 442). Ribozymes may be developed to optimize catalytic activity and contribute to any strategy that employs RNA-cleaving ribozymes for the purpose of regulating gene expression, such as, for example, the hammerhead ribozyme, modified hammerhead ribozymes, and other artificial “RNA ligase” ribozymes.

In some embodiments, the polynucleotide is modified. For example, the polynucleotide may be modified to include one or more modified bonds or bases. A number of modified bases may include phosphorothioate, methylphosphonate, peptide nucleic acids, or locked nucleic acid (LNA) molecules. A polynucleotide may be fully modified, while others may be chimeric and contain two or more chemically distinct regions, each made up of at least one nucleotide. These inhibitory nucleic acids may contain at least one region of modified nucleotides that confers one or more beneficial properties (such as, for example, increased nuclease resistance, increased uptake into cells, increased binding affinity for the target) and a region that is a substrate for enzymes capable of cleaving RNA: DNA or RNA: RNA hybrids. Chimeric inhibitory nucleic acids may be formed as composite structures of two or more oligonucleotides, modified oligonucleotides, oligonucleosides, and/or oligonucleotide mimetics as described above. Such chimeric inhibitory nucleic acids may be referred to as hybrids or gapmers. In some embodiments, the polynucleotide is a gapmer, which contains a central stretch (gap) of DNA monomers sufficiently long to induce RNase H cleavage, flanked by blocks of LNA modified nucleotides (see, for example, Stanton et al. Nucleic Acid Ther. 2012, 22, 344-359; Nowotny et al. Cell, 2005, 121, 1005-1016; Kurreck, European Journal of Biochemistry 2003, 270, 1628-1644; Fluiter et al., Mol. Biosyst. 2009, 5, 838-843; incorporated herein by reference). In some embodiments, the polynucleotide is a mixmer, which includes alternating short stretches of LNA and DNA (see, for example, Naguibneva et al., Biomed Pharmacother. 2006, 60, 633-638; Orom et al. Gene 2006, 372, 137-141; incorporated herein by reference). Representative United States patents that disclose the preparation of such hybrid structures may include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is incorporated herein by reference.

In some embodiments, the modified polynucleotide comprises at least one nucleotide modified at the 2′ position of the sugar, such as a 2′-O-alkyl, 2′-O-alkyl-O-alkyl, or 2′-fluoro-modified nucleotide. In other embodiments, RNA modifications include 2′-fluoro, 2′-amino, and 2′ O-methyl modifications on the ribose of pyrimidines, abasic residues, or an inverted base at the 3′ end of the RNA. Such modifications are routinely incorporated into oligonucleotides, and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide. These modified polynucleotides may survive intact for a longer period of time than unmodified polynucleotides. Specific examples of modified polynucleotides may include those comprising modified backbones, for example, phosphorothioates, phosphotriesters, methyl phosphonates, short chain alkyl or cycloalkyl intersugar linkages, or short chain heteroatomic or heterocyclic intersugar linkages. Modified polynucleotides may also include phosphorothioate backbones and those with heteroatom backbones, particularly CH2-NH—O—CH2, CH, —N(CH3)-O—CH2 (known as a methylene(methylimino) or MMI backbone), CH2-O—N(CH3)-CH2, CH2-N(CH3)-N(CH3)-CH2, and O—N(CH3)-CH2-CH2 backbones, wherein the native phosphodiester backbone is represented as O—P—O—CH); amide backbones (see, for example, De Mesmaeker et al. Ace. Chem. Res. 1995, 28, 366-374); morpholino backbone structures (see, for example, Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone; see, for example, Nielsen et al., Science 1991, 254, 1497), all references incorporated herein by reference. Phosphorus-containing linkages may include, but are not limited to, phosphorothioates, chiral phosphorothioates, phosphorodithioates, phosphotriesters, aminoalkylphosphotriesters, methyl, and other alkyl phosphonates comprising 3′-alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′ (see, for example, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, incorporated herein by reference). Morpholino-based oligomeric compounds are described in Dwaine A. Braasch and David R. Corey, Biochemistry 2002, 41, 4503-4510); Genesis, volume 30, issue 3, 2001; Heasman, J., Dev. Biol. 2002, 243, 209-214; Nasevicius et al. Nat. Genet. 2000, 26, 216-220; Lacerra et al. Proc. Natl. Acad. Sci. 2000, 97, 9591-9596; and U.S. Pat. No. 5,034,506, all incorporated herein by reference. Cyclohexenyl nucleic acid oligonucleotide mimetics are described in Wang et al. J. Am. Chem. Soc. 2000, 122, 8595-8602, incorporated herein by reference.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These may comprise those having morpholino linkages, formed in part from the sugar portion of a nucleoside; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH2 component parts; see U.S. Pat. Nos. 5,034,506; 5,166,315; 5,185,444; 5,214,134; 5,216,141; 5,235,033; 5,264,562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, for example, one of the following at the 2′ position: OH, SH, SCH₃, F, OCN, OCH3 OCH3, OCH3 O(CH2)n CH3, O(CH2)n NH2 or O(CH2)n CH3 where n is from 1 to about 10; C1 to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A modification may include 2′-methoxyethoxy [2′-O—CH2CH2OCH3, also known as 2′-O-(2-methoxyethyl)] (Martin et al, Helv. Chim. Acta, 1995, 78, 486). Other modifications may include 2′-methoxy (2′-O—CH3), 2′-propoxy (2′-OCH2CH2CH₃) and 2′-fluoro (2′-F). Similar modifications may also be made at other positions on the oligonucleotide, such as the 3′ position of the sugar on the 3′ terminal nucleotide and the 5′ position of 5′ terminal nucleotide. Oligonucleotides may also have sugar mimetics such as cyclobutyls in place of the pentofuranosyl group.

Polynucleotides can include, additionally or alternatively, one or more nucleobase modifications or substitutions. As used herein, “unmodified” or “natural” nucleobases comprise the purine bases adenine (A) and guanine (G), and the pyrimidine bases thymine (T), cytosine (C), and uracil (U). Modified nucleobases may include nucleobases found only infrequently or transiently in natural nucleic acids, such as hypoxanthine, 6-methyladenine, 5-Me pyrimidines, 5-methylcytosine (also referred to as 5-methyl-2′ deoxycytosine and 5-Me-C), 5-hydroxymethylcytosine (HMC), glycosyl HMC, gentobiosyl HMC. Modified nucleobases may also include synthetic nucleobases, such as 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine, 2,6-diaminopurine, xanthine, hypoxanthine, 6-methyl and other alkyl derivatives of adenine and guanine, 2-propyl and other alkyl derivatives of adenine and guanine, 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine (see, for example, Komberg, DNA Replication, W. H. Freeman & Co., San Francisco, 1980, pp 75-77; Gebeyehu, G., et al. Nucl. Acids Res. 1987, 15, 4513). A “universal” base known in the art, such as inosine, can also be included. 5-Me-C substitutions may also be included and have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278). Some nucleobases may be useful for increasing the binding affinity of the polynucleotides. These may include 5-substituted pyrimidines, 6-azapyrimidines, and N-2, N-6, and 0-6 substituted purines, comprising 2-aminopropyladenine, 5-propynyluracil, and 5-propynylcytosine. 5-methylcytosine substitutions may be combined with 2′-O-methoxyethyl sugar modifications.

It is not necessary for all positions in a given oligonucleotide to be uniformly modified. More than one of the aforementioned modifications may be incorporated in a single oligonucleotide or even at within a single nucleoside within an oligonucleotide.

In some embodiments, both a sugar and an internucleoside linkage, that is, the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone.

Representative United States patents that teach the preparation of PNA compounds include U.S. Pat. Nos. 5,539,082; 5,714,331; and 5,719,262, each of which is herein incorporated by reference. Further teaching of PNA compounds can be found in Nielsen et al. Science 1991, 254, 1497-1500, incorporated herein by reference. Nucleobases are further described in U.S. Pat. No. 3,687,808; “The Concise Encyclopedia of Polymer Science And Engineering”, pages 858-859, Kroschwitz, J. I., ed. John Wiley & Sons, 1990; Englisch et al., Angewandle Chemie, International Edition’, 1991, 30, page 613; Sanghvi, Y. S., Chapter 15, Antisense Research and Applications’, pages 289-302, Crooke, S. T.; and Lebleu, B. ea., CRC Press, 1993. Modified nucleobases are also described in U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692; and 5,681,941, each of which is herein incorporated by reference.

In some embodiments, the polynucleotide is chemically linked to one or more moieties or conjugates that enhance the activity, cellular distribution, or cellular uptake of the oligonucleotide. Such moieties may include but are not limited to, lipid moieties such as a cholesterol moiety (Letsinger et al. Proc. Natl. Acad. Sci. USA 1989, 86, 6553-6556), cholic acid (Manoharan et al., Bioorg. Med. Chem. Let., 1994, 4, 1053-1060), a thioether, such as hexyl-S-tritylthiol (Manoharan et al. Ann. N. Y. Acad. Sci. 1992, 660, 306-309; Manoharan et al. Bioorg. Med. Chem. Let. 1993, 3, 2765-2770), a thiocholesterol (Oberhauser et al., Nucl. Acids Res. 1992, 20, 533-538), an aliphatic chain, such as dodecandiol or undecyl residues (Kabanov et al. FEBS Lett. 1990, 259, 327-330; Svinarchuk et al. Biochimie. 1993, 75, 49-54), a phospholipid such as di-hexadecyl-rac-glycerol or triethylammonium 1,2-di-O-hexadecyl-rac-glycero-3-H-phosphonate (Manoharan et al. Tetrahedron Lett. 1995, 36, 3651-3654; Shea et al. Nucl. Acids Res. 1990, 18, 3777-3783), a polyamine or a polyethylene glycol chain (Mancharan et al. Nucleosides & Nucleotides, 1995, 14, 969-973), or adamantane acetic acid (Manoharan et al., Tetrahedron Lett. 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther. 1996, 277, 923-937), incorporated herein by reference. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599,928; and 5,688,941, each of which is herein incorporated by reference.

These moieties or conjugates can include conjugate groups covalently bound to functional groups such as primary or secondary hydroxyl groups. Conjugate groups may include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups may include cholesterols, lipids, phospholipids, biotin, phenazine, folate, phenanthridine, anthraquinone, acridine, fluoresceins, rhodamines, coumarins, and dyes. Groups that enhance the pharmacodynamic properties may include groups that improve uptake, enhance resistance to degradation, and/or strengthen sequence-specific hybridization with the target nucleic acid. Groups that enhance the pharmacokinetic properties may include groups that improve uptake, distribution, metabolism or excretion of the inhibitors. Representative conjugate groups are also disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether such as hexyl-5-tritylthiol, a thiocholesterol, an aliphatic chain such as dodecandiol or undecyl residues, a phospholipid such as di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety (see, for example, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941, incorporated herein by reference.

In some embodiments, the polynucleotides comprise locked nucleic acid (LNA) molecules, such as those [alpha]-L-LNAs. LNAs comprise ribonucleic acid analogues wherein the ribose ring is “locked” by a methylene bridge between the 2′-oxgygen and the 4′-carbon, such as oligonucleotides containing at least one LNA monomer, that is, one 2′-0,4′-C-methylene-. beta.-D-ribofuranosyl nucleotide. LNA bases may form standard Watson-Crick base pairs but the locked configuration increases the rate and stability of the basepairing reaction (Jensen et al., Oligonucleotides, 2004, 14, 130-146, incorporated herein by reference). LNAs may also have increased affinity to base pair with RNA as compared to DNA. These properties may render LNAs especially useful as probes for fluorescence in situ hybridization (FISH) and comparative genomic hybridization, as knockdown tools for miRNAs, and as antisense oligonucleotides to target mRNAs or other RNAs such as the RNAs as described herein.

LNA molecules can include molecules comprising 10-30 nucleotides, or 12-24 nucleotides, such as 12, 13, 14, 15, 16, 17, 18, 19, 20, 21, 22, 23, 24, 25, 26, 27, 28, 29, or 30 nucleotides in each strand. One of the strands may be substantially identical to a target region in the RNA. One of the strands may be at least 80%, at least 85%, at least 90%, at least 95%, or 100% identical to a target region in the RNA. One of the strands may have 3, 2, 1, or 0 mismatched nucleotide(s) relative to a target region in the RNA. The LNA molecules can be chemically synthesized using methods known in the art.

LNA molecules can be designed using any method known in the art; a number of algorithms are known and are commercially available (for example see exiqon.com; You et al., Nuc. Acids. Res. 2006, 34, e60; McTigue et al., Biochemistry 2004, 43, 5388-5405; and Levin et al., Nuc. Acids. Res. 2006, 34, e14; incorporated herein by reference). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, such as gaps of 5-10 nucleotides or more, can be left between the LNAs to reduce the number of oligonucleotides synthesized and tested. GC content may be, for example, between about 30-60%. General guidelines for designing LNAs are known in the art; for example, LNA sequences may bind very tightly to other LNA sequences, so it may be preferable to avoid significant complementarity within an LNA. Contiguous runs of more than four LNA residues may be avoided where possible (for example, it may not be possible with very short (such as about 9-10 nt) oligonucleotides). In some embodiments, the LNAs are xylo-LNAs. For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 1998, 54, 3607-3630; Obika et al. Tetrahedron Lett. 1998, 39, 5401-5404; Jepsen et al. Oligonucleotides 2004, 14, 130-146; Kauppinen et al. Drug Disc. Today 2005, 2, 287-290; and Ponting et al. Cell 2009, 136, 629-641, and references cited therein, all incorporated by reference.

In some embodiments, the inhibitor comprises an antisense oligonucleotide, SIRNA, RNAI, shRNA, LNA, and/or PNA. In some embodiments, the inhibitor comprises SIRNA. In some embodiments, the modulator includes a polynucleotide comprising one or more of a modified internucleoside linkage, a modified sugar moiety, and/or a modified nucleobase as detailed herein.

b. DNA Targeting Systems

In some embodiments, the agent comprises a DNA targeting composition or at least one component thereof. A “DNA Targeting System” as used herein is a system capable of specifically targeting a particular region of DNA and modulating gene expression by binding to that region. Non-limiting examples of these systems are CRISPR-Cas-based systems, meganucleases, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

Each of these systems comprises a DNA-binding portion or domain, such as a Cas protein and guide RNA, or a meganuclease, or a ZF, or a TALE, that specifically recognizes and binds to a particular target region of a target DNA. The DNA-binding portion (for example, Cas protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity, to form a fusion protein. Exemplary second polypeptide domains are detailed further below (see “Cas Fusion Protein”). For example, the DNA-binding portion can be linked to an activator and thus guide the activator to a specific target region of the target DNA. Similarly, the DNA-binding portion can be linked to a repressor and thus guide the repressor to a specific target region of the target DNA.

In some embodiments, the DNA targeting composition comprises a meganuclease. A meganuclease is an endodeoxyribonuclease characterized by a large recognition site, such as double-stranded DNA sequences of 12 to 40 base pairs. The recognition site may occur only once in any given genome. A meganuclease may be a homing endonuclease selected from an intron endonuclease or an intein endonuclease. Meganucleases may include, for example, the LAGLIDADG family of homing endonucleases.

In some embodiments, the DNA-binding portion comprises a Cas protein, such as a Cas9 protein. Some CRISPR-Cas-based systems can operate to activate or repress expression using the Cas protein alone, not linked to an activator or repressor. For example, a nuclease-null Cas9 can act as a repressor on its own, a nuclease-active Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor. Some CRISPR-Cas-based systems can operate to activate or repress expression using the Cas protein linked to a second protein domain, such as, for example, an activator or repressor.

### i) DNA Binding Protein

The DNA Targeting System may include a DNA binding protein. The DNA binding protein may comprise, for example, a zinc finger protein or a transcription activator-like effector (TALE). The zinc finger protein or TALE may target a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a regulatory element thereof.

### (1) Zinc Finger Protein

A zinc finger protein is a protein that includes one or more zinc finger domains. Zinc finger domains are relatively small protein motifs that contain multiple finger-like protrusions that make tandem contacts with their target molecule such as a DNA target molecule. A zinc finger domain may bind one or more zinc ions or other metal ion such as iron, or in some cases a zinc finger domain forms salt bridges to stabilize the finger-like folds. The zinc binding portion of a zinc finger protein may include one or more cysteine residues and/or one or more histidine residues to coordinate the zinc or other metal ion. A zinc finger protein recognizes and binds to a particular DNA sequence via the zinc finger domain. In some embodiments, a zinc finger protein is fused to or includes a nuclease domain and may be referred to as a zinc finger nuclease (ZFN). The nuclease domain may include, for example, the endonuclease Fokl. ZFNs may recognize target sites that consist of two zinc-finger binding sites that flank a 5- to 7-base pair (bp) spacer sequence recognized by the endonuclease Fokl cleavage domain.

### (2) Transcription Activator-Like Effector (TALE)

A TALE is another type of protein that recognizes and binds to a particular DNA sequence. The DNA-binding domain of a TALE includes an array of tandem 33-35 amino acid repeats, also known as RVD modules. Each RVD module specifically recognizes a single base pair of DNA. RVD modules may be arranged in any order to assemble an array that recognizes a defined DNA sequence. The binding specificity of a TALE DNA-binding domain is determined by the RVD array followed by a single truncated repeat of, for example, 20 amino acids. A TALE DNA-binding domain may have an array of 12 to 27 RVD modules, each RVD module recognizing a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors and/or nucleases. In some embodiments, a TALE is fused to or includes a nuclease domain and may be referred to as a TALE nuclease (TALEN). The nuclease domain may include, for example, the endonuclease Fokl. TALENs may recognize target sites that consist of two TALE DNA-binding sites that flank a 12-bp to 20-bp spacer sequence recognized by the Fokl cleavage domain.

### (3) DNA Binding Fusion Protein

Additionally or alternatively, a zinc finger protein or TALE can be fused to a polypeptide domain and referred to as a DNA binding fusion protein or fusion protein. The fusion protein may act as a synthetic transcription factor. The fusion protein comprises two heterologous polypeptide domains, including a first polypeptide domain comprising the zinc finger protein or the TALE or a Cas protein as further detailed below, and a second polypeptide domain having an activity selected from transcription activation activity, transcription repression activity, nuclease activity, transcription release factor activity, histone modification activity, nucleic acid association activity, methylase activity, and demethylase activity. A zinc finger protein or TALE can be fused to a polypeptide domain having epigenetic modifying activity to mediate targeted gene regulation. A fusion protein comprising a zinc finger protein or TALE, and a second polypeptide domain having transcription repression activity, may mediate targeted gene repression. A fusion protein comprising a zinc finger protein or TALE, and a second polypeptide domain having transcription activation activity, may mediate targeted gene activation. The second polypeptide domain is further detailed below (see “Cas Fusion Protein”).

### ii) CRISPR/Cas-based Gene Editing System

Provided herein are CRISPR/Cas-based gene editing systems. The CRISPR/Cas-based gene editing system may be used to induce differentiation of a cell into a hepatocyte or promote maturation of a hepatocyte. The CRISPR/Cas-based gene editing system may include a Cas protein or a fusion protein, and at least one gRNA, and may also be referred to as a “CRISPR-Cas system.”

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, Cas13, and Cascade proteins. Cas12a may also be referred to as “Cpf1.” Cas12a causes a staggered cut in double stranded DNA, while Cas9 produces a blunt cut. In some embodiments, the Cas protein comprises Cas12a. Cas12a is described in, for example, WO 2018/017754, which is incorporated herein by reference. Cas13 is an RNA-guided RNA endonuclease. Cas13 cleaves single-stranded RNA, and it does not cleave DNA. In some embodiments, the Cas protein comprises Cas13. In some embodiments, the Cas protein comprises Cas9. Cas9 forms a complex with the 3′ end of the sgRNA (which may be referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9: crRNA-tracrRNA complex. Cas12a systems include crRNA for successful targeting, whereas Cas9 systems include both crRNA and tracrRNA.

The Cas9: orRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

iii) Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithil, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotil, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthil, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 26. In certain embodiments, the Cas9 molecule is a Staphylococcus aureus Cas9 molecule (also referred herein as “SaCas9”). SaCas9 may comprise an amino acid sequence of SEQ ID NO: 27.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi: 10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi: 10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G; V=A or C or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 20).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 28. A S. pyogenes Cas9 protein with D10A and H849A mutations may comprise an amino acid sequence of SEQ ID NO: 29. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 30. In certain embodiments, the mutant S. aureus Cas9 molecule comprises a N580A mutation. The nucleotide sequence encoding this mutant S. aureus Cas9 molecule is set forth in SEQ ID NO: 31.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 32. Exemplary codon optimized nucleic acid sequences encoding a Cas9 molecule of S. aureus, and optionally containing nuclear localization sequences (NLSs), are set forth in SEQ ID NOs: 33-39. Another exemplary codon optimized nucleic acid sequence encoding a Cas9 molecule of S. aureus comprises the nucleotides 1293-4451 of SEQ ID NO: 40.

### iv) Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain has a different activity than what is endogenous to Cas protein. The second polypeptide domain may have any DNA editing activity. The second polypeptide domain may have an activity such as transcription activation activity, transcription repression activity, transcription release factor activity, histone modification activity, nuclease activity, nucleic acid association activity, histone methylase activity, DNA methylase activity, histone demethylase activity, DNA demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. In some embodiments, the fusion protein comprises more than one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser) n, wherein n is an integer between 0 and 10 (SEQ ID NO: 21). In a GS linker, n can be adjusted to optimize the linker length and achieve appropriate separation of the functional domains. Other examples of linkers may include, for example, Gly-Gly-Gly-Gly-Gly (SEQ ID NO: 22), Gly-Gly-Ala-Gly-Gly (SEQ ID NO: 23), Gly/Ser rich linkers such as Gly-Gly-Gly-Gly-Ser-Ser-Ser (SEQ ID NO: 24), or Gly/Ala rich linkers such as Gly-Gly-Gly-Gly-Ala-Ala-Ala (SEQ ID NO: 25).

In some embodiments, the agent and/or Cas protein and/or the Cas fusion protein and/or gRNAs detailed herein may be used in compositions and methods for modulating expression of gene. Modulating may include, for example, increasing or enhancing expression of the gene, or reducing or inhibiting expression of the gene. The expression of the gene may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5-95%, 10-90%, 15-85%, 20-80%, or 1.5-fold to 10-fold, relative to a control. The expression of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5-95%, 10-90%, 15-85%, 20-80%, or 1.5-fold to 10-fold, relative to a control. The expression of the gene may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control.

### (1) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 41 or SEQ ID NO: 42. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 43, encoded by the polynucleotide of SEQ ID NO: 44. VPH may comprise a polypeptide having the amino acid sequence of SEQ ID NO: 53, encoded by the polynucleotide of SEQ ID NO: 54. VPR may comprise a polypeptide having the amino acid sequence of SEQ ID NO: 55, encoded by the polynucleotide of SEQ ID NO: 56.

### (2) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4× repressor domain, Mxil repressor domain, SUV39H1, SUV39H2, G9A, ESET/SETBD1, Cir4, Su (var) 3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, Mxil repressor domain activity, SID4× repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. KRAB may comprise a polypeptide having the amino acid sequence of SEQ ID NO: 45, encoded by a polynucleotide comprising the sequence of SEQ ID NO: 46. For example, the fusion protein may be S. pyogenes dCas9-KRAB (protein sequence comprising SEQ ID NO: 47; polynucleotide sequence comprising SEQ ID NO: 48). The fusion protein may be S. aureus dCas9-KRAB (protein sequence comprising SEQ ID NO: 49; polynucleotide sequence comprising SEQ ID NO: 50).

### (3) Transcription Release Factor Activity

The second polypeptide domain can have transcription release factor activity. The second polypeptide domain can have eukaryotic release factor 1 (ERF1) activity or eukaryotic release factor 3 (ERF3) activity.

### (4) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 41 or SEQ ID NO: 42.

### (5) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease. In some embodiments, the second polypeptide domain includes a meganuclease, as detailed above. In some embodiments, the polypeptide domain having nuclease activity comprises Fokl.

### (6) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

### (7) Base Editing Activity

The second polypeptide domain may have base editing activity. Base editing enables the direct, irreversible conversion of a specific DNA base into another base at a targeted genomic locus without requiring double-stranded DNA breaks (DSB). A base editing domain has sequence requirements for activity. In a 20 nucleotide protospacer, the target base may be within 4-8 nucleotides from the PAM-distal end. An exemplary splice acceptor is an “AG” immediately before the exon, and an exemplary splice donor is a “GT” immediately following the exon. Cas9 molecules from different species may use different PAMs, and thereby provide some flexibility in selecting the base to edit. Disruption of canonical splice sites can lead to exon skipping or activation of cryptic splice sites. Both adenine and cytosine base editors may be capable of disrupting an “AG” splice acceptor, converting it to either a “GG” or “AA”, respectively. In some embodiments, the base-editing domain includes an adenine base editor (ABE). Adenine base editors may include, for example, ecTadA, including wild-type and mutants thereof. The adenine base editor may be as described in Gaudelli et al. (Nature 2017, 551, 464-471), Koblan et al. (Nature Biotech. 2018, 36, 843-846), Richter et al. (Nature Biotech. 2020, 38, 883-891), and Gaudelli et al. (Nature Biotech. 2020, 38, 892-900), each of which is incorporated herein by reference. The ABE may comprise a polypeptide selected from SEQ ID NOs: 57-64 and/or be encoded by a polynucleotide comprising a sequence selected from SEQ ID NOs: 65-72, respectively. In some embodiments, the base-editing domain includes a cytidine deaminase domain. A cytidine deaminase domain can convert the DNA base cytosine to uracil. In some embodiments, the cytidine deaminase domain can include an apolipoprotein B mRNA-editing enzyme, catalytic polypeptide-like (APOBEC) family deaminase. In some embodiments, the cytidine deaminase domain can include an APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, APOBEC3H deaminase, or a combination thereof. Base editing domains are detailed in, for example, WO 2020/210776 and WO 2022/081612, each of which is incorporated herein by reference.

### (8) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

### (9) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; amino acid sequence comprising SEQ ID NO: 51; polynucleotide sequence comprising SEQ ID NO: 52). In some embodiments, the second polypeptide domain has histone demethylase activity. In some embodiments, the second polypeptide domain has DNA demethylase activity.

v) Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 (RNA), which is encoded by a sequence comprising SEQ ID NO: 18 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target the Cas9 protein or fusion protein to a gene or a regulatory element thereof. The gRNA may target the Cas protein or fusion protein to a non-open chromatin region, an open chromatin region, a transcribed region of the target gene, a region upstream of a transcription start site of the target gene, a regulatory element of the target gene, an intron of the target gene, or an exon of the target gene, or a combination thereof. In some embodiments, the gRNA targets the Cas9 protein or fusion protein to a promoter of a gene. In some embodiments, the target region is located between about 1 to about 1000 base pairs upstream of a transcription start site of a target gene. In some embodiments, the DNA targeting composition comprises two or more gRNAs, each gRNA binding to a different target region.

The gRNA may target a region of a gene to promote differentiation of a cell into a hepatocyte or maturation of a hepatocyte. The gRNA may target a region of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a regulatory element thereof. The gRNA may target a region of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a regulatory element thereof. The gRNA may target a region of a gene selected from FOXA2, HNF4A, NR5A2, FOXA3, or HNF4G, or a regulatory element thereof. In some embodiments, the gRNA targets a gene and is used in combination with a Cas9 fusion protein wherein the second polypeptide domain has transcription activation activity, to activate or enhance expression of the gene. In some embodiments, the gRNA targets a gene and is used in combination with a Cas9 fusion protein wherein the second polypeptide domain has transcription repression activity, to inhibit or reduce or decrease expression of the gene. The gRNA may comprise a polynucleotide selected from at least one of SEQ ID NOs: 439-718, or a complement thereof, or a variant thereof, or a truncation thereof. The gRNA may be encoded by a polynucleotide sequence comprising at least one of SEQ ID NOs: 159-438, or a complement thereof, or a variant thereof, or a truncation thereof. The gRNA may bind and target a polynucleotide sequence comprising at least one of SEQ ID NOs: 159-438, or a complement thereof, or a variant thereof, or a truncation thereof. A truncation may be 1, 2, 3, 4, 5, 6, 7, 8, or 9 nucleotides shorter than the sequence of any one of SEQ ID NOs: 159-718. Exemplary gRNA sequences for target genes are shown in TABLE 3.

TABLE 3
Genes and gRNA sequences for producing mature
hepatocytes.

| | | SEQ | | SEQ |
|---|---|---|---|---|
| | DNA sequences for | ID | | ID |
| Gene | gRNAs | NO: | gRNAs | NO: |
| NHLH2 | CTGGACATGAGTCACTTAAT | 159 | CUGGACAUGAGUCACUUAAU | 439 |
| | CAATTAAGTGACTCATGTCC | 160 | CAAUUAAGUGACUCAUGUCC | 440 |
| | TTTATGGCACAGATTCAGTG | 161 | UUUAUGGCACAGAUUCAGUG | 441 |
| | CTTGACATCTGTCACAGTGT | 162 | CUUGACAUCUGUCACAGUGU | 442 |
| | AATCCTATTGGACAGAAACC | 163 | AAUCCUAUUGGACAGAAACC | 443 |
| | ACTTGACATCTGTCACAGTG | 164 | ACUUGACAUCUGUCACAGUG | 444 |
| | GAGATAAAGGCTTAGGAAGG | 165 | GAGAUAAAGGCUUAGGAAGG | 445 |
| | ACGGCTAGATACTGTTTAGG | 166 | ACGGCUAGAUACUGUUUAGG | 446 |
| | TGGACATGAGTCACTTAATG | 167 | UGGACAUGAGUCACUUAAUG | 447 |
| | TATTTAATGGGAAATCCTAT | 168 | UAUUUAAUGGGAAAUCCUAU | 448 |
| KLF7 | TCTGCTCCGGGCTCCCCATG | 169 | UCUGCUCCGGGCUCCCCAUG | 449 |
| | TGCGCGGAGAACCGAACGGG | 170 | UGCGCGGAGAACCGAACGGG | 450 |
| | CGAGCGCAAGGGACAGGGAG | 171 | CGAGCGCAAGGGACAGGGAG | 451 |
| | GGGAAGACTACAAACTAGCA | 172 | GGGAAGACUACAAACUAGCA | 452 |
| | ACTACAAACTAGCAAGGATG | 173 | ACUACAAACUAGCAAGGAUG | 453 |
| | GACTACAAACTAGCAAGGAT | 174 | GACUACAAACUAGCAAGGAU | 454 |
| | ACCATGTAAGGTAAACAGGG | 175 | ACCAUGUAAGGUAAACAGGG | 455 |
| | CAAACTAGCAAGGATGGGGT | 176 | CAAACUAGCAAGGAUGGGGU | 456 |
| | AGACTACAAACTAGCAAGGA | 177 | AGACUACAAACUAGCAAGGA | 457 |
| | ACAAACTAGCAAGGATGGGG | 178 | ACAAACUAGCAAGGAUGGGG | 458 |
| GSC2 | GCCCTGCCTCCGCGCGCGGG | 179 | GCCCUGCCUCCGCGCGCGGG | 459 |
| | AGGCAGGGCCGCCGCAGTCG | 180 | AGGCAGGGCCGCCGCAGUCG | 460 |
| | GCCCTGCCAGCTTGAGCGGG | 181 | GCCCUGCCAGCUUGAGCGGG | 461 |
| | GGTCGGGGCGCCCTCCGAGG | 182 | GGUCGGGGCGCCCUCCGAGG | 462 |
| | CGCTGCGGGATTAACCCGCG | 183 | CGCUGCGGGAUUAACCCGCG | 463 |
| | GCGCTAATCCTCGACTGCGG | 184 | GCGCUAAUCCUCGACUGCGG | 464 |
| | GCCGGGCGTCCAGTCCACGC | 185 | GCCGGGCGUCCAGUCCACGC | 465 |
| | GGCCGGGCGTCCAGTCCACG | 186 | GGCCGGGCGUCCAGUCCACG | 466 |
| | AACGCGCTAATCCTCGACTG | 187 | AACGCGCUAAUCCUCGACUG | 467 |
| | GGTGGGGCAGGAGGACGCGG | 188 | GGUGGGGCAGGAGGACGCGG | 468 |
| PPARG | CCTGGGAGCCTAACTGAGGG | 189 | CCUGGGAGCCUAACUGAGGG | 469 |
| | AGGTCCCTTGGTGGAAGGTG | 190 | AGGUCCCUUGGUGGAAGGUG | 470 |
| | AGTCGTGGTACTTTACGCCT | 191 | AGUCGUGGUACUUUACGCCU | 471 |
| | TTACGCCTCGGTGTTTAGGG | 192 | UUACGCCUCGGUGUUUAGGG | 472 |
| | AGGCTCCTCCCTAAACACCG | 193 | AGGCUCCUCCCUAAACACCG | 473 |
| | CCTCCTGGGAGCCTAACTGG | 194 | CCUCCUGGGAGCCUAACUGG | 474 |
| | GGAAGGTGGGTGTGTAGTCG | 195 | GGAAGGUGGGUGUGUAGUCG | 475 |
| | AGAAATCTCAGGTCCCTTGG | 196 | AGAAAUCUCAGGUCCCUUGG | 476 |
| | TACACACCCACCTTCCACCA | 197 | UACACACCCACCUUCCACCA | 477 |
| | GCCTCTACCCCCCTCAGTTG | 198 | GCCUCUACCCCCCUCAGUUG | 478 |
| SALL4 | AGCCCTCATTGATCAGGGAG | 199 | AGCCCUCAUUGAUCAGGGAG | 479 |
| | GCTTATTTAAATGATCTCTG | 200 | GCUUAUUUAAAUGAUCUCUG | 480 |
| | TAAGCCCTCATTGATCAGGG | 201 | UAAGCCCUCAUUGAUCAGGG | 481 |
| | GGGACACGGGTCACATCTCG | 202 | GGGACACGGGUCACAUCUCG | 482 |
| | AAGCCCTCATTGATCAGGGA | 203 | AAGCCCUCAUUGAUCAGGGA | 483 |
| | AAATAAGCCCTCATTGATCA | 204 | AAAUAAGCCCUCAUUGAUCA | 484 |
| | ACAAAGGCGTGTTCATCAGG | 205 | ACAAAGGCGUGUUCAUCAGG | 485 |
| | ACCCCTCCCTGATCAATGAG | 206 | ACCCCUCCCUGAUCAAUGAG | 486 |
| | TCACCCCTCCCTGATCAATG | 207 | UCACCCCUCCCUGAUCAAUG | 487 |
| | GGCGGGGACGAAATCAGAGG | 208 | GGCGGGGACGAAAUCAGAGG | 488 |
| ESRRG | TTCTCCTCGGGTGGTTACGG | 209 | UUCUCCUCGGGUGGUUACGG | 489 |
| | GTCGCGGGAGCCCAGTTAAG | 210 | GUCGCGGGAGCCCAGUUAAG | 490 |
| | CCTCCCTCCTGATTGGGCAG | 211 | CCUCCCUCCUGAUUGGGCAG | 491 |
| | ATCAGGAGGGAGGCGACGGG | 212 | AUCAGGAGGGAGGCGACGGG | 492 |
| | CCTCTGCCCAATCAGGAGGG | 213 | CCUCUGCCCAAUCAGGAGGG | 493 |
| | AGGCCCCCCAATCGGCTGCG | 214 | AGGCCCCCCAAUCGGCUGCG | 494 |
| | CAGCCGCGCAGCCGATTGGG | 215 | CAGCCGCGCAGCCGAUUGGG | 495 |
| | CTTTACATATGCAGTCCCAC | 216 | CUUUACAUAUGCAGUCCCAC | 496 |
| | TAACCACCCGAGGAGAACCG | 217 | UAACCACCCGAGGAGAACCG | 497 |
| | AGGCTGCCAGGTTCTCCTCG | 218 | AGGCUGCCAGGUUCUCCUCG | 498 |
| ESRRB | AAAGTGACAGAATCAAGCTG | 219 | AAAGUGACAGAAUCAAGCUG | 499 |
| | CCCATGGTTCCAGAGTCGGG | 220 | CCCAUGGUUCCAGAGUCGGG | 500 |
| | GAGGGGCAACCCCCGACTCG | 221 | GAGGGGCAACCCCCGACUCG | 501 |
| | TGACAGAATCAAGCTGGGGT | 222 | UGACAGAAUCAAGCUGGGGU | 502 |
| | GTGACAGAATCAAGCTGGGG | 223 | GUGACAGAAUCAAGCUGGGG | 503 |
| | GGCCCTCAAACATGGAGAGG | 224 | GGCCCUCAAACAUGGAGAGG | 504 |
| | GACAGAATCAAGCTGGGGTG | 225 | GACAGAAUCAAGCUGGGGUG | 505 |
| | TTAAAGTGACAGAATCAAGC | 226 | UUAAAGUGACAGAAUCAAGC | 506 |
| | TAAAGTGACAGAATCAAGCT | 227 | UAAAGUGACAGAAUCAAGCU | 507 |
| | ACCCCCGACTCTGGAACCAG | 228 | ACCCCCGACUCUGGAACCAG | 508 |
| SMAD2 | CAGCCGCCCGTTAGGGCGCG | 229 | CAGCCGCCCGUUAGGGCGCG | 509 |
| | CACGCCGGCCGAAAGCCGCG | 230 | CACGCCGGCCGAAAGCCGCG | 510 |
| | TGCCTCGCGCCCTAACGGGG | 231 | UGCCUCGCGCCCUAACGGGG | 511 |
| | TCTTCCATACCCGCCCCGCG | 232 | UCUUCCAUACCCGCCCCGCG | 512 |
| | GTGCCTCGCGCCCTAACGGG | 233 | GUGCCUCGCGCCCUAACGGG | 513 |
| | GGGCGGGTATGGAAGACGGA | 234 | GGGCGGGUAUGGAAGACGGA | 514 |
| | AAGCCGCGCGGGGGGGGTAG | 235 | AAGCCGCGCGGGGGGGGUAG | 515 |
| | GGCGGGTATGGAAGACGGAG | 236 | GGCGGGUAUGGAAGACGGAG | 516 |
| | AGGCCGAGCACCGCGGGAAG | 237 | AGGCCGAGCACCGOGGGAAG | 517 |
| | CGCGGGGGGGGTATGGAAGA | 238 | CGCGGGGGGGGUAUGGAAGA | 518 |
| KLF6 | GGGCGAGTCACGTGGGGGGG | 239 | GGGCGAGUCACGUGGGGGGG | 519 |
| | GCGGGGCGAGTCACGTGGGG | 240 | GCGGGGCGAGUCACGUGGGG | 520 |
| | CGCGGGGCGAGTCACGTGGG | 241 | CGCGGGGCGAGUCACGUGGG | 521 |
| | GGGCGCGGGGCGAGTCACGT | 242 | GGGCGCGGGGCGAGUCACGU | 522 |
| | TGACCGAGACTCTCCATCAG | 243 | UGACCGAGACUCUCCAUCAG | 523 |
| | CGCAGGCGGCGACACACAGG | 244 | CGCAGGCGGCGACACACAGG | 524 |
| | ATCCCGTGGCTCCCGGCCCG | 245 | AUCCCGUGGCUCCCGGCCCG | 525 |
| | ACGGGATTGGGCCGCGCACG | 246 | ACGGGAUUGGGCCGCGCACG | 526 |
| | TCCCGTGGCTCCCGGCCCGG | 247 | UCCCGUGGCUCCCGGCCCGG | 527 |
| | CCGTGCGCGGCCCAATCCCG | 248 | CCGUGCGCGGCCCAAUCCCG | 528 |
| TCF7 | GAGGCCGGGACGCGCCCACT | 249 | GAGGCCGGGACGCGCCCACU | 529 |
| | CTCTCCCGGGAGAGGAGAGG | 250 | CUCUCCCGGGAGAGGAGAGG | 530 |
| | GTCTCTCTCAGGGTGCGCTC | 251 | GUCUCUCUCAGGGUGCGCUC | 531 |
| | CGGAAGAGCGAGCCCTTTAG | 252 | CGGAAGAGCGAGCCCUUUAG | 532 |
| | CTCCCGGGAGAGGAGAGGCG | 253 | CUCCCGGGAGAGGAGAGGCG | 533 |
| | CCCTTTAAGGCTATGCCGAG | 254 | CCCUUUAAGGCUAUGCCGAG | 534 |
| | GACCCCCGCCCTCCACAGCG | 255 | GACCCCCGCCCUCCACAGCG | 535 |
| | CCTTTAAGGCTATGCCGAGT | 256 | CCUUUAAGGCUAUGCCGAGU | 536 |
| | AGGCCGGGACGCGCCCACTG | 257 | AGGCCGGGACGCGCCCACUG | 537 |
| | CCACTCGGCATAGCCTTAAG | 258 | CCACUCGGCAUAGCCUUAAG | 538 |
| SMARCA2 | GAACCCGCGCCCGGACGGGC | 259 | GAACCCGCGCCCGGACGGGC | 539 |
| | GGGAGCCCGAGTTTAGGAAG | 260 | GGGAGCCCGAGUUUAGGAAG | 540 |
| | GCGTCTTCCGGCGCCCGCGG | 261 | GCGUCUUCCGGCGCCCGCGG | 541 |
| | GCCAACTGCGGCGCGTCTTC | 262 | GCCAACUGCGGCGCGUCUUC | 542 |
| | GCGCCAACCCGCGCCCGGAC | 263 | GCGCCAACCCGCGCCCGGAC | 543 |
| | GTCGCCTCCTCCGCGGGGGC | 264 | GUCGCCUCCUCCGCGGGCGC | 544 |
| | GCCCGAGTTTAGGAAGAGGA | 265 | GCCCGAGUUUAGGAAGAGGA | 545 |
| | GCTCCCTGCCCGTCCGGGCG | 266 | GCUCCCUGCCCGUCCGGGCG | 546 |
| | GCCGCTCCTCCCTGCCCGTC | 267 | GCCGCUCCUCCCUGCCCGUC | 547 |
| | GGCGCCGCGGGGCTCCTCCA | 268 | GGCGCCGCGGGGCUCCUCCA | 548 |
| SMAD3 | TCTGCGCATCAAAGCTAGCG | 269 | UCUGCGCAUCAAAGCUAGCG | 549 |
| | AGAGGGGCCTCGGGCGGGAG | 270 | AGAGGGGCCUCGGGGGGGAG | 550 |
| | ACCCGGGCGGCGGCAGTCCG | 271 | ACCCGGGCGGCGGCAGUCCG | 551 |
| | CCCCGCCACCCTCCTCCTCC | 272 | CCCCGCCACCCUCCUCCUCC | 552 |
| | CCGCCCGGGTCGCCCACGTG | 273 | CCGCCCGGGUCGCCCACGUG | 553 |
| | CGGGAGGAGGAGGGTGGCGG | 274 | CGGGAGGAGGAGGGUGGCGG | 554 |
| | GGTGAGGCCGCAGAGGCGGA | 275 | GGUGAGGCCGCAGAGGCGGA | 555 |
| | CGCAGATCCCTCCGCCTCTG | 276 | CGCAGAUCCCUCCGCCUCUG | 556 |
| | GCGGGGCGGTGAGGCCGCAG | 277 | GCGGGGCGGUGAGGCCGCAG | 557 |
| | CCGGGAGGAGGAGGGTGGCG | 278 | CCGGGAGGAGGAGGGUGGCG | 558 |
| TFEC | AAAAGAGAAGTGGCTCATTG | 279 | AAAAGAGAAGUGGCUCAUUG | 559 |
| | GTGCTCTTTCTCAAATTAGG | 280 | GUGCUCUUUCUCAAAUUAGG | 560 |
| | TTAGGGCATTTCCTGAACCC | 281 | UUAGGGCAUUUCCUGAACCC | 561 |
| | GGTTGGAATCCAGTTAGTTG | 282 | GGUUGGAAUCCAGUUAGUUG | 562 |
| | GTTAATTATGGAGCCACAAG | 283 | GUUAAUUAUGGAGCCACAAG | 563 |
| | GGCATTTCCTGAACCCTGGT | 284 | GGCAUUUCCUGAACCCUGGU | 564 |
| | CTTAACTGAAATTCCAACCA | 285 | CUUAACUGAAAUUCCAACCA | 565 |
| | CCTTAACTGAAATTCCAACC | 286 | CCUUAACUGAAAUUCCAACC | 566 |
| | CCTGGTTGGAATTTCAGTTA | 287 | CCUGGUUGGAAUUUCAGUUA | 567 |
| | CCAACAAGAGCTTTTACTGG | 288 | CCAACAAGAGCUUUUACUGG | 568 |
| PURB | GcgggcggcAAGCTGAGAGT | 289 | GcgggcggcAAGCUGAGAGU | 569 |
| | GCGCAGAGGGGTGACGTCAg | 290 | GCGCAGAGGGGUGACGUCAg | 570 |
| | GTAAGGCTGGCAAGTCCCCA | 291 | GUAAGGCUGGCAAGUCCCCA | 571 |
| | GGGGCGGCTGAGTCACGCTA | 292 | GGGGCGGCUGAGUCACGCUA | 572 |
| | GGGCGCGTAGACAGACGCAG | 293 | GGGCGCGUAGACAGACGCAG | 573 |
| | GCGCGCGCCGGAAGTGGCTA | 294 | GCGCGCGCCGGAAGUGGCUA | 574 |
| | GGCCCCGGAAGCGAGAAGGA | 295 | GGCCCCGGAAGCGAGAAGGA | 575 |
| | GCTCGCGGCCGGAAGCGCCG | 296 | GCUCGCGGCCGGAAGCGCCG | 576 |
| | GGCGGGACGGAGTGACTTCA | 297 | GGCGGGACGGAGUGACUUCA | 577 |
| | GCGCGGCGGCGTGGCGCAGA | 298 | GCGCGGCGGCGUGGCGCAGA | 578 |
| ZNF398 | CACCGAAGCCCAAGAGCTGG | 299 | CACCGAAGCCCAAGAGCUGG | 579 |
| | TGTGGGGAGGGTTGTTATTG | 300 | UGUGGGGAGGGUUGUUAUUG | 580 |
| | GTGAGCTGAACCTTCTCGTG | 301 | GUGAGCUGAACCUUCUCGUG | 581 |
| | ACCGAAGCCCAAGAGCTGGG | 302 | ACCGAAGCCCAAGAGCUGGG | 582 |
| | GCGCCCCCTGCCGGCCCCTG | 303 | GCGCCCCCUGCCGGCCCCUG | 583 |
| | CCGAAGCCCAAGAGCTGGGG | 304 | CCGAAGCCCAAGAGCUGGGG | 584 |
| | CCAGCTCTTGGGCTTCGGTG | 305 | CCAGCUCUUGGGCUUCGGUG | 585 |
| | CACACCGAAGCCCAAGAGCT | 306 | CACACCGAAGCCCAAGAGCU | 586 |
| | AGTGCACCGGGAACCGCGCG | 307 | AGUGCACCGGGAACCGCGCG | 587 |
| | AGCTCTTGGGCTTCGGTGTG | 308 | AGCUCUUGGGCUUCGGUGUG | 588 |
| ASCL2 | CAAACGGGTGGCCCACGATG | 309 | CAAACGGGUGGCCCACGAUG | 589 |
| | CCAGGGTCTCAGCCAATCGT | 310 | CCAGGGUCUCAGCCAAUCGU | 590 |
| | CCAAACGGGTGGCCCACGAT | 311 | CCAAACGGGUGGCCCACGAU | 591 |
| | GAGCCGCGCCCTGCGCGATG | 312 | GAGCCGCGCCCUGCGCGAUG | 592 |
| | GCGTACGCGCTTCCTCAATG | 313 | GCGUACGCGCUUCCUCAAUG | 593 |
| | TGCGCGATTGGCCAAACGGG | 314 | UGCGCGAUUGGCCAAACGGG | 594 |
| | CGTACGCGCTTCCTCAATGG | 315 | CGUACGCGCUUCCUCAAUGG | 595 |
| | GCGCCCAGCCCAGTCGCGGG | 316 | GCGCCCAGCCCAGUCGCGGG | 596 |
| | GCCAGGGTCTCAGCCAATCG | 317 | GCCAGGGUCUCAGCCAAUCG | 597 |
| | CCCGTTTGGCCAATCGCGCA | 318 | CCCGUUUGGCCAAUCGCGCA | 598 |
| NFIB | CATGGTTCTGTAGCACCGGT | 319 | CAUGGUUCUGUAGCACCGGU | 599 |
| | AGCTCGAGCGGGGTCCAGAG | 320 | AGCUCGAGCGGGGUCCAGAG | 600 |
| | CCTCTCCCTCATTTGTTAAG | 321 | CCUCUCCCUCAUUUGUUAAG | 601 |
| | CACCGAAATACCTCTTCCAG | 322 | CACCGAAAUACCUCUUCCAG | 602 |
| | AGGAGGAAGGTGGGGGGGCG | 323 | AGGAGGAAGGUGGGGGGGCG | 603 |
| | GAATGCGCGTTTGCAGGAGG | 324 | GAAUGCGCGUUUGCAGGAGG | 604 |
| | TTCCAGGGCGGGTTTTCGGG | 325 | UUCCAGGGCGGGUUUUCGGG | 605 |
| | TGTGGAGAGGCTGGTGCAAA | 326 | UGUGGAGAGGCUGGUGCAAA | 606 |
| | AAGGAATGCGCGTTTGCAGG | 327 | AAGGAAUGCGCGUUUGCAGG | 607 |
| | ATTTGTTAAAGGTATCCTAC | 328 | AUUUGUUAAAGGUAUCCUAC | 608 |
| ZIC4 | GCCGTGAGCTCCCCGATTGG | 329 | GCCGUGAGCUCCCCGAUUGG | 609 |
| | CCCCGATTGGGGCTCGGTCG | 330 | CCCCGAUUGGGGCUCGGUCG | 610 |
| | GCCCCAATCGGGGAGCTCAG | 331 | GCCCCAAUCGGGGAGCUCAG | 611 |
| | TGAGCTCCCCGATTGGGGCT | 332 | UGAGCUCCCCGAUUGGGGCU | 612 |
| | GGCCGTGAGCTCCCCGATTG | 333 | GGCCGUGAGCUCCCCGAUUG | 613 |
| | GGCCGGACCGAGCCCCAATG | 334 | GGCCGGACCGAGCCCCAAUG | 614 |
| | AGCCCCAATCGGGGAGCTCA | 335 | AGCCCCAAUCGGGGAGCUCA | 615 |
| | AATTCGCTGACGTGTAGGAG | 336 | AAUUCGCUGACGUGUAGGAG | 616 |
| | TACACGTCAGCGAATTCAGC | 337 | UACACGUCAGCGAAUUCAGC | 617 |
| | GCCGGACCGAGCCCCAATCG | 338 | GCCGGACCGAGCCCCAAUCG | 618 |
| ZNF618 | CTCGGCGGCAGCGGCTGCCG | 339 | CUCGGCGGCAGCGGCUGCCG | 619 |
| | CCCCCACCCCCGGCCCGCAC | 340 | CCCCCACCCCCGGCCCGCAC | 620 |
| | GAGGGGCGGCTCGGCGGCAG | 341 | GAGGGGCGGCUCGGCGGCAG | 621 |
| | GGGGCGCGCGGGGGGGGCGC | 342 | GGGGCGCGCGGGGGGGGCGC | 622 |
| | TGCCGCGGCGCGGCCGGTGC | 343 | UGCCGCGGCGCGGCCGGUGC | 623 |
| | CGGCAGCGGCTGCCGCGGCG | 344 | CGGCAGCGGCUGCCGCGGCG | 624 |
| | CGGAACGCAAGAGGTTAATG | 345 | CGGAACGCAAGAGGUUAAUG | 625 |
| | CCCGCGAGGGGCGGCTCGGG | 346 | CCCGCGAGGGGCGGCUCGGG | 626 |
| | GCCGCCGAGCCGCCCCTCGG | 347 | GCCGCCGAGCCGCCCCUCGG | 627 |
| | TCAGGGAGCCGGCTCTCGGG | 348 | UCAGGGAGCCGGCUCUCGGG | 628 |
| FOXA2 | GAGGGAGCGCGAGAGAGGGA | 349 | GAGGGAGCGCGAGAGAGGGA | 629 |
| | AGAGGGAGCGCGAGAGAGGG | 350 | AGAGGGAGCGCGAGAGAGGG | 630 |
| | CTCTCGCGCTCCCTCTCCCT | 351 | CUCUCGCGCUCCCUCUCCCU | 631 |
| | GGAGGCGGTAGTTGGAAGTG | 352 | GGAGGCGGUAGUUGGAAGUG | 632 |
| | ATGGGCTGCCCCGGGTCTGG | 353 | AUGGGCUGCCCCGGGUCUGG | 633 |
| | CAGGCCAGCGCCCCGCGGTG | 354 | CAGGCCAGCGCCCCGCGGUG | 634 |
| | CGGCAGCCCGTGAGCGCGGG | 355 | CGGCAGCCCGUGAGCGCGGG | 635 |
| | GGGAGGAGGGGACGGTGCTT | 356 | GGGAGGAGGGGACGGUGCUU | 636 |
| | CGGAGGCGGTAGTTGGAAGT | 357 | CGGAGGCGGUAGUUGGAAGU | 637 |
| | TGATTGCTGGTCGTTTGTTG | 358 | UGAUUGCUGGUCGUUUGUUG | 638 |
| NR5A1 | AAGAGAAACACCAACAAAGA | 359 | AAGAGAAACACCAACAAAGA | 639 |
| | AGGCCTGCAGAGTCACGTGG | 360 | AGGCCUGCAGAGUCACGUGG | 640 |
| | ACACCAACAAAGAAGGCGAG | 361 | ACACCAACAAAGAAGGCGAG | 641 |
| | AGAGGCCTGCAGAGTCACGT | 362 | AGAGGCCUGCAGAGUCACGU | 642 |
| | TGCCCCCACGTGACTCTGCG | 363 | UGCCCCCACGUGACUCUGCG | 643 |
| | GGCCTGCAGAGTCACGTGGG | 364 | GGCCUGCAGAGUCACGUGGG | 644 |
| | AATACCCCTATCTATCTGGG | 365 | AAUACCCCUAUCUAUCUGGG | 645 |
| | AGAGAAACACCAACAAAGAG | 366 | AGAGAAACACCAACAAAGAG | 646 |
| | GAGAGGCCTGCAGAGTCACG | 367 | GAGAGGCCUGCAGAGUCACG | 647 |
| | GAGGCCTGCAGAGTCACGTG | 368 | GAGGCCUGCAGAGUCACGUG | 648 |
| FOXA3 | GGGCGGCGCGCTCGGACTGG | 369 | GGGCGGCGCGCUCGGACUGG | 649 |
| | ATCGCCAGGAGACAGACCTC | 370 | AUCGCCAGGAGACAGACCUC | 650 |
| | AGCCCCAGGACGCGACAGCG | 371 | AGCCCCAGGACGCGACAGCG | 651 |
| | AGACAGACCTCAGGCGCCGG | 372 | AGACAGACCUCAGGCGCCGG | 652 |
| | CCCTCCCGCCTCCCCGAGGG | 373 | CCCUCCCGCCUCCCCGAGGG | 653 |
| | GCTCCCCTCCCGCCTCCCCG | 374 | GCUCCCCUCCCGCCUCCCCG | 654 |
| | AGACAGACCTCAGGCGCCGG | 375 | AGACAGACCUCAGGCGCCGG | 655 |
| | GTCTCCTGGCGATCCCGCAG | 376 | GUCUCCUGGCGAUCCCGCAG | 656 |
| | GAGACAGACCTCAGGCGCCG | 377 | GAGACAGACCUCAGGCGCCG | 657 |
| | GGGGGTGATTCAGGAGTGAG | 378 | GGGGGUGAUUCAGGAGUGAG | 658 |
| HNF4A | GCCTGACTTGGGGTGACAAG | 379 | GCCUGACUUGGGGUGACAAG | 659 |
| | GTGCTAATTACAACTGCTGG | 380 | GUGCUAAUUACAACUGCUGG | 660 |
| | GGGAACCGGGAAACTGCGGG | 381 | GGGAACCGGGAAACUGCGGG | 661 |
| | GTTAATCGGTCCCCCGCCGG | 382 | GUUAAUCGGUCCCCCGCCGG | 662 |
| | CCAGCCTATCCACCGGCGGG | 383 | CCAGCCUAUCCACCGGGGGG | 663 |
| | CCGCCGGTGGATAGGCTGGG | 384 | CCGCCGGUGGAUAGGCUGGG | 664 |
| | CCGCCCAGCCTATCCACCGG | 385 | CCGCCCAGCCUAUCCACCGG | 665 |
| | GCCCCGCCCAGCCTATCCAC | 386 | GCCCCGCCCAGCCUAUCCAC | 666 |
| | ATGGTTAATCGGTCCCCCGC | 387 | AUGGUUAAUCGGUCCCCCGC | 667 |
| | GCCCAGCCTATCCACCGGCG | 388 | GCCCAGCCUAUCCACCGGCG | 668 |
| HNF4G | ACAGGGAAATGCGCGCGCCG | 389 | ACAGGGAAAUGCGCGCGCCG | 669 |
| | GGGAAATGCGCGCGCCGAGG | 390 | GGGAAAUGCGCGCGCCGAGG | 670 |
| | GCCCCGCGCTGGACCGCTCG | 391 | GCCCCGCGCUGGACCGCUCG | 671 |
| | TCCCTGTTCCCAAGGCCCCG | 392 | UCCCUGUUCCCAAGGCCCCG | 672 |
| | TCCCTGTTCCCAAGGCCCCA | 393 | UCCCUGUUCCCAAGGCCCCA | 673 |
| | AATGAAAACTGAAAGCAGCG | 394 | AAUGAAAACUGAAAGCAGCG | 674 |
| | CCCGACCGCCAGTCGCCCTG | 395 | CCCGACCGCCAGUCGCCCUG | 675 |
| | GGGCTCCCCAGGGTGTAGGG | 396 | GGGCUCCCCAGGGUGUAGGG | 676 |
| | CAAGGCCCCAGGGCGACTGG | 397 | CAAGGCCCCAGGGCGACUGG | 677 |
| | CGGCCTCCCGAGACCGCAGG | 398 | CGGCCUCCCGAGACCGCAGG | 678 |
| HBP1 | GGACCACGGTACCACCCAAT | 399 | GGACCACGGUACCACCCAAU | 679 |
| | GGTACCACCCAATGGGAAAG | 400 | GGUACCACCCAAUGGGAAAG | 680 |
| | GACCACGGTACCACCCAATG | 401 | GACCACGGUACCACCCAAUG | 681 |
| | GTACCACCCAATGGGAAAGG | 402 | GUACCACCCAAUGGGAAAGG | 682 |
| | TCTTCCCCTTTCCCATTGGG | 403 | UCUUCCCCUUUCCCAUUGGG | 683 |
| | CGGACCACGGTACCACCCAA | 404 | CGGACCACGGUACCACCCAA | 684 |
| | GGAAAGGGGAAGAACCGAAG | 405 | GGAAAGGGGAAGAACCGAAG | 685 |
| | TGTCCCCGAAATGGATTGTG | 406 | UGUCCCCGAAAUGGAUUGUG | 686 |
| | ACGGTACCACCCAATGGGAA | 407 | ACGGUACCACCCAAUGGGAA | 687 |
| | TTTCCCATTGGGTGGTACCG | 408 | UUUCCCAUUGGGUGGUACCG | 688 |
| NHLH1 | CCCCGCCCCCAGCTCCCGAG | 409 | CCCCGCCCCCAGCUCCCGAG | 689 |
| | TCACGCTGGGACTCCGAATG | 410 | UCACGCUGGGACUCCGAAUG | 690 |
| | CTGTTTAATATTGATGAGGG | 411 | CUGUUUAAUAUUGAUGAGGG | 691 |
| | AGGGATCTGGGAACCCCTCG | 412 | AGGGAUCUGGGAACCCCUCG | 692 |
| | GGCTGTTTAATATTGATGAG | 413 | GGCUGUUUAAUAUUGAUGAG | 693 |
| | CACACCCCCAGCTCTCCATG | 414 | CACACCCCCAGCUCUCCAUG | 694 |
| | GCTGTTTAATATTGATGAGG | 415 | GCUGUUUAAUAUUGAUGAGG | 695 |
| | CCAGCCAATGGAGAGCTGGG | 416 | CCAGCCAAUGGAGAGCUGGG | 696 |
| | CTCATCAATATTAAACAGCC | 417 | CUCAUCAAUAUUAAACAGCC | 697 |
| | GCCAGGCAGCCATTGGCAGA | 418 | GCCAGGCAGCCAUUGGCAGA | 698 |
| NR5A2 | ACTCTATGCAGGTTGATAAG | 419 | ACUCUAUGCAGGUUGAUAAG | 699 |
| | AAAGAGGTGATGACATCAAG | 420 | AAAGAGGUGAUGACAUCAAG | 700 |
| | CTCATCACATGACTCTATGC | 421 | CUCAUCACAUGACUCUAUGC | 701 |
| | TGATTAACTCAGAAGTGGAG | 422 | UGAUUAACUCAGAAGUGGAG | 702 |
| | TCATCACATGACTCTATGCG | 423 | UCAUCACAUGACUCUAUGCG | 703 |
| | TGCTATGCAGCACAGAGAGG | 424 | UGCUAUGCAGCACAGAGAGG | 704 |
| | CCATGTATCCAGTTTCATGA | 425 | CCAUGUAUCCAGUUUCAUGA | 705 |
| | ATGGTTTCTAATCAGAAGAA | 426 | AUGGUUUCUAAUCAGAAGAA | 706 |
| | TGGAAAGTTGTAAAACTGAG | 427 | UGGAAAGUUGUAAAACUGAG | 707 |
| | AGAAATGATTAACTCAGAAG | 428 | AGAAAUGAUUAACUCAGAAG | 708 |
| YAF2 | GTGGCCGCGTTTGAAGTCTC | 429 | GUGGCCGCGUUUGAAGUCUC | 709 |
| | GGCAGCCGGGGGTACCAAGG | 430 | GGCAGCCGGGGGUACCAAGG | 710 |
| | GGCTGCTGGTTGGCCGGCGA | 431 | GGCUGCUGGUUGGCCGGCGA | 711 |
| | GcccgccggcTAACTCGCCG | 432 | GcccgceggQUAACUCGCCG | 712 |
| | GCCCGCGGCGAGTTAgccgg | 433 | GCCCGCGGCGAGUUAgccgg | 713 |
| | GGCGCTCCAGAGCCCCTCTG | 434 | GGCGCUCCAGAGCCCCUCUG | 714 |
| | GGCGCCGGAGACTTCAAACG | 435 | GGCGCCGGAGACUUCAAACG | 715 |
| | GGGTACCAAGGGGGCAACGG | 436 | GGGUACCAAGGGGGCAACGG | 716 |
| | GGGGCGCACGTGACCCTCGC | 437 | GGGGCGCACGUGACCCUCGC | 717 |
| | GGGGTAGCTAAGGACAGCGG | 438 | GGGGUAGCUAAGGACAGCGG | 718 |

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

### vi) Repair Pathways

The CRISPR/Cas9-based gene editing system may be used to introduce site-specific double strand breaks at targeted genomic loci, such as a gene for promoting differentiation of a cell into a hepatocyte or maturation of a hepatocyte, as detailed herein. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

### (1) Homology-Directed Repair (HDR)

Restoration of protein expression from a gene may involve homology-directed repair (HDR). A donor template may be administered to a cell. A donor sequence comprises a polynucleotide sequence to be inserted into a genome. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

### (2) Non-Homologous End Joining (NHEJ)

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 3. Genetic Constructs

The CRISPR/Cas9-based gene editing system may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as polynucleotide encoding a reporter protein and/or a selectable marker, such as hygromycin (“Hygro”). The reporter protein may include any protein or peptide that is suitably detectable, such as, by fluorescence, chemiluminescence, enzyme activity such as beta galactosidase or alkaline phosphatase, and/or antibody binding detection. The reporter protein may comprise a fluorescent protein. The reporter protein may comprise a protein or peptide detectable with an antibody. For example, the reporter protein may comprise green fluorescent protein (“GFP”), YFP, RFP, CFP, DsRed, luciferase, and/or Thy1.

The genetic construct may be useful for transfecting cells with a nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (IPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV218G9 (Shen et al. J. Biol. Chem. 2013, 288, 28814-28823).

### 4. PHARMACEUTICAL COMPOSITIONS

Further provided herein are pharmaceutical compositions comprising the above-described modulators or genetic constructs or gene editing systems. In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 5. ADMINISTRATION

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid: nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector Ilb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed modulators, a variety of effects may be elicited, such as, for example, an induced pluripotent stem cell (IPSC) may be induced or promoted to differentiate into a hepatocyte, or an embryonic stem cell (ESC) may be induced or promoted to differentiate into a hepatocyte, or a hepatocyte may be induced or promoted to mature, or a combination thereof. Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (TEs). The cell may be a stem cell such as a human stem cell. In some embodiments, the cell is an embryonic stem cell (ESC) or a hematopoietic stem cell. The stem cell may be a human induced pluripotent stem cell (IPSCs). Further provided are stem cell-derived neurons, such as neurons derived from IPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. In some embodiments, the cell is an iPSC. In some embodiments, the cell is an ESC. In some embodiments, the cell is a hepatocyte. In some embodiments, the cell is an IPSC-derived hepatocyte (iHep). In some embodiments, the cell is an ESC-derived hepatocyte.

### 6. KITS

Provided herein is a kit, which may be used to induce or promote differentiation of a cell into a hepatocyte and/or maturation of a hepatocyte. The kit comprises genetic constructs or a composition comprising the same, as described above, and instructions for using said composition. The kit includes a modulator of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2. In some embodiments, the modulator is an inhibitor. In some embodiments, the modulator is an activator. In some embodiments, the kit comprises at least one polynucleotide sequence selected from SEQ ID NOs: 83-120 or 159-718, a complement thereof, a variant thereof, or fragment thereof. In some embodiments, the kit comprises at least one polypeptide sequence selected from SEQ ID NOs: 121-158, a variant thereof, or fragment thereof. In some embodiments, the kit comprises at least one gRNA comprising a polynucleotide sequence selected from SEQ ID NOs: 439-718, a complement thereof, a variant thereof, or fragment thereof, or gRNA targeting or encoded by a polynucleotide comprising a sequence selected from SEQ ID NOs: 159-438, a complement thereof, a variant thereof, or fragment thereof. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof may include a modified AAV vector that includes a gRNA molecule(s) and a Cas9 protein or fusion protein, as described above, that specifically binds a region of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a regulatory element thereof.

### 7. METHODS

a. Methods of Inducing Differentiation of a Cell into a Hepatocyte

Provided herein are methods of inducing differentiation of an induced pluripotent stem cell (iPSC) or an embryonic stem cell (ESC) into a hepatocyte. The methods may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof. In some embodiments, the composition modulates gene expression within the IPSC or the ESC or the hepatocyte. In some embodiments, the composition increases expression of albumin, alpha-1 antitrypsin, ASGR1, SERPINA1, Foxa2, a p450 enzyme, or Hnf4a, or a combination thereof, in the IPSC or the ESC or the iPSC-derived hepatocyte or the ESC-derived hepatocyte.

b. Methods of Promoting Maturation of a Hepatocyte

Provided herein are methods of promoting maturation of a hepatocyte. The methods may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof. In some embodiments, the composition modulates gene expression within the IPSC or the ESC or the hepatocyte. In some embodiments, the composition increases expression of albumin, alpha-1 antitrypsin, ASGR1, SERPINA1, Foxa2, a p450 enzyme, or Hnf4a, or a combination thereof, in the IPSC or the ESC or the IPSC-derived hepatocyte or the ESC-derived hepatocyte.

### 8. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

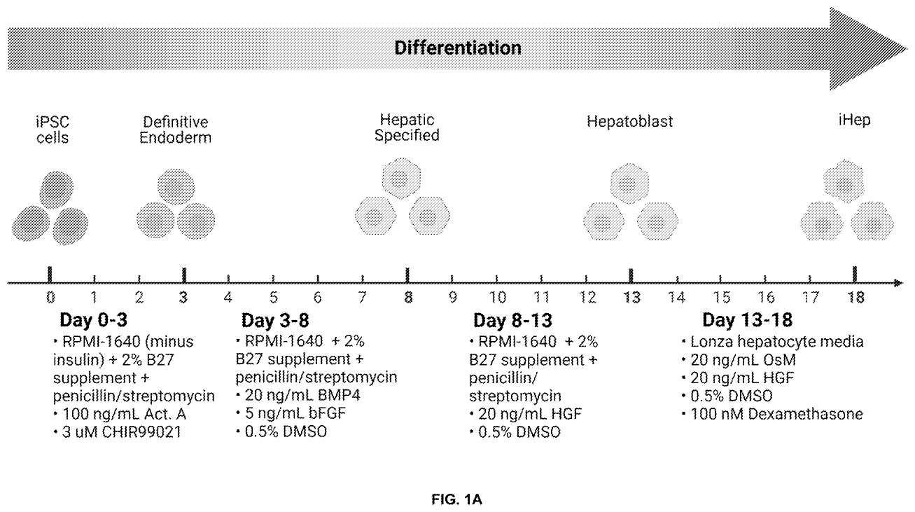

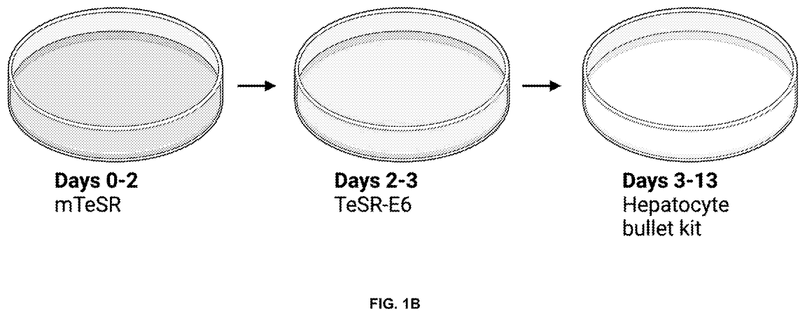

iHep Characterization. iHeps were generated using the protocol shown in FIG. 1A. To assess transcriptional differences between iHeps and PHHs, RNAseq profiles were generated for IPSCs, IHeps, and PHHs. In addition, a 10× Genomics Multiomic RNA and ATACseq profile was generated for the iHeps to understand heterogeneity and TFs driving maturity (FIG. 1B).

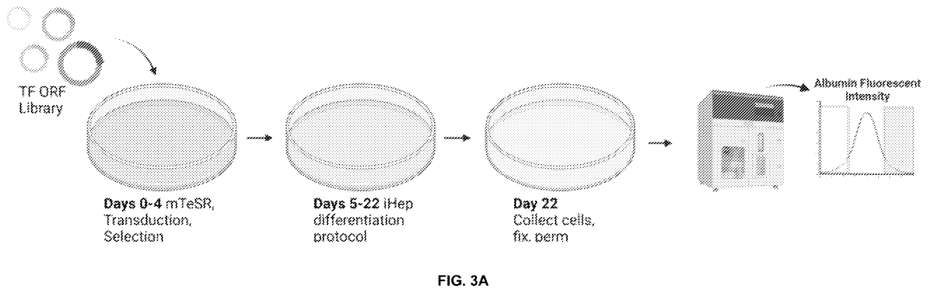

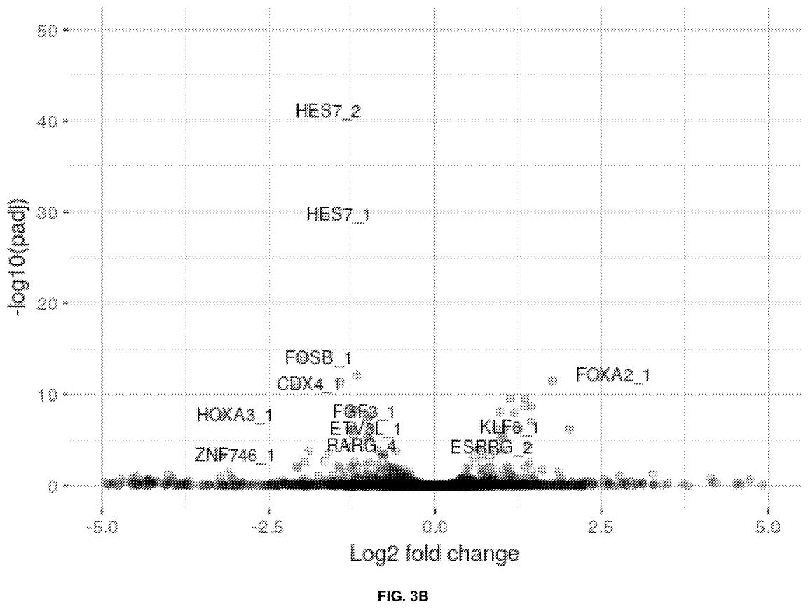

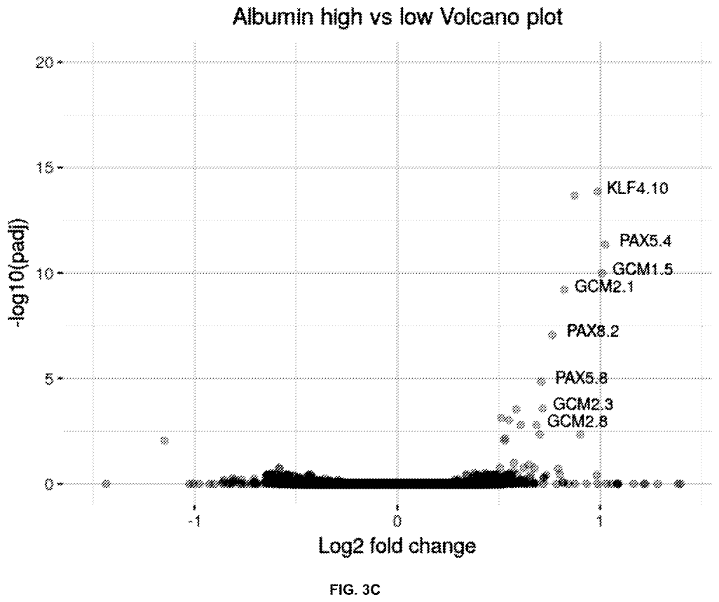

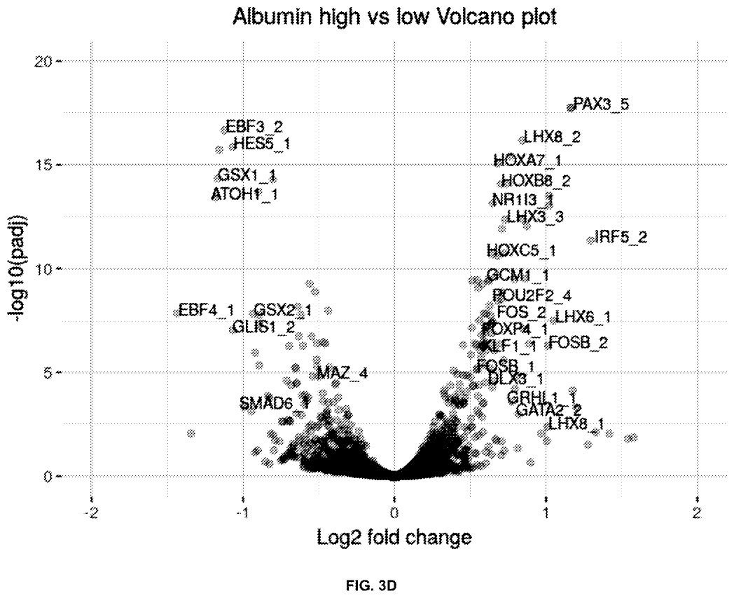

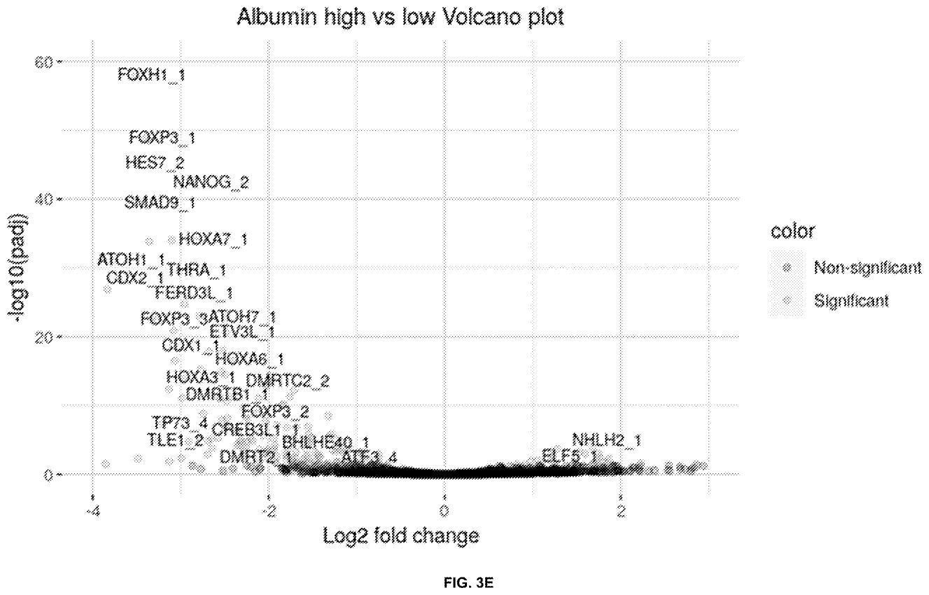

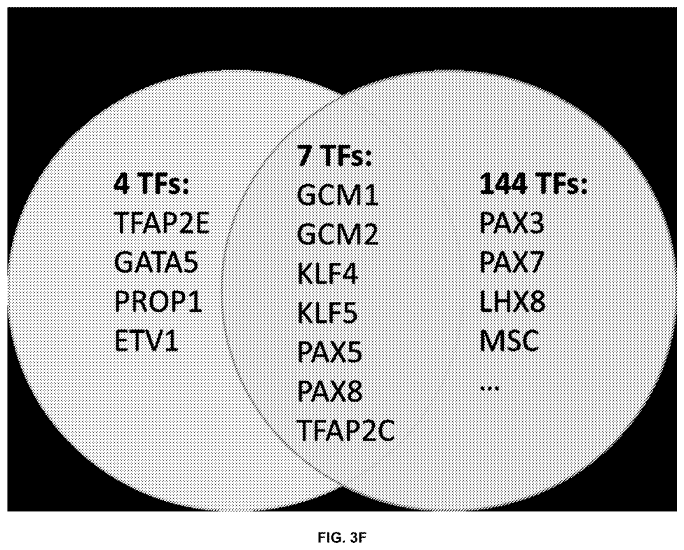

In Vitro Albumin Screens. To screen for iHep maturity, WTC11 IPSCs stably expressing dCas9-2XVP64 (VP64-dCas9-VP64) were lentivirally transduced at a low MOI with a TF CRISPRa library (TABLE 4), or WTC11 IPSCs were lentivirally transduced at a low MOI with a TF cDNA overexpression library (TABLE 5; Joung, J. et al. Cell 2023, 186, 209-229, incorporated herein by reference). Following transduction, cells were cultured using the protocol shown in FIGS. 1A-1B and FIG. 3A and sorted at day 13 on albumin staining (Tomaz, R. A. et al. eLife 2022, 11, e71591, incorporated herein by reference). The cells from the top and bottom 10% bins were sequenced for gRNA abundance.

TABLE 4
CRISPRa Library.

| | |
|---|---|
| TFs, Ems | 1,905 |
| gRNAs per TSS | 10 |
| Non-target | 150 |
| Total | 19,200 |

TABLE 5
ORF Library.

| | |
|---|---|
| TFs | 1,836 |
| ORFs | 3,548 |

### Example 2

### Assess the Current Deficiencies in iHep Protocols and Use Transcription Factor (TF) Screening to Determine a Combination of TFs that Improves iHep Maturity and Function

Due to the shortage of liver donors for transplantations and the scarcity of primary human hepatocytes for in vitro toxicology studies, there is a growing interest in the in vitro reprogramming of induced pluripotent stem cells (IPSCs) to mature hepatocytes using chemical and genetic methods, termed iPSC-derived hepatocytes (iHeps). However, current methods for producing iHeps yield cells with significant differences in gene expression and epigenetic signatures compared to primary human hepatocytes (PHHs). Further, the generated single-cell multiomic RNA- and ATAC-seq profile of IHeps herein indicates substantial transcriptomic and epigenetic heterogeneity within the final cell population. These epigenetic differences, particularly in relation to transcription factor promoter accessibility, likely contribute to the reported deficiencies in using IHeps. Epigenetic editing using CRISPR-dCas9 allows the precise dissection and programming of intricate cellular pathways while cDNA overexpression gives insight into specific isoforms and their downstream effect on epigenetic state. To address the limitations of current protocols for iHep generation, CRISPR activation and cDNA overexpression screens of transcription factors and epigenetic modifier genes were used herein.

Fluorescence-activated cell sorting (FACS) for cells that upregulate albumin generated a list of guide RNAs (gRNAs) and open reading frames (ORFs) putatively implicated in mature liver phenotypes. Several independent gRNA hits from the screens target the same transcription factor promoters while independent ORFs from the screen are transcribed from the same transcription factors, strongly implicating these transcription factors in iHep differentiation and/or maturity. Validation experiments, including scRNAseq of screen hits, have confirmed the ability of screen hits to upregulate the expression of albumin along with other hepatocyte maturation markers such as alpha-1 antitrypsin and Hnf4a.

The following 30 genes are of particular interest: KLF7, HNF4A, FOXA2, PPARG, HNF4G, TCF7, GSC2, YAF2, KLF6, SMAD2, SMARCA2, NR5A2, ESRRA, ESRRG, FOXA3, ZNF398, SOX17, SALL4, GATA4, NFIB, ASCL2, HBP, TFEC, NR5A1, NHLH2, SMAD3, NHLH1, ESRRB, ZNF618, and ZIC4.

iHeps were generated using the protocol shown in FIG. 1A and the IHeps were characterized as shown in FIG. 1B. To assess transcriptional differences between iHeps and PHHs, qPCR was performed for various markers and RNAseq profiles were generated for iPSCs, iHeps, and PHHS.

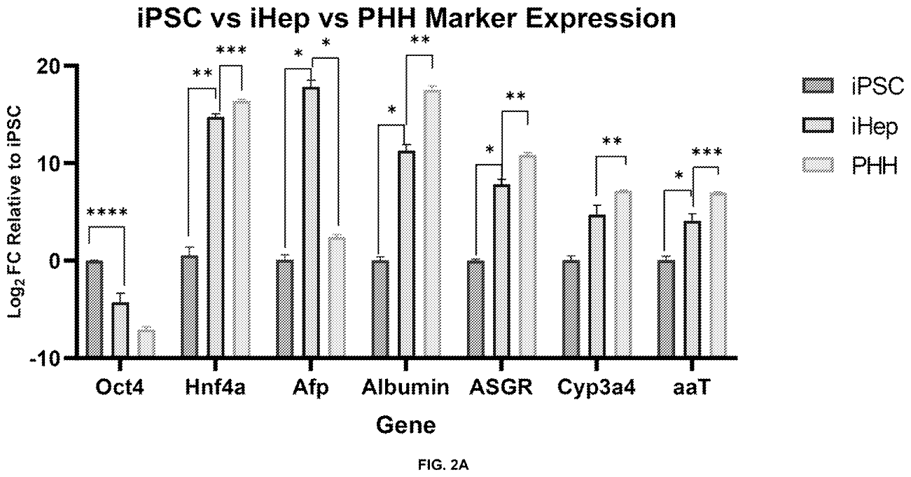

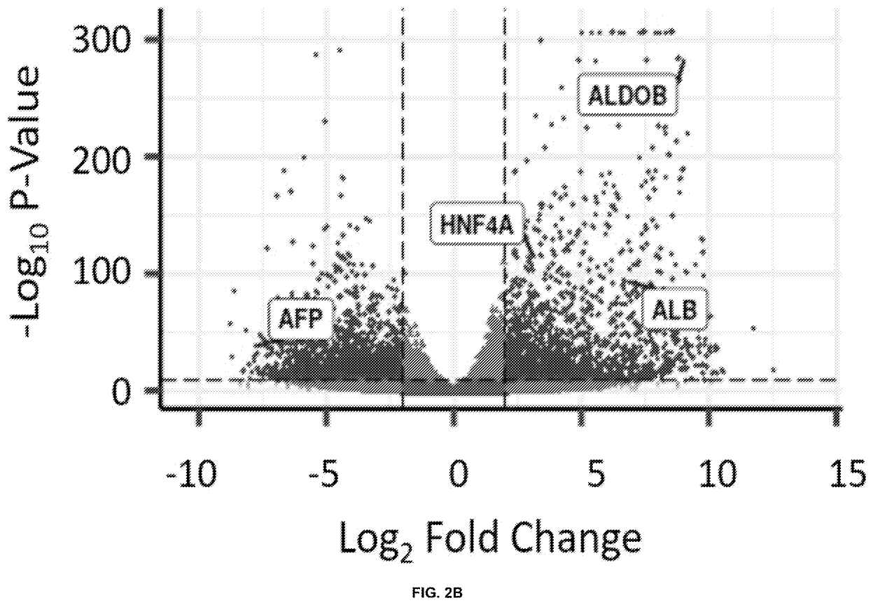

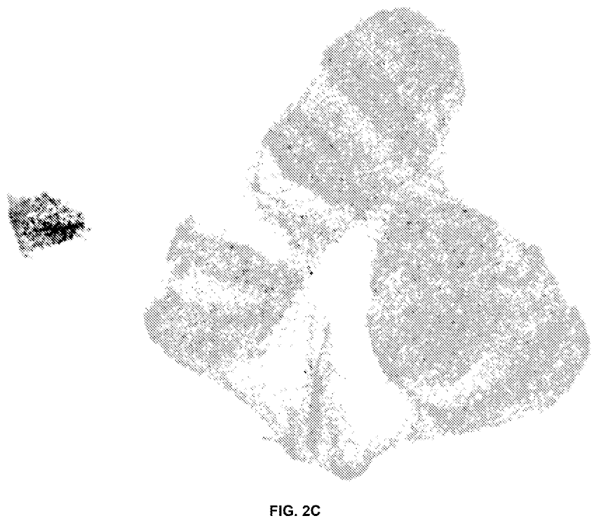

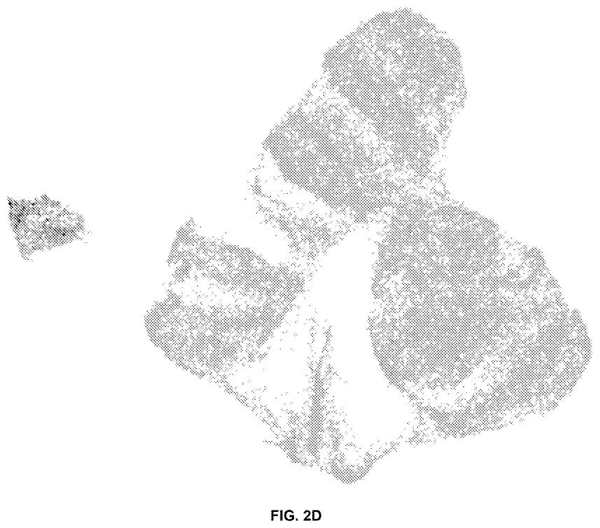

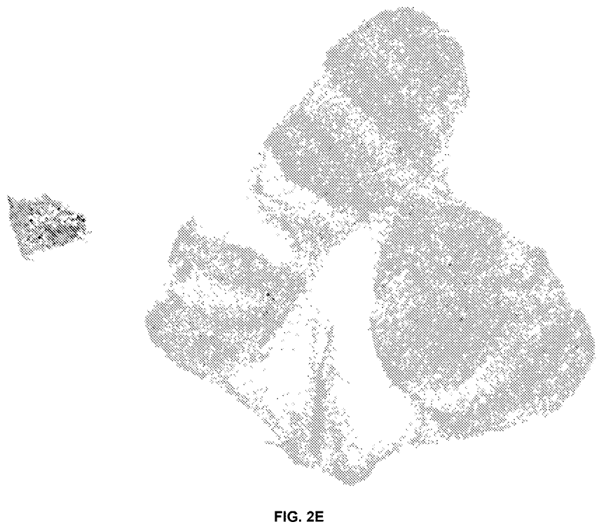

FIG. 2A shows the qPCR results comparing iPSCs, iHeps, and PHHs relative to IPSCs for various markers. Expression of the various markers differed between the cell types. Shown in FIG. 2B is a volcano plot showing DESeq2 results of PHH vs iHep RNAseq transcript fold changes that show that the genes are differentially expressed between the different cell types. Shown in FIGS. 2C-2E are UMAP representations of iHeps assessed through multiomic RNA and ATACseq clustered by RNAseq with AFP, Hnf4a, and Foxa2 gene expression highlighted.

### Example 3

### Transcription Factor CRISPRa and ORF Albumin Screens Generated Overlapping and Distinct Hits

Shown in FIG. 3B is a volcano plot generated from DESeq2 results comparing the ORF distributions in the high and low bins from a FACS screen. 39 hits came out as being significantly overrepresented in the high bin while 71 hits came out as being significantly overrepresented in the low bin. Shown in FIG. 3C is a DESeq2 violin plot generated from the top and bottom 10% bin gRNA abundance of the CRISPRa albumin screen with gRNAs with adjusted p-values <0.05 and non-targets. Positive fold change represents overrepresentation in the high 10% albumin bin. Shown in FIG. 3D is a violin plot for the ORF albumin screen. Shown in FIG. 3E is a volcano plot from DESeq2 results in MGH2069 IPSCs comparing the ORF distributions in the high and low bins from the FACS screen. 27 hits came out as being significantly overrepresented in the high bin while 178 hits came out as being significantly overrepresented in the low bin. Shown in FIG. 3F is a Venn diagram comparing the significant TFs from the albumin screens.

### Example 4

### Screen Validation

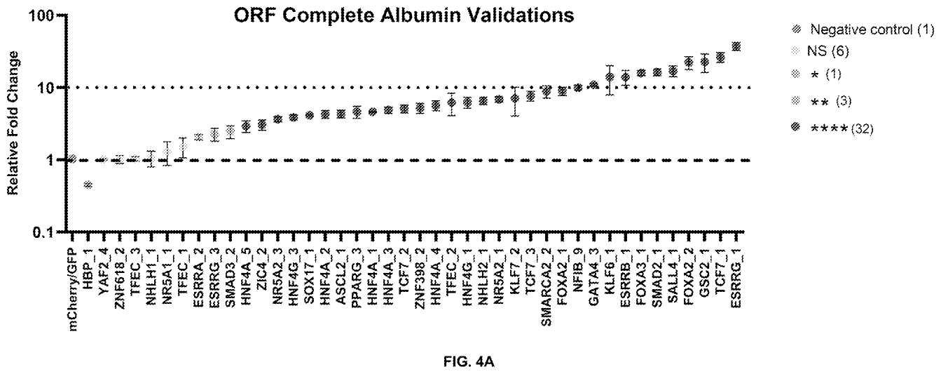

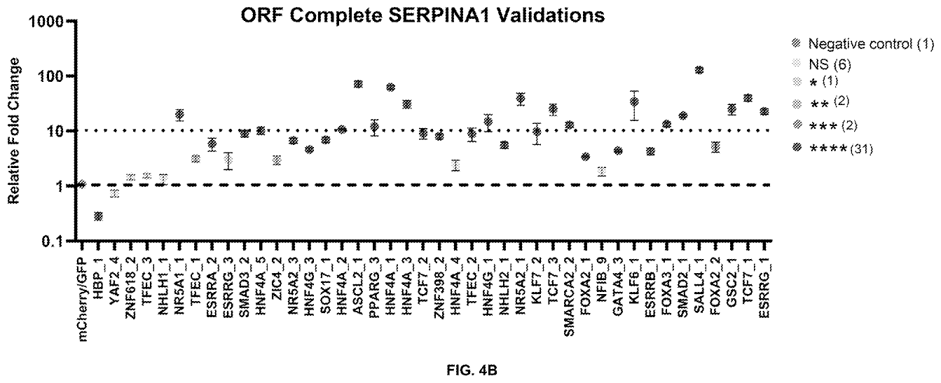

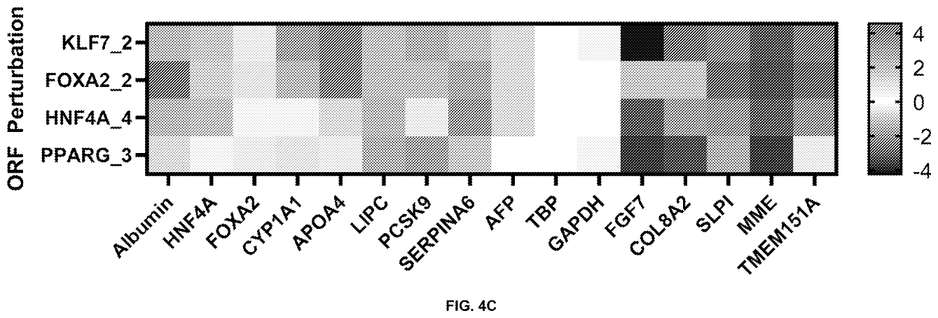

Hits that were overrepresented significantly in the high bin by DESeq2 were validated individually by qPCR for albumin and SERPINA1. Four hits were selected for further RNAseq validation. Albumin (FIG. 4A) and SERPINA1 (FIG. 4B) qPCR fold change results for high bin hits relative to mCherry/GFP negative controls are shown in FIGS. 4A-4B. Significance from ordinary one-way ANOVAs with multiple comparisons is indicated by the colors in the legend with asterisks corresponding to significance is shown in FIG. 4C. Select hits were analyzed by RNAseq relative to GFP with DESeq2. Fold change for genes of interest is indicated by color.

### Example 5

### Validations of CRISPRa Screen Demonstrates Albumin and AFP Upregulation

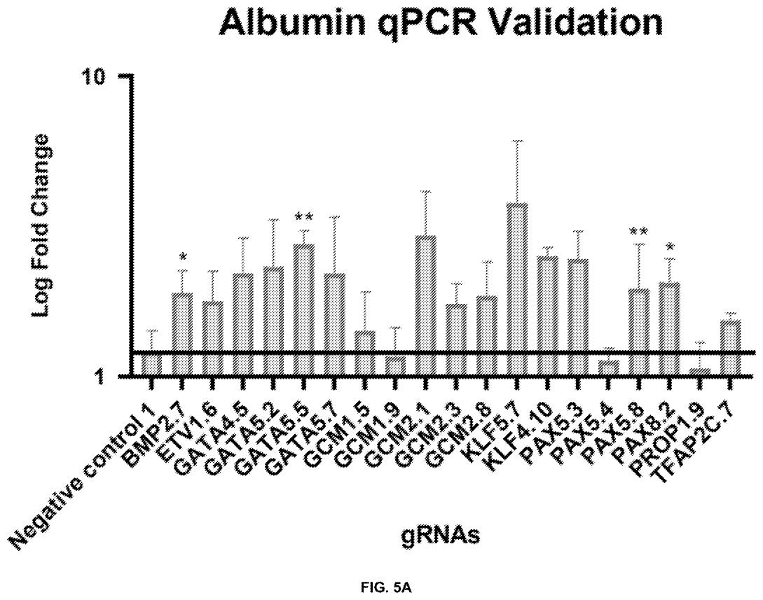

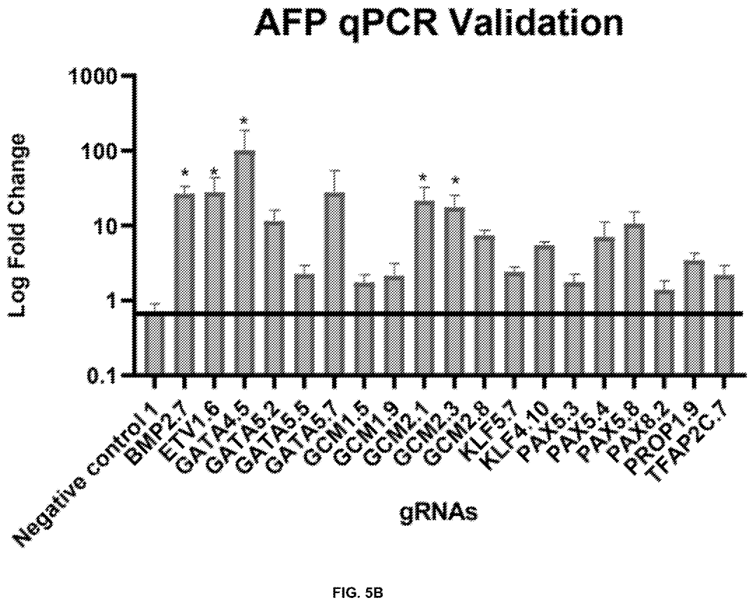

Shown in FIG. 5A are the albumin qPCR results for hit gRNAs with significance shown with asterisks compared to the negative control gRNA. Shown in FIG. 5B are AFP qPCR results with fold change shown relative to negative control.

### Example 6

### Functional Validations

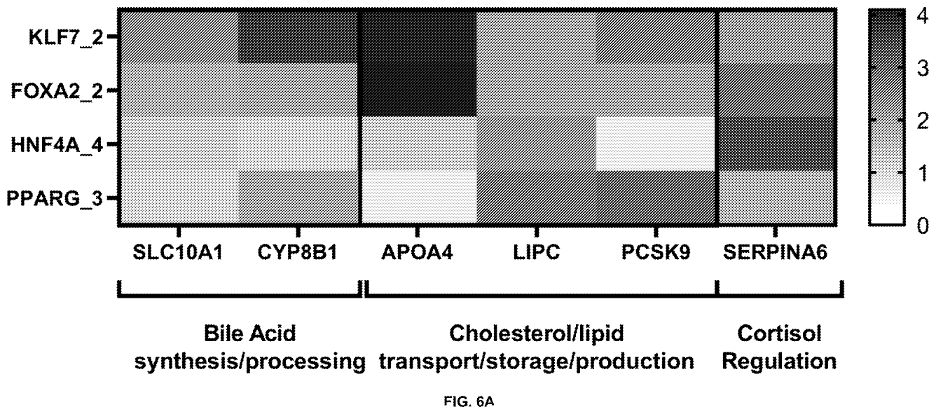

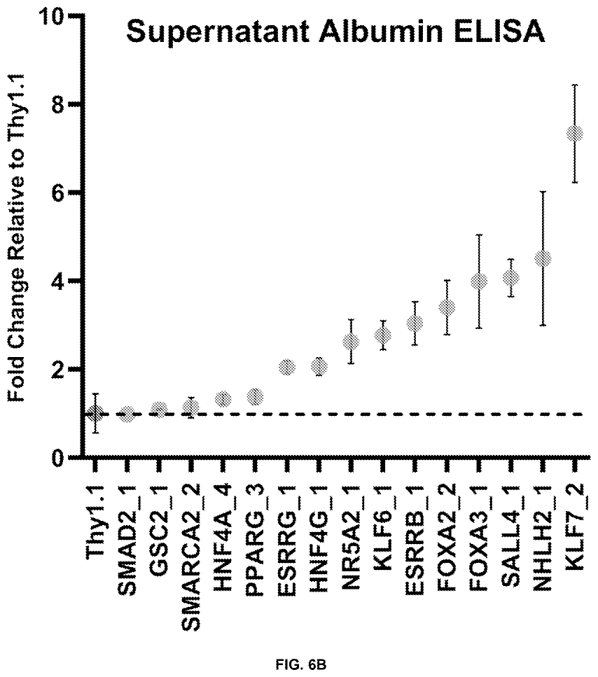

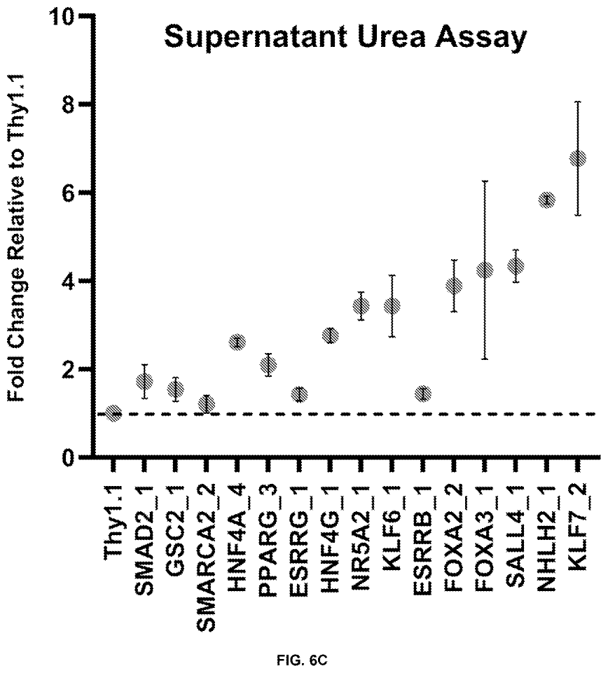

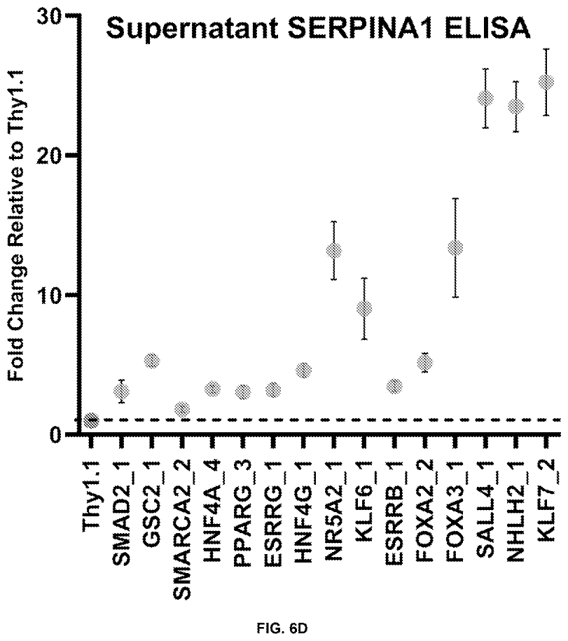

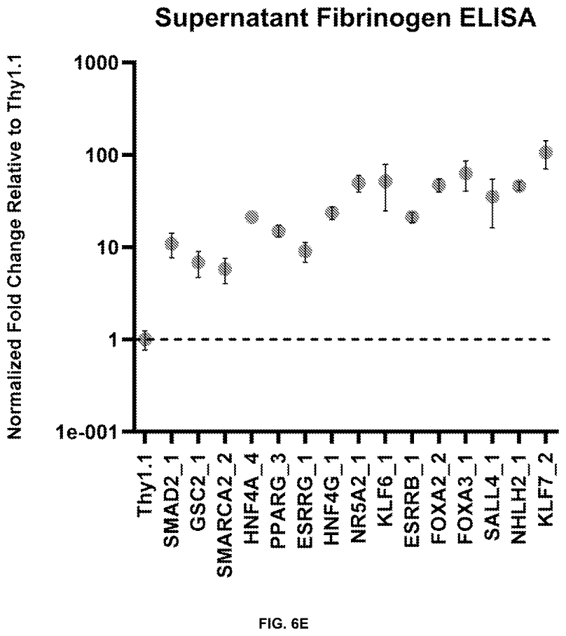

RNAseq results indicated that screen hits push hepatocytes towards mature function. To validate functional iHep maturity, select top hits were analyzed for albumin, SERPINA1, and urea supernatant secretion. Select hits were analyzed by RNAseq relative to GFP with DESeq2 (FIG. 6A). Fold change for functional genes of interest is indicated by color. Shown in FIG. 6B is a supernatant albumin ELISA normalized by cell number relative to Thy1.1. Shown in FIG. 6C is a supernatant urea assay normalized by cell number relative to Thy1.1. Shown in FIG. 6D is a graph of results from an ELISA using supernatant SERPINA1 normalized by cell number relative to Thy1.1. Shown in FIG. 6E is a graph of results from an ELISA using supernatant fibrinogen for hit ORFs normalized by cell number relative to Thy1.1.

### Example 7 iPSC and ESC Validations

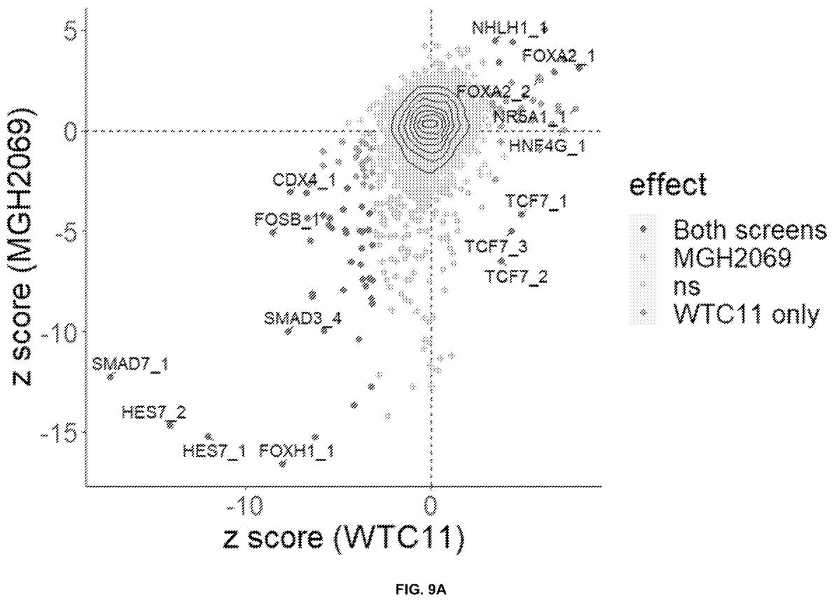

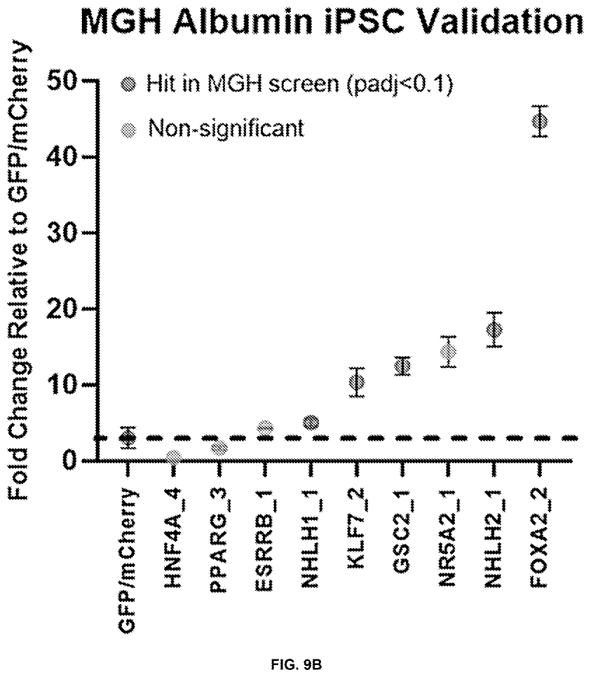

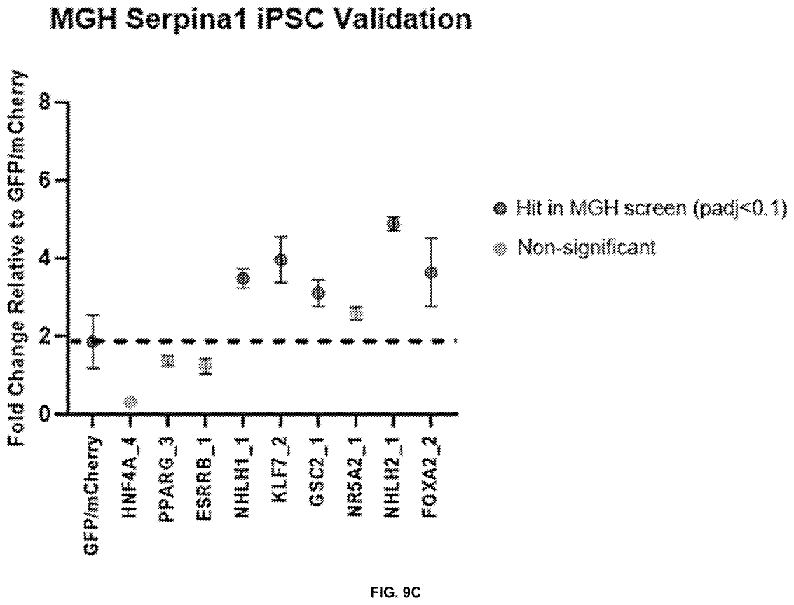

Shown in FIG. 9A is a correlation plot of a WTC11 vs MGH2069 IPSC screen with coloring indicating whether a given ORF was a hit in both, one, or neither screen (“ns”). Shown in FIG. 9B is a validation of hit ORFs in MGH2069 albumin IPSC line with fold change relative to mCherry/GFP negative control. Shown in FIG. 9C is a validation of hit ORFs in MGH2069 Serpina1 IPSC line with fold change relative to mCherry/GFP negative control.

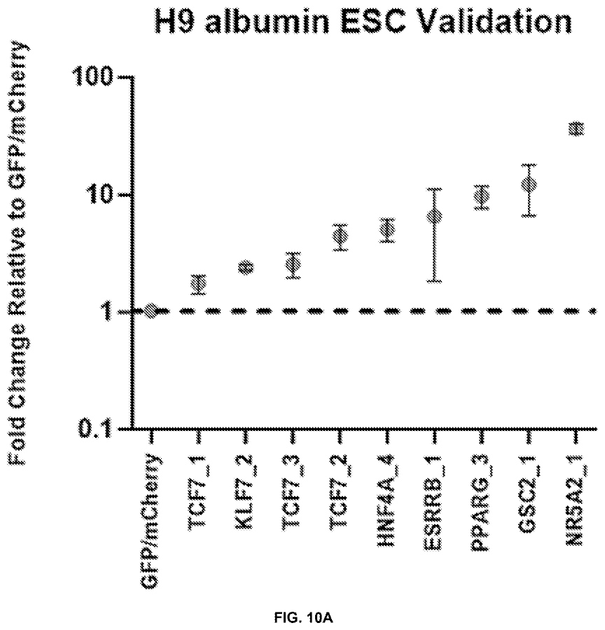

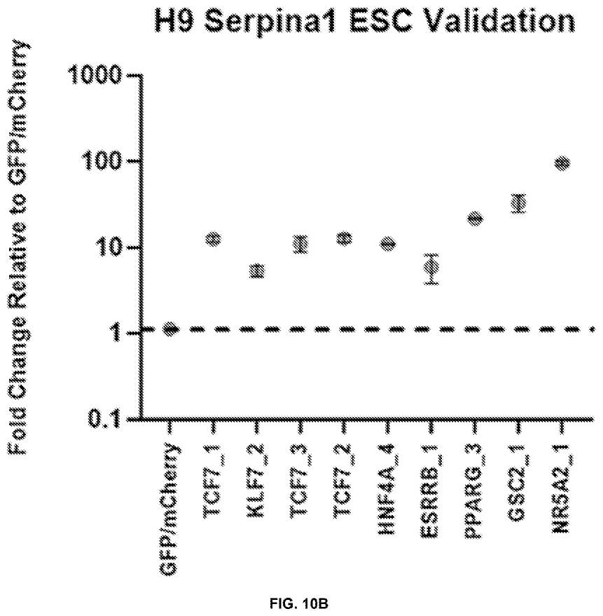

Shown in FIG. 10A is a validation of hit ORFs in H9 albumin ESC line with fold change relative to mCherry/GFP negative control. Shown in FIG. 10B is a validation of hit ORFs in H9 Serpina1 ESC line with fold change relative to mCherry/GFP negative control.

### Example 8

### Hit Orfs Display a Polygonal Homogenous Albumin Stain Indicative of a Hepatocyte

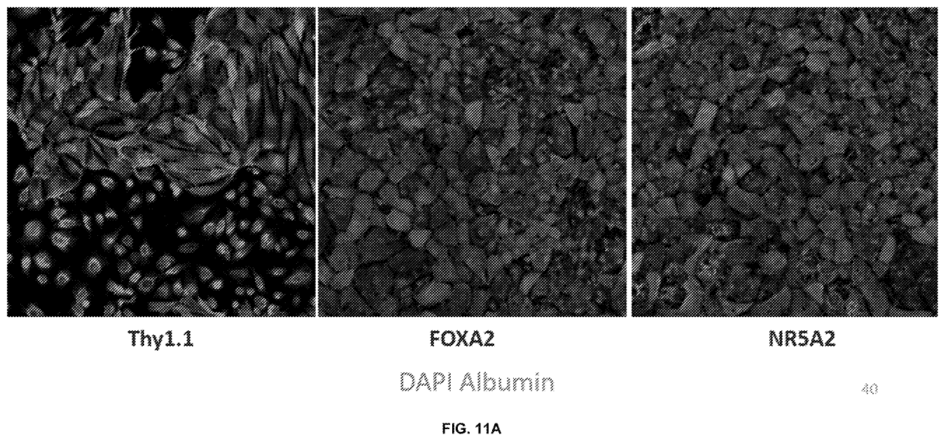

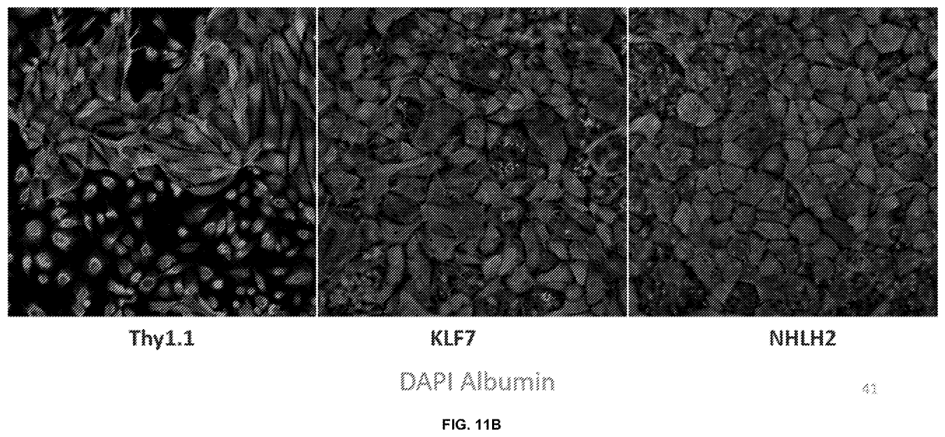

Shown in FIG. 11A is 20× immunofluorescence images of hit ORFs FOXA2 and NR5A2 as well as negative control Thy1.1 stained for DAPI in blue and albumin in red. Shown in FIG. 11B is 20× immunofluorescence images of hit ORFs KLF7 and NHLH2 as well as negative control Thy1.1 stained for DAPI in blue and albumin in red.

### Example 9

### Additional Studies

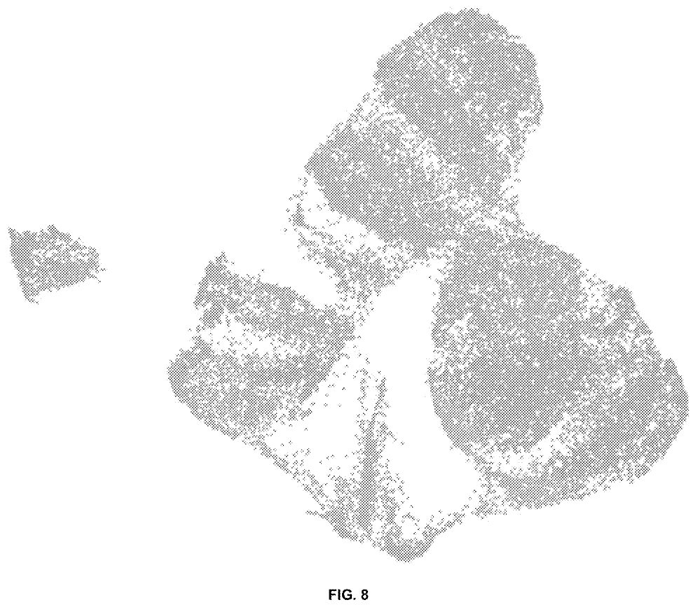

As detailed herein, we have characterized iHeps as immature and heterogeneous, screened TFs through CRISPRa and cDNA overexpression to find gRNAs and ORFs that significantly lead to upregulation of albumin, and validated hit gRNAs as upregulating AFP and albumin. The results detailed herein generated a protocol in which iHeps are more mature and less heterogeneous, improved toxicology studies using iHeps to lead to more accurate preclinical trials, and moved the field closer to having IHeps available as an alternative liver transplantation source. See FIG. 8.

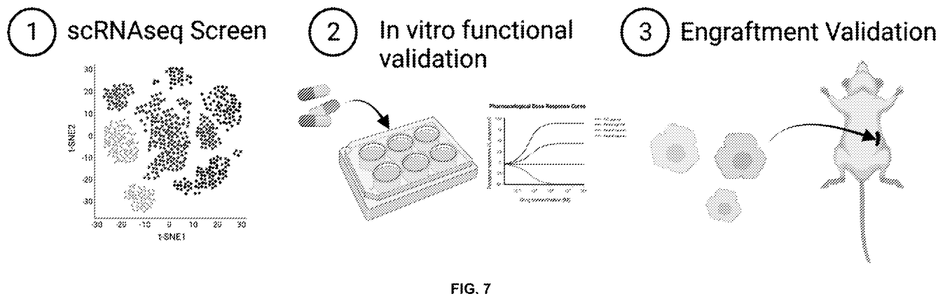

A broader range of mature markers may be examined by studying hit gRNAs and ORFs through a scRNAseq screen. The hits from the screen may be further functionally validated by examining, for example, cytochrome p450 induction, LDL uptake, and mouse humanization engraftment. See FIG. 7.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A composition comprising a modulator of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof.

Clause 2. The composition of clause 1, wherein the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof.

Clause 3. The composition of clause 1, wherein the gene is selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, NR5A1, HBP1, NHLH1, or YAF2, or a combination thereof, and the composition further comprises a modulator of a gene selected from FOXA2, HNF4A, NR5A2, FOXA3, or HNF4G, or a combination thereof, or wherein the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof, and the composition further comprises a modulator of a gene selected from FOXA2, HNF4A, NR5A2, FOXA3, or HNF4G, or a combination thereof.

Clause 4. The composition of any one of clauses 1-3, wherein the modulator induces differentiation of an induced pluripotent stem cell (IPSC) or an embryonic stem cell (ESC) into a hepatocyte, or promotes maturation of an IPSC-derived hepatocyte, or promotes maturation of an ESC-derived hepatocyte, or a combination thereof.

Clause 5. The composition of clause 4, wherein the composition modulates gene expression within the IPSC or the ESC or the hepatocyte.

Clause 6. The composition of any one of clauses 1-5, wherein the composition increases expression of albumin, alpha-1 antitrypsin, ASGR1, SERPINA1, Foxa2, a p450 enzyme, or Hnf4a, or a combination thereof, in the IPSC or in the ESC or in the iPSC-derived hepatocyte or in the ESC-derived hepatocyte.

Clause 7. The composition of any one of clauses 1-6, wherein the modulator is an activator.

Clause 8. The composition of clause 7, wherein the activator comprises a polypeptide, or a polynucleotide, or a combination thereof.

Clause 9. The composition of clause 8, wherein the activator comprises a polypeptide sequence selected from SEQ ID NOs: 121-158 or a fragment thereof, or polynucleotide sequence selected from SEQ ID NOs: 83-120 or a fragment thereof.

Clause 10. The composition of any one of clauses 1-9, wherein the modulator comprises a DNA targeting composition, the DNA targeting composition comprising: (a) a Cas9 protein and at least one guide RNA (gRNA) that targets the Cas9 protein to the gene or a regulatory element thereof; or (b) a fusion protein, wherein the fusion protein comprises two heterologous polypeptide domains, wherein the first polypeptide domain comprises a zinc finger protein or a TALE or a Cas protein selected from a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity, wherein when the first polypeptide domain comprises a Cas9 protein the DNA targeting composition further comprises at least one guide RNA (gRNA) that targets the Cas9 protein to the gene or a regulatory element thereof.

Clause 11. A DNA targeting composition comprising: a Cas9 protein or a fusion protein, wherein the fusion protein comprises two heterologous polypeptide domains, wherein the first polypeptide domain comprises a zinc finger protein or a TALE or a Cas protein selected from a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity thereof; and at least one guide RNA (gRNA) that targets the Cas protein to a target gene or a regulatory element thereof when the DNA targeting composition comprises a Cas protein, wherein the gene is selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof.

Clause 12. The composition of clause 11, wherein the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4.

Clause 13. The composition of clause 11, wherein the composition comprises a first gRNA targeting a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, NR5A1, HBP1, NHLH1, or YAF2 or a combination thereof, and a second gRNA targeting a gene selected from FOXA2, HNF4A, NR5A2, FOXA3, or HNF4G, or a combination thereof, or wherein the composition comprises a first gRNA targeting a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof, and a second gRNA targeting a gene selected from FOXA2, HNF4A, NR5A2, FOXA3, or HNF4G, or a combination thereof.

Clause 14. The composition of any one of clauses 11-13, wherein the gRNA is encoded by a polynucleotide comprising a sequence selected from SEQ ID NOs: 159-438, or comprises a sequence selected from SEQ ID NOs: 439-718.

Clause 15. The composition of any one of clauses 11-14, wherein the Cas protein comprises a Streptococcus pyogenes Cas9 protein, or a Staphylococcus aureus Cas9 protein, or any fragment thereof.

Clause 16. The composition of clause 15, wherein the Cas9 protein comprises the amino acid sequence of one of SEQ ID NOs: 26-29, or any fragment thereof, and/or wherein the Cas9 protein is encoded by a polynucleotide comprising a sequence selected from SEQ ID NOs: 30-39, and/or wherein the Cas9 protein comprises an amino acid sequence having at least 90% or greater identity to a sequence selected from SEQ ID NOs: 26-29, or any fragment thereof, and/or wherein the Cas9 protein is encoded by a polynucleotide comprising a sequence having at least 90% or greater identity to a sequence selected from SEQ ID NOs: 30-39, or any fragment thereof, and/or wherein the Cas9 protein comprises an amino acid sequence having one, two, three, four, five or more changes selected from amino acid substitutions, insertions, or deletions, relative to a sequence selected from SEQ ID NOs: 26-29, or any fragment thereof, and/or wherein the Cas9 protein is encoded by a polynucleotide comprising a sequence having one, two, three, four, five or more changes selected from nucleotide substitutions, insertions, or deletions, relative to a sequence selected from SEQ ID NOs: 30-39, or any fragment thereof.

Clause 17. The composition of any one of clauses 11-16, wherein the fusion protein comprises more than one second polypeptide domain.

Clause 18. The composition of any one of clauses 11-17, wherein the second polypeptide domain comprises a VP16 protein, or VP64, or p65 domain of NF kappa B transcription activator activity, or Tet1, or VPH, or VPR, or Rta, or a p300 protein, or a fragment thereof.

Clause 19. The composition of any one of clauses 11-18, wherein the second polypeptide domain comprises the amino acid sequence of SEQ ID NO: 41, 42, 53, or 55, or any fragment thereof, and/or wherein the second polypeptide domain is encoded by a polynucleotide comprising the sequence of SEQ ID NO: 54 or 56, and/or wherein the second polypeptide domain comprises an amino acid sequence having at least 90% or greater identity to SEQ ID NO: 41, 42, 53, or 55, or any fragment thereof, and/or wherein the second polypeptide domain is encoded by a polynucleotide comprising a sequence having at least 90% or greater identity to SEQ ID NO: 54 or 56, or any fragment thereof, and/or wherein the second polypeptide domain comprises an amino acid sequence having one, two, three, four, five or more changes selected from amino acid substitutions, insertions, or deletions, relative to SEQ ID NO: 41, 42, 53, or 55, or any fragment thereof, and/or wherein the second polypeptide domain is encoded by a polynucleotide comprising a sequence having one, two, three, four, five or more changes selected from nucleotide substitutions, insertions, or deletions, relative to SEQ ID NO: 54 or 56, or any fragment thereof.

Clause 20. The composition of any one of clauses 11-19, wherein the fusion protein comprises the amino acid sequence of SEQ ID NO: 43, or any fragment thereof, and/or wherein the fusion protein is encoded by a polynucleotide comprising the sequence of SEQ ID NO: 44, and/or wherein the fusion protein comprises an amino acid sequence having at least 90% or greater identity to SEQ ID NO: 43, or any fragment thereof, and/or wherein the fusion protein is encoded by a polynucleotide comprising a sequence having at least 90% or greater identity to SEQ ID NO: 44, or any fragment thereof, and/or wherein the fusion protein comprises an amino acid sequence having one, two, three, four, five or more changes selected from amino acid substitutions, insertions, or deletions, relative to SEQ ID NO: 43, or any fragment thereof, and/or wherein the fusion protein is encoded by a polynucleotide comprising a sequence having one, two, three, four, five or more changes selected from nucleotide substitutions, insertions, or deletions, relative to SEQ ID NO: 44.

Clause 21. An isolated polynucleotide sequence encoding the composition of any one of clauses 1-20.

Clause 22. A vector comprising the isolated polynucleotide sequence of clause 21.

Clause 23. An isolated cell comprising the composition of any one of clauses 1-20, or the isolated polynucleotide of clause 21, or the vector of clause 22, or a combination thereof.

Clause 24. A pharmaceutical composition comprising the composition of any one of clauses 1-21, or the isolated polynucleotide of clause 21, or the vector of clause 22, or the isolated cell of clause 23, or a combination thereof.

Clause 25. A therapy for inducing differentiation of an induced pluripotent stem cell (IPSC) or an embryonic stem cell (ESC) into a hepatocyte and/or for promoting maturation of a hepatocyte, the therapy comprising the composition of any one of clauses 1-20, or the isolated polynucleotide of clause 21, or the vector of clause 22, or the isolated cell of clause 23, or the pharmaceutical composition of clause 24, or a combination thereof.

Clause 26. A method of inducing differentiation of an induced pluripotent stem cell (IPSC) or an embryonic stem cell (ESC) into a hepatocyte, the method comprising administering to a cell or a subject the composition of any one of clauses 1-20, or the isolated polynucleotide sequence of clause 21, or the vector of clause 22, or the pharmaceutical composition of clause 24, or a combination thereof.

Clause 27. The method of clause 26, wherein the cell is an induced pluripotent stem cell (IPSC).

Clause 28. The method of clause 26, wherein the cell is an embryonic stem cell (ESC).

Clause 29. A method of promoting maturation of a hepatocyte, the method comprising administering to a cell or a subject the composition of any one of clauses 1-20, or the isolated polynucleotide sequence of clause 21, or the vector of clause 22, or the pharmaceutical composition of clause 24, or a combination thereof.

Clause 30. The method of any one of clauses 26-29, wherein the composition or isolated polynucleotide sequence or vector or pharmaceutical composition is administered to an IPSC or an ESC or a hepatocyte, and wherein the expression of albumin, alpha-1 antitrypsin, ASGR1, SERPINA1, Foxa2, a p450 enzyme, or Hnf4a, or a combination thereof, is thereby increased in the IPSC or the ESC or the IPSC-derived hepatocyte or the ESC-derived hepatocyte.

1. A composition comprising a modulator of a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof.

2. The composition of claim 1, wherein the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof.

3. The composition of claim 1, wherein the gene is selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, NR5A1, HBP1, NHLH1, or YAF2, or a combination thereof, and the composition further comprises a modulator of a gene selected from FOXA2, HNF4A, NR5A2, FOXA3, or HNF4G, or a combination thereof,
or wherein the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof, and the composition further comprises a modulator of a gene selected from FOXA2, HNF4A, NR5A2, FOXA3, or HNF4G, or a combination thereof.

4. The composition of any one of claims 1-3, wherein the modulator induces differentiation of an induced pluripotent stem cell (IPSC) or an embryonic stem cell (ESC) into a hepatocyte, or promotes maturation of an iPSC-derived hepatocyte, or promotes maturation of an ESC-derived hepatocyte, or a combination thereof.

5. The composition of claim 4, wherein the composition modulates gene expression within the IPSC or the ESC or the hepatocyte.

6. The composition of any one of claims 1-5, wherein the composition increases expression of albumin, alpha-1 antitrypsin, ASGR1, SERPINA1, Foxa2, a p450 enzyme, or Hnf4a, or a combination thereof, in the IPSC or in the ESC or in the IPSC-derived hepatocyte or in the ESC-derived hepatocyte.

7. The composition of any one of claims 1-6, wherein the modulator is an activator.

8. The composition of claim 7, wherein the activator comprises a polypeptide, or a polynucleotide, or a combination thereof.

9. The composition of claim 8, wherein the activator comprises a polypeptide sequence selected from SEQ ID NOs: 121-158 or a fragment thereof, or polynucleotide sequence selected from SEQ ID NOs: 83-120 or a fragment thereof.

10. The composition of any one of claims 1-9, wherein the modulator comprises a DNA targeting composition, the DNA targeting composition comprising:
(a) a Cas9 protein and at least one guide RNA (gRNA) that targets the Cas9 protein to the gene or a regulatory element thereof; or
(b) a fusion protein, wherein the fusion protein comprises two heterologous polypeptide domains, wherein the first polypeptide domain comprises a zinc finger protein or a TALE or a Cas protein selected from a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity, wherein when the first polypeptide domain comprises a Cas9 protein the DNA targeting composition further comprises at least one guide RNA (gRNA) that targets the Cas9 protein to the gene or a regulatory element thereof.

11. A DNA targeting composition comprising:
a Cas9 protein or a fusion protein, wherein the fusion protein comprises two heterologous polypeptide domains, wherein the first polypeptide domain comprises a zinc finger protein or a TALE or a Cas protein selected from a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity thereof; and
at least one guide RNA (gRNA) that targets the Cas protein to a target gene or a regulatory element thereof when the DNA targeting composition comprises a Cas protein,
wherein the gene is selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, FOXA2, NR5A1, FOXA3, HNF4A, HNF4G, HBP1, NHLH1, NR5A2, or YAF2, or a combination thereof.

12. The composition of claim 11, wherein the gene is NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4.

13. The composition of claim 11, wherein the composition comprises a first gRNA targeting a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, ZIC4, ZNF618, NR5A1, HBP1, NHLH1, or YAF2 or a combination thereof, and a second gRNA targeting a gene selected from FOXA2, HNF4A, NR5A2, FOXA3, or HNF4G, or a combination thereof,
or wherein the composition comprises a first gRNA targeting a gene selected from NHLH2, KLF7, GSC2, PPARG, SALL4, ESRRG, ESRRB, SMAD2, KLF6, TCF7, SMARCA2, SMAD3, TFEC, PURB, ZNF398, ASCL2, NFIB, or ZIC4, or a combination thereof, and a second gRNA targeting a gene selected from FOXA2, HNF4A, NR5A2, FOXA3, or HNF4G, or a combination thereof.

14. The composition of any one of claims 11-13, wherein the gRNA is encoded by a polynucleotide comprising a sequence selected from SEQ ID NOs: 159-438, or comprises a sequence selected from SEQ ID NOs: 439-718.

15. The composition of any one of claims 11-14, wherein the Cas protein comprises a Streptococcus pyogenes Cas9 protein, or a Staphylococcus aureus Cas9 protein, or any fragment thereof.

16. The composition of claim 15, wherein the Cas9 protein comprises the amino acid sequence of one of SEQ ID NOS: 26-29, or any fragment thereof,
and/or wherein the Cas9 protein is encoded by a polynucleotide comprising a sequence selected from SEQ ID NOs: 30-39,
and/or wherein the Cas9 protein comprises an amino acid sequence having at least 90% or greater identity to a sequence selected from SEQ ID NOs: 26-29, or any fragment thereof,
and/or wherein the Cas9 protein is encoded by a polynucleotide comprising a sequence having at least 90% or greater identity to a sequence selected from SEQ ID NOS: 30-39, or any fragment thereof,
and/or wherein the Cas9 protein comprises an amino acid sequence having one, two, three, four, five or more changes selected from amino acid substitutions, insertions, or deletions, relative to a sequence selected from SEQ ID NOs: 26-29, or any fragment thereof,
and/or wherein the Cas9 protein is encoded by a polynucleotide comprising a sequence having one, two, three, four, five or more changes selected from nucleotide substitutions, insertions, or deletions, relative to a sequence selected from SEQ ID NOs: 30-39, or any fragment thereof.

17. The composition of any one of claims 11-16, wherein the fusion protein comprises more than one second polypeptide domain.

18. The composition of any one of claims 11-17, wherein the second polypeptide domain comprises a VP16 protein, or VP64, or p65 domain of NF kappa B transcription activator activity, or Tet1, or VPH, or VPR, or Rta, or a p300 protein, or a fragment thereof.

19. The composition of any one of claims 11-18, wherein the second polypeptide domain comprises the amino acid sequence of SEQ ID NO: 41, 42, 53, or 55, or any fragment thereof,
and/or wherein the second polypeptide domain is encoded by a polynucleotide comprising the sequence of SEQ ID NO: 54 or 56,
and/or wherein the second polypeptide domain comprises an amino acid sequence having at least 90% or greater identity to SEQ ID NO: 41, 42, 53, or 55, or any fragment thereof,
and/or wherein the second polypeptide domain is encoded by a polynucleotide comprising a sequence having at least 90% or greater identity to SEQ ID NO: 54 or 56, or any fragment thereof,
and/or wherein the second polypeptide domain comprises an amino acid sequence having one, two, three, four, five or more changes selected from amino acid substitutions, insertions, or deletions, relative to SEQ ID NO: 41, 42, 53, or 55, or any fragment thereof,
and/or wherein the second polypeptide domain is encoded by a polynucleotide comprising a sequence having one, two, three, four, five or more changes selected from nucleotide substitutions, insertions, or deletions, relative to SEQ ID NO: 54 or 56, or any fragment thereof.

20. The composition of any one of claims 11-19, wherein the fusion protein comprises the amino acid sequence of SEQ ID NO: 43, or any fragment thereof,
and/or wherein the fusion protein is encoded by a polynucleotide comprising the sequence of SEQ ID NO: 44,
and/or wherein the fusion protein comprises an amino acid sequence having at least 90% or greater identity to SEQ ID NO: 43, or any fragment thereof,
and/or wherein the fusion protein is encoded by a polynucleotide comprising a sequence having at least 90% or greater identity to SEQ ID NO: 44, or any fragment thereof,
and/or wherein the fusion protein comprises an amino acid sequence having one, two, three, four, five or more changes selected from amino acid substitutions, insertions, or deletions, relative to SEQ ID NO: 43, or any fragment thereof,
and/or wherein the fusion protein is encoded by a polynucleotide comprising a sequence having one, two, three, four, five or more changes selected from nucleotide substitutions, insertions, or deletions, relative to SEQ ID NO: 44.

21. An isolated polynucleotide sequence encoding the composition of any one of claims 1-20.

22. A vector comprising the isolated polynucleotide sequence of claim 21.

23. An isolated cell comprising the composition of any one of claims 1-20, or the isolated polynucleotide of claim 21, or the vector of claim 22, or a combination thereof.

24. A pharmaceutical composition comprising the composition of any one of claims 1-21, or the isolated polynucleotide of claim 21, or the vector of claim 22, or the isolated cell of claim 23, or a combination thereof.

25. A therapy for inducing differentiation of an induced pluripotent stem cell (IPSC) or an embryonic stem cell (ESC) into a hepatocyte and/or for promoting maturation of a hepatocyte, the therapy comprising the composition of any one of claims 1-20, or the isolated polynucleotide of claim 21, or the vector of claim 22, or the isolated cell of claim 23, or the pharmaceutical composition of claim 24, or a combination thereof.

26. A method of inducing differentiation of an induced pluripotent stem cell (IPSC) or an embryonic stem cell (ESC) into a hepatocyte, the method comprising administering to a cell or a subject the composition of any one of claims 1-20, or the isolated polynucleotide sequence of claim 21, or the vector of claim 22, or the pharmaceutical composition of claim 24, or a combination thereof.

27. The method of claim 26, wherein the cell is an induced pluripotent stem cell (IPSC).

28. The method of claim 26, wherein the cell is an embryonic stem cell (ESC).

29. A method of promoting maturation of a hepatocyte, the method comprising administering to a cell or a subject the composition of any one of claims 1-20, or the isolated polynucleotide sequence of claim 21, or the vector of claim 22, or the pharmaceutical composition of claim 24, or a combination thereof.

30. The method of any one of claims 26-29, wherein the composition or isolated polynucleotide sequence or vector or pharmaceutical composition is administered to an iPSC or an ESC or a hepatocyte, and wherein the expression of albumin, alpha-1 antitrypsin, ASGR1, SERPINA1, Foxa2, a p450 enzyme, or Hnf4a, or a combination thereof, is thereby increased in the IPSC or the ESC or the IPSC-derived hepatocyte or the ESC-derived hepatocyte.
